SEQUENCE LISTING (2 sequences)

SEQ ID NO: 1 - DNA, 24 bp, synthetic construct
  source 1..24: mol_type = other DNA
cttggtattgaaactgtgggaggt

SEQ ID NO: 2 - DNA, 24 bp, synthetic construct
  source 1..24: mol_type = other DNA
agatctgagacttcttggtaggca

(19) United States
(12) Patent Application Publication
(10) Pub. No.: US 2024/0209345 A1
(43) Pub. Date: Jun. 27, 2024

(54) COMPOSITIONS AND METHODS FOR HIGH STRINGENCY ISOLATION OF NUCLEIC ACIDS

(71) Applicant: Duke University, Durham, NC (US)

(72) Inventors: Christopher Nicchitta, Durham, NC (US); Johncarlo Kristoflch, Durham, NC (US)

(21) Appl. No.: 18/510,007

(22) Filed: Nov. 15, 2023

Related U.S. Application Data
(60) Provisional application No. 63/425,850, filed on Nov. 16, 2022.

(51) Int. Cl. C12N 15/10; G01N 33/68

(52) U.S. Cl. CPC C12N 15/1003; G01N 33/6848

(57) ABSTRACT

The present disclosure provides compositions and methods related to the isolation of nucleic acids from a sample. In particular, the disclosure provides compositions comprising an alcohol and a monovalent salt and methods of us thereof for isolation of nucleic acids, including RNA-protein complexes (RNPs), from a biological sample.

### PRIORITY STATEMENT

This application claims priority to U.S. Provisional Patent Application No. 63/425,850, filed Nov. 16, 2022, the entire contents of which are incorporated herein by reference.

### GOVERNMENT FUNDING

This invention was made with government support under Federal Grant No. GM139480 awarded by the National Institutes of Health. The government has certain rights in the invention.

### SEQUENCE LISTING

The text of the computer readable sequence listing filed herewith, titled “DUKE-42465-202_SQL”, created Feb. 19, 2024, having a file size of 2,859 bytes, is hereby incorporated by reference in its entirety.

### FIELD

The present disclosure provides compositions and methods related to the isolation of nucleic acids from a sample. In particular, the disclosure provides compositions comprising an alcohol and a monovalent salt and methods of us thereof for isolation of nucleic acids, including RNA-protein complexes (RNPs), from a biological sample.

### BACKGROUND

Recent efforts towards the comprehensive identification of RNA-bound proteomes have revealed a large, surprisingly diverse family of candidate RNA-binding proteins (RBPs). Quantitative metrics for characterization and validation of protein-RNA interactions and their dynamic interactions have, however, proven analytically challenging and prone to error. Accordingly, what is needed are methods for accurate isolation and identification of RBPs in a sample.

### SUMMARY

Embodiments of the present disclosure include a method for isolating nucleic acids from a sample comprising contacting the sample with a precipitation buffer comprising a monovalent salt and an alcohol, thereby isolating the nucleic acids. In some embodiments, the monovalent salt is sodium chloride (NaCl) or lithium chloride (LiCl) and the alcohol is isopropanol or ethanol. In some embodiments, the precipitation buffer comprises from about 0.5M to about 10 M LiCl or NaCl, and about 20%-80% isopropanol or ethanol. In some embodiments, the precipitation buffer comprises from about 0.5M to about 7.5M LiCl or NaCl, and about 25% to about 75% isopropanol or ethanol. In some embodiments, the precipitation buffer comprises from about 0.5M to about 2M NaCl and about 25% to about 75% isopropanol or ethanol. In some embodiments, the precipitation buffer comprises from about 1M to about 7.5M LiCl and about 25% to about 75% isopropanol or ethanol.

In some embodiments, the monovalent salt is LiCl. In some embodiments, the precipitation buffer comprises from about 1M-5M LiCl and about 25%-75% isopropanol or ethanol. In some embodiments, the precipitation buffer comprises from about 3M-4.5M LiCl and about 40%-60% isopropanol or ethanol. In some embodiments, the precipitation buffer comprises from about 3.5M-4M LiCl and about 45%-55% isopropanol or ethanol. In some embodiments, the precipitation buffer comprises from about 3.7M-3.8M LiCl and about 47.5%-52.5% isopropanol or ethanol. In some embodiments, the precipitation buffer comprises about 3.75M LiCl and about 50% isopropanol or ethanol. In some embodiments, the precipitation buffer comprises LiCl and isopropanol.

In some embodiments, contacting the sample with the precipitation buffer comprises mixing the sample with the precipitation buffer and incubating the sample with the precipitation buffer for at least about 10 seconds. In some embodiments, contacting the sample with the precipitation buffer comprises incubating the sample with the precipitation buffer for about 10 seconds to about 10 minutes. In some embodiments, contacting the sample with the precipitation buffer comprises incubating the sample with the precipitation buffer for about 20 seconds to about 5 minutes. In some embodiments, contacting the sample with the precipitation buffer comprises incubating the sample with the precipitation buffer for about 30 seconds to about 2 minutes. In some embodiments, contacting the sample with the precipitation buffer comprises incubating the sample with the precipitation buffer for 40 seconds to 80 seconds. In some embodiments, contacting the sample with the precipitation buffer comprises incubating the sample with the precipitation buffer for about 50 seconds to about 70 seconds. In some embodiments, contacting the sample with the precipitation buffer comprises incubating the sample with the precipitation buffer for about 60 seconds.

In some embodiments, the method comprises performing at least two rounds of mixing and incubating the sample with the precipitation buffer. In some embodiments, the method comprises performing at least four rounds of mixing and incubating the sample with the precipitation buffer. In some embodiments, the method further comprises centrifuging the sample after contacting the sample with the precipitation buffer to form a pellet comprising the nucleic acids. One or more wash steps may be performed on the pellet to remove contaminants/further purify the isolated nucleic acids. In some embodiments, the nucleic acids comprise RNA. In some embodiments, the acids comprise RNA-protein complexes (RNPs).

In some embodiments, the method further comprises performing at least one round of acidic guanidium thiocyanate-phenol-chloroform (AGPC) biphasic extraction on the sample prior to contacting the sample with the precipitation buffer.

In some embodiments, the sample comprises acidic guanidium thiocyanate and phenol (AGP). In some embodiments, the sample comprises AGP and a solvent (e.g. chloroform).

Additional embodiments of the present disclosure include methods of isolating RNA-protein complexes (RNPs) from a sample. In some embodiments, methods of isolating RNPs from a sample comprise performing at least one round of acidic guanidium thiocyanate-phenol-chloroform (AGPC) biphasic extraction on the sample and isolating an interphase portion after each round of extraction, thereby obtaining a sample fraction enriched in RNA-protein complexes (RNPs); and contacting the sample fraction enriched in RNPs with a precipitation buffer comprising a monovalent salt and an alcohol, thereby isolating the RNPs.

In some embodiments, the monovalent salt is sodium chloride (NaCl) or lithium chloride (LiCl) and the alcohol is isopropanol or ethanol. In some embodiments, the precipitation buffer comprises from about 0.5M to about 10 M LiCl or NaCl, and about 20%-80% isopropanol or ethanol. In some embodiments, the precipitation buffer comprises from about 0.5M to about 7.5M LiCl or NaCl, and about 25% to about 75% isopropanol or ethanol. In some embodiments, the precipitation buffer comprises from about 0.5M to about 2M NaCl and about 25% to about 75% isopropanol or ethanol. In some embodiments, the precipitation buffer comprises from about 1M to about 7.5M LiCl and about 25% to about 75% isopropanol or ethanol.

In some embodiments, the monovalent salt is LiCl. In some embodiments, the precipitation buffer comprises from about 1M-5M LiCl and about 25%-75% isopropanol or ethanol. In some embodiments, the precipitation buffer comprises from about 3M-4.5M LiCl and about 40%-60% isopropanol or ethanol. In some embodiments, the precipitation buffer comprises from about 3.5M-4M LiCl and about 45%-55% isopropanol or ethanol. In some embodiments, the precipitation buffer comprises from about 3.7M-3.8M LiCl and about 47.5%-52.5% isopropanol or ethanol. In some embodiments, the precipitation buffer comprises about 3.75M LiCl and about 50% isopropanol or ethanol. In some embodiments, the precipitation buffer comprises LiCl and isopropanol.

In some embodiments, contacting the sample fraction enriched in RNPs with the precipitation buffer comprises mixing the sample fraction enriched in RNPs with the precipitation buffer and incubating the sample with the precipitation buffer for at least about 10 seconds. In some embodiments, contacting the sample fraction enriched in RNPs with the precipitation buffer comprises incubating the sample fraction enriched in RNPs with the precipitation buffer for about 10 seconds to about 10 minutes. In some embodiments, contacting the sample fraction enriched in RNPs with the precipitation buffer comprises incubating the sample fraction enriched in RNPs with the precipitation buffer for about 20 seconds to about 5 minutes. In some embodiments, contacting the sample fraction enriched in RNPs with the precipitation buffer comprises incubating the sample fraction enriched in RNPs with the precipitation buffer for about 30 seconds to about 2 minutes. In some embodiments, contacting the sample fraction enriched in RNPs with the precipitation buffer comprises incubating the sample fraction enriched in RNPs with the precipitation buffer for 40 seconds to 80 seconds. In some embodiments, contacting the sample fraction enriched in RNPs with the precipitation buffer comprises incubating the sample fraction enriched in RNPs with the precipitation buffer for about 50 seconds to about 70 seconds. In some embodiments, contacting the sample fraction enriched in RNPs with the precipitation buffer comprises incubating the sample fraction enriched in RNPs with the precipitation buffer for about 60 seconds.

In some embodiments, the method comprises performing at least two rounds of mixing and incubating the sample fraction enriched in RNPs with the precipitation buffer. In some embodiments, the method comprises performing at least four rounds of mixing and incubating the sample fraction enriched in RNPs with the precipitation buffer. In some embodiments, the method further comprises centrifuging the sample fraction enriched in RNPs after contacting the sample fraction enriched in RNPs with the precipitation buffer to form a pellet comprising the RNPs. One or more wash steps may be performed on the pellet to remove contaminants/further purify the isolated RNPs.

Additional embodiments of the present disclosure include methods of identifying RNA binding proteins in a sample. In some embodiments, methods of identifying RNA binding proteins in a sample comprise obtaining a composition comprising RNA-protein complexes (RNPs) isolated from a sample; depleting DNA from the composition, and identifying RNA binding proteins in the composition by mass spectrometry. In some embodiments, RNPs are isolated from the sample by performing at least one round of acidic guanidium thiocyanate-phenol-chloroform (AGPC) biphasic extraction on the sample and isolating an interphase portion after each round of extraction, thereby obtaining a sample fraction enriched in RNA-protein complexes (RNPs); and contacting the sample fraction enriched in RNPs with a precipitation buffer comprising a monovalent salt and an alcohol, thereby isolating the RNPs.

In some embodiments, mass spectrometry comprises LC-MS/MS.

In some embodiments, the monovalent salt is sodium chloride (NaCl) or lithium chloride (LiCl) and the alcohol is isopropanol or ethanol. In some embodiments, the precipitation buffer comprises from about 0.5M to about 10 M LiCl or NaCl, and about 20%-80% isopropanol or ethanol. In some embodiments, the precipitation buffer comprises from about 0.5M to about 7.5M LiCl or NaCl, and about 25% to about 75% isopropanol or ethanol. In some embodiments, the precipitation buffer comprises from about 0.5M to about 2M NaCl and about 25% to about 75% isopropanol or ethanol. In some embodiments, the precipitation buffer comprises from about 1M to about 7.5M LiCl and about 25% to about 75% isopropanol or ethanol.

In some embodiments, the monovalent salt is LiCl. In some embodiments, the precipitation buffer comprises from about 1M-5M LiCl and about 25%-75% isopropanol or ethanol. In some embodiments, the precipitation buffer comprises from about 3M-4.5M LiCl and about 40%-60% isopropanol or ethanol. In some embodiments, the precipitation buffer comprises from about 3.5M-4M LiCl and about 45%-55% isopropanol or ethanol. In some embodiments, the precipitation buffer comprises from about 3.7M-3.8M LiCl and about 47.5%-52.5% isopropanol or ethanol. In some embodiments, the precipitation buffer comprises about 3.75M LiCl and about 50% isopropanol or ethanol. In some embodiments, the precipitation buffer comprises LiCl and isopropanol.

In some embodiments, contacting the sample fraction enriched in RNPs with the precipitation buffer comprises mixing the sample fraction enriched in RNPs with the precipitation buffer and incubating the sample with the precipitation buffer for at least about 10 seconds. In some embodiments, contacting the sample fraction enriched in RNPs with the precipitation buffer comprises incubating the sample fraction enriched in RNPs with the precipitation buffer for about 10 seconds to about 10 minutes. In some embodiments, contacting the sample fraction enriched in RNPs with the precipitation buffer comprises incubating the sample fraction enriched in RNPs with the precipitation buffer for about 20 seconds to about 5 minutes. In some embodiments, contacting the sample fraction enriched in RNPs with the precipitation buffer comprises incubating the sample fraction enriched in RNPs with the precipitation buffer for about 30 seconds to about 2 minutes. In some embodiments, contacting the sample fraction enriched in RNPs with the precipitation buffer comprises incubating the sample fraction enriched in RNPs with the precipitation buffer for 40 seconds to 80 seconds. In some embodiments, contacting the sample fraction enriched in RNPs with the precipitation buffer comprises incubating the sample fraction enriched in RNPs with the precipitation buffer for about 50 seconds to about 70 seconds. In some embodiments, contacting the sample fraction enriched in RNPs with the precipitation buffer comprises incubating the sample fraction enriched in RNPs with the precipitation buffer for about 60 seconds.

In some embodiments, the method comprises performing at least two rounds of mixing and incubating the sample fraction enriched in RNPs with the precipitation buffer. In some embodiments, the method comprises performing at least four rounds of mixing and incubating the sample fraction enriched in RNPs with the precipitation buffer. In some embodiments, the method further comprises centrifuging the sample fraction enriched in RNPs after contacting the sample fraction enriched in RNPs with the precipitation buffer to form a pellet comprising the RNPs. One or more wash steps may be performed on the pellet to remove contaminants/further purify the isolated RNPs prior to identifying RBPs within the complexes.

### BRIEF DESCRIPTION OF THE DRAWINGS

The patent or application file contains at least one drawing executed in color. Copies of this patent or patent application publication with color drawing(S) will be provided by the Office upon request and payment of the necessary fee.

FIGS. 1A-1D: RNase-dependent SDS-PAGE mobility provides a key diagnostic for identifying RNA-binding proteins. AGPC: acidic guanidinium thiocyanate-phenol-chloroform; SRA: SRA analysis. FIG. 1A shows a schematic illustration of experimental approach. Repeated AGPC extraction enriches RBPs over non-RBPs; effect of repeated AGPC extraction on S/N was tested. Graphical representation of SRA: RNase-treated samples are compared to equivalent amounts of untreated samples by SDS-PAGE. RNA-bound proteins in untreated samples migrate at a higher molecular weight than their unbound counterparts. RNase-digestion liberates RNA-bound RBPs allowing their mobilization into the separating gel. FIG. 1B shows a comparison of AGPC interphase samples isolated by methanol precipitation (95% v/v) following 1-6 AGPC extraction(S) of UV-crosslinked or non-crosslinked HeLa cells by SRA and Coomassie Blue (protein), SYBR Safe (RNA&DNA) staining; parallel gels. Protein (BCA) and RNA&DNA (UV-spectrophotometry) yields shown in the adjoining bar charts represent the mean±1 SD of 3 biologically independent samples, pooled (equivalent % fraction) for SRA analysis. FIG. 1C shows Immunoblot analysis of samples from b. Sample compositions, target protein RBP RNA interactome category, and target protein GO:RBP annotation status indicated in FIG. panel. FIG. 1D shows a schematic illustration of enhanced S/N output. SRA is unable to distinguish non-RBPs from RBPs with low S/N in RNP fractions isolated by methanol precipitation (95% v/v) from the final AGPC interphase. SRA supports the identification of RNA enrichment methods that further enhance S/N, evidenced by a marked decrease in RNase-insensitive protein. Graphics (a, b) were created with BioRender.com. Experiments (b, c) were performed four times with similar results. Source data are provided as a Source Data file.

FIGS. 2A-C: LEAP-RBP provides rapid and efficient recovery of RNA-bound protein and identification of RBPs by SDS-PAGE RNase-sensitivity Assay (SRA). AGPC: acidic guanidinium thiocyanate-phenol-chloroform; M: methanol precipitation (95% v/v); I: INP; L: LEAP-RBP; SRA: SRA analysis. FIG. 2A shows a comparison of RNP fractions isolated by M, I, and L methods from final AGPC interphase suspensions of UV-crosslinked cells by SRA and Coomassie Blue (protein), SYBR Safe (RNA&DNA) staining. Protein (BCA) and RNA (UV-spectrophotometry) yields shown in the adjoining bar charts represent the mean±1 SD of 3 biologically independent samples, pooled (equivalent % fraction) for SRA analysis. Effect of isolation method on protein yield analyzed using one-way ANOVA (Fisher's PLSD post-hoc test, unpaired, two-tailed, homoscedastic, no correction): significant main effect of isolation method (p*), F(2, 6)=41.91, p<0.001 (MvI*, p=0.018; IvL*, p=0.001; MvL*, p<0.001). Effect of isolation method on RNA yield analyzed using a one-way ANOVA (Fisher's PLSD post-hoc test, unpaired, two-tailed, homoscedastic, no correction): non-significant main effect of isolation method (n.s.), F(2, 6)=0.04, p=0.965 (post-hoc testing not applicable). FIG. 2B shows immunoblot analysis of samples from a, sample compositions indicated in FIG. panel. FIG. 2C shows a pictorial representation of LEAP-RBP method: A) Addition of chloroform and vortexing. B) Addition of precipitation solution (LiCl and isopropanol), inversion and incubation for 1 minute; repeated 5+times. C) Vortexing D) Centrifugation and rinsing with 95% methanol (v/v). Experiments (a, b) were performed twice with similar results. Source data are provided as a Source Data file.

FIGS. 3A-3B: Repeated AGPC extraction and DNA depletion step improves S/N and sensitivity of SRA. AGPC: acidic guanidinium thiocyanate-phenol-chloroform; L: LEAP-RBP. Sample preps: s.p.1, L; s.p.2, L w/DNA depletion step; s.p.3, repeated AGPC extraction and L w/DNA depletion step. FIG. 3A shows SRA analysis of L fractions isolated from AGP input suspensions containing equivalent amounts of UV-crosslinked or non-crosslinked cells by s.p.1, s.p.2, s.p.3; parallel gels; Coomassie Blue (protein), SYBR Safe (RNA&DNA). Protein (BCA) and RNA&DNA (UV-spectrophotometry) yields shown in the adjoining bar charts represent the mean±1 SD of 3 biologically independent samples, pooled (equivalent % fraction) for SRA analysis. Effect of UV-crosslinking and sample prep on protein yield of L analyzed using two-way ANOVA: significant interaction, F(2, 12)=4.61, p=0.033. Subdivided by largest main effect (m.e.), UV-crosslinking (m.e.1*, dashed line), F(1, 12)=93.66, p<0.001. Effect of sample prep on protein yield of L for UV-crosslinked and non-crosslinked subgroups analyzed using independent one-way ANOVAs (Fisher's PLSD post-hoc test, unpaired, two-tailed, homoscedastic, no correction): non-crosslinked, significant m.e. of sample prep (m.e.2*), F(2, 6)=6.07, p=0.036 (e.1*, p=0.023; e.2*, p=0.023; n.s.1, non-significant, p=0.998); UV-crosslinked, significant m.e. of sample prep (m.e.3*), F(2, 6)=6.86, p=0.028 (e.3*, p=0.010; n.s.2, non-significant, p=0.076; n.s.3, non-significant, p=0.173). Effect of UV-crosslinking and sample prep on RNA&DNA yield of L analyzed using two-way ANOVA: significant interaction, F(2, 12)=719.45, p<0.001. Subdivided by largest m.e., sample prep (m.e.4*, dashed lines), F(1, 12)=998.29, p<0.001. Effect of UV-crosslinking on RNA&DNA yield of L for s.p.1, s.p.2, s.p.3 subgroups analyzed using independent one-way ANOVAs (Fisher's PLSD post-hoc test, unpaired, two-tailed, homoscedastic, no correction): s.p.1, non-significant m.e. of UV-crosslinking (n.s.4), F(1, 4)=0.14,p=0.729; s.p.2, non-significant m.e. of UV-crosslinking (n.s.5), F(1, 4)=0.91, p=0.393; s.p.3, significant m.e. of UV-crosslinking (m.e.5*), F(1, 4)=746.88,p<0.001. FIG. 3B shows Immunoblot analysis of samples from a, sample compositions indicated in FIG. panel. Experiments (a, b) were performed four times with similar results. Source data are provided as a Source Data file.

FIGS. 4A-4F: Enhanced S/N decreases UV-enrichment* specificity. AGPC: acidic guanidinium thiocyanate-phenol-chloroform; I: INP; L: LEAP-RBP; SILAC: SILAC LC-MS/MS analysis; SRA: SRA analysis; E*: significantly UV-enriched*; NE: not E*. FIG. 4A shows a schematic illustration of experimental approach. Heavy SILAC-labeled UV-crosslinked and light SILAC-labeled non-crosslinked cells were mixed prior to repeated AGPC extraction and isolation of RNP fractions by L or I methods. Graphic prepared in BioRender. FIG. 4B shows volcano plots showing proteins identified as E* (red) or NE (blue) in I or L fractions by SILAC. Log₂(CL/nCL) ratios were generated with SPI_{CL} values and average SPI_{nCL} values. Tested against the null hypothesis that protein log₂(CL/nCL) ratios (n=3 biologically independent samples) are equal to 0 using unpaired, upper-tailed, heteroscedastic t tests (RNA-binding proteins are expected to be recovered from UV-crosslinked cells in greater amounts). Correction for multiple hypothesis testing was performed using the Benjamini-Hochberg approach and a false-discovery rate of 5%. FIG. 4C shows GO-enrichment analysis of proteins identified as E* in I or L fractions by SILAC (Fisher's Exact, two-tailed, correction for multiple hypothesis testing performed using the Benjamini-Hochberg approach and a false-discovery rate of 5%). FIG. 4D shows a Venn diagram showing overlap of total and E* proteins identified in I and/or L fractions by SILAC. Pie charts showing the number of RBPs and non-RBPs identified as E* or NE in I or L fractions by SILAC. FIG. 4E shows stacked bar charts showing the number of proteins with annotated RNA-binding or RNA-related functions identified as E* or NE in I or L fractions by SILAC. FIG. 4F shows Histograms showing average log₂(CL/nCL) ratios of RBPs and non-RBPs identified in I or L fractions by SILAC. Font color for individual proteins reflects conclusions from SRA and immunoblot analysis of total clRNP fractions; red text: RNase-sensitive RBP; blue text: undetected or RNase-insensitive protein. Asterisks: E*. I and L SILAC LC-MS/MS experiments (a) were performed once with 3 biologically independent samples for each SILAC label group (CL, nCL). Source data are provided as Source Data file.

FIGS. 5A-5H: SILAC LC-MS/MS analysis of LEAP-RBP fractions demonstrates high RNA-bound protein enrichment. I: INP; L: LEAP-RBP; SILAC: SILAC LC-MS/MS analysis; SRA: SRA analysis; E*: significantly UV-enriched*; NE: not E*. FIG. 5A shows the predicted relationship between CL/nCL and S/N ratios for a specific RBP or non-RBP identified in RNP fractions isolated from pooled UV-crosslinked (red) and non-crosslinked (blue) samples by SILAC. FIG. 5B shows the relationship from a shown as a continuous function. FIG. 5C shows the predicted relationship between log₂(S/N) and the percentage (%) of observed protein quantity during SILAC LC-MS/MS experiments that is RNA-bound. FIG. 5D shows the predicted change in observed quantity Δ log₂(S+N) in response to a change in RNA-bound quantity Δ log₂(S) for proteins displaying increasing S/N ratios. FIG. 5E shows the estimated Δ log₂(S) to successfully reject the null hypothesis that Δ log₂(S+N)=0 increases with decreasing S/N and increasing SD. FIG. 5F shows Log₂(S/N) ratios quantified by SILAC were used to estimate the observed quantity of nucleolin and three non-canonical endoplasmic reticulum candidate RBPs in RNase-treated (|S|+N) and untreated (N) I and L fractions by SDS-PAGE and immunoblot. Immunoblots were selected from FIG. 2b; performed twice with similar results. FIG. 5G shows estimated amount of RNA-bound and free protein in I and L fractions by SILAC is more accurate when assuming equal noise-partitioning between SILAC channels. FIG. 5H shows Stacked bar charts showing the number of RBPs and non-RBPs identified as E* or NE by each method, or the estimated % TP_{S} and % TP_{N} contributions of RBPs and non-RBPs in each RNP fraction. Graphics prepared in BioRender (a, b, c). Source data are provided as a Source Data file.

FIGS. 6A-6H: High method specificity for RNA-bound RBPs allows accurate RCS ranking of RNA-binding proteins. I: INP; L: LEAP-RBP; SILAC: SILAC LC-MS/MS analysis; SRA: SRA analysis. Specificity of I (FIG. 6A) and L (FIG. 6B) methods for enrichment of RNA-bound RBPs was evaluated by comparing observed abundances of RBPs and non-RBPs as a function of their log₂(S/N) ratios (SILAC). Distributions of immunoblot targets reported in FIG. 5f demonstrates significance of vertical intercept. FIG. 6C shows L exclusives fall within the lower range of detection (% TP range). FIG. 6D shows panel b with labeled immunoblot targets; red font: RNase-sensitive RBP; blue text: undetected or RNase-insensitive protein. FIG. 6E shows cumulative frequency curves for RBPs (red) and non-RBPs (blue) identified in I (dashed) and L (solid) fractions as a function of log₂(S/N) or log₁₀(% TP) (SILAC). Effect of GO-annotation (GO:RBP) on analyzed using independent Kruskal-Wallis tests: significant effect of GO-annotation on log₂(S/N) ratios of proteins identified in I and L fractions (SILAC), H(1)=194.63, p<0.001, H(1)=436.62,p<0.001, respectively; significant effect of GO-annotation on log₁₀(% TP) of proteins identified in I and L fractions (SILAC), H(1)=111.11, p<0.001, H(1)=632.29, p<0.001, respectively. FIG. 6F shows Panel b with color overlay based on ordinal RCS rank. FIG. 6G shows RCS as a function of RCS rank. Protein IDs represent proteins examined by SRA and immunoblot in FIG. 2b; red font: RNase-sensitive RBP; blue font: undetected or RNase-insensitive proteins with positive RCS (i.e., log₂(S/N)>0) are considered more representative of their RNA-bound counterparts. FIG. 6H shows the top 10 RCS ranked enigmRBPs.

FIGS. 7A-7J: Profiling the relationship between mRNA CDS ribosome occupancy state and RBP-RNA interactome dynamics. SILAC: SILAC LC-MS/MS analysis; non-SILAC: LC-MS/MS analysis; SRA: SRA analysis; clRNP: in clRNP fraction; input: in input sample. FIG. 7A shows ribosome profiling of HeLa cells after 30-minute treatment with 2 μg/mL harringtonine (HT) or DMSO control, n=3 biologically independent samples. FIGS. 7B-7F and 7H: Red markers: proteins which display S/N ratios>3 (clRNP, SILAC) shown in FIG. 6b; blue markers: other; additional labels and color overlay (“label”: color marker) for proteins displaying a significant difference in RNA occupancy (HT/DMSO, clRNP, unpaired, two-tailed, homoscedastic t test; n=3 biologically independent samples) after FDR-correction (Benjamini-Hochberg approach, false-discovery rate of 5%) with S/N limit (“gold hits”: gold marker), without S/N limit (“teal hits”: teal marker), or both (“purple hits”: purple marker). FIG. 7B shows a volcano plot showing significance values of proteins before FDR-correction as a function of log₂(HT/DMSO), (clRNP, non-SILAC). FIG. 7C shows a scatterplot comparing log₂(HT/DMSO) ratios of proteins identified in both input and clRNP fractions (shared, non-SILAC). FIG. 7D shows observed protein abundances (clRNP, non-SILAC) as a function of log₂(S/N) (clRNP, SILAC). FIG. 7E shows GO-enrichment analysis of gold, teal, and purple hits; shown are the top 10 most enriched GO terms for purple hits (Fisher's Exact, two-tailed, no correction). FIG. 7F shows category-distributed comparison of hits showing % RNA-bound (clRNP, SILAC), observed fold-change (clRNP, non-SILAC), presence of RNA-binding domain (blue: no; red: yes); and RBP-annotation (GO:RBP) status (blue: no; red: yes; red with blue outline: yes, inferred from UV-enrichment* in RIC-like (non-SILAC) experiments. FIG. 7G shows SRA and immunoblot of pooled (equivalent μg protein) input and clRNP fractions quantified in FIG. 15d-g and analyzed by LC-MS/MS (non-SILAC); includes observed FC(HT/DMSO) and S/N ratios (clRNP, SILAC); asterisk: significant FC(HT/DMSO) after FDR-correction with S/N limit. FIG. 7H is a scatterplot comparing observed protein abundances in input and clRNP fractions (shared, non-SILAC). FIG. 7I shows a comparison of input and clRNP fractions isolated from four different cell lines by SRA and Coomassie Blue (protein), SYBR Safe (RNA&DNA) staining. FIG. 7J shows immunoblot analysis of samples from i, n=3 biologically independent samples, pooled (equivalent % fraction) for SRA. Experiments were performed once (a-h), or three times with similar results (i, j). Source data are provided as a Source Data file.

FIGS. 8A-8E: Benchmark comparisons illustrate utility of LEAP-RBP and S/N, % TP_{S} metrics. N/AGPC: neutral/acidic guanidinium thiocyanate-phenol-chloroform; AGP: acidic guanidinium thiocyanate-phenol; L: LEAP-RBP; X: XRNAX; O: OOPs; P: Ptex; T: TRAPP; R: RIC; SILAC: SILAC LC-MS/MS analysis; SRA: SRA analysis; E*: significantly UV-enriched*; NE: not E*. FIG. 8A shows experimental flow outlining the main steps of LEAP-RBP and five referenced RNA-centric methods. TBE gel analysis was performed on 1 μg of RNA isolated by NGPC extraction of proteinase K treated RNP fractions isolated from UV-crosslinked cells and an equivalent % fraction of their corresponding non-crosslinked samples. UV-dependence of protein and RNA recovery as well as S/N were evaluated by SRA with SYBR Safe (RNA&DNA), Coomassie Blue (protein), and silver stain (RNA, DNA, and protein) staining (FIG. 8B) or immunoblot (FIG. 8C). Sample compositions and/or normalization values indicated in panels b, c. Immunoblot targets found UV-enriched* in referenced studies were marked with gold asterisks in c; black asterisk (T): no yeast homologue, hence n/a; n.d.: not detected. FIG. 8D shows Specificity and selectivity of the different methods for RNA-bound RBPs was evaluated by comparing observed abundances of RBPs and non-RBPs as a function of their log₂(S/N) ratios (SILAC and non-SILAC). Differences in corresponding frequency curves plotted as a function of log₂(S/N) (solid) or log₁₀(% TP) (dashed) indicate differences in protein-RNA adduct enrichment efficiency (S/N) or abundance (% TP) respectively. FIG. 8E shows stacked bar charts showing the number of RBPs and non-RBPs identified as E* or NE by each method, or the estimated % TP_{S} and % TP_{N} contributions of RBPs and non-RBPs in each RNP fraction. Experiments were performed once (a, b, c); n=1 (X, O, P, T, R); n=3 biologically independent samples (L), pooled (equivalent % fraction) for SRA. Input samples isolated from AGP input suspensions containing UV-crosslinked and/or non-crosslinked HeLa cells were used as inter-run controls during SRA analysis, n=1. Source data are provided as a Source Data file.

FIGS. 9A-9N: LEAP-RBP provides a high stringency, comprehensive portrait of the RNA-bound proteome. L: LEAP-RBP: TRAPP; R: RIC: E: eRIC: SILAC: SILAC LC-MS/MS analysis; SRA: SRA analysis; E*: significantly UV-enriched*; NE: not E*. FIG. 9A, FIG. 9B show UV-dependent enrichment and S/N were evaluated by SRA with SYBR Safe (RNA&DNA), Coomassie Blue (protein), and Silver Stain (RNA, DNA, and protein) staining (a) or immunoblot (b). Sample compositions and/or normalization values included in FIG. panel b. FIG. 9C shows stacked bar charts showing the number of RBPs and non-RBPs identified as E* or NE by each method, or the estimated % TP_{S} and % TP_{N} contributions of RBPs and non-RBPs in each RNP fraction. FIG. 9D shows evaluation of method performance by RCS rank analysis. Proteins are ranked by their RBP-confidence scores and binned (n=100 per bin). The number (#) of GO-annotated RBPs, RBP-confidence scores (RCS), % TP contributions, log₂(S/N) ratios, # of unique peptides (i.e., coverage), and detectable Δ log₂(S) are compared. For methods with high performance and/or % TP_{S}, RCS rank predicts log₂(S/N), % TP, coverage, and detectable Δ log₂(S). FIG. 9E, FIG. 9F, and FIG. 9G show method specificity for RNA-bound RBPs was evaluated by comparing observed abundances of RBPs and non-RBPs as a function of their log₂(S/N) ratios (SILAC and non-SILAC). FIG. 9H shows a comparison of the 40 most abundant (% TP) proteins identified by each method with their estimated % TP_{S} and % TP_{N} contributions in each RNP fraction. FIG. 9I shows stacked bar charts showing the number of proteins for different classes of RNA-binders and their estimated % TP_{S} and % TP_{N} contributions in each RNP fraction. FIG. 9J shows a comparison of TRAPP experiments performed at differing UV-crosslinking energies as described in c. FIG. 9K shows a comparison of RIC (R) and eRIC (E) as described in c shows eRIC achieves higher % TP_{S} than RIC. FIG. 9L shows an evaluation of eRIC specificity as done in e-g. FIG. 9M shows a comparison of mRNA and rRNA binders identified by RIC (R) and eRIC (E) as described in i. FIG. 9N shows a comparison of exclusive DNA binders identified by TRAPP (T), RIC (R), eRIC (E), INP (I), and LEAP-RBP (L) as described in i. Experiments were performed once (a, b); n=1 (T, R); n=3 biologically independent samples (L), pooled (equivalent % fraction) for SRA. Source data are provided as a Source Data file.

FIGS. 10A-10B: LEAP-RBP allows determinations of RBP-specific UV-crosslinking efficiencies and S/N ratios. Estimation of RBP-specific UV-crosslinking efficiencies (FIG. 10A) and S/N ratios (FIG. 10B) by comparing serial dilutions of input and RNase-treated clRNP fractions, or serially diluted clRNP fractions to a corresponding untreated sample with SDS-PAGE and immunoblot respectively; n=3 biologically independent samples, pooled (equivalent % fraction) for SRA analysis. % TP rank or S/N of proteins (SILAC LC-MS/MS) based on results of LEAP-RBP SILAC LC-MS/MS experiment. Experiments (a) were performed twice with similar results. Source Data are provided as Source Data file.

FIGS. 11A-11D. Validation of LEAP-RBP clRNP recovery efficiency and RNA-centricity. AGPC: acidic guanidinium thiocyanate-phenol-chloroform. FIG. 11A shows a comparison of methanol, LEAP-RBP, and LEAP-RBP supernatant (“SN”) fractions isolated from final AGPC interphase suspensions of UV-crosslinked cells (0.4 J/cm², 254 nm) by SRA and Coomassie Blue (protein), SYBR Safe (RNA&DNA) staining. RNA recovery shown in adjoining bar chart represents the mean of 2 independent samples. Most of the observed absorbance during UV-spectrophotometric analysis of LEAP-RBP supernatant fractions is from protein, not RNA (see provided Source Data file). FIG. 11B shows that performing repeated LEAP steps does not decrease RNA recovery (i.e., RNA yield). RNA recovery shown in bar chart represents the mean±1 SD of 2 independent samples isolated from separate aliquots of the same final AGPC interphase suspension (biological variability was not of interest). Effect of isolation method (methanol, 1-3 LEAPs) on RNA recovery analyzed using one-way ANOVA: non-significant main effect of isolation method (n.s.1), F(3, 4)=0.73, p=0.585. FIG. 11C shows gentle inversion(S) and emulsion formation enhances RNA recovery. RNA recovery shown in bar chart represents the mean±1 SD of 3 independent samples isolated from separate aliquots of the same final AGPC interphase suspension (biological variability is not of interest). Effect of mixing method (inversions, vortex) on RNA recovery analyzed using one-way ANOVA: significant main effect of mixing method (m.e.1), F(1, 4)=30.88, p=0.005. Note that this is equivalent to an unpaired, two-tailed, homoscedastic t test (see provided Source Data file). FIG. 11D shows validation of LEAP-RBP RNA-centricity. Performing LEAP-RBP on RNase-treated LEAP-RBP fractions results in a loss of detectable protein by SDS-PAGE and Coomassie Blue (protein) staining. Performing LEAP-RBP on proteinase K treated LEAP-RBP fractions results in recovery of visible RNA by SDS-PAGE and SYBR Safe (RNA&DNA) staining but no protein was detected by Coomassie Blue staining. Experiments were performed once (a, d), or three times with similar results (b, c). Source data are provided as a Source Data file.

FIGS. 12A-12B: LEAP-RBP fraction display high UV-dependent enrichment. AGPC: acidic guanidinium thiocyanate-phenol-chloroform. FIG. 12A shows UV-dependent enrichment of RNA and DNA as well as S/N of protein isolated by LEAP-RBP from repeat AGPC extraction interphase suspensions were evaluated by SRA with SYBR Safe (RNA&DNA) and Coomassie Blue (protein) staining or immunoblot; n=3 biologically independent samples, pooled (equivalent % fraction) for SRA analysis; sample compositions and normalization values indicated in FIG. panels. FIG. 12B shows validation of Turbo DNase digest efficiency and depletion of RNase-digested RNA fragments by methanol precipitation for downstream LC-MS/MS analysis: Turbo DNase digestion of LEAP-RBP fractions depletes visible RNase-insensitive DNA retained in the stacker during SDS-PAGE. Methanol precipitation (95% v/v) of RNase-treated LEAP-RBP fractions depletes RNA fragments visible by SDS-PAGE and SYBR Safe staining. DNA digestion efficiency determined by qPCR of Turbo DNase treated and untreated LEAP-RBP fractions; data represents the mean of two independently prepared samples as a percentage of DNA remaining compared to the average of untreated samples. Experiments (a, b) were performed three times with similar results. Source data are provided as a Source Data file.

FIGS. 13A-13D: UV-dose or signal-dependent recovery of noise. AGP: acidic guanidinium thiocyanate-phenol. FIG. 13A, FIG. 13B, FIG. 13C, and FIG. 13D show AGP input suspensions containing equivalent amounts of HeLa cells irradiated with increasing UV-energy (254 nm) were split before isolation of input and LEAP-RBP fractions and thus treated as independent samples (n=2 independent samples for each UV-dose). Biological variability could only be measured across sample groups (n=5 biologically independent samples) thus post-hoc tests between sample groups (n=2) were omitted (a). Protein (BCA) and RNA (UV-spectrophotometry) yields shown in bar charts (a) represent the mean of two independent samples. Protein UV-crosslinking efficiencies were calculated using protein yields of the corresponding input samples and LEAP-RBP fractions of each independent sample, the bar chart (a) shows the mean. a Effect of UV-dose and fraction (input, LEAP-RBP) on protein yield analyzed two-way ANOVA: significant interaction, F(4, 10)=272.99, p<0.001. Subdivided by largest main effect (m.e.), fraction (separate graphs), F(1, 10)=17086.32, p<0.001. Effect of UV-dose on protein yield of input and LEAP-RBP subgroups analyzed using independent one-way ANOVAs: input, non-significant m.e. of UV-dose (n.s.1), F(4, 5)=0.62, p=0.668; LEAP-RBP, significant m.e. of UV-dose (m.e.1*), F(4, 5)=1015.12, p<0.001. To test whether the main effect of UV-dose on protein yield of LEAP-RBP was independent of RNA yield, the effect of UV-dose and macromolecule (protein, RNA) on yield (μg macromolecule/% fraction) of LEAP-RBP was analyzed using two-way ANOVA: significant interaction, F(4, 10)=848.30, p<0.001. Subdivided by largest m.e., macromolecule (separate graphs), F(1, 10)=11902.04,p<0.001. Effect of UV-dose on yield of LEAP-RBP for protein subgroup tested above. Effect of UV-dose on yield of LEAP-RBP for RNA subgroup analyzed using one-way ANOVA: non-significant m.e. of UV-dose (n.s.2), F(4, 5)=0.89, p=0.534. Independent samples from each sample groups were pooled (equivalent % fraction) and analyzed by SRA and SYBR Safe (RNA&DNA), Coomassie Blue (protein) staining, or immunoblot (c); sample compositions indicated in panel b. Experiments (a-c) were performed three times with similar results. Source data are provided as a Source Data file.

FIGS. 14A-14F: Enhanced S/N and improved specificity of LEAP-RBP method (% TP_{S}) increases detection sensitivity of Δ log₂(S) as compared to the INP method. FIGS. 14A-14F show summary of RCS rank analysis (a). For methods with high % TP_{S}, RCS rank predicts log₂(S/N) (b,c), % TP (d, e), coverage (# of unique peptides), or detectable Δ log₂(S). Color overlay based on ordinal RCS rank. f Estimated % TP_{S} and % TP_{N} contributions of the 300 most abundant proteins in LEAP-RBP or INP fractions. Source data are provided as a Source Data file.

FIGS. 15A-15G. LEAP-RBP allows robust and accurate comparisons of RNA-bound proteomes. AGP: acidic guanidinium thiocyanate-phenol; HT: harringtonine. RNA (UV-spectrophotometry) and protein (BCA) quantitation data represents the mean±1 SD of 3 biologically independent samples. FIG. 15A shows a comparison of input and RNP fractions isolated from corresponding AGP input suspensions of samples shown in FIG. 7g by SRA and immunoblot. FIG. 15B shows comparisons of samples shown in FIG. 7g and panel a by SRA and Coomassie Blue (protein) staining, parallel gels. FIG. 15C shows effect of cell line on clRNP composition (μg protein/μg RNA) or protein UV-crosslinking efficiency analyzed using independent one-way ANOVAs: clRNP composition, non-significant main effect of cell line (n.s.1), F(3, 8)=1.38, p=0.318; protein UV-crosslinking efficiency, non-significant main effect of cell line (n.s.2), F(3, 8)=2.97, p=0.097. FIG. 15D shows bar charts showing protein yields of input samples or compositions of RNP and clRNP fractions shown in panel b. FIG. 15E shows estimated RNA UV-crosslinking efficiencies of samples shown in panel b calculated using compositions of corresponding RNP and clRNP fractions shown in panel d. Adjustment of RNA and protein yields for clRNP fractions was performed by comparing the estimated RNA UV-crosslinking efficiency calculated using RNA yields vs composition (μg protein/μg RNA) of RNP and clRNP fractions. Additional information included in the provided Source Data file. FIG. 15F shows bar charts showing adjusted (*) or non-adjusted protein or RNA yields of cRNP and RNP fractions shown in panel b. FIG. 15G shows estimated protein UV-crosslinking efficiencies of samples shown in panel b calculated using protein yield of input samples shown in panel d and adjusted (*) or non-adjusted protein yields of clRNP and RNP fractions shown in panel f; note how adjustment of protein yields for clRNP fractions results in equivalent comparison of estimated protein UV-crosslinking efficiencies between sample groups (DMSO vs HT) with either fraction (RNP or clRNP). Experiments were performed once (a, b, d-g), or three times with similar results (c). Adjustment of clRNP fraction yields using the strategy included in the provided Source Data for panels d-g was performed during three independent experiments with similar results (b). Source data are provided as a Source Data file.

FIGS. 16A-16C. Validation of RNA and DNA depletion steps for proteomic sample preparation. AGPC: acidic guanidinium thiocyanate-phenol-chloroform; AGP: acidic guanidinium thiocyanate-phenol. FIG. 16A shows bar charts showing protein (BCA) or RNA (UV-spectrophotometry) yields of clRNP fractions and/or input samples with or without DNA and/or RNA depletion steps; data represents the mean±1 SD of 3 independent samples isolated from separate aliquots of the same AGP input suspension (input) and corresponding final AGPC interphase suspension (clRNP). Biological variability was not of interest for this experiment. Replicate samples were pooled (equivalent % fraction) and evaluated by SRA with SYBR Safe (RNA&DNA), Coomassie Blue (protein), and Silver Stain (RNA, DNA, and protein) staining (FIG. 16B) or TBE gel analysis (FIG. 16C); sample normalization values indicated in FIG. panels. Experiments (a-c) were performed three times with similar results. Source data are provided as a Source Data file.

FIGS. 17A-17F. Repeated AGPC extraction concentrates clRNPs. A/NGPC: acidic/neutral guanidinium thiocyanate-phenol-chloroform; AGP: acidic guanidinium thiocyanate-phenol. a-g Analytical summary: AGP input suspensions contained either 10 million UV-crosslinked (0.4 J/cm², 254 nm) or 10 million non-crosslinked HeLa cells. Additional information on sample composition, conditions, and normalization is indicated in the FIG. panels. FIG. 17A shows a comparison of methanol-precipitated organic phase fractions normalized to % fraction by SDS-PAGE and Coomassie Blue (protein) staining. FIG. 17B shows a pictorial representation of the AGPC interphase following up to six AGPC extractions of UV-crosslinked and non-crosslinked samples from FIG. 1b, c. FIG. 17C shows select images from panel b with arrows pointing to the solid upper and dispersed lower layer of the AGPC interphase. FIG. 17D shows select images from panel b with arrows pointing to the upper and depleted lower layer of the AGPC interphase. FIG. 17E shows a pictorial representation of the final AGPC interphase resuspended in fresh AGP. FIG. 17F shows TBE gel analysis on RNA (equivalent % fraction) isolated by NGPC extractions of proteinase K treated AGPC interphase samples from panel b (i.e., FIG. 1b, c); parallel gels. Experiments (a, f) were performed once. Source data are provided as a Source Data file.

FIGS. 18A-18J. Isolation of RNPs from AGP suspensions with LEAP-RBP. AGPC: acidic guanidinium thiocyanate-phenol-chloroform; AGP: acidic guanidinium thiocyanate-phenol; SRA: SRA analysis; CL: of 10 million UV-crosslinked (0.4 J/cm², 254 nm); nCL: of million non-crosslinked HeLa cells. a-j Analytical summary: RNA (UV-spectrophotometry) and protein (BCA) quantitation data shown in bar charts represents the mean of two independent samples. Additional information on sample composition, conditions, and normalization is indicated in the FIG. panels. FIG. 18A shows a comparison of untreated LEAP-RBP (in progress) fractions isolated from final AGPC interphase suspensions (CL) by SRA and Coomassie Blue (protein), SYBR Safe (RNA&DNA) staining. FIG. 18B shows comparison of INP and LEAP-RBP (in progress) fractions isolated from final AGPC interphase suspensions (CL) by SRA and Coomassie Blue (protein), SYBR Gold (RNA&DNA) staining. FIG. 18C shows a bar chart showing the effect of chloroform concentration on % RNA recovery from final AGPC interphase suspensions (CL). Pictorial representations illustrating the qualitative change in sample appearance. FIG. 18D shows RNA recovery by LEAP-RBP from AGP suspensions containing varying amounts of protein-bound RNA. FIG. 18E shows % RNA recovery and comparison of RNP fractions isolated by methanol precipitation or 1-3 LEAP steps. FIG. 18F shows a pictorial representations illustrating sample appearance after each inversion during the LEAP-RBP method. FIG. 18G shows a pictorial representations illustrating sample appearance following centrifugation of LEAP-RBP samples using optimal, acceptable, or not acceptable (excessive) volumes of chloroform. FIG. 18H shows UV-spectrophotometric profile of LEAP-RBP fractions isolated from final AGPC interphase suspensions (CL) using 1-3 methanol rinse steps. FIG. 18I, FIG. 18J show pictorial representations illustrating appearance of LEAP-RBP fractions isolated from AGP input suspensions (CL or nCL) during DNA depletion (i) and subsequent LEAP-RBP step (j). Experiments were performed once (d), twice (b), or three times with similar results (a, e). Source data are provided as a Source Data file.

FIGS. 19A-19E: LEAP-RBP fractions contain many proteins previously identified as UV-enriched*. FIG. 19A, FIG. 19B show Venn diagrams showing overlap of total proteins (a) or GO-annotated RBPs (b) identified as UV-enriched* by LEAP-RBP and RNA-centric methods: XRNAX, OOPS, and Ptex 1.5. FIG. 19C, FIG. 19D, and FIG. 19E show Venn diagrams showing overlap of total protein IDs (c), GO-annotated RBPs (d), or GO-annotated mRNA binders (e) identified as UV-enriched* by LEAP-RBP and referenced RIC and eRIC studies.

### DETAILED DESCRIPTION

Section headings as used in this section and the entire disclosure herein are merely for organizational purposes and are not intended to be limiting.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(S),” “include(S),” “having,” “has,” “can,” “contain(S),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Component,” “components,” or “at least one component,” refer generally to a calibrator, a control, a sensitivity panel, a container, a buffer, a diluent, a salt, an enzyme, a co-factor for an enzyme, a detection reagent, a pretreatment reagent/solution, a substrate (e.g., as a solution), a stop solution, and the like that can be included in a kit for assessing a test sample, such as a urine, saliva, whole blood, serum or plasma sample, in accordance with the methods described herein and other methods known in the art. Some components can be in solution or lyophilized for reconstitution for use in an assay.

“Controls” as used herein generally refers to a reagent whose purpose is to evaluate the performance of a measurement system in order to assure that it continues to produce results within permissible boundaries (e.g., boundaries ranging from measures appropriate for a research use assay on one end to analytic boundaries established by quality specifications for a commercial assay on the other end). To accomplish this, a control should be indicative of patient results and optionally should somehow assess the impact of error on the measurement (e.g., error due to reagent stability, calibrator variability, instrument variability, and the like).

“Sample,” “test sample,” “specimen,” “sample from a subject,” and “patient sample” as used herein may be used interchangeably and may be a sample of blood, such as whole blood, tissue, skin, urine, serum, plasma, saliva, amniotic fluid, cerebrospinal fluid, placental cells or tissue, endothelial cells, leukocytes, or monocytes. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art.

### 2. Methods

The contributions of RNA-binding proteins (RBPs) to RNA biology have fostered the development of biochemical methods for the RNA-centric capture and identification of the RNA-interacting proteome. Through these efforts the universe of candidate RBPs has expanded dramatically, with RNA-binding functionality now attributed to a substantial fraction of the proteome, including glycolytic enzymes, regulatory kinases, and other proteins not previously implicated in RNA biology. With the growing catalog of candidate RBPs has come the challenge of establishing quantitative criteria for RNA-binding activity, metrics for distinguishing specific (signal) from random (noise) protein-RNA interactions, and experimental approaches to the study of protein-RNA interaction dynamics. However, significant methodological limitations in the study of RNA-protein interaction remain.

As with protein-protein interactions, protein-RNA interactions can vary substantially in their specificities, interaction lifetimes, and apparent affinities. This intrinsic biological property creates methodological hurdles to establishing biologically relevant interactions, particularly when interaction energies are weak and thus readily lost during biochemical isolation. In the case of RNA-protein complexes (RNPs), chemical- or UV cross-linking methods can capture physiologically relevant interactions though as with any cross-linking method, criteria for distinguishing specific from biologically irrelevant interactions should be used. UV cross-linking is preferred due to its high specificity, though it is also inefficient, with only a fraction of interactors forming a covalent adduct. The generally low cross-linking efficiencies present an analytical challenge because selective, quantitative recovery of the UV-crosslinked protein-RNA complexes (clRNPs) is needed for accurately determining RNA occupancy states in vivo.

The methods for isolating nucleic acids provided herein address these and other issues, and provide a method for high-stringency, efficient extraction of nucleic acids, including RNA-protein complexes (RNPs), from a sample. In some aspects, provided herein is a biochemical method termed LEAP-RBP (Liquid-Emulsion-Assisted-Purification of RNA-Bound Protein) for the selective isolation of total RNA-bound protein. SILAC LC-MS/MS analysis of LEAP-RBP fractions demonstrated high RNA-bound protein enrichment and through comparative analyses, revealed a key metric for evaluating method specificity for RNA-bound RBPs which is termed % TP_{S}, or RNA-bound protein abundance. High % TP_{S} is indicative of low free protein recovery and enables the accurate study of dynamic, cell state-determined changes in RBP occupancy state. Using this signal-based analytical framework, methods for evaluating RNA-bound proteomes and their dynamics are provided herein. The utility of this approach is established through benchmark comparisons of LEAP-RBP with current RNA-centric enrichment methods.

In some aspects, provided herein are methods of isolating nucleic acids from a sample. In some embodiments, methods for isolating nucleic acids from a sample comprise contacting the sample with a precipitation buffer comprising a monovalent salt and an alcohol, thereby isolating the nucleic acids from the sample.

In some aspects, provided herein are methods of isolating RNA-protein complexes (RNPs) from a sample. In some embodiments, methods of isolating RNA-protein complexes from a sample comprise performing at least one round of acidic guanidium thiocyanate-phenol-chloroform (AGPC) biphasic extraction on the sample and isolating an interphase portion after each round of extraction, thereby obtaining a sample fraction enriched in RNA-protein complexes (RNPs); and contacting the sample fraction enriched in RNPs with a precipitation buffer comprising isopropanol and lithium chloride (LiCl).

In some aspects, provided herein are methods of identifying RNA binding proteins (RBPs) in a sample. In some embodiments, methods of identifying RBPs in a sample comprise obtaining a composition comprising RNA-protein complexes (RNPs) isolated from a sample; depleting DNA from the composition; and identifying RNA binding proteins in the composition by mass spectrometry. In some embodiments, RNPs are isolated from the sample by performing at least one round of acidic guanidium thiocyanate-phenol-chloroform (AGPC) biphasic extraction on the sample and isolating an interphase portion after each round of extraction, thereby obtaining a sample fraction enriched in RNA-protein complexes (RNPs); and contacting the sample fraction enriched in RNPs with a precipitation buffer comprising isopropanol and lithium chloride (LiCl).

The methods of isolating nucleic acids, isolating RNPS, and identifying/evaluating RBPs in a sample provided herein, along with suitable compositions (e.g. precipitation buffers) for conducting the same, are further described in Kristofich J, Nicchitta CV. Signal-noise metrics for RNA binding protein identification reveal broad spectrum protein-RNA interaction frequencies and dynamics. Nat Commun. 2023 Sep. 21; 14(1):5868. doi: 10.1038/s41467-023-41284-9. PMID: 37735163: PMCID: PMC10514315.

### Precipitation Buffer

The methods provided herein involve contacting a sample with a precipitation buffer comprising a monovalent salt and an alcohol. In some embodiments, the monovalent salt is sodium chloride (NaCl) or lithium chloride (LiCl). In some embodiments, the alcohol is isopropanol or ethanol. In some embodiments, the monovalent salt is LiCl and the alcohol is isopropanol.

“Contacting the sample with a precipitation buffer” is inclusive of contacting the sample with a single buffer comprising both the monovalent salt (e.g. NaCl or LiCl) and the alcohol (e.g. isopropanol or ethanol), contacting the sample with a monovalent salt followed by contacting the sample with the alcohol, or contacting the sample with the alcohol followed by contacting the sample with the monovalent salt. Accordingly, the term “precipitation buffer” does not necessarily indicate that a single buffer contains both the monovalent salt and the alcohol. The following descriptions of the amount/concentration of the components of a precipitation buffer are intended to include the amount/concentration of the monovalent salt and the alcohol that can be present in a single buffer (e.g. in embodiments where the sample is contacted with the monovalent salt and the alcohol simultaneously), or the amount/concentration of the monovalent salt and the amount/concentration of the alcohol that are separately contacted with the sample (e.g. in embodiments where the sample is first contacted with the monovalent salt, and then contacted with the alcohol, or in embodiments where the sample is first contacted with the alcohol, and then contacted with the monovalent salt).

In some embodiments, the precipitation buffer comprises from about 0.5M to about 10M of the monovalent salt and about 20% to about 80% alcohol. In some embodiments, the precipitation buffer comprises from about 0.5M to about 7.5M of the monovalent salt and about 25% to about 75% alcohol.

In some embodiments, the monovalent salt is LiCl. In some embodiments, the precipitation buffer comprises from about 0.5M to about 10M LiCl and about 20% to about 80% alcohol. In some embodiments, the precipitation buffer comprises from about 1M to about 7.5M LiCl and about 20% to about 80% alcohol. In some embodiments, the precipitation buffer comprises from about 3M to about 6M LiCl and about 20% to about 80% alcohol. In some embodiments, the precipitation buffer comprises from about 3.5M to about 5M LiCl and about 20% to about 80% alcohol.

In some embodiments, the precipitation buffer comprises from about 0.5M to about 10M LiCl and about 25% to about 75% alcohol. In some embodiments, the precipitation buffer comprises from about 1M to about 7.5M LiCl and about 25% to about 75% alcohol. In some embodiments, the precipitation buffer comprises from about 3M to about 6M LiCl and about 25% to about 75% alcohol. In some embodiments, the precipitation buffer comprises from about 3.5M to about 5M LiCl and about 25% to about 75% alcohol.

In some embodiments, the precipitation buffer comprises from about 0.5M to about 10M LiCl and about 30% to about 60% alcohol. In some embodiments, the precipitation buffer comprises from about 1M to about 7.5M LiCl and about 30% to about 60% alcohol. In some embodiments, the precipitation buffer comprises from about 3M to about 6M LiCl and about 30% to about 60% alcohol. In some embodiments, the precipitation buffer comprises from about 3.5M to about 5M LiCl and about 30% to about 60% alcohol.

In some embodiments, the precipitation buffer comprises from about 0.5M to about 10M LiCl and about 45% to about 55% alcohol. In some embodiments, the precipitation buffer comprises from about 1M to about 7.5M LiCl and about 45% to about 55% alcohol. In some embodiments, the precipitation buffer comprises from about 3M to about 6M LiCl and about 45% to about 55% alcohol. In some embodiments, the precipitation buffer comprises from about 3.5M to about 5M LiCl and 45% to about 55% alcohol.

In some embodiments, the precipitation buffer comprises about 0.5M, about 1M, about 1.5M, about 2M, about 2.5M, about 3M, about 3.5M, about 4M, about 4.5M, about 5M, about 5.5M, about 6M, about 6.5M, about 7M, about 7.5M, about 8M, about 8.5M, about 9M, about 9.5M, or about 10M LiCl and about 20%, about 25%, about 30%, about 35%, about 40%, about 45%, about 50%, about 55%, about 60%, about 65%, about 70%, about 75%, about 80% alcohol (e.g. ethanol or isopropanol). In some embodiments, the precipitation buffer comprises about 3M, about 3.1M, about 3.2M, about 3.3M, about 3.4M, about 3.5M, about 3.6M, about 3.7M, about 3.8M, about 3.9M, about 4M, about 4.1M, about 4.2M, about 4.3M, about 4.4M, or about 4.5M LiCl and about 20%, about 25%, about 30%, about 35%, about 40%, about 45%, about 50%, about 55%, about 60%, about 65%, about 70%, about 75%, about 80% alcohol (e.g. ethanol or isopropanol).

In some embodiments, the monovalent salt is NaCl. In some embodiments, the precipitation buffer comprises from about 0.5M to about 10M NaCl and about 20% to about 80% alcohol. In some embodiments, the precipitation buffer comprises from about 0.5M to about 7.5M NaCl and about 20% to about 80% alcohol. In some embodiments, the precipitation buffer comprises from about 0.5M to about 5M NaCl and about 20% to about 80% alcohol. In some embodiments, the precipitation buffer comprises from about 0.5M to about 2.5M NaCl and about 20% to about 80% alcohol. In some embodiments, the precipitation buffer comprises from about 0.6M to about 2 M NaCl and about 20% to about 80% alcohol.

In some embodiments, the precipitation buffer comprises from about 0.5M to about 10M NaCl and about 25% to about 75% alcohol. In some embodiments, the precipitation buffer comprises from about 0.5M to about 7.5M NaCl and about 25% to about 75% alcohol. In some embodiments, the precipitation buffer comprises from about 0.5M to about 5M NaCl and about 25% to about 75% alcohol. In some embodiments, the precipitation buffer comprises from about 0.5M to about 2.5M NaCl and about 25% to about 75% alcohol. In some embodiments, the precipitation buffer comprises from about 0.6M to about 2 M NaCl and about 25% to about 75% alcohol.

In some embodiments, the precipitation buffer comprises from about 0.5M to about 10M NaCl and about 30% to about 60% alcohol. In some embodiments, the precipitation buffer comprises from about 0.5M to about 7.5M NaCl and about 30% to about 60% alcohol. In some embodiments, the precipitation buffer comprises from about 0.5M to about 5M NaCl and about 30% to about 60% alcohol. In some embodiments, the precipitation buffer comprises from about 0.5M to about 2.5M NaCl and about 30% to about 60% alcohol. In some embodiments, the precipitation buffer comprises from about 0.6M to about 2 M NaCl and about 30% to about 60% alcohol.

In some embodiments, the precipitation buffer comprises from about 0.5M to about 10M NaCl and about 45% to about 55% alcohol. In some embodiments, the precipitation buffer comprises from about 0.5M to about 7.5M NaCl and about 45% to about 55% alcohol. In some embodiments, the precipitation buffer comprises from about 0.5M to about 5M NaCl and about 45% to about 55% alcohol. In some embodiments, the precipitation buffer comprises from about 0.5M to about 2.5M NaCl and about 45% to about 55% alcohol. In some embodiments, the precipitation buffer comprises from about 0.6M to about 2 M NaCl and about 45% to about 55% alcohol.

In some embodiments, the alcohol is isopropanol. In some embodiments, the precipitation buffer comprises about 20% to about 80% isopropanol. In some embodiments, the precipitation buffer comprises about 20% to about 80% isopropanol, 25% to about 75% isopropanol, 30% to about 60% isopropanol, 45% to about 55% isopropanol, or about 50% isopropanol.

In some embodiments, the alcohol is ethanol. In some embodiments, the precipitation buffer comprises about 20% to about 80% ethanol. In some embodiments, the precipitation buffer comprises about 20% to about 80% ethanol, 25% to about 75% ethanol, 30% to about 60% ethanol, 45% to about 55% ethanol, or about 50% ethanol.

In some embodiments, contacting the sample with the precipitation buffer comprises mixing the sample with the precipitation buffer and incubating the sample with the precipitation buffer for at least 10 seconds. As described above, contacting the sample with the precipitation buffer is inclusive of contacting the sample with a single buffer comprising the monovalent salt and the alcohol, contacting the sample with the monovalent salt followed by contacting the sample with the alcohol, and contacting the sample with the alcohol followed by contacting the sample with the monovalent salt. “Incubating” the sample with the precipitation buffer commences once the sample has been contacted with both the alcohol and the monovalent salt, regardless of whether that contact occurs simultaneously or sequentially. In some embodiments, the sample is incubated with the precipitation buffer for about 10 seconds to about 24 hours. In some embodiments, the sample is incubated with the precipitation buffer for about 10 seconds to about 12 hours. In some embodiments, the sample is incubated with the precipitation buffer for about 10 seconds to about 6 hours. In some embodiments, the sample is incubated with the precipitation buffer for about 10 seconds to about 3 hours. In some embodiments, the sample is incubated with the precipitation buffer for about 10 seconds to about 1 hour. In some embodiments, the sample is incubated with the precipitation buffer for about 10 seconds to about 1 hour, about 10 seconds to about 30 minutes, about 10 seconds to about 10 minutes, about 20 seconds to about 5 minutes, about 30 seconds to about 2 minutes, for about 40 seconds to about 80 seconds, or for about 1 minute.

In some embodiments, at least two rounds of mixing and incubating the sample with the precipitation buffer are performed. For example, in some embodiments the sample is mixed with the sample buffer by inversion (e.g. by inverting a tube containing the sample and the precipitation buffer one or more times), incubated with the precipitation buffer for a suitable duration of time, and then mixed again (e.g. by inversion) and incubated again for a suitable duration of time. In some embodiments, at least three rounds, at least four rounds, or more than five rounds of mixing and incubating the sample with the precipitation buffer are performed.

In some embodiments, contacting the sample with the precipitation buffer precipitates nucleic acids from the sample. In some embodiments, the sample is centrifuged at a suitable speed to form a pellet containing the precipitated nucleic acids. Excess liquid can be removed from the pellet and one or more wash steps can be performed to further remove potential contaminants/excess liquid from the nucleic acids.

In some embodiments, the sample is contacted with the precipitation buffer (e.g. mixed and incubated with the precipitation buffer) at room temperature. In some embodiments, the sample is contacted with the precipitation buffer at a temperature ranging from about 0° C. to about 30° C. In some embodiments, the sample is contacted with the precipitation buffer at a temperature of from about 4° C. to about 22° C.

### Sample

The methods provided herein are suitable for use in a variety of sample types. The sample may be any suitable sample comprising nucleic acids (e.g. RNA, RNA binding proteins, RNA protein complexes). In some embodiments, the sample is a biological sample. The term “biological sample” refers to a sample obtained from a cell or subject. The subject may be human or non-human. In some embodiments, the sample comprises a bodily fluid (e.g. blood, serum, plasma, etc.) or a tissue sample. In some embodiments, the sample comprises cells or cell contents/products (e.g. cell lysates). In some embodiments, the sample is cross-linked (e.g. UV-crosslinked). In some embodiments, the sample comprises lysates obtained from UV crosslinked cells.

In some embodiments, the sample comprises nucleic acids, acidic guanidinium thiocyanate, and phenol. The combination of acidic guanidium thiocyanate and phenol is also referred to herein as acidic guanidium thiocyanate-phenol buffer or AGP. In some embodiments, the ratio (v/v) of acidic guanidium thiocyanate to phenol present in the AGP buffer is about 2:1 (e.g. about 2 parts by volume of acidic guanidium thiocyanate to about 1 part by volume of phenol). In some embodiments, the sample comprises at least 4 parts AGP by volume. In some embodiments, the sample comprises at least 6 parts AGP by volume.

In some embodiments, the sample comprises nucleic acids, AGP, and a solvent. In some embodiments, addition of a solvent to a sample comprising nucleic acids and AGP causes an emulsion to form. In some embodiments, the solvent is chloroform. In some embodiments, the solvent is dichloromethane. In some embodiments, the solvent is added to the sample at a final concentration of from about 1% to about 10% (v/v). In some embodiments, the solvent is added to the sample at a final concentration of from about 1% to about 8% (v/v). In some embodiments, the ratio (v/v) of solvent (e.g. chloroform) to AGP buffer added to a sample to induce separation into aqueous and organic phases with an interphase dispersed therebetween is about 1:10 (e.g. about 1 part solvent to about 10 parts AGP buffer) to about 1:1 (e.g. about 1 part solvent to about 1 part AGP buffer).

In some embodiments, the ratio (v/v) of the sample to the precipitation buffer added to the sample is from about 1:1 (e.g. 1 part by volume of the sample to 1 part by volume of the precipitation buffer) to about 1:9 (e.g. 1 part by volume of the sample to 9 parts by volume of the precipitation buffer). In some embodiments, the ratio (v/v) of the sample to the precipitation buffer is about 1:1, about 1:2, about 1:3, about 1:4. About 1:5, about 1:6, about 1:7, about 1:8, or about 1:9. The volume of the precipitation buffer refers to the volume of a single buffer comprising the monovalent salt and the alcohol or to the volume of monovalent salt and the volume of alcohol added separately to the sample. In some embodiments, the sample comprises nucleic acids and AGP, and the ratio (v/v) of the sample to the precipitation buffer added to the sample is about 1:1, about 1:2, about 1:3, about 1:4. About 1:5, about 1:6, about 1:7, about 1:8, or about 1:9. In some embodiments, the sample comprises nucleic acids, AGP, and a solvent (e.g. chloroform), and the ratio (v/v) of the sample to the precipitation buffer added to the sample is about 1:1, about 1:2, about 1:3, about 1:4. About 1:5, about 1:6, about 1:7, about 1:8, or about 1:9.

In some embodiments, the sample is an AGPC interphase sample (e.g. the interphase collected after at least one round of AGPC biphasic extraction). In some embodiments, the sample is an AGPC interphase sample produced after a single round of AGPC biphasic extraction. In some embodiments, the sample is an interphase sample resulting from two or more sequential rounds of AGPC biphasic extraction. In some embodiment, a biological sample is subjected to one or more processing steps to obtain an AGPC interphase sample, and the AGPC interphase sample is contacted with a precipitation buffer to isolate nucleic acids (e.g. RNA, RNA protein complexes) from the sample. In some embodiments, the sample is enriched in RNPs.

### AGPC Biphasic Extraction

In some embodiments, the methods provided herein comprise performing at least one round of acidic guanidium thiocyanate-phenol-chloroform (AGPC) biphasic extraction on a sample prior to contacting the sample with the precipitation buffer.

In some embodiments, AGPC biphasic extraction is performed on the sample to enrich for RNPs at the interphase prior to extracting nucleic acids from the sample. For example, one or more rounds of AGPC biphasic extraction may be performed on a sample, each subsequent round being performed on the isolated interphase produced by the previous extraction, to enrich for RBPs at the interphase. The interphase enriched in RNPs is then contacted with a precipitation butter to isolate RNPs from the interphase sample. In some embodiments, AGPC biphasic extraction is particularly useful in methods of isolating RNA-protein complexes or identifying RBPs in a sample, due to the enrichment of RNA protein complexes at the interphase during extraction.

In some embodiments, AGPC biphasic extraction comprises contacting a sample comprising nucleic acids with acidic guanidium thiocyanate-phenol buffer (AGP) and a solvent. In some embodiments, the solvent is chloroform. Alternative solvents may be used, such as dichloromethane. Addition of the solvent and mixing the sample induces separation into aqueous and organic phases with an interphase dispersed between the two phases. The interphase is enriched in RNA protein complexes. In some embodiments, the interphase is isolated after one AGPC biphasic extraction step, and contacted with the precipitation butter to isolate nucleic acids from the sample. In some embodiments, multiple AGPC biphasic extraction steps are performed to further enrich for RNPs at the interphase. Each AGPC biphasic extraction step comprises contacting the interphase obtained from the previous extraction step with AGP and a solvent (e.g. chloroform), mixing, and isolating the interphase.

In some embodiments, the ratio (v/v) of acidic guanidium thiocyanate to phenol present in the AGP buffer is about 2:1 (e.g. about 2 parts by volume of acidic guanidium thiocyanate to about 1 part by volume of phenol). In some embodiments, the ratio (v/v) of solvent (e.g. chloroform) to AGP buffer added to a sample to induce separation into aqueous and organic phases with an interphase dispersed therebetween is about 1:10 (e.g. about 1 part solvent to about 10 parts AGP buffer) to about 1:1 (e.g. about 1 part solvent to about 1 part AGP buffer).

In some embodiments, performing at least one round of AGPC biphasic extraction provides a sample fraction enriched in RNPs. In some embodiments, the sample fraction enriched in RNPs is contacted with a precipitation buffer comprising isopropanol and lithium chloride (LiCl), as described above, thereby isolating RNPs from the sample fraction (e.g. thereby providing a composition comprising RNPs isolated from a sample). RNA binding proteins present in the composition comprising RNPs may be further assessed, such as by mass spectrometry, to identify bona fide RBPs with high accuracy. In some embodiments, DNA is depleted from the composition prior to assessing RNA binding proteins. For example, DNA may be depleted from the composition prior to mass spectrometry. In some embodiments, DNA is depleted by addition of an enzyme that degrades DNA (e.g. a DNAse).

### Assessing RBPs

In some embodiments, the methods provided herein comprise assessing RBPs following isolation of nucleic acids (e.g. RNA protein complexes) from a sample. RBPs may be assessed by any suitable technique. In some embodiments, RBPs are assessed by mass spectrometry. Mass spectrometry refers to an analytical technique used to measure the mass-to-charge ratio of ions present in a sample. In some embodiments, RBPs are assessed by liquid chromatography with tandem mass spectrometry (LC-MS/MS). Exemplary mass spectrometry techniques and exemplary analytical strategies that can be used to assess RBPs are further described in the accompanying examples.

The methods provided herein are demonstrated to identify bona-fide RBPs with high accuracy compared to existing methods, which are ineffective for a variety of reasons including false-positives and inefficient recovery of RNA protein complexes and therefore loss of potential RBPs during sample processing. Indeed, results herein demonstrate that the methods provided herein recovery near 100% of RNA-bound protein from an interphase sample, which enables accurate and complete assessment of RNA binding proteins. Moreover, the methods provided herein achieve a high signal to noise ratio (S/N) for most RBS without significant signal loss, and clearly distinguish RBPs with low S/N (e.g., RPN1, TRAPα) from non-RBPs. Furthermore, the methods provided herein achieve UV-independent recovery of RNA. In other words, recovery is not biased towards free vs. bound RNA species. This is not obtainable by other current methods. The technical advantages of the methods described herein over existing methods, such as RNA centric methods, are further described in the accompanying examples, and are further described in Kristofich J. Nicchitta CV. Signal-noise metrics for RNA binding protein identification reveal broad spectrum protein-RNA interaction frequencies and dynamics. Nat Commun. 2023 Sep. 21:14(1):5868. doi: 10.1038/s41467-023-41284-9. PMID: 37735163: PMCID: PMC10514315.

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure has multiple aspects, illustrated by the following non-limiting examples.

### EXAMPLES

### Example 1

Recent efforts towards the comprehensive identification of RNA-bound proteomes have revealed a large, surprisingly diverse family of candidate RNA-binding proteins (RBPs). Quantitative metrics for characterization and validation of protein-RNA interactions and their dynamic interactions have, however, proven analytically challenging and prone to error. Provided herein is a novel method termed LEAP-RBP (Liquid-Emulsion-Assisted-Purification of RNA-Bound Protein) for the selective, quantitative recovery of UV-crosslinked RNA-protein complexes. By virtue of its high specificity and yield, LEAP-RBP distinguishes RNA-bound and RNA-free protein levels and reveals common sources of experimental noise in RNA-centric RBP enrichment methods. Provided herein are methods for accurate RBP identification and signal-based metrics for quantifying protein-RNA complex enrichment, relative RNA occupancy, and method specificity. In this work, the utility of the approach is validated by comprehensive identification of RBPs whose association with mRNA is modulated in response to global mRNA translation state changes and through in-depth benchmark comparisons with current methodologies.

### Results:

### RNA-Bound Proteins Display RNase-Dependent SDS-PAGE Mobility

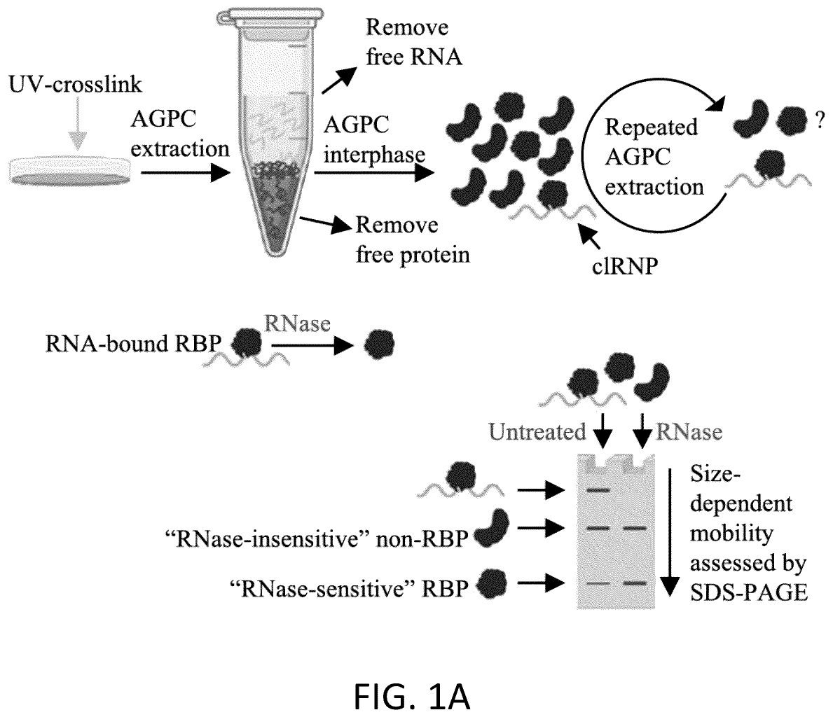

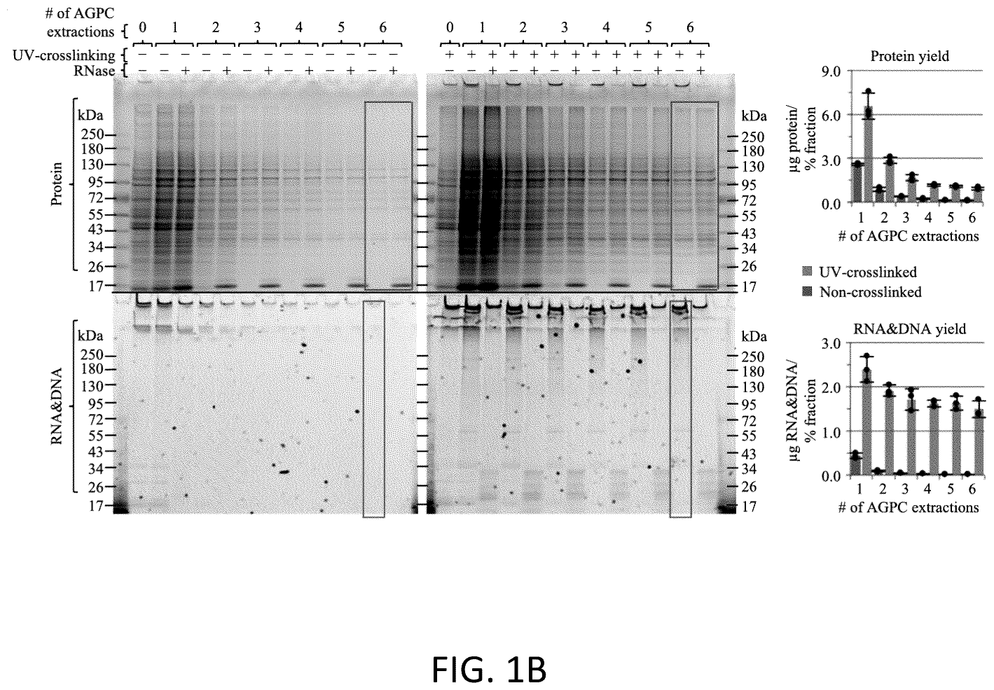

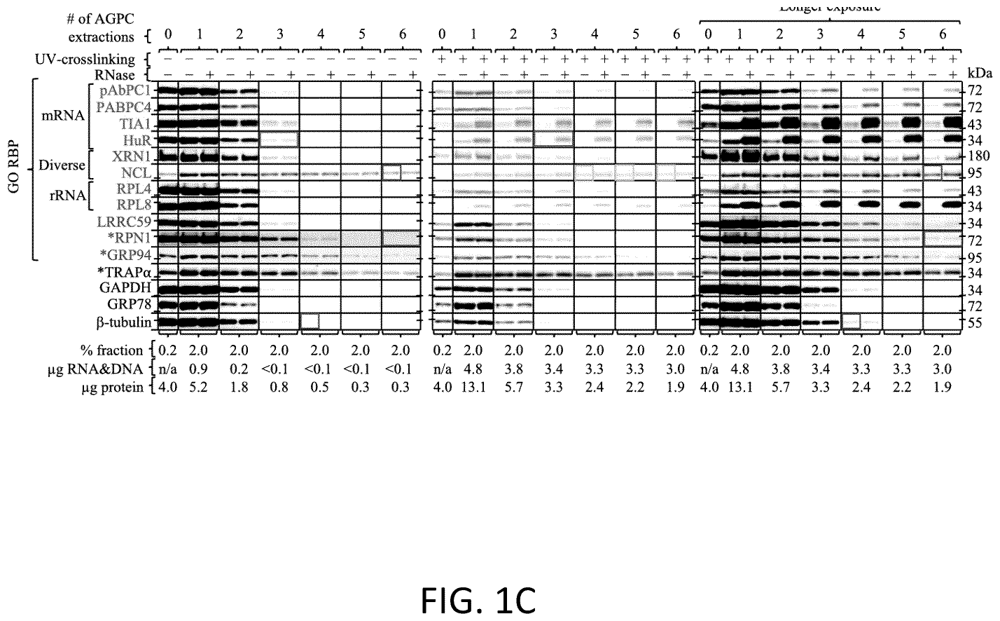

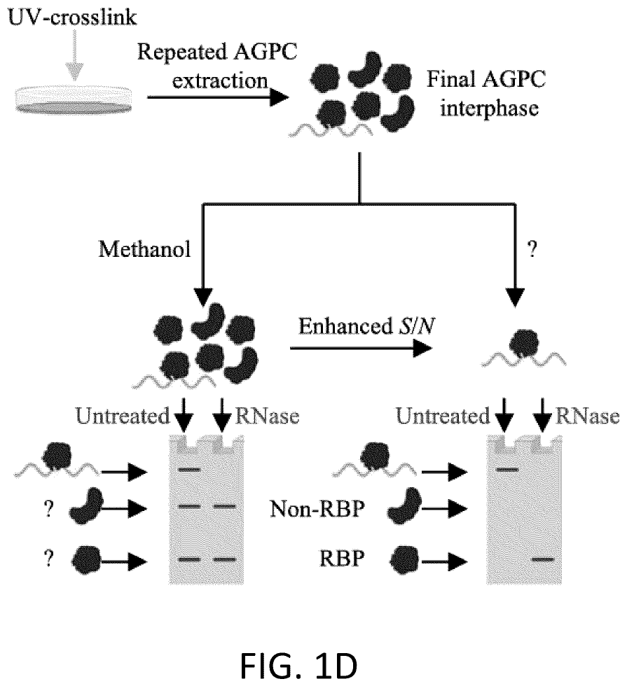

RBPs are shown herein to exhibit UV-dependent enrichment at the AGPC interphase. As such, repeated AGPC extraction was used to enhance S/N (RNA-bound protein/free protein) of AGPC interphase proteins using an SDS-PAGE RNase-sensitivity Assay (SRA) (FIG. 1a). As illustrated in FIG. 1a, UV cross-linked RNA-protein complexes, which comprise signal (═S), migrate at a higher apparent molecular weight than their unbound counterparts (noise=N) in SDS-PAGE RNase treatment liberates RNA-bound protein, increasing the amount of observed (═O) protein migrating at its expected molecular weight (ΔO=|S|). Consequently, a comparison of RNase-treated (S+N) and untreated control samples (N) by SDS-PAGE reveals RNase-sensitive RBPs (|S|>0) and enables the determination of S/N. As described in Eq. (1), log₂ transformation yields RNase-dependent fold-change (Supplementary Note 2).

RNase-dependent fold-change=Δ log₂(O)=log₂(|S|+N)_{RNase}−log₂(N)_{untreated} (1)

SRA was used to evaluate UV-dependent enrichment and S/N of proteins recovered from the AGPC interphase of UV-crosslinked (0.4 J/cm², 254 nm) and non-crosslinked HeLa cells, using sequential interphase extraction to maximize RBP enrichment (FIG. 1b). To track RNA enrichments and RNase digestion efficacy, parallel gels were run and stained with SYBR Safe. Interestingly, while RNA exhibited UV-enrichment in the AGPC interphase (lower gel, red boxes), proteins showed only modest AGPC interphase UV-enrichment and were largely RNase-insensitive (upper gel, blue boxes), indicating that most of the proteins recovered from the AGPC interphase fraction of UV-irradiated cells were not crosslinked to RNA. These data demonstrate that UV-irradiation-dependent enrichment at the AGPC interphase alone is insufficient evidence for RNA-binding activity. To distinguish between contributions of noise (RBPs lacking crosslinked RNA) and background (non-RBPs) to this phenomenon, known GO-annotated RBPs binding different RNA species were compared with non-RBPs by SRA and immunoblot. Here, proteins lacking GO-annotation (GO:RBP) are designated as non-RBPs, but this may not be due to a lack of RNA-binding activity (“Methods”). Protein targets were selected on the basis of prior literature, GO-RBP-annotation datasets, RNA-binding domain bioinformatic analysis, and UV cross-linking/CLIP sequencing frequency, to include canonical RBPs containing known RNA-binding domains (red font) and noncanonical RBPs (blue font) which were identified in previous studies as candidate RBPs (i.e., scored as UV-enriched) but which lack known RNA-binding domains (FIG. 1c) Intriguingly, all canonical RBPs exhibited some degree of UV-dependent RNase sensitivity by SRA (red box) and were recovered from the AGPC interphase, whereas noncanonical RBPs and non-RBPs were either RNase-insensitive or interphase depleted following repeat extraction. Most glycoproteins assayed (asterisks) exhibited UV-independent enrichment at the AGPC interphase, although ribophorin I (RPN1) did not (purple box; FIG. 1c). The finding that known RBPs lacking crosslinked RNA, e.g., nucleolin (NCL), and non-RBPs, e.g., β-tubulin (blue boxes) can score as UV-enriched provides a clear demonstration that orthogonal assays such as SRA have utility for validation of candidate RBPs.

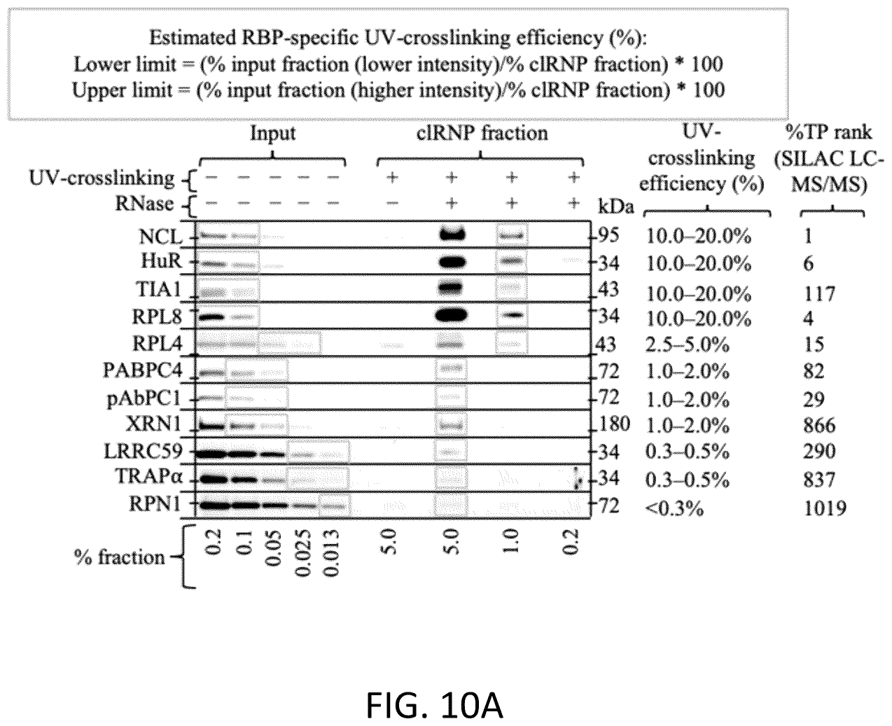

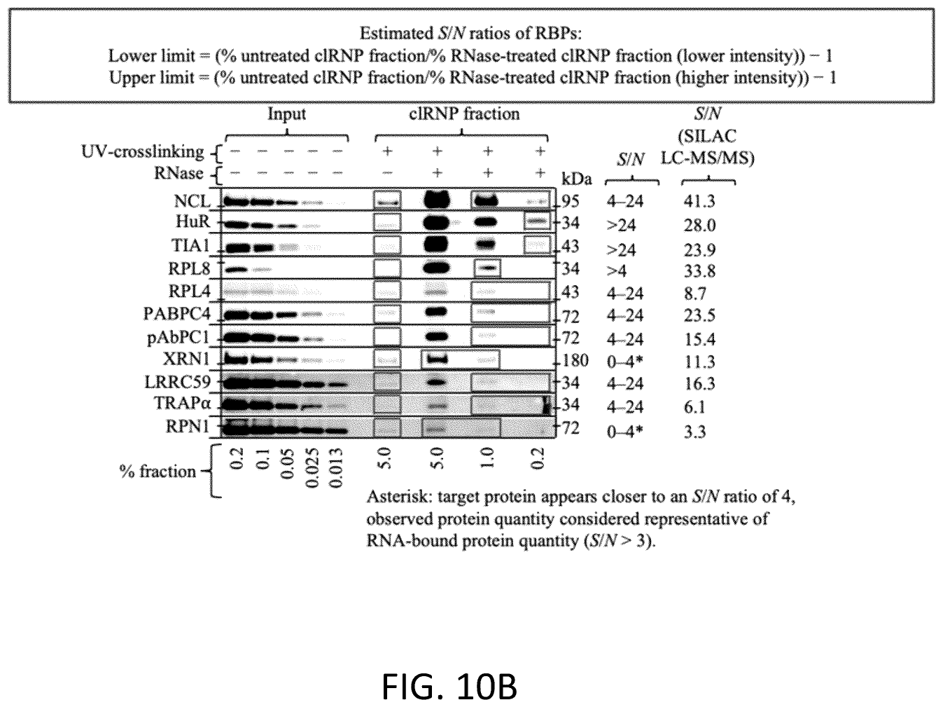

Even after six AGPC extractions, the established RBP nucleolin (NCL) reached an apparent S/N enrichment limit (gold box; FIG. 1c). This may be attributed to intrinsic limitations in the ability of repeated AGPC extraction to enhance the S/N of a given RBP (FIG. 10 and Supplementary Notes³ and ⁴). This finding suggested that a subset of RNase-insensitive proteins at the final AGPC interphase may be bona fide RBPs with intrinsically low S/N. Combined, these observations revealed a need for enrichment methods that are selective for RNA-bound RBPs and thereby support quantitative S/N determinations (FIG. 1d).

### LEAP-RBP Provides Efficient Recovery of RNA-Bound Protein

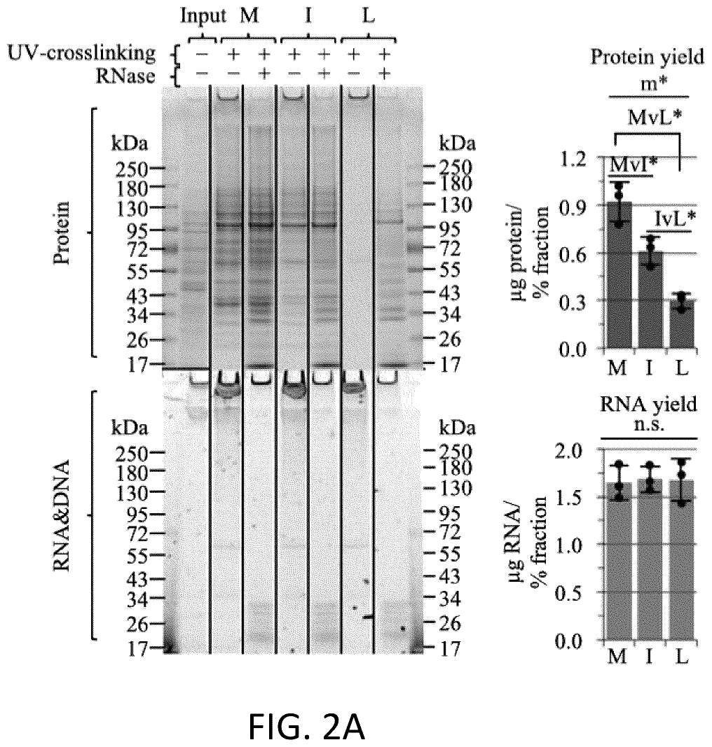

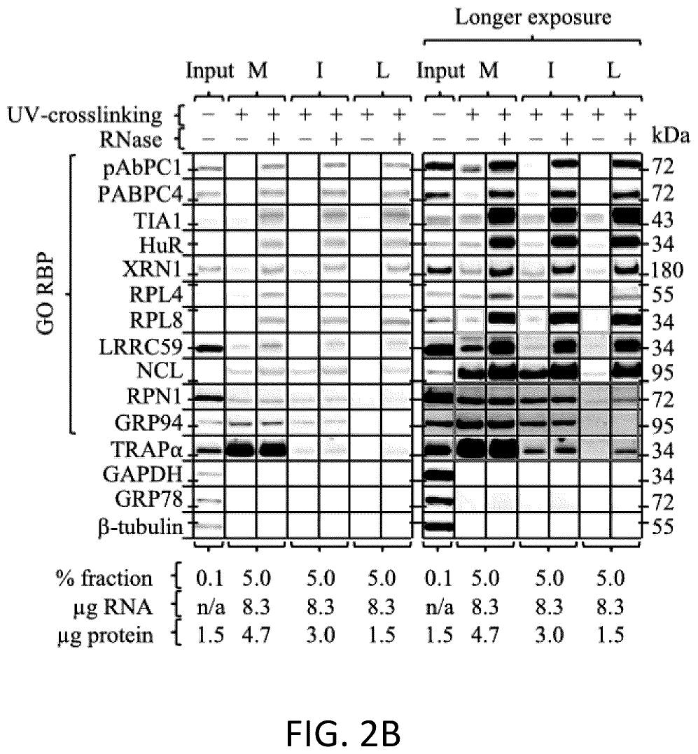

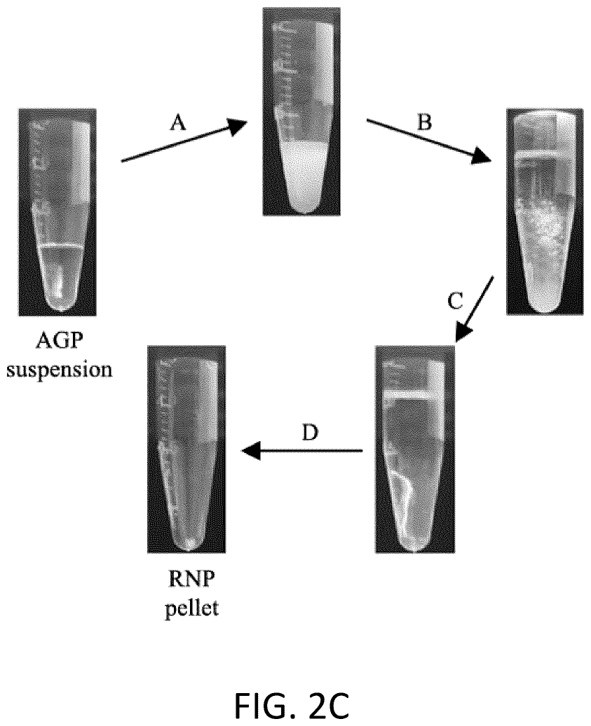

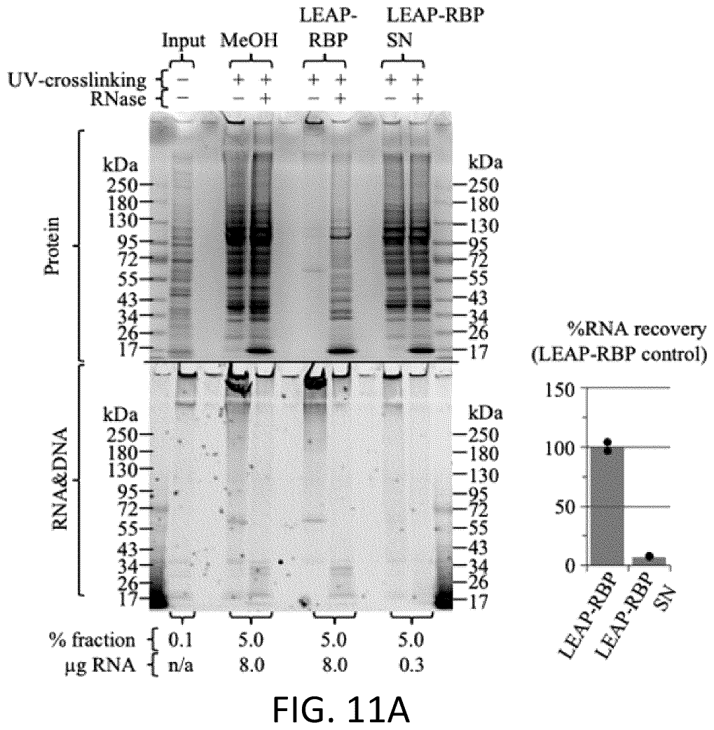

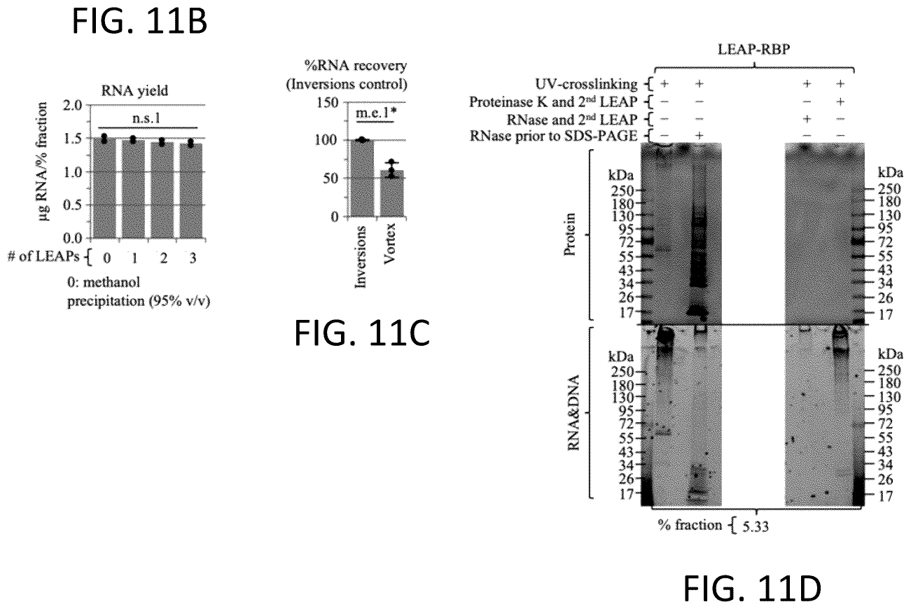

Efforts to identify methods for selectively isolating RNA-bound RBPs to support quantitative S/N determinations yielded two approaches, INP (Isopropanol NaCl Precipitation) and LEAP-RBP (Liquid-Emulsion-Assisted-Purification of RNA-Bound Protein). Both are RNA-centric enrichment methods that enhance S/N by SRA without significant signal loss (FIG. 2a, b), but only LEAP-RBP (L) clearly distinguishes RBPs with low S/N (e.g., RPN1, TRAPα) from non-RBPs (red box; FIG. 2b). LEAP-RBP is notable for its high selectivity for RNA-protein complexes, with only negligible protein quantities displaying SDS-PAGE mobility in the absence of RNase digestion (FIG. 2). This conclusion is further supported by assays of protein and RNA yields in the different enrichment methods, where LEAP-RBP yielded a substantially higher total RNA/protein than that obtained by methanol or isopropanol/NaCl precipitation. The utility of LEAP-RBP is readily apparent by SRA re-evaluation of a subset of previously identified candidate RBPs where for example the previously GO-annotated RBP and endoplasmic reticulum (ER) chaperone GRP94 scores as a false positive and the non-RBP ER integral membrane protein TRAPα as a false negative (FIG. 2). The enhanced S/N of the LEAP-RBP method was achieved using a heterogeneous, lithium-supplemented solvent system which provides rapid and selective precipitation of RNA-bound proteins (FIG. 2c, FIG. 11a-c, Methods).

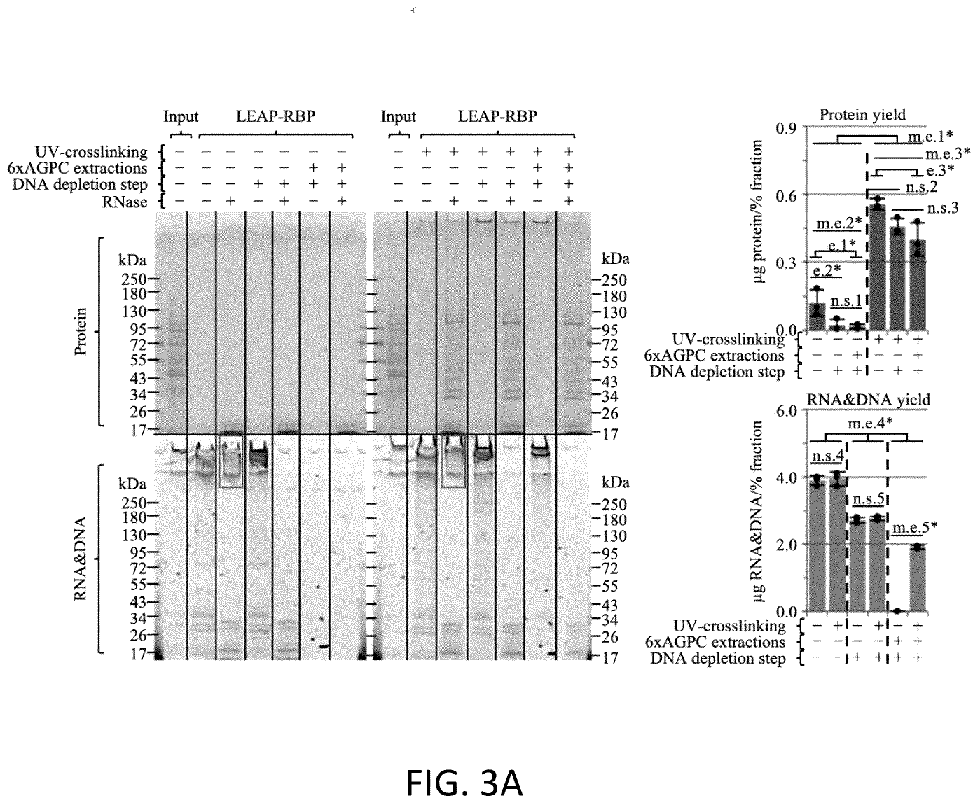

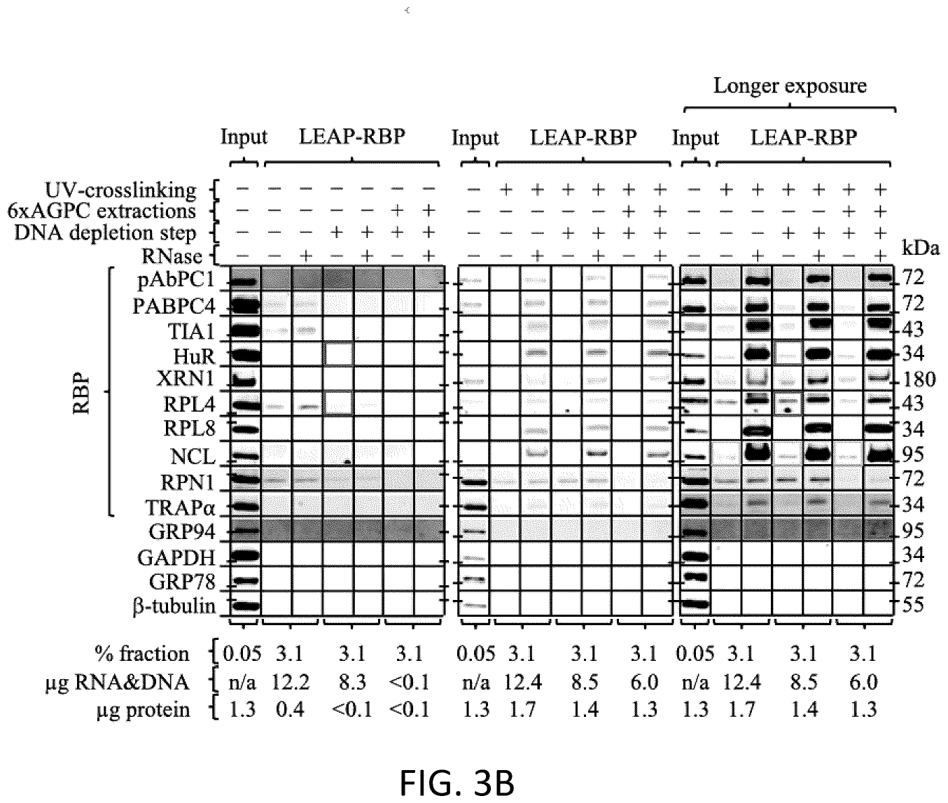

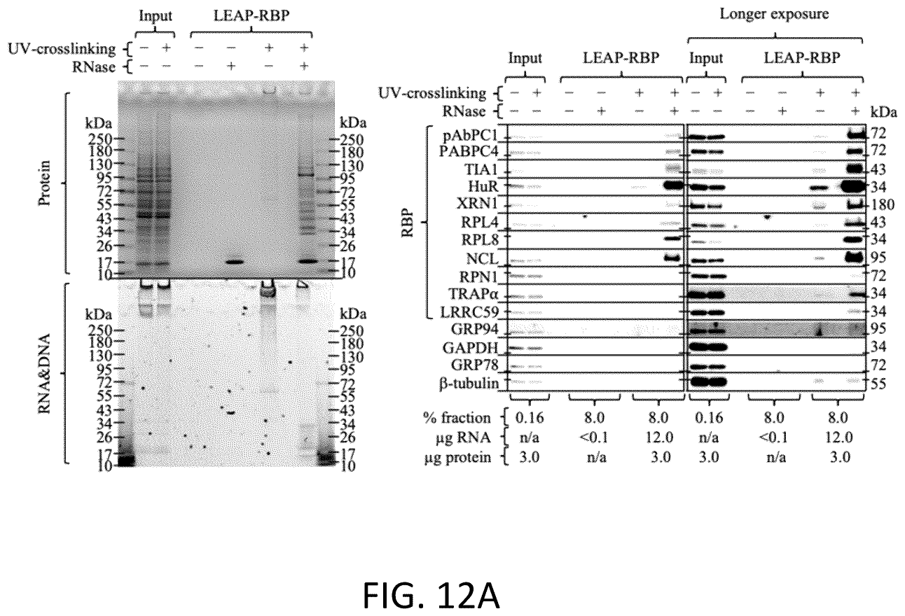

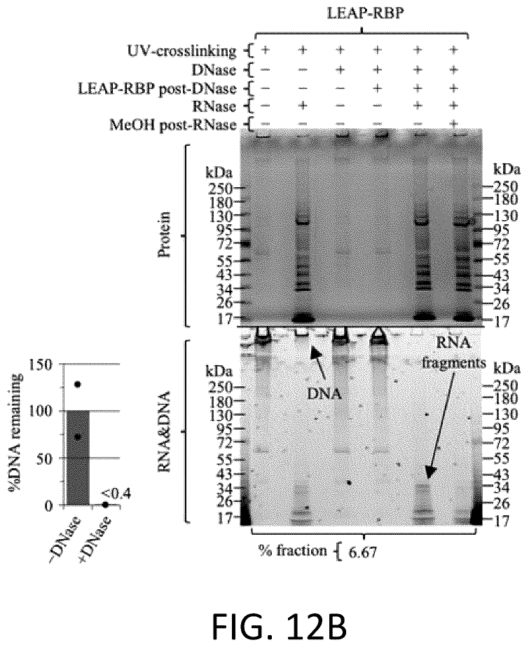

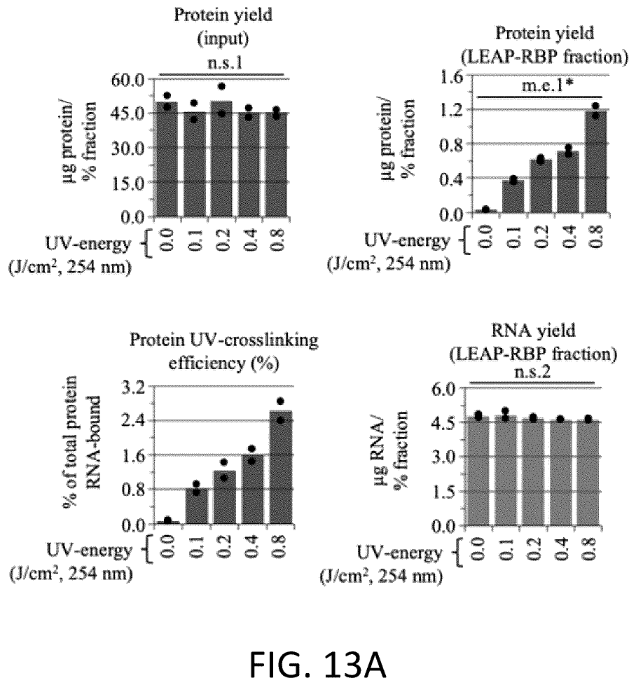

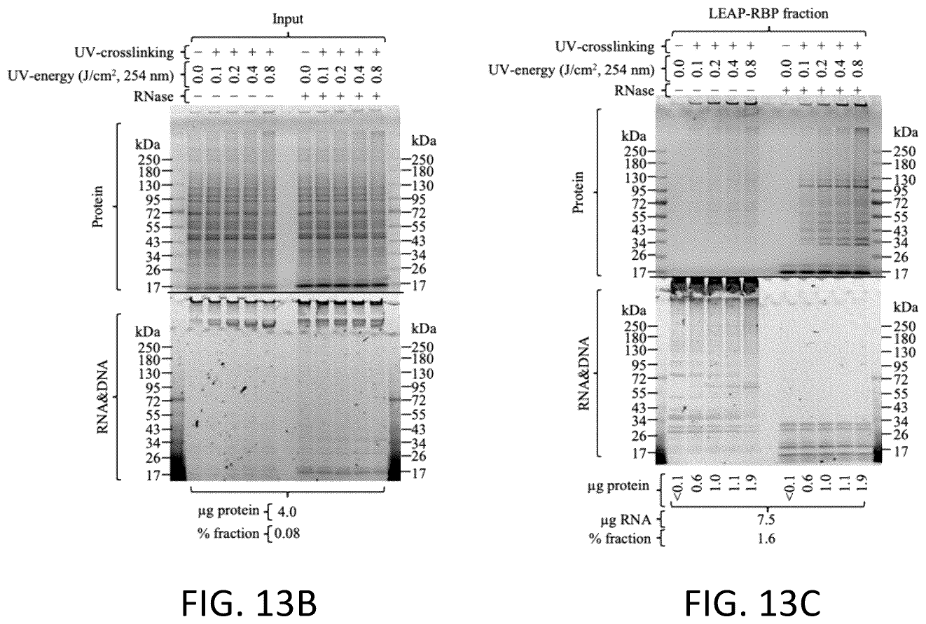

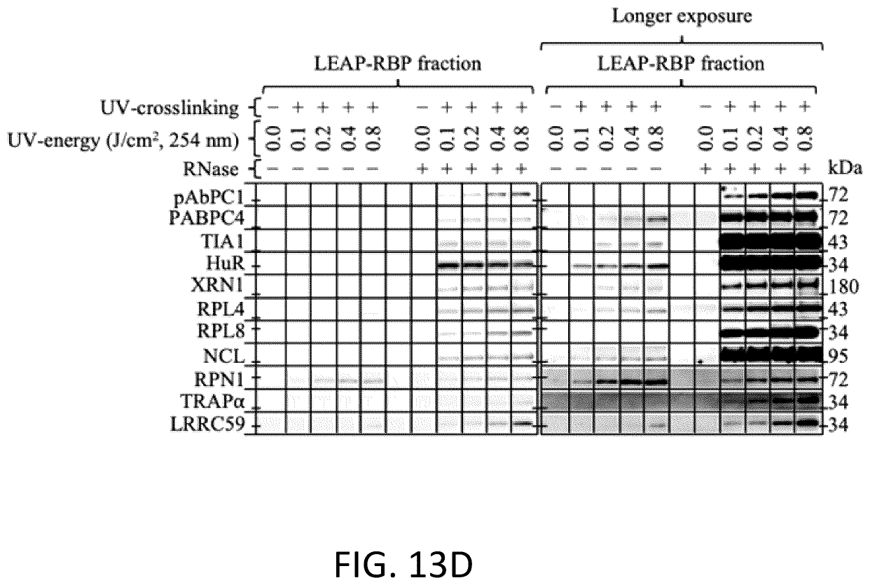

By virtue of high specificity, LEAP-RBP did not recover detectable protein from the final AGPC interphase suspension of non-crosslinked cells (FIG. 12A). This finding allowed for determination of whether protein recovery in the final AGPC interphase suspension of UV-crosslinked cells is restricted to protein-RNA adducts and/or can be UV-irradiation (signal)-dependent but independent of RNA cross-linking. In these experiments, LEAP-RBP was applied to AGP input suspensions containing UV cross-linked or non-cross-linked cells (FIG. 3a, b). Here the identification of contaminant DNA (red box; FIG. 3a) prompted development of a DNA depletion step utilizing a second LEAP step to further enhance S/N (FIG. 12B) Recovery of protein in the AGPC interphase was primarily UV-dependent, with a small but significant difference between crosslinked samples prepared using a single LEAP and those prepared using the full protocol noted above (FIG. 3a). RNA recovery was mainly dependent on sample prep although UV-dependent following six AGPC extractions. Comparisons of the fractions prepared using the different protocols revealed similar RNase-sensitive protein profiles, thereby demonstrating that all RNA-bound protein partitions to the AGPC interphase (FIG. 3a, b). Remarkably, and as a clear demonstration of the specificity and selectivity of LEAP-RBP for protein-RNA adducts, the S/N of most RBPs isolated by direct LEAP-RBP treatment of AGP input suspensions were comparable to those isolated following six AGPC extractions (gold boxes; FIG. 3b). Together, these data establish the efficacy of the LEAP-RBP method and suggest that recovery of noise is signal-dependent, not RNA-dependent (red boxes; FIG. 3b). In subsequent experiments, RNA-free RBP recovery from AGP input suspensions was observed to be dependent on UV-dose and independent of total RNA and protein quantity (FIG. 13^{a-c} and Supplementary Note⁴).

### Enhanced S/N Decreases UV-Enrichment* Specificity

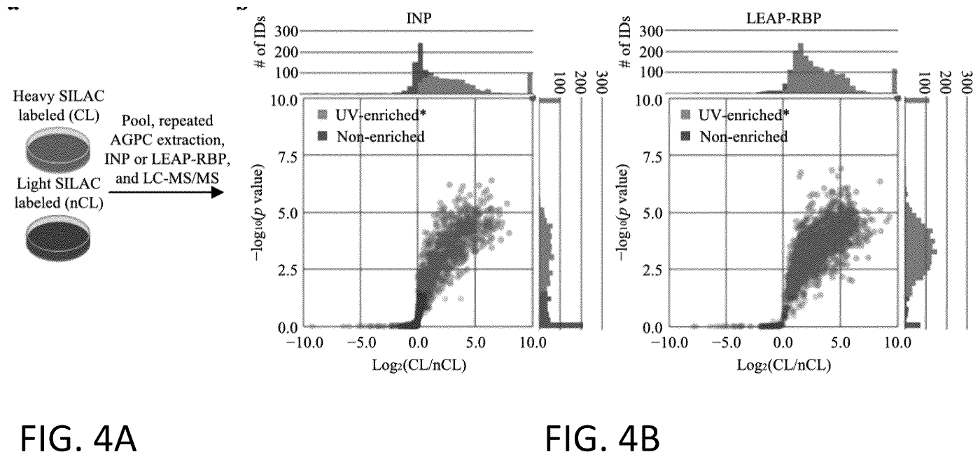

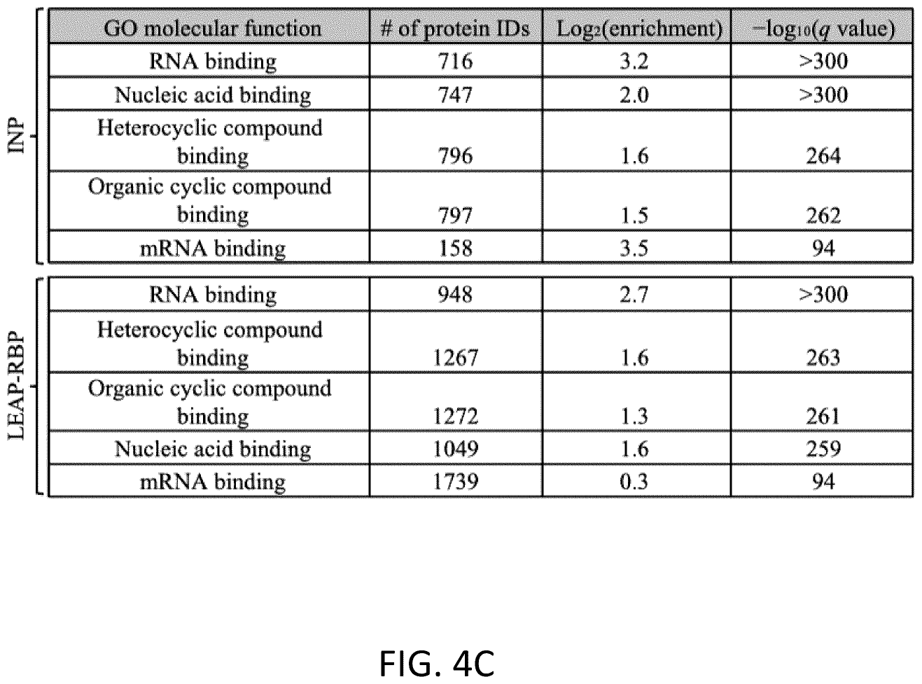

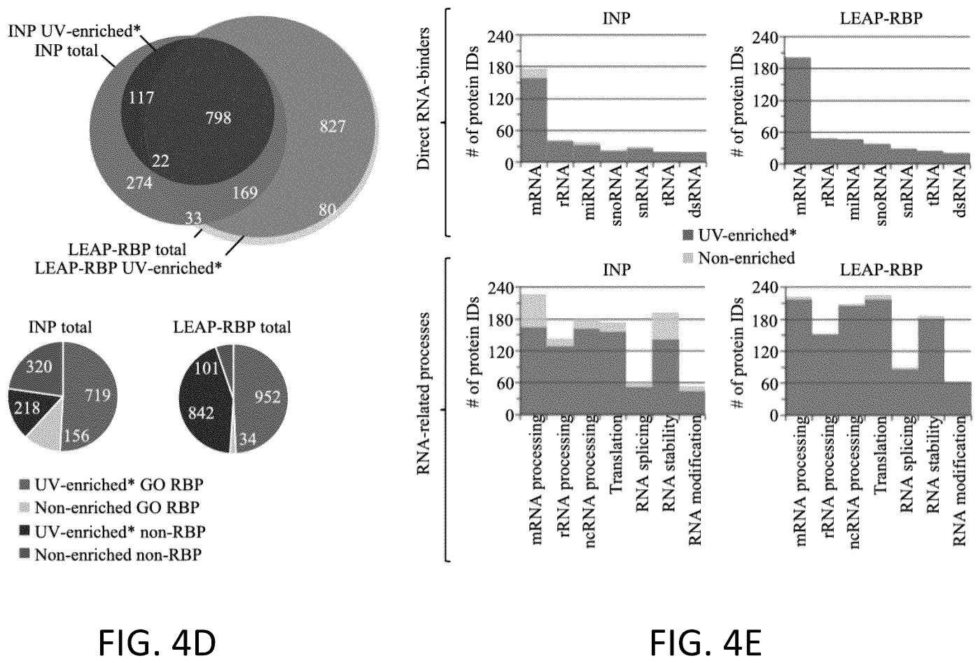

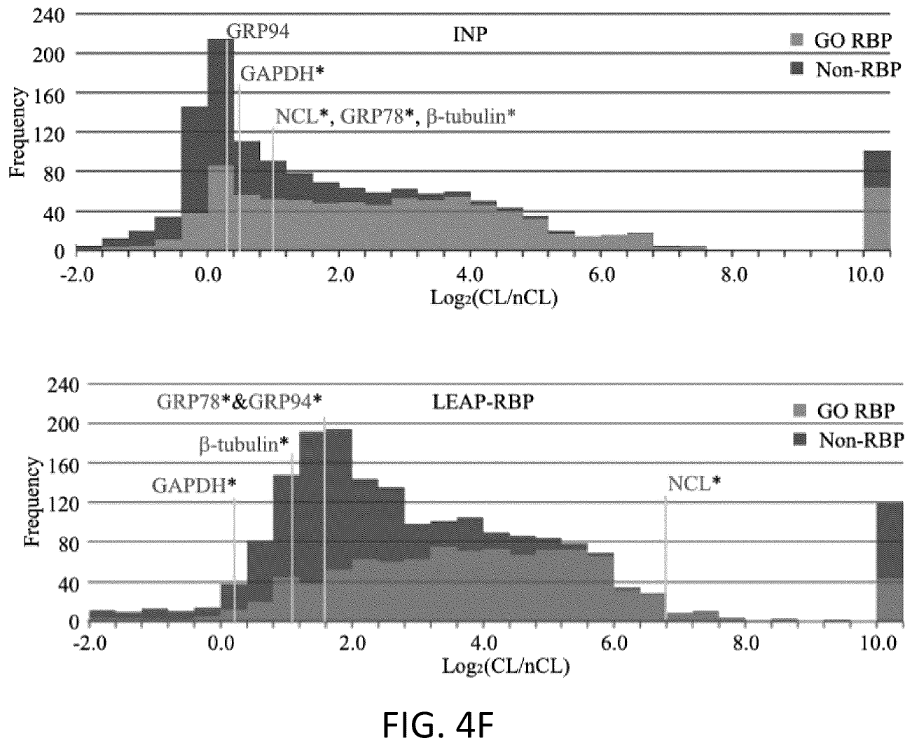

The improvements in S/N conferred by LEAP-RBP provided an opportunity to determine the effect of enhanced S/N on UV-enrichment* specificity for GO-annotated RBPs, where asterisks denote statistical significance. To this end, heavy SILAC-labeled crosslinked (CL) and light SILAC-labeled non-crosslinked (nCL) cells were pooled prior to processing to accurately quantify UV-dependent free protein recovery by LC-MS/MS and evaluate S/N (FIG. 4a and Supplementary Notes⁵ and ⁶). UV-enriched* proteins were identified by generating log₂(CL/nCL) ratios with sum peptide intensities (SPI) as shown in Eq. (2) and testing against the null hypothesis that log₂(CL/nCL)=0 (FIG. 4b).

$$C L / nCL = {SPI}_{CL} / {SPI}_{nCL} \qquad ( 2 )$$

For comparative purposes, INP isolation was performed on parallel samples. As shown in FIG. 4c, both INP and LEAP-RBP methods enriched for GO-annotated RBPs, but LEAP-RBP identified nearly twice as many UV-enriched* proteins (1794 vs 937) including 682 of the 719 UV-enriched* and 106 of the 156 non-enriched RBPs identified by INP (FIG. 4d). Notably, the LEAP-RBP method identified a greater number of UV-enriched* RBPs that bind all classes of RNA and/or regulate various RNA processes (FIG. 4e). However, only 270 of the 996 proteins exclusively UV-enriched* by LEAP-RBP were GO-annotated RBPs, leading to a paradoxical decrease in UV-enrichment* specificity as compared to the INP method (719/937 vs 952/1794). Exploring this further, a large cluster of UV-enriched* proteins was observed, many lacking prior GO-annotation (i.e., non-RBPs), which were largely absent in INP fractions (FIG. 4f). These may represent false-positives arising from low-frequency, non-specific UV cross-linking which are UV-enriched* by the LEAP-RBP method by virtue of its high selectivity for protein-RNA complexes (FIG. 13^{b} and Supplementary Note⁷)^{29_31}. As a relevant example, the ER chaperone GRP94 was detected by immunoblot in INP fractions as an RNase-insensitive band but was exclusively UV-enriched* by LEAP-RBP method while undergoing a 50-fold decrease in abundance. Non-RBPs (GAPDH, GRP78, and β-tubulin) were UV-enriched* and undetectable by SRA in both fractions (FIGS. 2b and 4f), highlighting the utility of this orthogonal validation method for assigning RNA-binding activity to UV-enriched* proteins and providing a method for identifying likely low-frequency cross-linking events (“Methods”).

### Enhanced S/N Allows the Detection of RBP-RNA Occupancy Dynamics

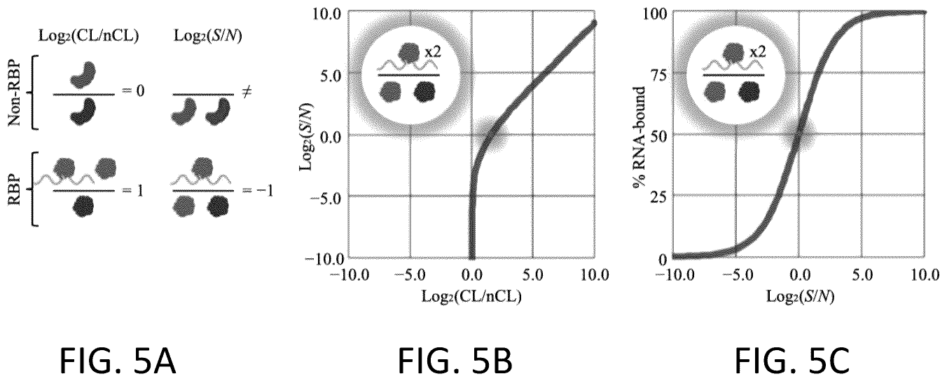

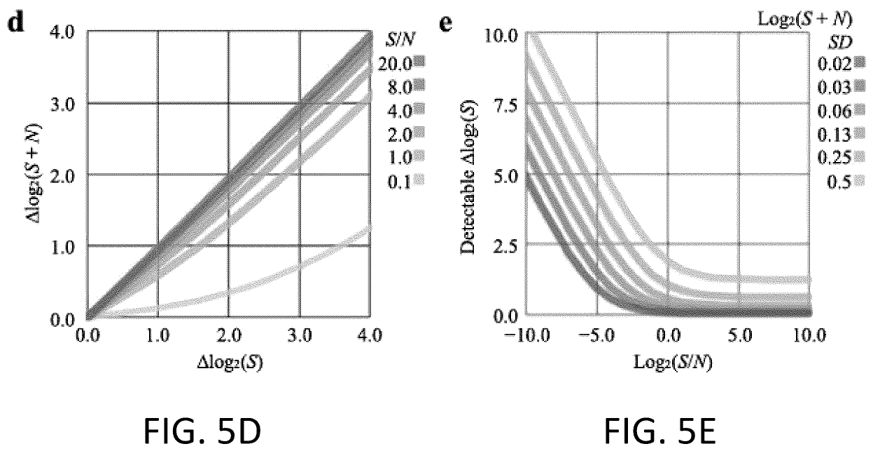

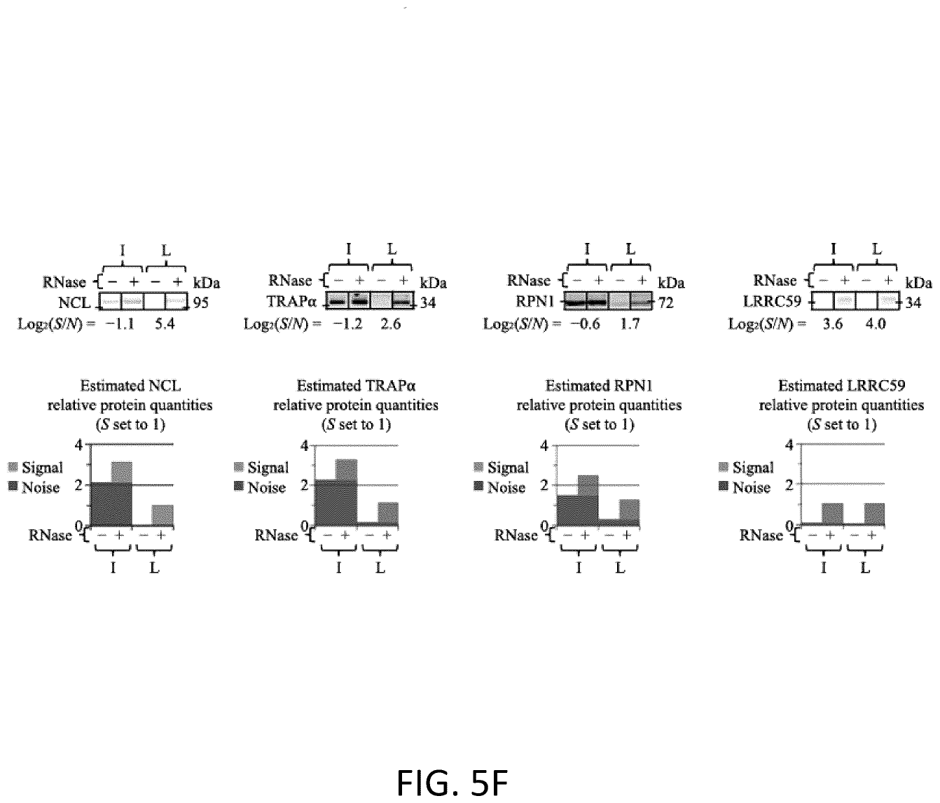

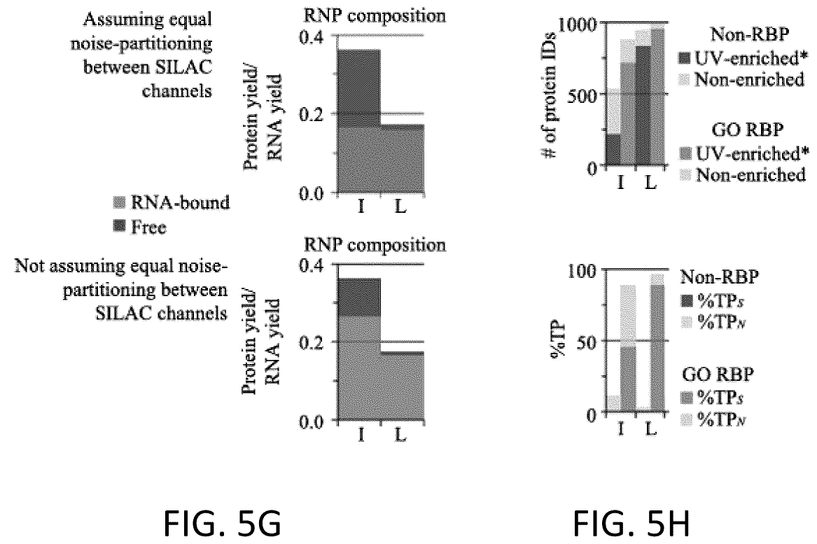

One observation from the SILAC LC-MS/MS studies noted above is that while CL/nCL ratios provide a measure of UV-dependent enrichment, S/N ratio determinations reveal RNA-bound protein contributions across SILAC channels. This relationship is depicted in FIG. 5a and described by Eq. (3).

$$S / N = {SPI}_{S} / {SPI}_{N} = ( {SPI}_{CL} - {SPI}_{nCL} ) / ( 2 * {SPI}_{nCL} ) \qquad ( 3 )$$

As graphically depicted in FIG. 5b, c, a threefold enrichment or log₂(S/N) ratio of 0 indicates equal amounts of RNA-bound (S) and unbound (N) counterparts (50% RNA-bound) while a log₂(CL/nCL)<0 indicates an absence of RNA-binding activity (when S=0, N=B or background); this relationship is described by Eq. (4):

$$\% R N A - bound = S / ( S + N ) * 100 \qquad ( 4 )$$

As is apparent in the LC-MS/MS analysis, S/N is inextricably linked to the ability to detect a change in observed quantity Δ log₂(SPI_{O}) in response to a change in RNA-bound quantity Δ log₂(SPI_{S}). This relationship is depicted in FIG. 5d, illustrating a large decrease in statistical power for proteins displaying negative log₂(S/N) ratios; these relationships are described by Eqs. (5-7) (FIG. 5e, “Methods”).

$$Δ {log}_{2} ( O ) = {{log}_{2} ( S + N )}_{final} - {{log}_{2} ( S + N )}_{initial} \qquad ( 5 )$$

$$Detectable {Δlog}_{2} ( O ) = {t}_{0.95} * SEM of {log}_{2} ( O ) \qquad ( 6 )$$

$$Detectable {Δlog}_{2} ( S ) = {log}_{2} ( {2}^{∧} detectable {Δlog}_{2} ( O ) - N / O ) - {log}_{2} ( S / O ) \qquad ( 7 )$$

This analysis reveals that RBPs displaying different S/N ratios can be UV-enriched* but the ability to detect Δ log₂(S) could differ substantially. These concepts are illustrated by comparing the RNase sensitivity (S/N) of RBPs by SRA with their SILAC LC-MS/MS-derived log₂(S/N) ratios. In principle, RNase sensitivity represents a change in RNA-bound quantity (|S|) when noise is constant (N_{untreated}=N_{RNase}); Eq. (1). This is analogous to Eq. (5) if Sᵢₙᵢₜᵢₐₗ=0 and Nᵢₙᵢₜᵢₐₗ=N_{final}. Experimental examples of these relationships are depicted in FIG. 5f, where both the INP and LEAP-RBP methods identified NCL, and the ER membrane proteins RPN1 and TRAPα as UV-enriched*, but RNase sensitivity was more clearly evident in the LEAP-RBP fractions, which also display higher log₂(S/N) ratios by SILAC LC-MS/MS. In contrast, RBPs displaying comparable RNase sensitivity (e.g., the ER membrane protein LRRC59) have comparable log₂(S/N) ratios. Therefore, a change in RNA-bound quantity (S) for RBPs such as NCL, RPN1, and TRAPα will have a larger effect on their observed quantity (O) in LEAP-RBP fractions by LC-MS/MS (Supplementary Notes⁵ and ⁶)

### LEAP-RBP Displays High Method Specificity for RNA-Bound RBPs

As illustrated in FIG. 1b, e, current AGPC methods are hampered by considerable noise contributions; the interphase fractions contain many RNase-insensitive (free) proteins evident by SRA and Coomassie Blue staining. In contrast, AGPC interphase fractions enriched by the LEAP-RBP protocol would be expected to be both depleted in free proteins and thus contain a higher percentage of RNA-bound protein. This hypothesis was tested on INP and LEAP-RBP fractions using the Total Protein Approach (TPA), which provides protein abundance as a percentage of the total protein in the sample (total SPI), as described by Eq. (8)^{32_34}.

$$\% TP = ( SPI / total SPI ) * 100 \qquad ( 8 )$$

By extension of Eqs. (3) and (4), TPA can be used to determine the abundance of RNA-bound (% TP_{S}) and free proteins (% TP_{N}) as a percentage of total SPI, as described by Eqs. (9-11).

$$\% {TP}_{S} = {SPI}_{S} / {SPI}_{O} * \% TP \qquad ( 9 )$$

$$\% {TP}_{N} = \% TP - \% {TP}_{S} \qquad ( 10 )$$

$$\% {TP}_{N} = \% {TP}_{B} when S = 0 \qquad ( 11 )$$

Cumulatively, % TP_{S} and % TP_{N} represent the abundance of total RNA-bound (total SPI_{S}) and free protein (total SPI_{N}) in the sample. By this approach, 91% of the total protein in LEAP-RBP fractions is RNA-bound compared to 47% for INP. This is consistent with differences in RNP composition (μg protein/μg RNA), though assumes equal noise-partitioning between SILAC channels (FIG. 5g). This finding validates Eq. (3); not assuming equal noise-partitioning overestimates the amount of RNA-bound protein in INP fractions by˜60% (Source Data FIG. 5g). Remarkably, GO-annotated RBPs represent 53% of proteins identified as UV-enriched* in LEAP-RBP fractions but contribute 98.3% of total SPI_{S} (FIG. 5h). By comparison, the % TP_{S} of INP fractions is lower (47), but RBPs still contribute 98.6% of total SPI_{S}.

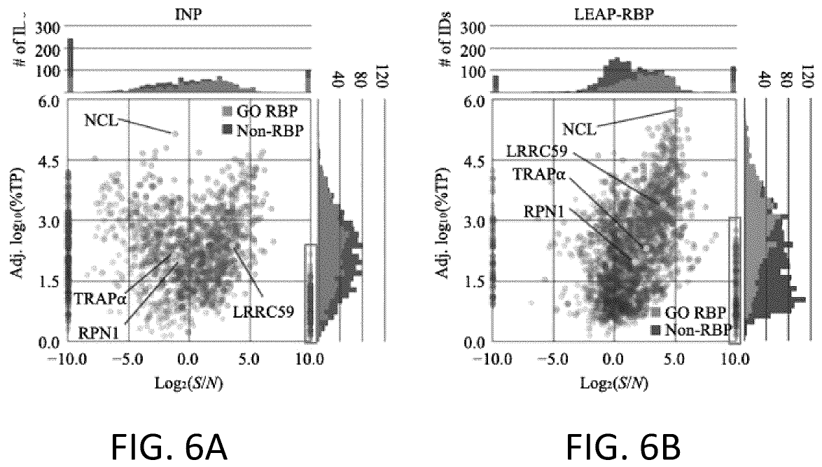

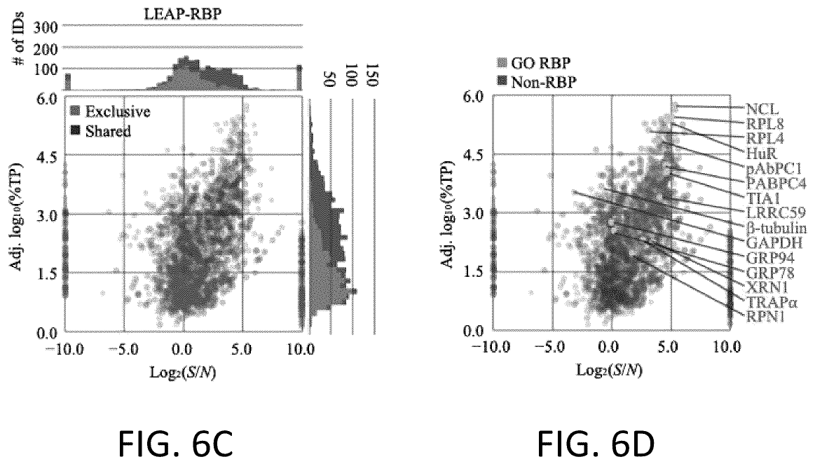

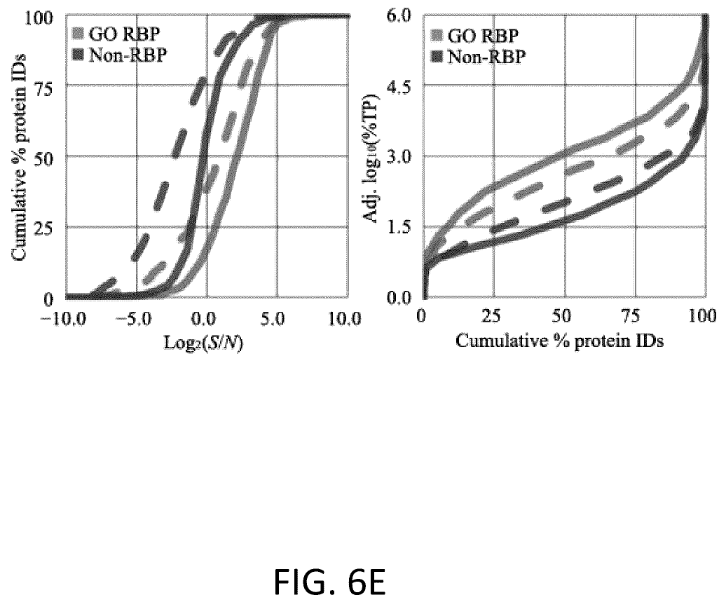

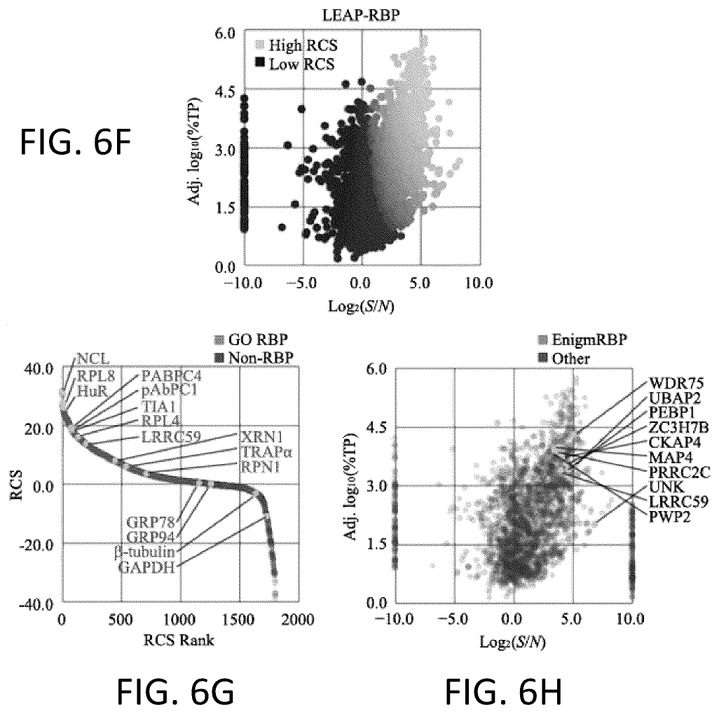

Estimating the abundance of RNA-bound proteins as a percentage of total RNA-bound protein in the sample (total SPI_{S}) can be represented by % TP(S), where the parenthetical text denotes the identity of the total protein population (“Methods”). While % TP_{S} of INP fractions (47) is less than LEAP-RBP fractions (91), both methods recover near 100% of RNA-bound protein (I or L vs M, RNA yield; FIG. 2a); therefore, comparable % TP_{(S)} contributions from RBPs (˜98.5) and non-RBPs (˜1.5) in both fractions is indicative of high UV cross-linking specificity. The disparity in % TP_{S} between LEAP-RBP and INP fractions is not, however, readily illustrated by current analytical methods (FIG. 4b-f). To address this discrepancy, the abundance (log₁₀(% TP)) of RBPs and non-RBPs identified by INP and LEAP-RBP were evaluated as a function of their S/N ratios (log₂(S/N)) (FIG. 6a, b). An increase in % TP_{S} is illustrated by the enhanced enrichment efficiency (S/N) of both RBPs and non-RBPs in LEAP-RBP fractions as compared to INP and higher abundance (% TP) of RBPs relative to non-RBPs (Supplementary Note⁸). The increase in % TP_{S} was attributed to a marked decrease in free protein recovery by the LEAP-RBP method. Consequently, LEAP-RBP provides a lower limit of detection (% TP range), thus resulting in identification of many low-abundance proteins not observed in INP fractions (exclusive; FIG. 6c). Because most of these low-abundance proteins are not GO-annotated as RBPs and UV-enriched*, enhanced S/N results in the paradoxical decrease in UV-enrichment* specificity despite a favorable increase in % TP_{S} (“Methods”).

### High S/N and % TP Distinguish Bona Fide RBPs

As noted above, it was postulated that non-specific UV-crosslinking, combined with the enhanced S/N provided by the LEAP-RBP method, results in the UV-enrichment* of low-abundance non-RBPs. In support of this hypothesis, all non-RBPs (undetectable by SRA) were UV-enriched* but display low S/N ratios and are less abundant than the majority of RNase-sensitive RBPs (FIG. 6d). Indeed, GO-annotated RBPs display significantly higher S/N ratios and were significantly more abundant than non-RBPs by either method (FIG. 6e), but the latter was more apparent with LEAP-RBP (high % TP_{S}). These results demonstrate that UV-enriched* RBPs can be distinguished from UV-enriched* non-RBPs in LEAP-RBP fractions by their enrichment efficiencies (S/N) and abundance (% TP).

To help distinguish high and low confidence RNA binding proteins, a ranking system based on an RBP-confidence score or RCS was used, where RCS=log₂(S/N)*log₁₀(% TP). In practice, RCS ranking prioritizes S/N over protein abundance and places proteins with S/N ratios<1 at lower ordinal rank (FIG. 6f). This scoring system accurately ranked proteins assayed by immunoblot (FIG. 6g) and placed the non-canonical ER RNA-binding protein LRRC59 among the top ten most confident “enigmRBPs” of the 163 detected in LEAP-RBP fractions (FIG. 6h). To facilitate rapid discovery of other non-canonical RBPs by SRA analysis of LEAP-RBP fractions, a full list of identified proteins, their RCS rank, and parameters discussed are included in the provided Source Data.

### LEAP-RBP Allows Robust and Sensitive Detection of Δ Log₂(S)

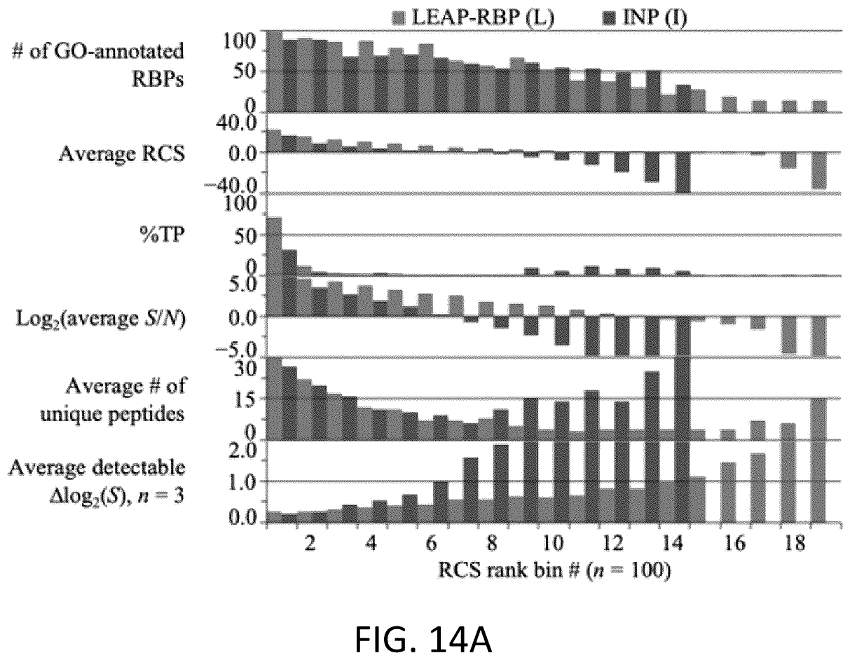

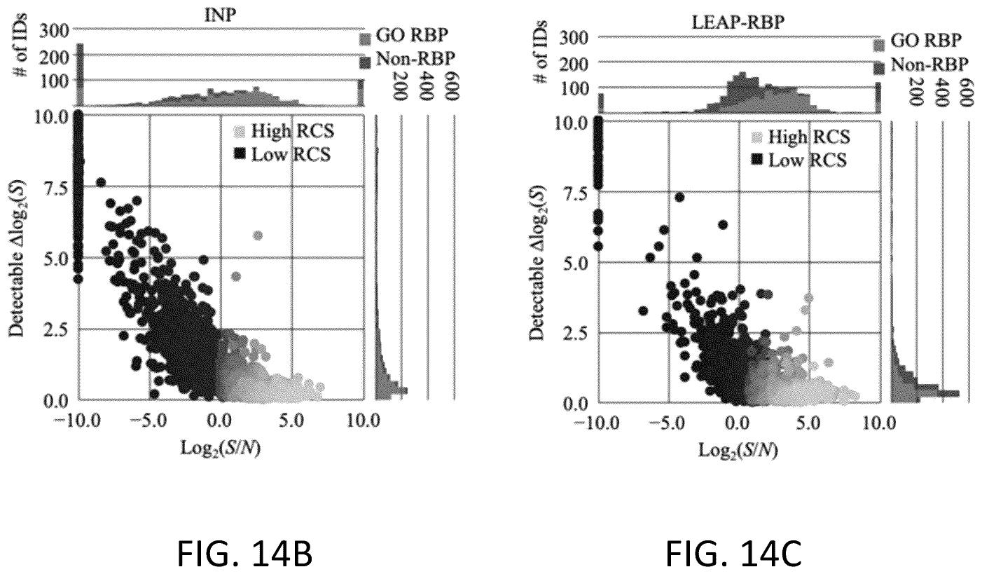

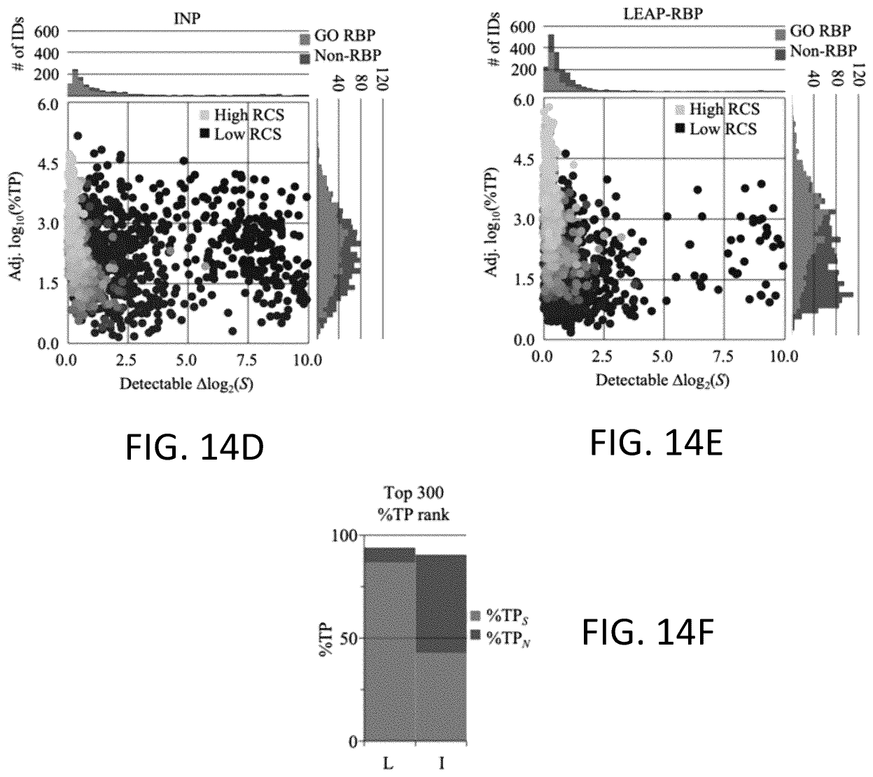

During comparative RBP profiling experiments, enhanced S/N and high % TP_{S} allows accurate assessment of RNA-bound protein abundance. By comparison to INP, which mirrors current AGPC methods, the LEAP-RBP method allows more sensitive detection of Δ log₂(S), representing the fold-change in RNA-bound protein quantity (S) necessary to reject the null-hypothesis that Δ log₂(S+N)=0 (FIGS. 14A-C). This enables the use of stringent S/N-based criteria (S/N>3, 75% RNA-bound) to limit detection of Δ log₂(N) and increase statistical power by reducing multiple hypothesis testing (Supplementary Note 6b, c). In addition, the high % TP_{S} of LEAP-RBP fractions decreases the variability of mean-normalized samples by decreasing free protein contributions (% TP_{N}) of the most abundant proteins in the sample (FIGS. 14D-F). This allows the least computationally intensive and most accurate label-free LC-MS/MS approach for detection of Δ log₂(S) (Supplementary Note 4f).

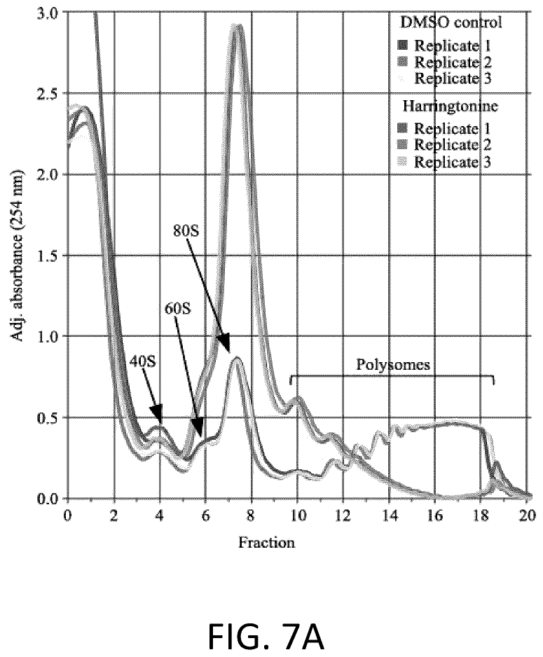

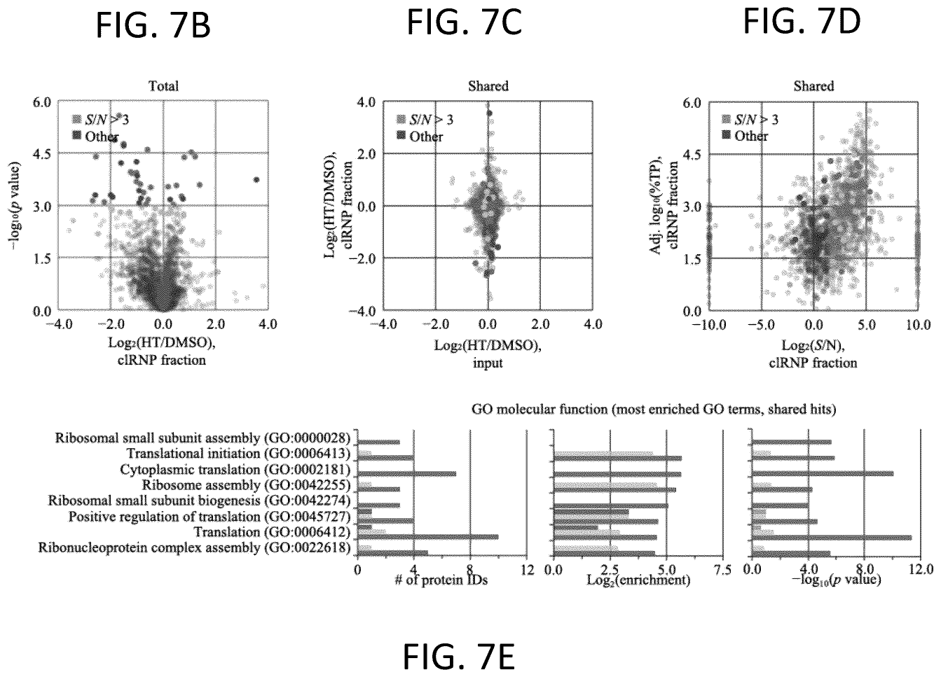

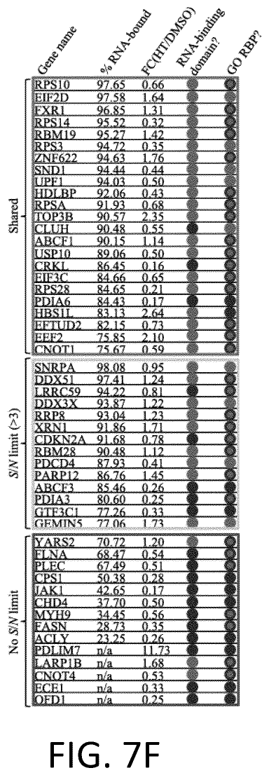

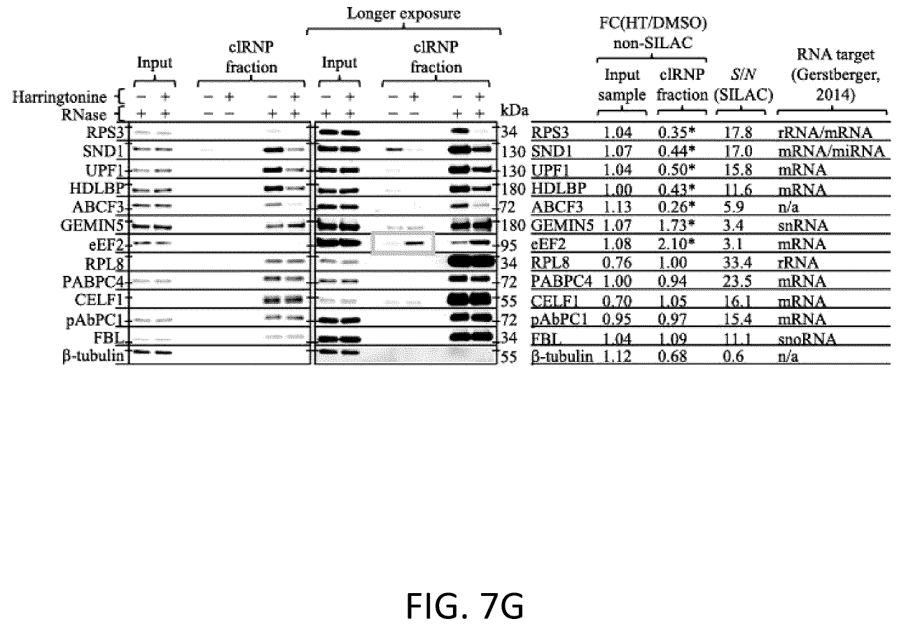

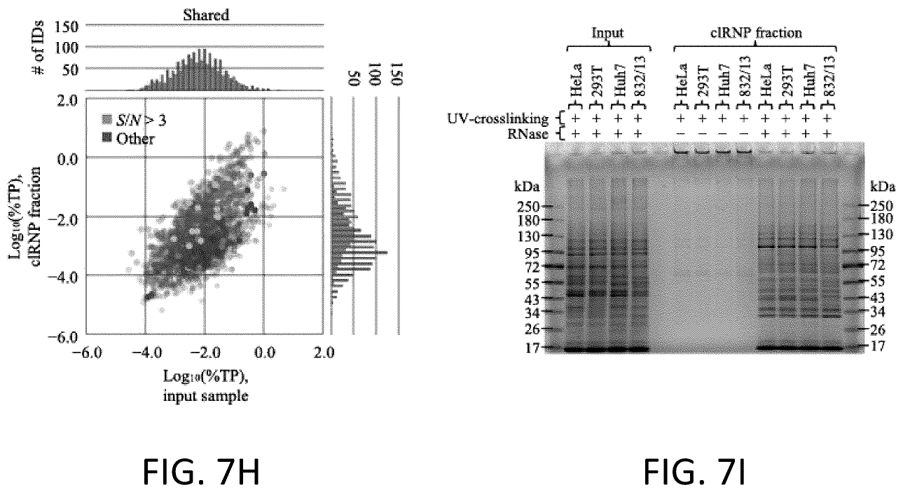

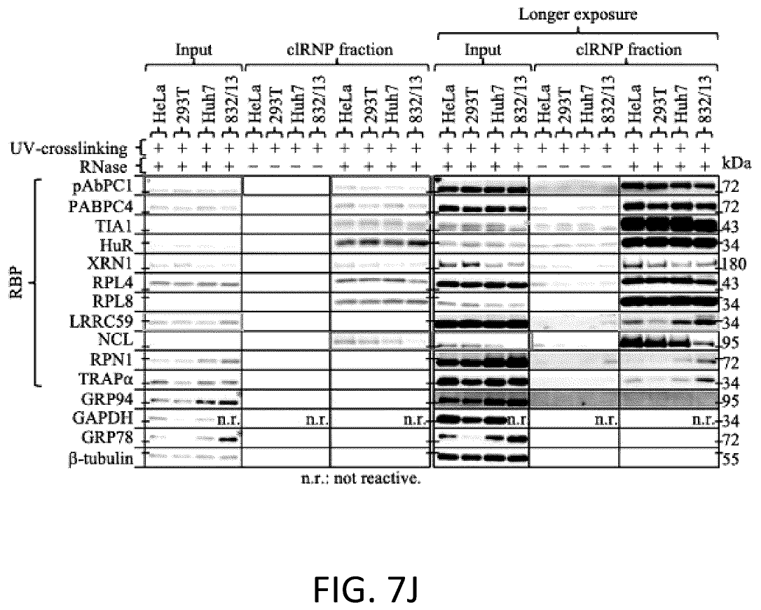

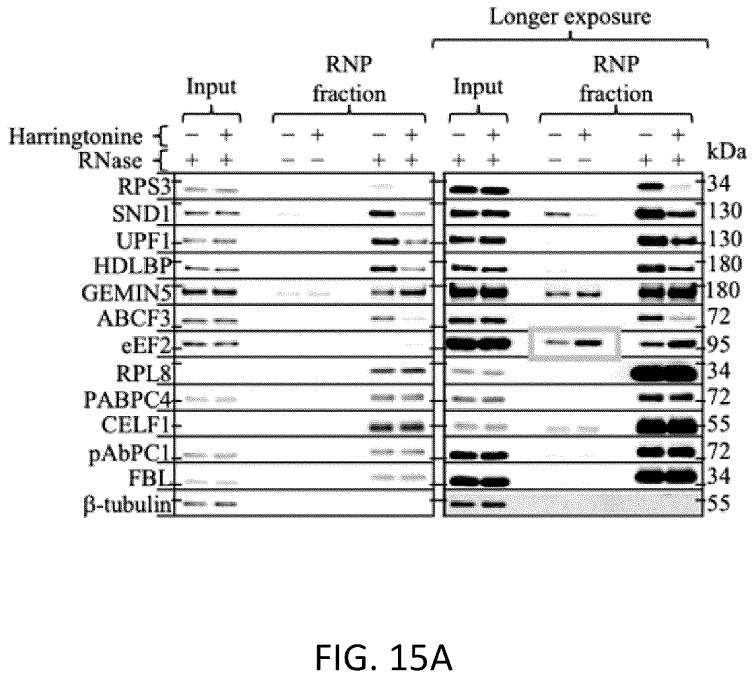

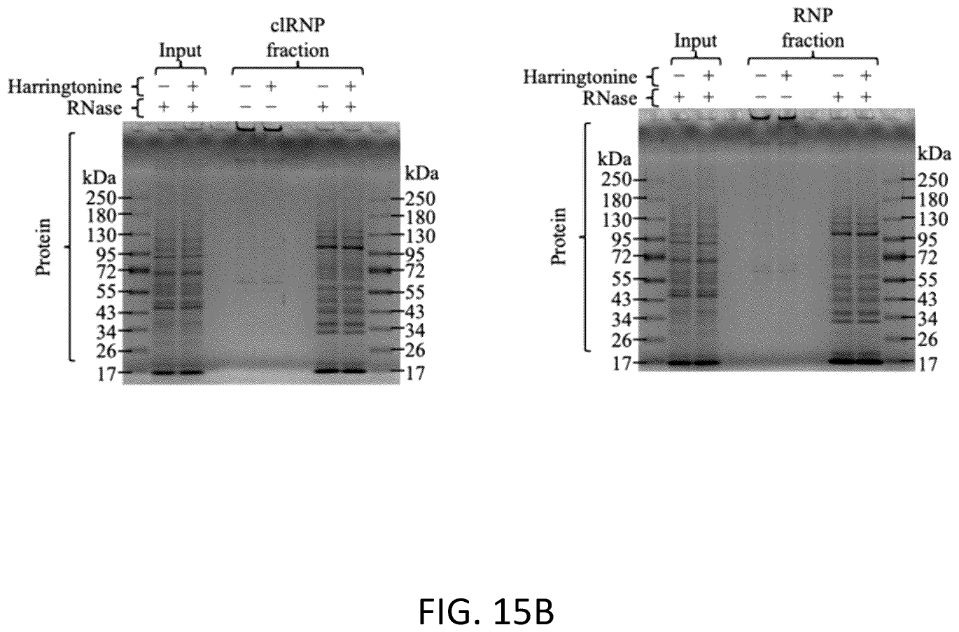

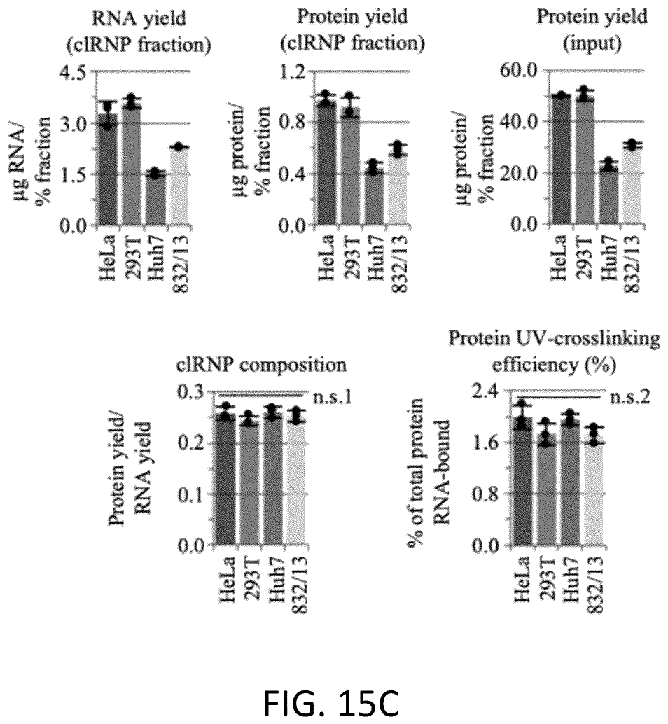

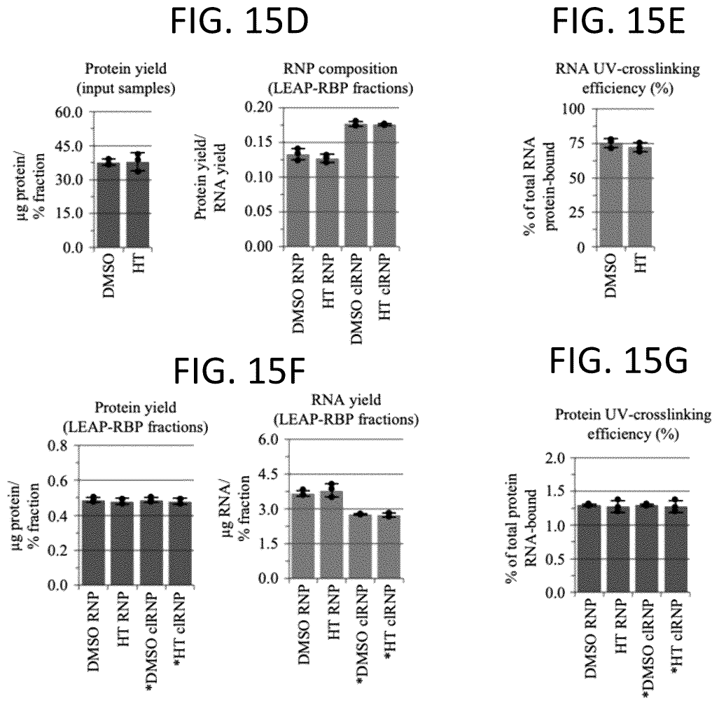

To illustrate these points, a comparative LEAP-RBP experiment was performed to examine the effect of dynamic translatome remodeling on global RBP RNA occupancy states. Using harringtonine (HT), a selective inhibitor of translation initiation, RBPs whose interactions with mRNAs were either sensitive to ribosome occupancy (=translation-state-dependent interactors) or whose mRNA association was not sensitive to ribosome occupancy status (=translation-state-independent interactors were identified). Through inhibitory interactions at the ribosomal A-site, HT induces global polyribosome runoff, to yield monosomes bearing initiation codon locked 80S ribosomes. Harringtonine efficacy was first confirmed by sucrose gradient density gradient polyribosome profiling (FIG. 7a, Methods). HT treatment resulted in the pronounced accumulation of 80S monosome/mRNA complexes. Biological triplicate control and HT-treated cell cultures were then prepared by UV-irradiation, LEAP-RBP fractions isolated, and comparisons of input (total protein) and clRNP fractions (total RNA-bound protein) were performed by LC-MS/MS analysis. Analysis of the proteomic data sets, with or without the proposed S/N limit, identified 23 RBPs displaying a significant change in RNA-occupancy (purple data points; FIG. 7b, c). Application of the S/N-based criteria introduced above significantly improved the specificity and sensitivity of this analysis by limiting inclusion of proteins with significant free protein contributions and revealing additional RBPs with known roles in translation and ribonucleoprotein assembly, as demonstrated by GO analysis (gold vs teal markers; FIG. 7d, e). These findings are further detailed in FIG. 7f, which depicts protein gene name, fractional RNA occupancy, fold-change occupancy in response to HT treatment, and the presence of known RNA binding domains and/or GO-annotated RBP status. It's noteworthy that both the observed fold-change differences as well as the fraction of RBPs whose RNA association is modulated by CDS ribosome occupancy status are generally quite modest. This suggests that CDS translation is largely determined by initiation rate frequency and regulation of ribosome processivity, with RBP-dependent regulation largely biased to 5′/3′ UTR interactions. Subsequent comparisons of the LEAP-RBPs fractions by SRA analysis confirmed that the RBPs whose association with mRNAs was modulated by ribosome loading status were due to differences in RNA-bound abundance (FIG. 7g); comparing RNP fractions yielded similar results (FIG. 15a, b). Notably, RBPs such as RPS3, UPF1, SND1, and HDLBP were identified which were previously reported to display decreased RNA binding following treatment with the oxidative stressor sodium arsenite, and include additional proteins such as ABCF3 whose RNA-binding is unannotated and which was identified exclusively using the more stringent S/N-based criteria (FIG. 7f, g). As a demonstration of the utility of the signal-based analytical approach, RBPs lying near the proposed S/N limit of 3, such as the translation elongation factor eEF2, displayed translation state-dependent differences in free protein recovery (gold box; FIG. 7g, FIG. 15a). The LEAP-RBP method does not bias towards more abundant proteins, as those displaying S/N ratios>3 in LEAP-RBP fractions were found to be significantly less abundant than others in total protein (input) fractions; Kruskal-Wallis, H(1)=7.82, p=0.005; Source Data FIG. 7h).

As an additional demonstration of the utility of LEAP-RBP method for studying context- or cell type-dependent differences in RNA-bound proteomes, a LEAP-RBP analysis was performed on four different cell lines: human cervical cancer cells (HeLa), human embryonic kidney cells (293T), human hepatocyte-derived carcinoma cells (Huh7), and a rat pancreatic insulinoma cell line (832/13) (FIG. 15c). Of note, input and clRNP fractions isolated from the different cell lines displayed discernible differences in total and RNA-bound proteomes by SRA analysis (FIG. 7i). Immunoblot analysis revealed more constitutive RNA-binders (blue boxes), differentially abundant RNA-binders (red boxes), and dynamic RNA-binders (gold boxes) whose relative RNA-bound abundance differs from their relative total abundance (FIG. 7j, Methods). This last category includes the TIA-1a isoform displaying lower total abundance in rat 832/13 cells but comparable RNA-bound abundance (gold asterisk) (Supplementary Note 6d).

Interestingly, integral membrane ER resident RBPs (e.g., LRRC59, RPN1, TRAPα) consistently displayed higher RNA-bound protein abundance in rat insulinoma (pancreatic b) cells (832/13) without a comparable change in total abundance (FIG. 7j). As high secretory capacity cells capable of glucose-stimulated insulin secretion, 832/13 cells have high relative translation at the ER-membrane, further indicated by an increased abundance of the ER-luminal chaperones GRP94 and GRP78 (red asterisk). It is possible that their increased RNA-bound protein abundance indicates a regulatory role in translation at the ER and/or increased local interactions with their RNA components (rRNA and mRNA).

Benchmarking RNA-Centric Methods with Signal-Based Metrics

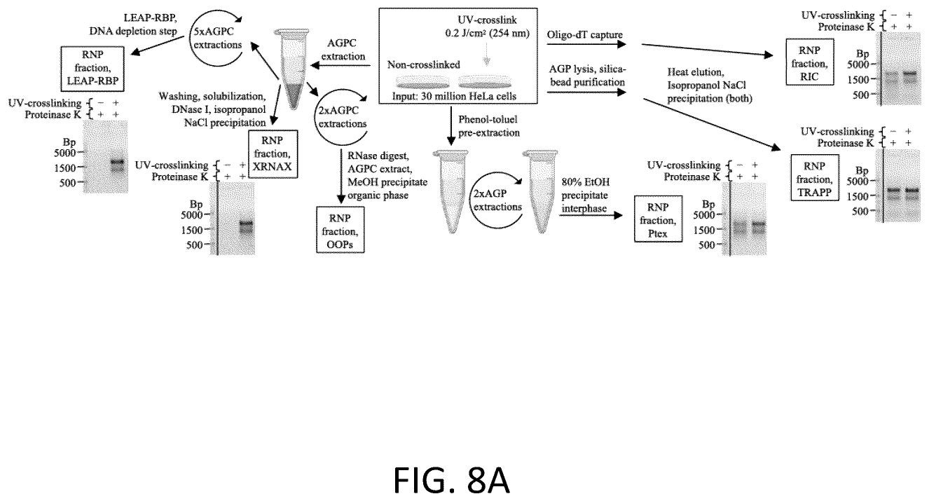

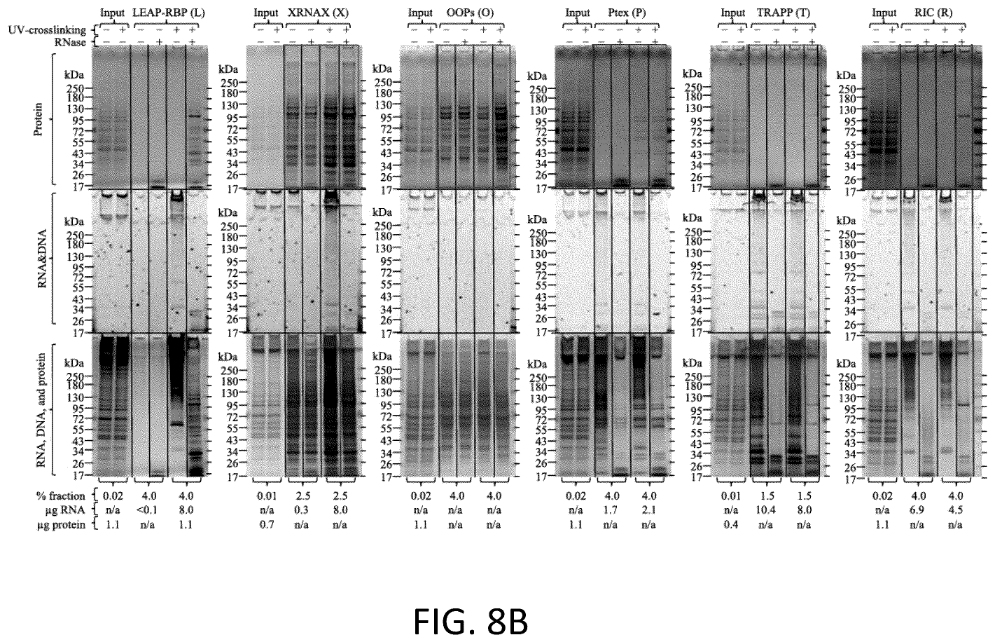

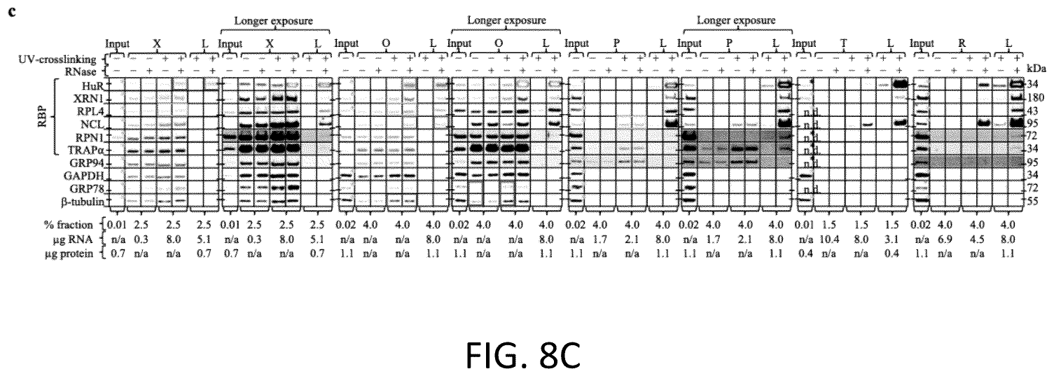

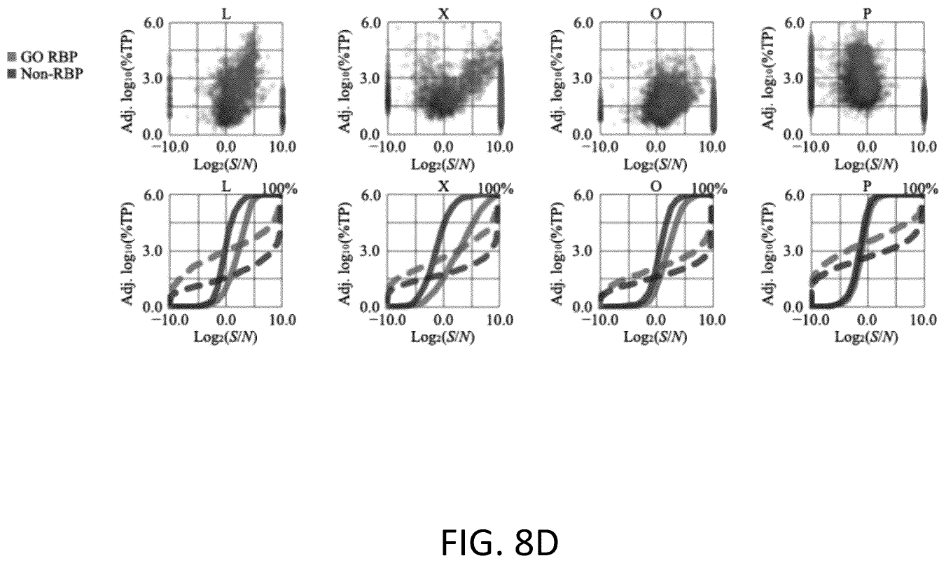

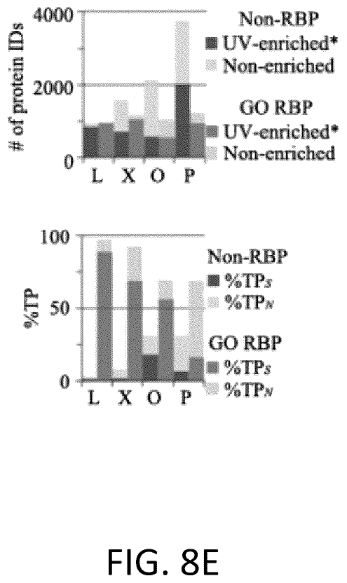

Comparisons of current RNA-centric approaches include overlap (Venn) analysis of UV-enriched* proteins but lack metrics such as S/N or % TP_{S} (Supplementary Note 7). To ascertain the broader utility of the LEAP-RBP method and S/N-based rubrics, benchmark comparisons of LEAP-RBP to multiple methods were performed. These methods included three organic phase separation methods, namely (1) XRNAX (Trendel, J., et al., The Human RNA-Binding Proteome and Its Dynamics during Translational Arrest. Cell, 2019. 176(1-2): p. 391-403 e19.), (2) OOPs (Queiroz, R. M. L., et al., Comprehensive identification of RNA-protein interactions in any organism using orthogonal organic phase separation (OOPS). Nat Biotechnol, 2019. 37(2): p. 169-178), and (3) Ptex ((Castello, A., et al., Insights into RNA biology from an atlas of mammalian mRNA-binding proteins. Cell, 2012. 149(6): p. 1393-406.) LEAP-RBP was also compared to one solid phase separation method (TRAPP) (Urdaneta, E. C., et al., Purification of cross-linked RNA-protein complexes by phenol-toluol extraction. Nat Commun, 2019. 10(1): p. 990), and one affinity-based separation method (RIC) (Shchepachev, V., et al., Defining the RNA interactome by total RNA-associated protein purification. Mol Syst Biol, 2019. 15(4): p. e8689; Hoefig, K. P., et al., Defining the RBPome of primary T helper cells to elucidate higher-order Roquin-mediated mRNA regulation. Nat Commun, 2021. 12(1): p. 5208; Perez-Perri, J. I., et al., Discovery of RNA-binding proteins and characterization of their dynamic responses by enhanced RNA interactome capture. Nat Commun, 2018. 9(1): p. 4408). Except for RIC, which selects for poly(A) RNA-binding proteins, these methods aim to isolate total RNA protein interactomes. RNP fractions were isolated from UV-crosslinked and non-crosslinked cells according to each of the published methods (FIG. 8a, Supplementary Note 9); S/N, % TP_{S}, and yield were evaluated by SRA analysis of RNP fractions (FIG. 8b, c) and the findings were compared with available MS data (FIG. 8d, e). Of note, UV-dependent enrichment of free protein is expected to increase non-specific (i.e., non-RBP) % TP(S) contributions during non-SILAC LC-MS/MS experiments (Supplementary Note 7e, 8).

By SRA analysis, XRNAX and OOPs display low to moderate UV-dependent enrichment of free protein (blue boxes; FIG. 8b) and low S/N without signal loss (gold and red boxes; FIG. 8c). Ptex shows moderate UV-dependent enrichment (blue boxes; FIG. 8b), but recovered protein is not RNA-bound (gold boxes; FIG. 8c). This is consistent with prior data (Supplementary Note 8b), and available MS data (non-SILAC) indicating low % TP_{S} (23.2) and high non-specific % TP(S) contributions (28.4; FIG. 8e). XRNAX utilizes partial tryptic digestion and repeated TiO₂/SiO₂ enrichment to further enrich RNA-bound peptides prior to SILAC LC-MS/MS. This procedure effectively enhances S/N, resulting in a favorable increase in % TP_{S} (70.5, % TP_{(S), non-RBPs}=2.6; FIG. 8d, e); however, proteins such as b-tubulin or GRP94 remain UV-enriched* and because of the trypsinolysis step cannot be subsequently orthogonally validated by methods such as SRA. OOPs distinguishes UV-enriched RBPs (gold boxes) from non-RBPs (blue boxes) at the interphase by their RNase-dependent partitioning into the organic phase (FIG. 8c) but continued partitioning of free protein decreases S/N. It should be noted that the reported methodology for the OOPs protocol includes non-SILAC comparisons which are expected to result in non-specific enrichment of free protein (purple boxes; FIG. 8c). Available MS data showing high non-specific % TP_{(S)} contributions (26.5; % TP_{S}=73.5) supports this assessment (FIG. 8d, e).

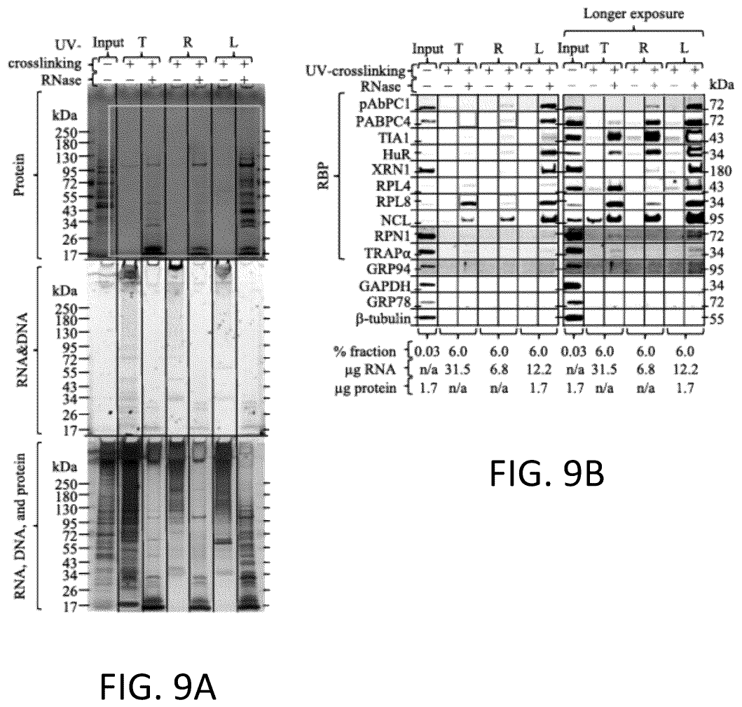

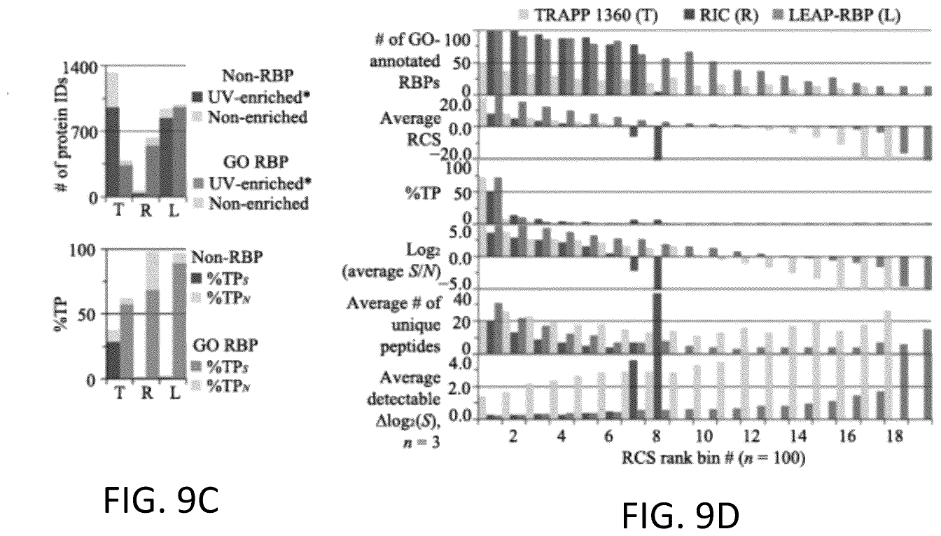

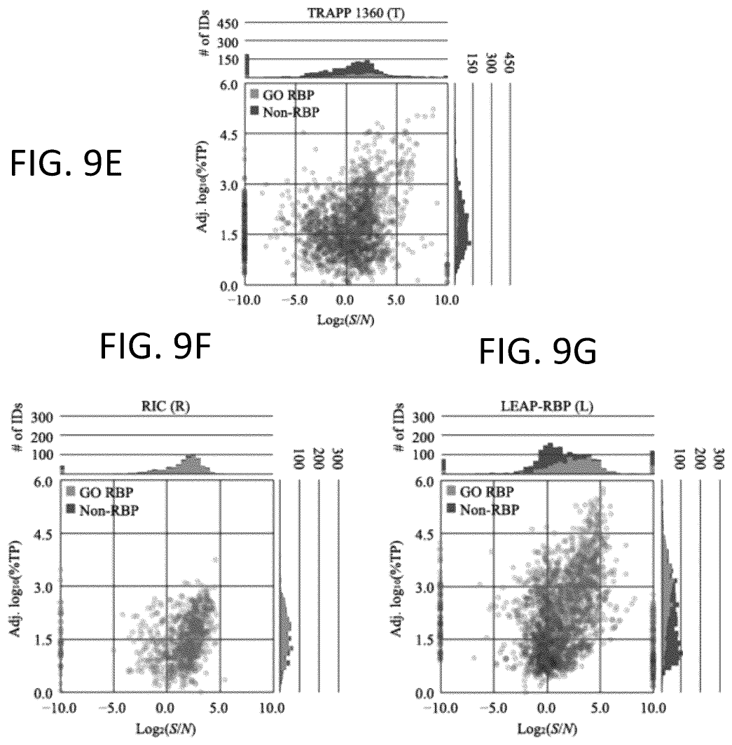

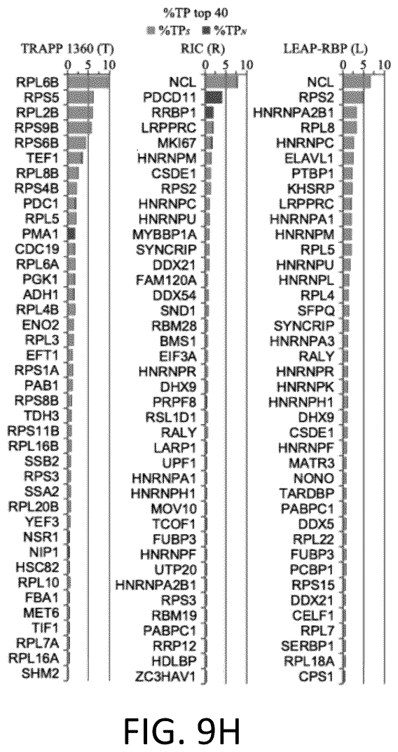

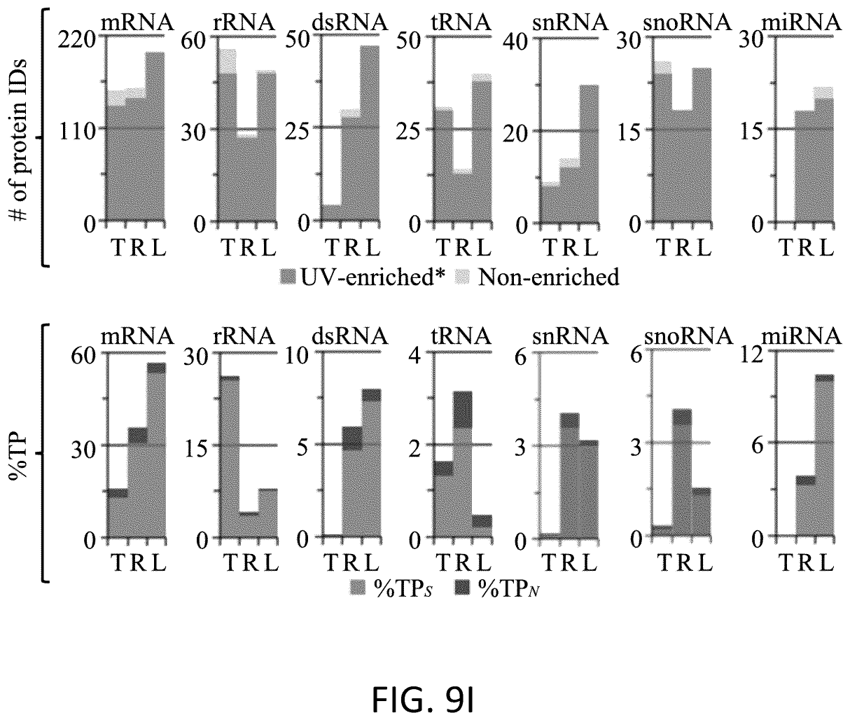

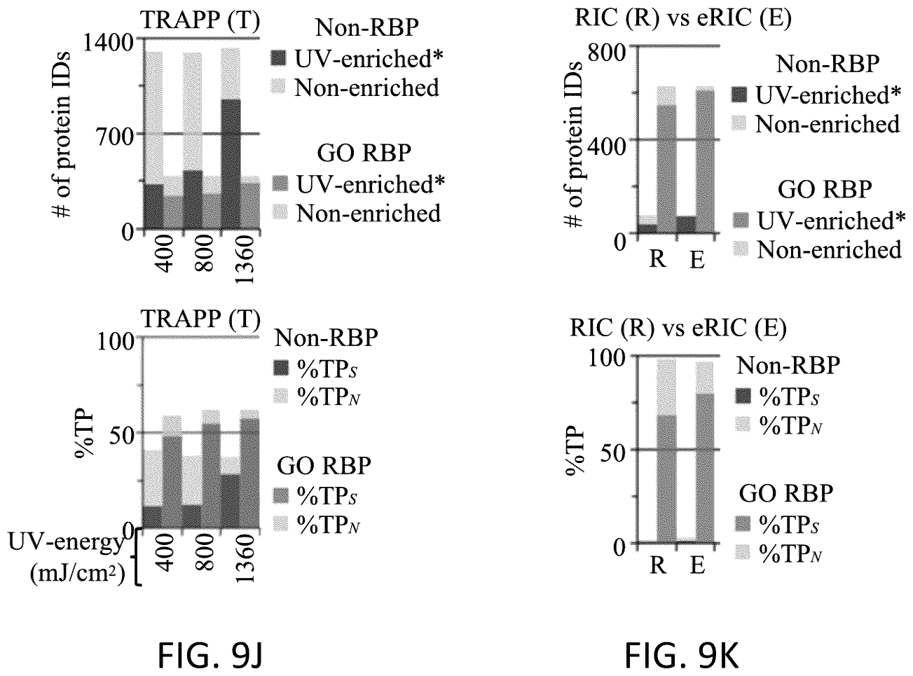

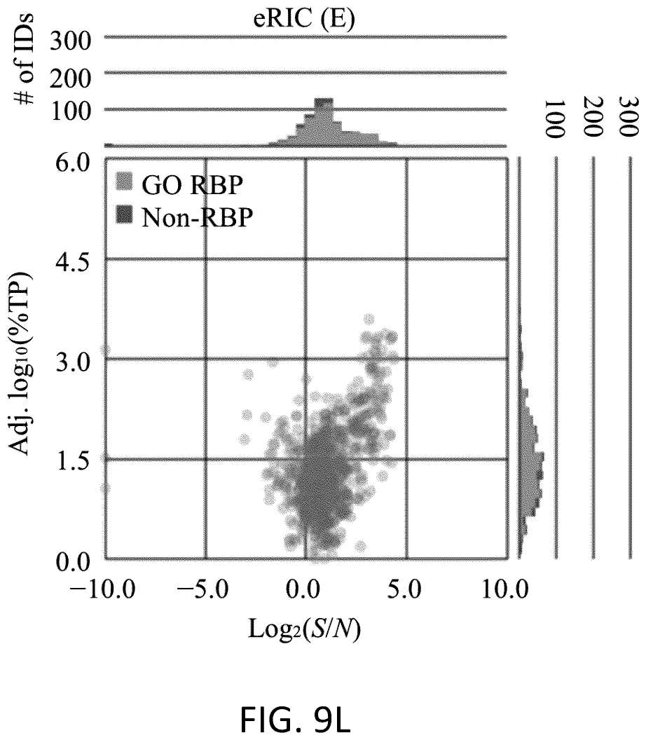

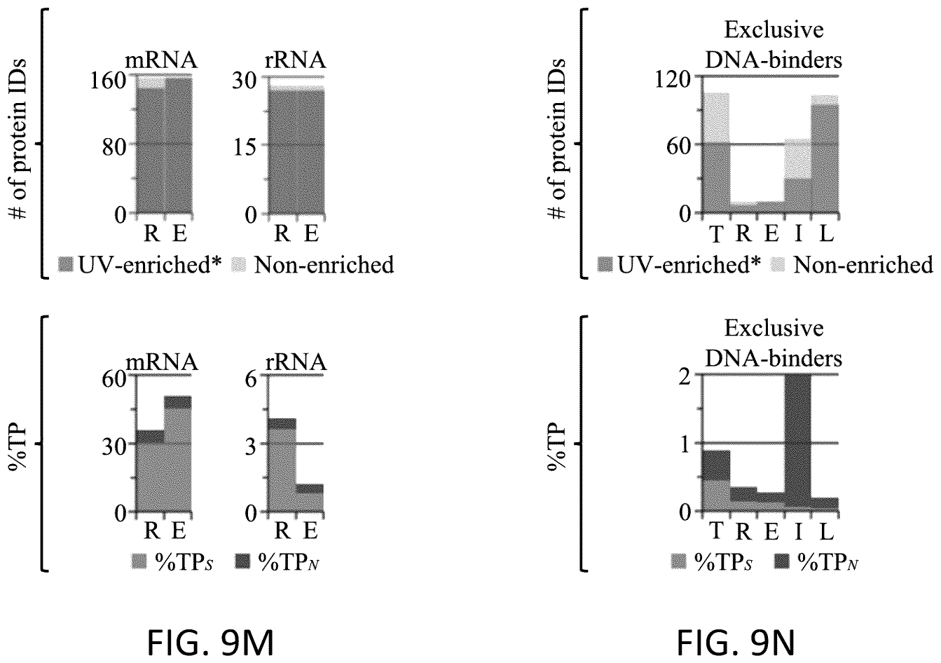

By SRA analysis, both TRAPP and RIC display high UV-dependent enrichment of RNase-sensitive protein (blue boxes; FIG. 8b), and signal-dependent recovery of noise (gold boxes; Source Data FIG. 5c), which are both indicative of high % TP_{S} (Supplementary Note 8a). Comparisons using a higher percentage of RNP fractions isolated from UV-crosslinked cells suggests LEAP-RBP achieves higher S/N, % TP_{S}, and yield than TRAPP or RIC methods (gold box; FIG. 9a, b). Available MS data from RIC (non-SILAC) and TRAPP (SILAC) experiments support these observations and indicates that the LEAP-RBP method provides more sensitive detection of Δ log₂(S) for a greater number of proteins (FIG. 9c-g). Of note, available MS data for TRAPP is from yeast; therefore, non-specific % TP_{(S)} contributions are due to incomplete RBP-annotations (GO:RBP) for ribosomal proteins (Supplementary Note 8b). Curiously, TRAPP shows efficient recovery of RNA-bound ribosomal proteins (blue boxes) but relatively poor recovery of others (red boxes; FIG. 9b), consistent with available MS data (FIG. 9h, i). This RBP-specific signal loss may occur because of the stringent denaturing washes employed in the purification process. The optimal amount of UV-energy (254 nm) for TRAPP was discussed by these authors, based on GO-analysis of UV-enriched* proteins, with “lower doses being potentially less noisy, but at the cost of recovering fewer proteins with annotated functions in RNA biology”. From an S/N perspective, higher UV doses increase % TP_{S} but decrease UV-enrichment* specificity (FIG. 9j).

RIC recovers RNA-bound mRNA binders more efficiently than TRAPP (red boxes; FIG. 9b), but still recovers RNA-bound ribosomal proteins (blue box) and rRNA (FIG. 8a). More stringent protocols utilizing LNA probe capture (eRIC) have been developed to address these concerns (FIG. 9k, l). However, unless rRNA is entirely removed from the sample, UV-enrichment* of rRNA-bound protein is likely to occur. Indeed, ribosomal proteins are less abundant in eRIC fractions compared to RIC (1.2 vs 4.1) but are detected and UV-enriched* (FIG. 9m). A similar trend is observed for exclusive DNA binders which are less abundant in LEAP-RBP fractions compared to other methods but were also UV-enriched* in greater numbers (FIG. 9n). Surprisingly, while RIC and eRIC are aimed at selective recovery of mRNA binders, LEAP-RBP identified a greater number of mRNA binders and they are more abundant in LEAP-RBP fractions (FIG. 9i, m). Nonetheless, ribosomal proteins are less abundant in RIC (4.1) and eRIC (1.2) fractions than LEAP-RBP fractions (7.9). These data indicate that eRIC—and to a lesser extent, RIC—are selective for mRNA binders, while LEAP-RBP provides a comprehensive assessment of the RNA-bound proteome.

LEAP-RBP is shown herein to be a highly selective and cost-efficient method for the purification of RNA-bound protein from biological samples. S/N and % TP_{S} (RNA-bound protein abundance) were identified as key metrics for evaluating RNA-bound protein enrichment and method specificity for RNA-bound RBPs. Practical, experimentally accessible strategies for the accurate determination of in vivo RNA-binding activity and for robust profiling of RNA-bound proteomes at steady-state and following dynamic cell state transitions are provided herein.

A S/N-based comparative analysis of RBP profiling data generated by LEAP-RBP and other RNA-centric methods revealed the complexity and challenges inherent in accurate identification of direct RNA-binders based on their UV-enrichment* and assessment of RNA-binding activity based on protein recovery alone. These method-intrinsic challenges can be compounded by low method specificity and/or non-SILAC comparisons, both of which result in apparent UV-dependent enrichment of free protein. While RBP enrichment methods utilizing SILAC LC-MS/MS and stringent sample washes achieve higher % TP_{S}, the benchmark comparisons performed here reveal both reduced yields and biases in signal recovery which were previously unrecognized. These observations provide insights into why non-poly(A) RNA binders such as ribosomal proteins can represent a large fraction of MS-spectra. The high selectivity of LEAP-RBP achieves high % TP_{S} without the need for high stringency washes, and thus provides a more specific, selective portrait RNA-interactomes.

RNA-binding proteins containing well-established canonical RNA-binding domains display higher S/N ratios and RNA-bound abundance, which greatly simplifies study of their RNA interactome dynamics, largely independent of limitations in existing methods. A primary challenge in the field however is the study of candidate RBPs lacking canonical RNA binding domains, known functions in RNA biology, relatively low UV crosslinking frequencies, and/or significant free protein contributions in phase separation-based RNA-centric methods, all of which can hinder interpretation as well as meta-analysis of RNA interactomes and their dynamic regulation (Supplementary Note 4-6). The signal-based analytical framework described here addresses these limitations and provides experimental avenues for the discovery and study of novel RNA-interactors with previously unknown roles in RNA biology. Non-canonical integral membrane RBP candidates LRRC59, TRAPa, and RPN1, all of which are resident proteins of the endoplasmic reticulum and which may function in mRNA and/or ribosome localization to the ER, are provided herein as a representative example of the utility of LEAP-RBP. Also described herein is the effect of selective reduction in CDS ribosome occupancy status on RNA interactome composition, where global inhibition of translation initiation and ribosome runoff elicited RNA occupancy changes in only a small fraction of the RNA interactome. For those RBPs whose RNA interactions were sensitive to global translation initiation inhibition, differences in RNA bound protein abundances were relatively modest, suggesting that for the supermajority of the RNA interactome, regulatory RBP-RNA interactions are biased to interactions at the 5′ and 3′ UTRs. The successful application of this approach to identify and validate the dynamic responses of bona fide RBPs involved in translation initiation and uncover additional RNA-interactors with previously unknown roles in RNA regulation provides strong experimental evidence of its utility for biological discovery.

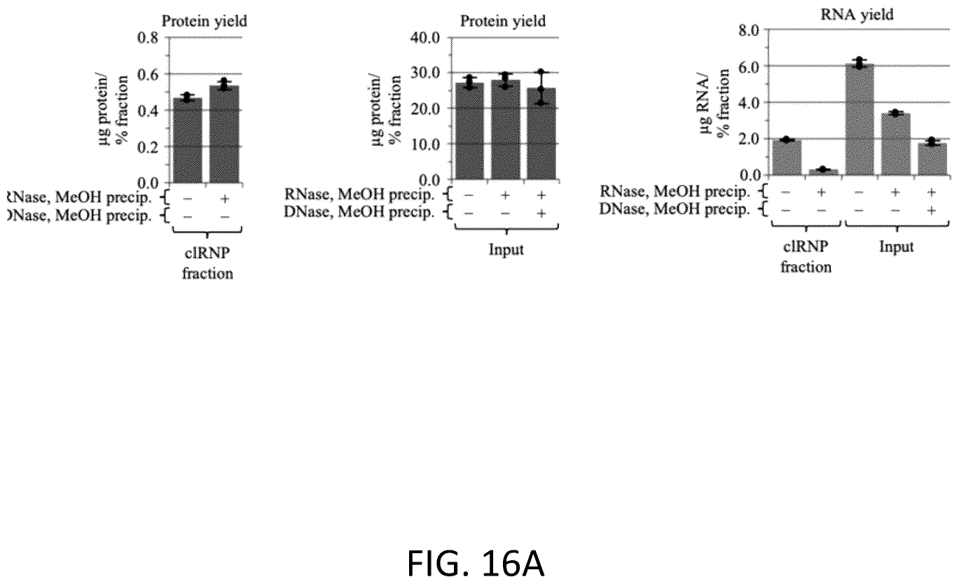

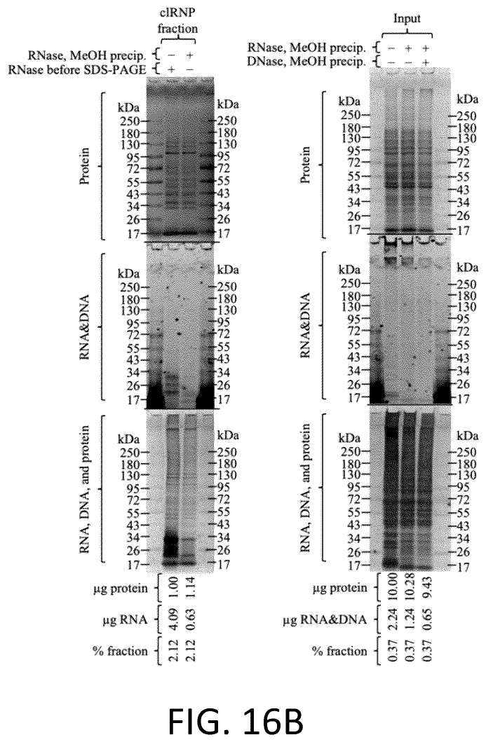

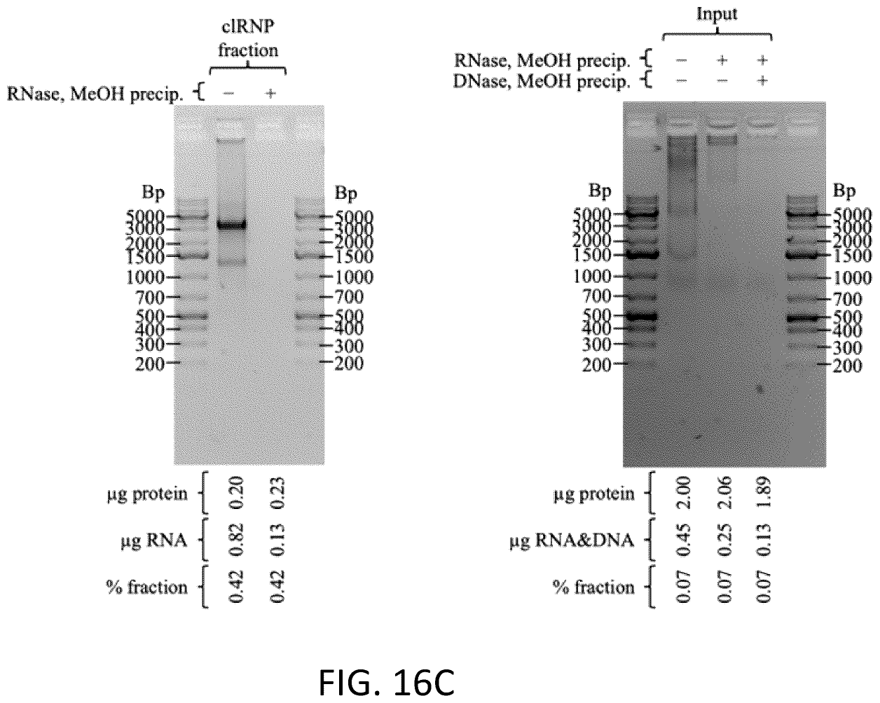

The results presented herein suggest that the number of RNA-binding proteins currently thought to comprise the RNA-interactome (˜4925 human RBPs) and/or those with GO RBP-annotations (˜1693) is an overestimation. LEAP-RBP combined with quantitative proteomic and SRA analysis provides direct experimental evidence of RNA-binding and orthogonal validation of RBP activity. RBP-RNA adduct recovery or low sensitivity (ISI/μg RNA) and/or low S/N can confound detection of many bona fide RBPs by SRA analysis alone (e.g., pAbPC1 and XRN1) (T; FIG. 9b). To that end, high % TP_{S} and efficient, unbiased recovery of RNA-bound protein is important for accurate identification of RNA interactomes and their state change dynamics, and is a goal met by LEAP-RBP (Supplementary Note 3). The high specificity and selectivity of the LEAP-RBP method for RNA-bound protein allows efficient capture of broad-spectrum RNA-interactors from biological samples. Potential applications beyond those demonstrated here including PAR and chemical crosslinking approaches are reasonable to consider using the provided strategies (FIG. 16, Supplementary Note 1-8, Methods).

### Methods

### Methodical and Analytical Framework

A description of sample types, terminologies, quantitative metrics, and analytical approaches are provided in the Supplementary Methods. Analytical approaches: evaluating UV-dependent enrichment and S/N by SDS-PAGE RNase-sensitivity Assay (SRA); estimating RBP-specific UV-crosslinking efficiencies and S/N ratios by SDS-PAGE and immunoblot; evaluating total protein and total RNA-bound protein abundance by SDS-PAGE; MS data analysis; RCS rank analysis.

### Criteria for Assignment of RNA-Binding Activity

Protein displaying CL/nCL ratios>0 in LEAP-RBP fractions by SILAC LC-MS/MS were considered high or low confidence RBPs based on their observed enrichment efficiency (S/N) and abundance (% TP). However, only those displaying discernible RNase-sensitivity by SRA and immunoblot were considered bona fide RNA-binding proteins. Proteins which remained RNase-insensitive by SRA or undetectable were not considered bona fide RBPs regardless of GO-annotation (e.g., GRP94, a GO-annotated RBP). However, because the inability to detect a protein by SRA and immunoblot could be due to their low RNA-bound protein abundance, negative data were not considered formal confirmation of an absence of RNA-binding activity. To this point, validation of RNA-binding activity with LEAP-RBP and SRA requires that RNA-protein interactors are susceptible to UV-crosslinking.

### LEAP-RBP Optimization and Quality Control

The ability of LEAP-RBP to rapidly (<5′) recover total RNA-bound protein from AGP suspensions with near 100% recovery is supported by a lack of quantifiable RNA and RNase-sensitive bands in the unprecipitated fraction by SRA and Coomassie Blue (protein) staining (FIG. 11A). Total RNA-bound protein recovery was further validated by performing repeated LEAP steps without a significant, discernible decrease in protein-bound RNA yield (FIG. 11B, Supplementary Note 1b). The significance of the liquid-liquid interphase during the LEAP step was evidenced by a significant decrease in RNA recovery (30-50%) when the solvents were quickly mixed (FIG. 11C). RNA-dependence of LEAP-RBP was validated by performing LEAP-RBP on RNase treated clRNP fractions resulting in a loss of detectable protein by SDS-PAGE and Coomassie Blue (protein) staining. Additionally, performing LEAP-RBP on proteinase K treated clRNP fractions resulted in recovery of RNA, but not protein (determined by Coomassie Blue staining), thereby demonstrating RNA-centricity (FIG. 11D). Efficiency of DNA depletion and signal recovery during MS sample prep steps were validated by qPCR and SRA analysis (FIG. 12B).

An RNA-seq analysis of small RNA composition was performed to determine if small RNA species are recovered by LEAP-RBP from final AGPC interphase suspensions of UV-crosslinked cells. RNA samples were found to be of high integrity (RIN>9) and contained diverse sRNA species displaying broad genome distributions. Small RNA species were expected to be depleted following repeated AGPC extraction relative to other larger RNA species due to lower UV-crosslinking efficiencies and depletion of free RNA. Therefore, assessing the abundance of different RNA biotypes in clRNP fractions relative to their abundance in total RNA samples was considered uninformative. Nonetheless, SDS-PAGE of LEAP-RBP fractions isolated from AGP input suspensions demonstrate recovery of 60-100 bp RNA species visible as RNase-sensitive bands by SYBR Safe (RNA&DNA) staining migrating between 17-30 kD (nCL, w/o repeated AGPC extractions; FIG. 3a). For additional supporting information on the LEAP-RBP method, see Supplementary Note 1-3.

### Cell Line and Culture Conditions

HeLa, 293T, and Huh7 cells were maintained in Dulbecco's Modified Eagle's Medium (D6428, Sigma) supplemented with 10% FBS (35-010-CV, Corning) at 37° C., 5% CO₂. 832/13 cells were maintained in RPMI1640 (11875-093, Invitrogen) supplemented with 2 mM L-glutamine (25030-081, Invitrogen), 1 mM Na-pyruvate (11360-070, Invitrogen), 10 mM HEPES (15630-080, Invitrogen), 0.05 mM 2-mercaptoethanol (M722, Sigma), and 10% FBS at 37° C., 5% CO₂. SILAC-labeling was done using the Pierce SILAC-protein quantitation kit (1863108, Thermo), supplemented with 2 mM L-glutamine (02-0131-0200, VWR), and 10 μg/mL L-proline (88211, Thermo). Cells were passaged at least 5 times in their respective SILAC-labeled media (>10 doublings). For the comparative LEAP-RBP experiment, HeLa cells were maintained as described above and treated with DMSO (negative control) or 2 μg/mL Harringtonine (15361, Cayman Chemical Company) for 30 minutes at 37° C., 5% CO₂; Harringtonine (HT) was prepared as a 1,000× stock in DMSO.

### Ribosome Profiling

HeLa cells were cultured in 150 mm dishes until 80-90% confluent and treated with DMSO or harringtonine as described above, were washed twice with ice-cold 1×PBS and harvested on ice with 3 mL fresh ice-cold DDM lysis buffer (200 mM KOAc, 25 mM K-HEPES pH 7.2, 15 mM Mg(OAc)₂, 1 mM DTT, 50 μg/mL CHX, 1× protease inhibitor cocktail (11836153001, Roche), 40 U/mL RNase OUT(10777019, Thermo), and 2% dodecylmaltoside (DDM) (w/v)). DDM Lysates were centrifuged at 5,000×g for 5 minutes at 4° C. and 1 mL of the clarified supernatants were resolved on a 10 mL sucrose gradients (15-40% w/v) containing DDM lysis buffer components noted above via centrifugation at 35,000×g for 3 hours at 4° C. Gradients were fractionated on a Teledyne Isco Lincoln (NE) gradient fractionator with continuous A₂₅₄ sampling.

### UV-Crosslinking and Cell Harvesting

Cells were cultured in 100- or 150-mm dishes until 60-90% confluent, washed twice with ice-cold 1×PBS, and UV-crosslinked on ice with 100-800 mJ/cm² at 254 nm. Cells were lysed on plate, scraped, and transferred to a 2.0 mL microcentrifuge tube using two 400 μL aliquots of guanidinium thiocyanate (w/o phenol) buffer. Guanidinium thiocyanate (GT) buffer (4 M GT, 25 mM sodium citrate pH 7.0, 0.5% N-lauryl sarcosine, 5 mM EDTA pH 8.0, and 0.1 M 2-mercaptoethanol) was prepared with the following stock solutions prepared in DEPC-treated DI water: 5 M guanidinium thiocyanate (00522, Chemimpex), 750 mM sodium citrate pH 7.0 (BDH-9288, VWR; C-0759, Sigma), 10% N-lauryl sarcosine (L9150, Sigma), 0.5 M EDTA pH 8.0 (0105, VWR). Stock solutions were filtered (0.2 μm) to remove insoluble particulates which accumulate at the AGPC interphase: GT was filtered twice using Whatman paper (1001-150, Whatman) or by standing incubation overnight and transferring of the clarified portion; sodium citrate and EDTA stock solutions were filtered using 0.2 μm syringe filters (28145-477, VWR).

### Acidic Guanidinium Thiocyanate-Phenol-Chloroform Extraction

400 μL of acidic phenol (0981, VWR) were added to 800 μL GT cell extracts. Alternatively, cells were lysed in 1.2 mL Trizol reagent (15596026, Invitrogen) and transferred to a 2 ml microcentrifuge tube. Cell lysates were prepared by passaging through a 19 ga 1½″ needle fifteen times (305187, BD). For AGPC extraction, 240 μL chloroform (CX-1060-1, Millipore) or ˜⅗^{th} vol of phenol were added to samples and vigorously vortexed for 10 sec. Samples were centrifuged at 10,000×g for 10 min at 4° C. with slow brake setting and ˜80% (v/v) of the aqueous and organic phases were removed. For repeated AGPC extraction, 800 μL of fresh acidic guanidinium thiocyanate-phenol (2:1) buffer and 160 μL chloroform were added to the AGPC interphase and the process was repeated. The final AGPC interphase was resuspended in 1.0-1.5 mL fresh acid guanidinium thiocyanate-phenol (2:1) buffer. If AGP suspensions appeared cloudy, an additional AGPC extraction was performed. Additional protocol information is included in the Supplementary Methods.

Precipitation of RNA from Aqueous Phase Samples

Sodium chloride (5 M) was added to aqueous phase samples to a final concentration of 0.6 M and mixed by brief vortexing. One part isopropanol was added to a final concentration of 50% and samples were mixed by brief vortexing. Samples were incubated on a rotator for 15 min at 4° C. and centrifuged at 18,000×g for 15 min at 4° C. with slow brake setting. Following removal of the supernatant, pellets were washed three times with ice-cold 75% ethanol (twice the volume of precipitation mixture), incubated for 5 min on ice with occasional agitation and centrifuged at 18,000×g for 5 min at 4° C. with slow brake setting. Pellets were air dried and resuspended at the desired volume with DEPC-treated water or TE buffer. For long-term storage, precipitates were stored in 75% ethanol at −80° C. Final working sample concentrations ranged from 0.2-2.0 μg of RNA/μL.

Methanol Precipitation (95% v/v)

Samples were mixed with 19 parts room temperature (RT) 100% methanol, incubated on a rotator for 1 hr at RT, and centrifuged at 20,000×g for 10 min at 20° C. with slow brake setting. Following removal of the supernatant fraction, precipitates were washed twice with 1.0 mL RT 95% methanol (for up to 100 μg protein). For each wash, samples were vortexed for 5 sec, incubated on a rotator for 10 min at RT, and centrifuged at 20,000×g for 10 min at 20° C. with slow brake setting. Three 400 μL aliquots of RT 95% methanol were used to recover precipitates adhering to the sides of the tubes and combined in a 1.5 mL microcentrifuge tube. The tubes were then stored vertically at 4° C. overnight or at RT for 30 min to allow precipitates to settle at the bottom of the tube. Samples were centrifuged at 20,000×g for 10 min at 20° C. with slow brake setting and supernatants were removed. Pellets were air dried and resuspended at the desired concentration with 1% lithium dodecyl sulfate (LiDS) (J32816, Thermo) in TE. For long-term storage, samples were stored as precipitates in 95% methanol or as 1% LiDS TE suspensions at −80° C. Working concentrations of methanol precipitated samples in 1% LiDS TE ranged from 0.1-5.0 μg protein/μL, 0.1-8.0 μg of RNA/μL, or 0.1-2.0 μg of protein-bound RNA/μL.

### Isolation of RNP Fractions by INP

Final AGPC interphase suspensions were split between 2 mL microcentrifuge tubes (160 μL each). AGP suspensions were either stored at −80° C. or used immediately for precipitation. For precipitation, the following reagents were added to each AGP suspension in order while mixing by brief vortexing (5 sec) after each addition: 3 μL of GlycoBlue (AM9515, Invitrogen), 640 μL of 1% LiDS TE, 96 μL 5.0 M NaCl, and 899 μL isopropanol. Samples were vortexed for 5 sec and incubated on a rotator for 15 min at 4° C. Samples were centrifuged at 14,000×g for 15 min at 4° C. with slow brake setting. Following removal of the supernatant fraction, samples were washed three times with 1 mL ice-cold 75% ethanol, incubated for 5 min on ice and centrifuged at 14,000×g for 5 min at 4° C. with slow brake setting. Samples were then washed twice with 1 mL RT 95% methanol, incubated on a rotator for 10 min at RT and centrifuged at 20,000×g for 10 min at 20° C. with slow brake setting. Supernatants were removed. Precipitates were air dried and resuspended at the desired concentration in 1% LiDS in TE. For long-term storage, precipitates were stored in 95% methanol or as 1% LiDS TE suspensions at −80° C. Working concentrations of INP precipitated RNPs ranged from 1.0-3.0 μg of protein-bound RNA/μL.

Isolation of RNP fractions by LEAP-RBP

AGP input suspensions or final AGPC interphase suspensions were aliquoted (200 μL) across 1.5 mL microcentrifuge tube and stored at −80° C. or used immediately for precipitation. Chloroform was added to a final concentration of ˜7% v/v and the sample was mixed by vortex to form an emulsion (after step A; FIG. 2c). Four parts of a precipitation solution containing 3.75 M LiCl (10515, VWR) and 50% isopropanol (v/v) were layered onto the AGPC mixtures, and the tubes were closed. Samples were slowly inverted to 90 degrees and/or until the AGPC mixture was displaced from the bottom of the tube, and then returned to an upright position followed by incubation for 1 min. This process was repeated at least four times, switching the direction of inversion, increasing the angle, and increasing the speed during reversion. Samples were then homogenized by vigorous vortexing, centrifuged at 14,000×g for 5 min at 20° C., and supernatants were removed. RNP pellets were rinsed twice with 1 mL RT 95% methanol by inverting the tube multiple times and removing the supernatant. RNP pellets were then washed with 1 mL RT 95% methanol by incubating for 5 min at RT with occasional inversion. Following removal of the final 95% methanol wash, pellets were air dried and resuspended at the desired concentration with 1% LiDS TE. Additional protocol information is included in the Supplementary Methods. Working concentrations of LEAP-RBP isolated RNP fractions ranged from 0.1-4.0 μg of protein-bound RNA/p L.

### LEAP-RBP DNA Depletion Step

DNA digestion was performed using the Turbo DNase kit (Thermo, AM2238). RNP pellets containing<55 μg RNA&DNA were fully resuspended in 15 μL of TE buffer and 5 μL of a master mix containing 10× Turbo DNase buffer, TE buffer, and Turbo DNase were added to a final concentration of 1× Turbo DNase buffer and 1 μL of Turbo DNase/10 μg DNA. Samples were incubated at 37° C. for 15 min and nine parts (180 μL) fresh acid guanidinium thiocyanate-phenol (2:1) buffer were added. Samples were precipitated according to the LEAP-RBP protocol using 14 μL of chloroform and resuspended in 1% LiDS TE at the desired concentration. Additional protocol information included as part of the Supplementary Methods.

### RNA and Protein Quantitation

Samples containing more than 1.5 μg RNA/μL were diluted 1:5 in their respective buffers for RNA quantitation by UV-spectrophotometry (Thermo Scientific, Nanodrop ND-1000). For samples where DNA contamination is expected to impact RNA quantitation by more than 10%, “RNA&DNA” was used in place of “RNA” for FIG. panels. Protein concentrations were determined by BCA protein assay (23225, Thermo) using a microplate 96-well format and BSA as a protein standard. Typically, 1% LiDS TE sample suspensions were clarified prior to protein quantitation: sample suspensions were incubated at 55° C. for 20 sec, mixed by brief vortex, centrifuged at 3,000×g for 20 sec at 20° C., and clarified supernatants (˜90% v/v) were transferred to a new tube. Two 2 μL aliquots of the clarified sample suspensions typically containing between 0.1-1.0 μg protein were added to separate wells and mixed with 200 μL working reagent (Pierce BCA kit, 50:1 A:B) for BCA quantitation.

### RNase Digestion for SDS-PAGE RNase-Sensitivity Assay (SRA)

RNase digestions were performed in separate 0.2 mL thermocycler tubes (10-12 μL reactions) using a maximum of 5 μL of 1% LiDS TE sample suspensions containing<4.0 μg RNA/μL. RNase Cocktail (AM2286, Invitrogen), 10× RNase digest buffer (100 mM Tris-HCl pH 7.5, 1 M NaCl, and 10 mM EDTA), and 25× protease inhibitors (11836153001, Roche) were added at the same time to a final concentration of 2 μL RNase Cocktail/15 μg RNA. 1× RNase digestion buffer, and 1× protease inhibitors. A minimum of 0.2 μL RNase Cocktail were added regardless of RNA concentration. Samples were mixed by brief vortexing followed by a brief spin in a mini centrifuge (Supplementary Note 2a). Untreated control samples were prepared without RNase Cocktail, and both were incubated for 2 hr at 37° C. in a thermocycler with heated lid (98° C.) unless indicated otherwise in the provided Source Data (e.g., FIG. 13b, c). Input samples suspended in 1% LiDS TE were setup as untreated control reactions for SDS-PAGE and were not incubated at 37° C. unless indicated otherwise in the provided Source Data (e.g., FIG. 7i, j, FIG. 13b).

### SDS-PAGE, SYBR Safe, Coomassie Blue, Silver Stain Staining

Sample loading buffer was prepared as a 5× stock (10% SDS, 50% glycerol, 312.5 mM Tris-HCl pH 6.8, and 0.1% (m/v) bromophenol blue (B8026, Sigma)) and diluted 3:1 with b-mercaptoethanol (v/v) for a working stock (LB WS). LB WS was added to samples to a final detergent concentration of 2% and denatured by incubating for 15 min at 65° C. Samples were separated on a 0.75 mm, 15-well, 4-12% gradient polyacrylamide gels (6, 8, 10, 12% (1:1:1:1) resolver, 4% stacker) at constant voltage (80 V) for 1.5 hours at RT (Supplementary Note 2c). SYBR Safe (S33102, Invitrogen), Coomassie Blue (1610406, Biorad), and Silver Stain (PROTSIL2, Sigma) staining of polyacrylamide gels was performed on an orbital shaker. Imaging was performed using an Amersham Imager 600 (see corresponding Source Data). Additional protocol information included as part of the Supplementary Methods.

### Immunoblot

Following separation by SDS-PAGE, samples were transferred to nitrocellulose membranes using Bjerrum and Schafer-Nielsen transfer buffer (48 mM Tris and 39 mM glycine supplemented with 10% methanol and 0.03% SDS) and a Trans-Blot SD semi-dry electrophoretic transfer cell (170-3940, Bio-Rad). Alternatively, samples were wet transferred to nitrocellulose membranes using wet-transfer buffer (25 mM Tris, 96 mM glycine, 0.05% SDS, and 20% methanol) and a Bio-Rad Mini-Protean II system. Blocking and blotting conditions were performed as follows: anti-pAbPC1 antibody (ABclonal, A14872, lot 1160820101, rabbit polyclonal, diluted 1:2,000 in 1×TBST+5.0% milk, blocked with 1× TBST+5.0% milk for 1 hr at RT), anti-PABPC4 antibody (ABclonal, A5948, lot 1150980101, rabbit polyclonal, diluted 1:2,000 in 1×TBST+5.0% milk, blocked with 1× TBST+5.0% milk for 1 hr at RT), anti-TIA1 antibody (ABclonal, A6237, lot 1150860101, rabbit polyclonal, diluted 1:2,000 in 1×TBST+5.0% milk, blocked with 1×TBST+5.0% milk for 1 hr at RT), anti-HuR antibody (Santa Cruz Biotechnology, Sc-5261, clone 3A2, lot n/a, mouse monoclonal, diluted 1:2,000 in 1×TBST+5.0% milk, blocked with 1×TBST+0.3% casein for 15 min at RT), anti-XRN1 antibody (Bethyl Laboratories, A300-443A, lot A300-443A-3, rabbit polyclonal, diluted 1:2,000 in 1×TBST+5.0% milk, blocked with 1× TBST+0.3% casein for 15 min at RT), anti-RPL4 antibody (Santa Cruz Biotechnology, Sc-100838, clone RQ-7, lot n/a, mouse monoclonal, diluted 1:500 in 1×TBST+5.0% milk, blocked with 1×TBST+0.1% casein for 15 min at RT), anti-RPL8 antibody (ABclonal, A10042, lot 0051990201, rabbit polyclonal, diluted 1:2,000 in 1×TBST+5.0% milk, blocked with 1×TBST+5.0% milk for 1 hr at RT), anti-LRRC59 antibody (Bethyl Laboratories, A305-076A, lot A305-076A-1, rabbit polyclonal, diluted 1:1,000 in 1×TBST+0.2% milk, blocked with 1×TBST+0.3% casein for 15 min at RT), anti-NCL antibody (ABclonal, A5904, lot 0015360101, rabbit polyclonal, diluted 1:2,000 in 1×TBST+5.0% milk, blocked with 1×TBST+0.2% casein for 15 min at RT), anti-RPN1 antibody (Nicchitta, aP3, lot bleed 1990/08/04, rabbit polyclonal, diluted 1:5,000 in 1×TBST+5.0% milk, blocked with 1×TBST+5.0% milk for 15 min at RT), anti-TRAPa antibody (Nicchitta, TRAPa, lot bleed 7, rabbit polyclonal, diluted 1:5,000 in 1×TBST+5.0% milk, blocked with 1×TBST+5.0% milk for 15 min at RT), anti-GRP94 antibody (Nicchitta, DU120, lot bleed 1998/11/11, rabbit polyclonal, diluted 1:5,000 in 1×TBST+5.0% milk, blocked with 1×TBST+5.0% milk for 15 min at RT), anti-GAPDH antibody (DSHB, DSHB-hGAPDH-2G7, clone 2G7, lot n/a, mouse monoclonal, diluted 1:250 in 1×TBST+5.0% milk, blocked with 1×TBST+0.1% casein for 15 min at RT), anti-GRP78 antibody (Santa Cruz Biotechnology, Sc-376768, clone A-10, lot n/a, mouse monoclonal, diluted 1:100 in 1×TBST+5.0% milk, blocked with 1×TBST+0.1% casein for 15 min at RT), anti-b-tubulin antibody (DSHB, E7-s, clone E7, lot n/a, mouse monoclonal, diluted 1:250 in 1× TBST+5.0% milk, blocked with 1×TBST+0.1% casein for 15 min at RT), anti-RPS3 antibody (ABclonal, A4872, clone ARC0302, lot 4000000302, rabbit monoclonal, diluted 1:2,000 in 1×TBST+5.0% milk, blocked with 1×TBST+5.0% milk for 1 hr at RT), anti-SND1 antibody (ABclonal, A5874, lot 0029220201, rabbit polyclonal, diluted 1:2,000 in 1× TBST+5.0% milk, blocked with 1×TBST+5.0% milk for 1 hr at RT), anti-UPF1/RENT1 antibody (ABclonal, A5071, clone ARC1268, lot 4000001268, rabbit monoclonal, diluted 1:2,000 in 1×TBST+5.0% milk, blocked with 1×TBST+5.0% milk for 1 hr at RT), anti-HDLBP antibody (ABclonal, A20896, clone ARC2855, lot 4000002855, rabbit monoclonal, diluted 1:2,000 in 1×TBST+5.0% milk, blocked with 1×TBST+5.0% milk for 1 hr at RT), anti-ABCF3 antibody (ABclonal, A15168, lot 0127370101, rabbit polyclonal, diluted 1:2,000 in 1×TBST+5.0% milk, blocked with 1×TBST+5.0% milk for 1 hr at RT), anti-GEMIN5 antibody (ABclonal, A17125, lot 0111800101, rabbit polyclonal, diluted 1:2,000 in 1×TBST+5.0% milk, blocked with 1×TBST+5.0% milk for 1 hr at RT), anti-eEF2 antibody (ABclonal, A9721, clone ARC1717, lot 4000001717, rabbit monoclonal, diluted 1:2,000 in 1×TBST+5.0% milk, blocked with 1×TBST+5.0% milk for 1 hr at RT), anti-CELF1 antibody (ABclonal, A5958, lot 0202600301, rabbit polyclonal, diluted 1:2,000 in 1× TBST+5.0% milk, blocked with 1×TBST+5.0% milk for 1 hr at RT), anti-Fibrillarin/U3 RNP antibody (ABclonal, A1136, lot 0002110201, rabbit polyclonal, diluted 1:2,000 in 1× TBST+5.0% milk, blocked with 1×TBST+5.0% milk for 1 hr at RT). Signal detection was performed using WesternBright ECL HRP substrate (K-12045, Advansta) and an Amersham Imager 600 (see corresponding Source Data). Additional protocol information included as part of the Supplementary Methods.

### Proteinase K Digestion

For proteinase K digestion, samples were diluted 1:2 with 2× proteinase K buffer (100 mM Tris HCl pH 7.5, 20 mM EDTA pH 8.0, 300 mM NaCl, 2% SDS), mixed with 2 μL proteinase K stock (20 mg/mL proteinase K (BIO-37037, Bioline), 20 mM Tris HCl pH 7.5, 1 mM CaCl₂), 50% glycerol v/v) per 10 μg of protein, and incubated at 55° C. for 15 min. For isolation of RNA and/or DNA samples were mixed with 4 parts neutral guanidinium thiocyanate-phenol (2:1) buffer (J75829, Affymetrix) and 1 part chloroform, vigorously vortexed for 10 sec, and centrifuged at 10,000×g for 10 min at 4° C. with slow brake setting. Aqueous phase samples were precipitated as outlined above (Precipitation of RNA from aqueous phase samples).

### TBE Gel Analysis of RNA Samples

RNA samples suspended in DEPC-treated water were mixed with 6× gel loading buffer (R0611, Thermo), incubated at 65° C. for 2 min, and chilled for 2 min on ice before being loaded on a 1.0% or 1.5% agarose TBE gel containing 0.5-1×SYBR Safe stain. Samples were separated under constant voltage at 140V or 140 V for 20-40 min and visualized using an Amersham Imager 600 (see corresponding Source Data for specific experimental conditions).

qPCR Analysis

qPCR was performed using the Luna Universal qPCR Master Mix (NEB, M3003) on a Bio-RAD Cfx96 real-time PCR system using a 96-well format and 20 μL reactions. DNA contamination was quantified using primers targeting the coding region of GRP78:

F-primer:
(SEQ ID NO: 1)
5′-CTTGGTATTGAAACTGTGGGAGGT-3′
R-primer:
(SEQ ID NO: 2)
5′-AGATCTGAGACTTCTTGGTAGGCA-3′

### Sample Preparation for MS Proteomic Analysis

Digestion and depletion of RNA and/or DNA from input samples and RNP fractions was necessary prior to MS-based proteomic analysis FIG. 12B, 9a-c). For SILAC LC-MS/MS experiments, RNP suspensions were normalized to 3 μg/μL protein-bound RNA in 1% LiDS TE and 33 μL were used per 100 μL reaction containing 13.3 μL RNase Cocktail, 1× RNase digest buffer (10 mM Tris-HCl pH 7.5, 100 mM NaCl, and 1 mM EDTA pH 8.0), and 1× protease inhibitors (11836153001, Roche). For the comparative LEAP-RBP experiment, 10 μL of input samples containing 2.0 μg protein/μL were used per 25 μL reaction containing 0.6 μL RNase Cocktail, 1× RNase digest buffer, and 1× protease inhibitors; 20 μL of clRNP fractions containing 0.2 μg RNA-bound protein/μL were used per 50 μL reaction containing 3.1 μL RNase Cocktail, 1× RNase digest buffer, and 1× protease inhibitors. RNase digests were incubated for 2 hr at 37° C. and then precipitated with 95% methanol v/v as described above. Each input sample was suspended in TE buffer and DNA was digested using 2.5 μL Turbo DNase (Thermo, AM2238) and a final concentration of 1× Turbo DNase buffer in a 50 μL reaction (15 min, 37° C.) and precipitated with 95% methanol v/v as described above. Pellets were air dried and submitted for proteomics analysis. For long-term storage, precipitates can be stored in 95% methanol at −80° C.

### SILAC LC-MS/MS Analysis of LEAP-RBP and INP Fractions

Prior to LC-MS/MS analysis, samples were supplemented with 50 μL 8.0 M urea in 50 mM ammonium bicarbonate and subjected to 2 rounds of probe sonication. Next, samples were spiked with either a total of 120 or 240 fmol of bovine casein, supplemented with 15 μL 20% SDS, reduced with 10 mM dithiolthreitol for 30 min at 45° C. and alkylated with 20 mM iodoacetamide for 45 min at RT. Then, samples were supplemented with a final concentration of 1.2% phosphoric acid and 543 μL of S-Trap (Protifi) binding buffer (90% methanol/100.0 mM TEAB). Proteins were collected on the S-Trap, digested using 20 ng/μL sequencing grade trypsin (Promega) for 1 hr at 47° C., and eluted using 50 mM TEAB, followed by 0.2% FA, and lastly using 50% ACN/0.2% FA. All samples were then lyophilized to dryness and resuspended in 12 μL 1% TFA/2% acetonitrile containing 12.5 fmol/μL yeast alcohol dehydrogenase (ADH_YEAST).

Quantitative LC-MS/MS was performed on 1 μg of each sample, using a nanoAcquity UPLC system (Waters Corp) coupled to a Thermo Orbitrap Fusion Lumos high resolution accurate mass tandem mass spectrometer (Thermo) equipped with a FAIMSPro device via a nanoelectrospray ionization source. Briefly, peptides were trapped on a Symmetry C18 20 mm×180 μm trapping column (5 μL/min at 99.9/0.1 v/v water/acetonitrile), after which the analytical separation was performed using a 1.8 μm Acquity HSS T3 C18 75 μM×250 mm column (Waters Corp.) with a 90-min linear gradient of 5 to 30% acetonitrile with 0.1% formic acid at a flow rate of 400 nanoliters/minute (nL/min) with a column temperature of 55° C. Data collection on the Fusion Lumos mass spectrometer was performed for three difference compensation voltages (40 V, 60 V, 80 V). Within each CV, a data-dependent acquisition (DDA) mode of acquisition with a r=120,000 (m/z 200) full MS scan from m/z 375-1500 with a target AGC value of 4e5 ions was performed. MS/MS scans were acquired in the ion trap in rapid mode from m/z 100 with a target AGC value of 2e4 and max fill time of 100 ms. The total cycle time for each CV was 1 s, with total cycle times of 3 sec between like full MS scans. A 45s dynamic exclusion was employed to increase depth of coverage. The total analysis cycle time for each fraction injection was approximately 2 hr.

Data were imported into Proteome Discoverer 2.5 (Thermo Scientific Inc.) and all LC-MS/MS runs were aligned based on the accurate mass retention time of detected ions (“features”) which contained MS/MS spectra using Minora Feature Detector algorithm in Proteome Discoverer. Relative peptide abundance was calculated based on area-under-the-curve (AUC) of the selected ion chromatograms of the aligned features across all runs. A filter was applied which required each peptide to be measured in at least 2 unique samples and in at least 50% of at least one of the unique biological groups. The MS/MS data was searched against the SwissProt H. sapiens database (downloaded November 2019) and an equal number of reversed sequence “decoys” for false discovery rate determination. Mascot Distiller and Mascot Server (v 2.5, Matrix Sciences) were utilized to produce fragment ion spectra and to perform the database searches. Database search parameters included fixed modification on Cys (carbamidomethyl) and variable modifications on Meth (+16, oxidation) and Arg/Lys (+10/+8 for heavy SILAC residues K+8, R+10). Peptide Validator and Protein FDR Validator nodes in Proteome Discoverer were used to annotate the data at a maximum 1% protein false discovery rate based on q-value calculations. Note that peptide homology was addressed using razor rules in which a peptide matched to multiple different proteins was exclusively assigned to the protein that has more identified peptides. Protein homology was addressed by grouping proteins that had the same set of peptides to account for their identification. Following database searching and peptide scoring using Proteome discoverer validation, the data was annotated at a 1% protein false discovery rate.

### SILAC LC-MS/MS Data Processing and Analysis

Initial data processing for identification of UV-enriched proteins and generation of sum peptide intensities were done separately for each method (INP vs LEAP-RBP). Peptide intensities of common contaminants and spike-ins (human keratins, BSA, porcine trypsin, yeast alcohol dehydrogenase) were manually curated from protein lists. The remaining peptide intensities were sorted by SILAC label and used to generate sum peptide intensities (SPI). Proteins not detected in all three UV-crosslinked samples were excluded from downstream sample normalization procedures and data analysis. Replicate samples were mean-normalized to total SPI and SPI_{nCL} values equal to 0 were replaced with the average non-zero SPI_{nCL} value of the same protein ID. Proteins only detected in UV-crosslinked samples were scored as UV-enriched*, omitted from statistical analysis, and given the following pseudo-value: −log₁₀(p value)=10, log₂(CL/nCL)=10. For the remaining proteins, log₂(CL/nCL), ratios were generated with SPI_{CL} values and average SPI_{nCL} values according to equations (2). UV-enriched* proteins were identified by testing against the null hypothesis that the average log₂(CL/nCL) ratio equals zero using a heteroscedastic upper-tailed t test. Correction for multiple hypothesis testing was performed using the Benjamini-Hochberg approach and a false-discovery rate of 5%.

### Data Processing and Analysis of Referenced MS Datasets

Maxquant output files (.txt) for XRNAX, OOPs, Ptex, and TRAPP were downloaded from the ProteomeXchange using the identifiers PXD010520, PXD026716, PXD009571, and PXD011071 respectively. Protein identifiers, unique peptide counts, and sum peptide intensities were obtained from their respective proteingroup.txt file; proteins marked as potential contaminants were removed. MS datasets for RIC and eRIC including protein identifiers, unique peptide counts, and sum peptide intensities were obtained from Perez-Perri, J. I., et al., Discovery of RNA-binding proteins and characterization of their dynamic responses by enhanced RNA interactome capture. Nat Commun, 2018. 9(1): p. 4408. Protein identifiers (Uniport IDs and gene names) starting with “Majority protein IDs” were used to generate primary Uniprot IDs for comparative analyses. For RIC and eRIC, a pseudo-third replicate was added by averaging non-zero SPI values of replicates 1 and 2. Because XRNAX was performed without replicates, samples were first mean normalized and the average non-zero SPI values of 12 different samples were used for MS data analyses; MCF7, HEK293, and HeLa; half-confluent and confluent; 15 min and 30 min partial digestion prior to silica purification (3×2×2=12 different samples). For the remaining MS datasets, proteins not detected in all UV-crosslinked samples were excluded from downstream sample normalization procedures and data analysis. Replicate samples were mean-normalized to total SPI and SPI_{nCL} values equal to 0 were replaced with the average non-zero SPI_{nCL} value of the same protein ID.

### LC-MS/MS Analysis (Comparative LEAP-RBP Experiment)

Prior to LC-MS/MS analysis, samples were supplemented with 50 μL 8.0 M and subjected to 2 rounds of probe sonication. Next, samples were spiked with either a total of 120 or 240 fmol of bovine casein, supplemented with 7.9 μL 20% SDS, reduced with 10 mM dithiolthreitol for 30 min at 32° C. and alkylated with 20 mM iodoacetamide for 45 min at RT. Then, samples were supplemented with a final concentration of 1.2% phosphoric acid and 472 μL of S-Trap (Protifi) binding buffer (90% methanol/100.0 mM TEAB). Proteins were collected on the S-Trap, digested using 4 or 20 ng/μL (for clRNP fractions containing 4 μg protein or input samples containing 20 μg protein respectively) sequencing grade trypsin (Promega) for 1 hr at 47° C., and eluted using 50 mM TEAB, followed by 0.2% FA, and lastly using 50% ACN/0.2% FA. All samples were then lyophilized to dryness and resuspended in 12 or 60 μL (for clRNP fractions or input samples respectively) of 1% TFA/2% acetonitrile containing 12.5 fmol/μL yeast alcohol dehydrogenase (ADH_YEAST).

Quantitative LC-MS/MS was performed on 3 μL (1 μg) of each sample, using a nanoAcquity UPLC system (Waters Corp) coupled to a Thermo Orbitrap Fusion Lumos high resolution accurate mass tandem mass spectrometer (Thermo) equipped with a FAIMSPro device via a nanoelectrospray ionization source. Briefly, peptides were trapped on a Symmetry C18 20 mm×180 μm trapping column (5 μL/min at 99.9/0.1 v/v water/acetonitrile), after which the analytical separation was performed using a 1.8 μm Acquity HSS T3 C18 75 μM×250 mm column (Waters Corp.) with a 90-min linear gradient of 5 to 30% acetonitrile with 0.1% formic acid at a flow rate of 400 nanoliters/minute (nL/min) with a column temperature of 55° C. Data collection on the Fusion Lumos mass spectrometer was performed for three difference compensation voltages (40 V, 60 V, 80 V). Within each CV, a data-dependent acquisition (DDA) mode of acquisition with a r=120,000 (m/z 200) full MS scan from m/z 375-1500 with a target AGC value of 4e5 ions was performed. MS/MS scans were acquired in the Orbitrap at r=50,000 (m/z 200) from m/z 100 with target AGC value of 1e5 and max fill time of 35 ms. The total cycle time for each CV was 1 s, with total cycle times of 3 sec between like full MS scans. A 45s dynamic exclusion was employed to increase depth of coverage. The total analysis cycle time for each fraction injection was approximately 2 hr.

Following 15 total UPLC-MS/MS analyses (excluding conditioning runs, but including 3 replicate SPQC samples), data were imported into Proteome Discoverer 3.0 (Thermo Scientific Inc.) and individual LCMS data files were aligned based on the accurate mass retention time of detected precusor ions (“features”) using Minora Feature Detector algorithm in Proteome Discoverer. Relative peptide abundance was measured based on peak intensities of the selected ion chromatograms of the aligned features across all runs. The MS/MS data was searched against the SwissProt H. sapiens database (downloaded August 2022), a common contaminant/spiked protein database (bovine albumin, bovine casein, yeast ADH, etc.), and an equal number of reversed sequence “decoys” for false discovery rate determination. Sequest was utilized to produced fragment ion spectra and to perform the database searches. Database search parameters included fixed modification on Cys (carbamidomethyl) and variable modification on Met (oxidation). Search tolerances were 2 ppm and 0.8 Da product ion with full trypsin enzyme rules. Peptide Validator and Protein FDR Validator nodes in Proteome Discoverer were used to annotate the data at a maximum 1% protein false discovery rate based on q-value calculations. Note that peptide homology was addressed using razor rules in which a peptide matched to multiple different proteins was exclusively assigned to the protein that has more identified peptides. Protein homology was addressed by grouping proteins that had the same set of peptides to account for their identification. A master protein within a group was assigned based on % coverage.

### LC-MS/MS Data Processing (Comparative LEAP-RBP Experiment)

Initial data processing and generation of sum peptide intensities were done separately for each fraction and each sample group (input or clRNP and DMSO or HT). Peptide intensities of common contaminants and spike-ins (human keratins, BSA, porcine trypsin, yeast alcohol dehydrogenase) were manually curated from protein lists. Proteins not detected in all three replicates of both sample groups (DMSO and HT) for a given fraction (input or clRNP) and containing at least 2 unique peptide matches were excluded from downstream sample normalization and data analysis. Samples were mean-normalized to total SPI and log₂ normalized SPI values were used to test for differences in protein recovery between samples groups (DMSO vs HT) for each fraction (input or clRNP) using independent two-tailed homoscedastic t tests. Correction for multiple hypothesis testing was performed with the Benjamini-Hochberg approach and a false-discovery rate of 5% on total protein IDs (no S/N limit) or only those which displayed S/N ratios>3 in LEAP-RBP (clRNP) fractions by SILAC LC-MS/MS analysis.

### Gene-Ontology (GO) Enrichment Analysis

GO enrichment analyses were performed for UV-enriched* proteins identified by INP and LEAP-RBP using PANTHER V17.0. The resulting GO-annotated protein lists were used to sort protein IDs (e.g., RBP vs non-RBP) for downstream analyses.

Sample Preparation for sRNA-Seq and Data Analysis

Two independent samples (HeLa) were UV-crosslinked with 0.4 J/cm² (254 nm). clRNPs were isolated from the final (6^{th}) AGPC interphase suspension by LEAP-RBP and resuspended in TE buffer. Ca. 6 μg of protein-bound RNA was treated with Turbo DNase as outlined above (LEAP-RBP DNA depletion step) without performing the second LEAP step. Then, 20 μL of 2× proteinase K buffer and 3 μL proteinase K stock (20 mg/mL) were added, and samples were processed as described above (Proteinase K digestion).

Library Construction, Quality Control, and sRNA Sequencing

For sRNA library construction, 3′ and 5′ adaptors were ligated to 3′ and 5′ ends of small RNAs, respectively. First strand cDNA was synthesized after hybridization with a reverse transcription primer and double-stranded cDNA libraries generated via PCR enrichment. After purification and size selection, libraries with insertions between 18-40 bp were selected. Library concentrations and QC was performed via Qubit and real-time PCR for quantitation and Bioanalyzer for size distribution analysis. Quantified libraries were pooled and sequenced on Illumina platforms in SE50 mode.

Data Analysis (sRNA-Seq)

Raw data (raw reads) in fastq format were processed through custom (Novogene) perl and python scripts to remove read sequences containing poly-N, 5′ adapter contaminants, lacking 3′ adapter or the insert tag, containing polyA, T, G or C, and low quality reads. Small RNA read data were mapped to reference sequence using Bowtie version 0.12.9, without mismatch. Mapped small RNA tags were examined for known miRNA homologies using miRDeep2 version 0.0.5. To remove tags originating from protein-coding genes, repeat sequences, rRNA, tRNA, snRNA, and snoRNA, small RNA tags were mapped with RepeatMasker version 4.0.3 and Rfam version 11.0. Novel miRNA predictions were performed using miRDeep2 version 0.0.5 modified with miREvo version 1.1 and ViennaRNA version 2.1.1 through exploration of secondary structure, Dicer cleavage sites, and the minimum free energy of the small RNA tags unannotated in the former steps. For alignment and annotations, some small RNA tags may map to more than one category. To ensure that small RNAs mapped to only one annotation, the following priority rules were used: known miRNA>rRNA>tRNA>snRNA>snoRNA>repeat>gene>NAT-siRNA>gene>novel miRNA>ta-siRNA. miRNA expression levels were estimated by TPM (transcript per million) through the following criteria: Normalization formula: Normalized expression=mapped reads*1,000,000.

### Statistical Analysis

All statistical analyses were performed using JMP Pro 14.0, exported test results included as part of the provided Source Data.

### Data Availability

Raw data and Protein Discoverer results files from LEAPR-RBP and INP SILAC, and non-SILAC LC-MS/MS experiments are available on the MassIVE repository [massive.ucsd.edu]. Small RNA sequencing data are available at NCBI GEO, series record GSE235647 [ncbi.nlm.nih.gov]. Maxquant output files for XRNAX, OOPs, Pte, and TRAPP were downloaded from the ProteomeXchange using the following accession codes; XRNAX: PXD010520 [ebi.ac.uk/pride/archive/projects/PXD010520] (proteinGroups.txt file located in the txt_ihRBP.zip file); OOPs: PXD021169 [ebi.ac.uk/pride/archive/projects/PXD021169](proteinGroups.txt file located in the txt.zip file); Ptex: PXD009571 [ebi.ac.uk/pride/archive/projects/PXD009571] (proteinGroups.txt file located in the txt_Human.zip file); TRAPP: PXD011071 [ebi.ac.uk/pride/archive/projects/PXD011071](Maxquant_proteinGroups.txt files located in the TRAPP_cerevisiae_400.zip, TRAPP_cerevisiae_800.zip, and TRAPP_cerevisiae_1360.zip files). MS datasets for RIC and eRIC including protein identifiers, unique peptide counts, and sum peptide intensities were obtained from [nature.com/articles/s41467-018-06557-8].

The main data supporting the findings of this study are available within the main Manuscript and Supplementary Information, or in the Source data provided with this paper. Specific p values are included within the Source Data file as well. Additional details on datasets and protocols that support the findings of this study will be made available by the corresponding author upon request.

### Code Availability

Custom scripts used during the small RNA sequencing experiment to clean reads are propriety script of Novogene. The remaining software is publicly available: Bowtie version 0.12.9 [sourceforge.net/projects/bowtie-bio/files/bowtie/0.12.9/]; RepeatMasker version 4.0.3 [repeatmasker.org/]; Rfam version 11.0 [xfam.org/]; miRDeep2 version 0.0.5 [github.com/rajewsky-lab/mirdeep2]; miREvo version 1.1 [github.com/akahanaton/miREvo]; ViennaRNA version 2.1.1 [https://www.tbi.univie.ac.at/RNA/#download

### Supplementary Methods

### 1. Description of Sample Types.

“UV-crosslinked cells” and “non-crosslinked cells” refers to UV-irradiated or non-irradiated samples containing “total cellular mass” (i.e., “total cellular protein”, “total cellular RNA”, “total cellular DNA”, etc.).

“UV-crosslinked samples” and “non-crosslinked samples”, contained or were derived from UV-crosslinked or non-crosslinked cells.

“AGP suspensions” contained samples suspended in acidic guanidinium thiocyanate-phenol (2:1) buffer. In this study, “AGP input suspensions” refers to AGP suspensions containing UV-crosslinked or non-crosslinked cells (i.e., total cellular mass). However, it was considered reasonable for AGP input suspensions to represent any “starting sample” resuspended and/or mixed with >6 parts acidic guanidinium thiocyanate-phenol (2:1) buffer (e.g., cytosolic fractions; Supplementary Note 1).

“Aqueous phases” and “organic phases” refers to the upper (aqueous) and lower (organic) phases during AGPC extraction.

“AGPC interphase” refers to the insoluble material remaining after AGPC extraction and removal of the aqueous and organic phases.

“AGPC interphase samples” contained protein recovered from the AGPC interphase by methanol precipitation (95% v/v).

“AGP interphase suspensions” contained the AGPC interphase resuspended in fresh acidic guanidinium thiocyanate-phenol (2:1) buffer.

“Final AGPC interphase” refers to the AGPC interphase at maximum % TP_{S} (Supplementary Note 4e).

“Final AGPC interphase suspensions” contained the final AGPC interphase resuspended in acidic guanidinium thiocyanate-phenol (2:1) buffer.

“AGPC mixtures” or “AGPC samples” refers to samples during AGPC extraction prior to centrifugation, or AGP suspensions after the addition of chloroform during the LEAP step.

“RNA samples” contained RNA isolated by any process (with or without DNA depletion step). “Protein samples” contained protein isolated by any process.

“RNP samples” contained mixtures of crosslinked ribonucleoproteins (“clRNPs”) and/or free RNA and protein isolated by any process but excluded samples where DNA contamination impacted RNA (UV-spectrophotometry) quantitation>10%.

“Input” refers to samples containing total protein isolated by methanol precipitation (95% v/v) of AGP input suspensions.

“clRNP fractions” refers to LEAP-RBP fractions isolated from final AGPC interphase suspensions of UV-crosslinked samples (with or without DNA depletion step). For simplicity, clRNP fractions were labeled as RNP fractions when compared to other RNP fractions (e.g., FIG. 8a-c). Where appropriate, samples or sets of samples were described by the isolation process with or without the word “fraction(S)” (e.g., LEAP-RBP fractions).

### 2. Description of Terminologies.

“Signal” (quantity=S) refers to “RNA-bound proteins” while “noise” (quantity=N) refers to their “unbound counterparts” or “unbound proteins”.

“Background” (quantity=B) refers to “background proteins” without “RNA-bound counterparts” (S=0). For simplicity, “free proteins” refer to both unbound and background proteins; and unbound proteins included background proteins (i.e., N=B when S=0). However, true noise was considered distinguishable from true background (Supplementary Note 4d).

“Observable proteins” refers to proteins identifiable by MS-based proteomic analysis via peptide mapping or proteins migrating at their expected (unbound) molecular weight during SDS-PAGE.

“Observed proteins” or “Obs.” (quantity=O) refers to proteins that were observable (e.g., observed proteins during SDS-PAGE of UV-crosslinked samples only included free proteins).

In the absence of adjectives (e.g., RNA-bound), “proteins” refers to observable proteins.

“Protein quantities” refers to their quantitative amounts.

“Protein profiles” refers to “relative quantities” of proteins in the sample.

“SILAC LC-MS/MS analysis” refers to MS-based proteomic analysis of protein samples isolated from pooled input samples containing equivalent amounts of differentially SILAC-labeled UV-crosslinked and non-crosslinked samples.

“LC-MS/MS analysis” refers to MS-based proteomic analysis of protein samples isolated from non-pooled input samples containing equivalent amounts of UV-crosslinked or non-crosslinked samples.

For simplicity, “SILAC” and “non-SILAC” referred to SILAC LC-MS/MS and LC-MS/MS analysis respectively.

“MS data analysis” refers to the analysis of MS datatsets generated by LC-MS/MS and SILAC LC-MS/MS experiments.

“Specific” or “Non-specific UV-crosslinking” refers to photo-crosslinking of proteins to RNA or non-RNA substrates respectively.

“UV-dependent enrichment of RNA” refers to the fold-enrichment of RNA in UV-crosslinked samples when compared to an equivalent % fraction of non-crosslinked sample.

“UV-dependent enrichment of proteins” refers to the fold-enrichment of proteins in UV-crosslinked samples when compared to an equivalent % fraction of non-crosslinked sample and is represented by CL/nCL ratios.

“Significantly UV-enriched*” or “UV-enriched*” proteins displayed CL/nCL ratios significantly greater than 1 by statistical hypothesis testing during MS-based proteomic analysis (SILAC and non-SILAC).

“RNA-bound protein enrichment” refers to the enrichment of RNA-bound proteins over their unbound counterparts.

“S/N ratios” or “S/N of proteins” represents the ratio of RNA-bound to unbound counterparts.

“Enrichment efficiency” refers to the magnitude of CL/nCL and/or S/N ratios. SRA and SILAC LC-MS/MS analysis were considered S/N-based analyses because they distinguish RNA-bound proteins from unbound proteins and evaluated S/N (Supplementary Note 8a).

Compared to SILAC LC-MS/MS, evaluating S/N by SRA was considered more accurate because non-specific UV-crosslinking does not contribute to the displayed S/N of proteins (i.e., S/N of observable protein quantities). Therefore, proteins which appeared “RNase-sensitive” (|S|>0) by SRA analysis were considered “RNase-sensitive RBPs” or “bona fide RBPs” while proteins displaying “positive S/N ratios” by SILAC LC-MS/MS analysis were considered “UV-enriched” (i.e., CL/nCL ratios>1).

The “S/N of RBPs” refers to the S/N of GO-annotated RBPs or RNase-sensitive RBPs, while the “S/N of non-RBPs” refers to the S/N of proteins without prior GO-annotations (GO:RBP).

During MS data analysis, S/N ratios for non-RBPs represented the ratio of RNA-bound to unbound counterparts (SILAC and non-SILAC) despite the premise that non-specific UVcrosslinking to non-RNA substrates and/or UV-dependent enrichment of free protein can result in their apparent UV-enrichment (i.e., CL/nCL ratios>1 and S/N ratios>0). Observed quantities of proteins displaying S/N ratios greater than or less than 1 by S/N-based analyses were considered more representative of their RNA-bound or unbound quantities respectively. Observed quantities of proteins displaying S/N ratios>3 by S/N-based analyses were considered representative of their RNA-bound quantities (>75% RNA-bound). Conversely, observed quantities of proteins displaying S/N greater less than 0.33 were considered representative of their unbound quantities (<25% RNA-bound); supplementary note 6.

### 3. Description of Quantitative Metrics.

“Yield” represents the quantity of RNA or protein recovered by a given process expressed as micrograms per one percent of starting sample fraction (e.g., μg/% fraction).

“Recovery” represents the quantity of RNA or protein isolated by a given process expressed as a percentage of the starting quantity (e.g., % RNA recovery).

“Comparable recovery” refers to a non-significant difference in yield.

“Near 100% recovery” refers to a non-significant difference in yield when compared to a suitable control.

“Signal recovery” refers to RNA-bound protein or protein-bound RNA recovery. While signal referred to RNA-bound proteins, evaluating signal recovery by comparing protein-bound RNA yield was considered more accurate (Supplementary Note 4e, f).

“Recovery of noise” refers to unbound protein recovery (Supplementary Note 4a).

“Without signal loss” refers to an indiscernible decrease in signal recovery as compared to a suitable control.

“RBP-specific signal loss” refers to discernible, varied decrease in recovery of RNA-bound RBPs.

“High yield” and “low yield” methods were considered processes with high and low recovery respectively.

The following metrics were used when the isolation processes employed recovered near 100% of RNA and/or protein from samples, and when the indicated population contributed>90% of recovered RNA (UV-spectrophotometry) and/or protein (BCA) by mass (Supplementary Note 3). These RNA and protein populations can be described as total populations of starting samples, or herein, total cellular populations. RNA and protein recovered from AGP input suspensions by LEAP-RBP (with DNA depletion step) were considered representative of “total RNA” and “total RNA-bound protein” respectively. RNA recovered from the final AGPC interphase (with or without resuspension) by any process capable of near 100% RNA recovery (with or without DNA depletion step) was considered representative of “total protein-bound RNA”. However, only protein recovered from final AGPC interphase suspensions by LEAP-RBP (with or without DNA depletion step) was considered representative of total RNA-bound protein (i.e., “total clRNPs”).

Protein recovered from AGP input suspensions by methanol (95% v/v) precipitation was considered representative of “total protein”, or herein, total cellular protein. For clarity, total protein was distinguished from “total protein isolated” or “total protein in X” where X denotes the sample or fraction (e.g., total protein in LEAP-RBP fractions was considered representative of total RNA-bound protein).

“Total protein abundance” represents protein quantity per μg of total protein but was distinguished from “protein abundance” representing protein quantity as a percentage of “total protein in the sample”. Nonetheless, protein abundances estimated as a percentage of total protein were considered equivalent to their total protein abundances.

“Total RNA-bound protein abundance” represents RNA-bound protein quantity per μg of total RNA-bound protein but was distinguished from “RNA-bound protein abundance” which represents RNA-bound protein quantity (SPIS) as a percentage of total protein in the sample (% TPS). RNA-bound protein abundances estimated as a percentage of total RNA-bound protein were considered equivalent to their total RNA-bound protein abundances (Supplementary Note 4f).

“RNP compositions” represents the ratio of protein to RNA in RNP fractions and was estimated by dividing protein yields with their corresponding RNA yields.

“clRNP compositions” represents the ratio of RNA-bound protein to protein-bound RNA in clRNP fractions and was estimated by dividing protein yields with their corresponding RNA yields.

“Protein UV-crosslinking efficiency” represents the percentage of total protein UV-crosslinked to RNA and was estimated by dividing total RNA-bound protein yield with the corresponding total protein yield, multiplied by 100.

“RNA UV-crosslinking efficiency” represents the percentage of total RNA UV-crosslinked to protein and was estimated by dividing total protein-bound RNA yield with the corresponding total RNA yield, multiplied by 100 (Supplementary Note 3).

### 4. Evaluating UV-Dependent Enrichment and S/N by SDS-PAGE RNase-Sensitivity Assay (SRA).

To evaluate S/N by SRA, RNase-treated samples were compared to equivalent amounts of untreated samples by SDS-PAGE with SYBR Safe (RNA&DNA), Coomassie Blue (protein), and Silver Stain (RNA, DNA, and protein) staining, or immunoblot (Supplementary Note 2).

“RNase-dependent fold-change” or “RNase-sensitivity” refers to the fold-change in observed protein quantity (denoted by Δ log 2(O)) between RNase-treated and untreated samples as shown in equation (1). Proteins were considered “RNase-sensitive” if they displayed discernible RNase-sensitivity and “RNase-insensitive” if they did not. All RNase-sensitive proteins were considered RNase-sensitive RBPs or bona fide RBPs while “RNase-insensitive proteins” were considered non-RBPs or RBPs with low S/N. UV-enriched proteins displaying CL/nCL ratios>1 by SILAC LC-MS/MS and which remained undetectable by SRA and immunoblot were not considered bona fide RBPs regardless of GO-annotation status (e.g., GRP94, a GO-annotated RBP). However, because this could be due to their low RNA-bound abundance, it was not considered confirmation that a protein lacks RNA-binding activity. The RNase-sensitivity of an RBP was considered linearly related to their S/N: (|S|+N)RNase/(N)untreated=S/N+1. Therefore, an increase in AO was considered indicative of “enhanced S/N”.

The “sensitivity of SRA” refers to the detectability of RNase-sensitive RBPs during SRA analysis (e.g., SRA with Coomassie Blue (protein) staining or immunoblot). Because the amount of RNA-bound protein analyzed by SRA was determined by RNA quantity, depleting free RNA and concentrating protein-bound RNA enhanced the sensitivity of SRA (i.e., |S|/μg RNA). The RNase-sensitivity of total protein in the sample analyzed by SRA and Coomassie Blue (protein) staining was considered directly related to % TP_{S}; the RNase-sensitivity (S/N) of individual RBPs was not (Supplementary Note 4e, 8a). To evaluate UV-dependent enrichment by SRA, RNase-treated and untreated samples isolated from UV-crosslinked and non-crosslinked cells were normalized to % fraction and analyzed by SDS-PAGE with SYBR Safe (RNA&DNA), Coomassie Blue (protein), and Silver Stain (RNA, DNA, and protein) staining, or immunoblot (e.g., FIG. 12A).

“UV-enrichment of RNA” or “UV-enrichment of protein” referred to the fold-enrichment of RNA or protein in UV-crosslinked samples as compared to non-crosslinked samples respectively.

### 5. Estimating RBP-Specific UV-Crosslinking Efficiencies and S/N Ratios by SDS-PAGE and Immunoblot.

In this study, S/N ratios of proteins were estimated by SILAC LC-MS/MS analysis of LEAP-RBP fractions (Supplementary Note 5, 6). However, estimating S/N of RBPs by comparing serially diluted RNase-treated samples to a corresponding untreated sample by SDS-PAGE and immunoblot was considered a reasonable alternative (FIG. 10).

“RBP-specific UV-crosslinking efficiencies” represents the percentage of total protein quantity that was UV-crosslinked to RNA and was estimated by comparing serial dilutions of non-crosslinked total protein and RNase-treated total RNA-bound protein by SDS-PAGE and immunoblot (FIG. 10, Supplementary Note 3c). Notably, the latter was only considered appropriate when the observed protein quantity (RNase) was representative of RNA-bound quantity (i.e., display S/N ratios>3.

### 6. Evaluating Total Protein and Total RNA-Bound Protein Abundance by SDS-PAGE.

To evaluate total protein abundance, non-crosslinked or RNase-treated UV-crosslinked input samples were normalized to μg of total protein and analyzed by SDS-PAGE with Coomassie Blue (protein) staining or immunoblot (e.g., input, RNase; FIG. 7i, j). To evaluate total RNA-bound protein abundance, RNase-treated RNP fractions containing total RNA-bound protein were normalized to μg of total RNA or total protein-bound RNA and analyzed by SDS-PAGE with Coomassie Blue (protein) staining or immunoblot (e.g., clRNP fractions, RNase; FIG. 7i, j). Notably, this was only considered appropriate when the observed protein quantity (RNase) was representative of RNA-bound quantity (i.e., display S/N ratios>3). Three different classes of RNA-binding proteins were observed in this study. This includes more constitutive RBPs whose total abundance and RNA-bound abundance were similar between experimental samples (e.g., pAbPC1 or RPL4; FIG. 7j); differentially abundant RBPs whose total abundance and RNA-bound abundance vary between experimental samples but whose relative abundance in each fraction appears directly related (e.g., NCL or RPN1); and dynamic RBPs exhibiting a difference in RNA-bound abundance without a discernible difference in total abundance or vice versa (e.g., TIA1 and LRRC59).

### 7. MS Data Analysis.

For MS-based proteomic analysis, protein quantities were estimated as the sum of their identified peptide intensities or sum peptide intensities and were represented by SPI values. The sum of all SPI values or “total SPI” was equal to the total MS signal and was considered representative of total protein in the sample as defined by the TPA method. Replicate samples were denoted by “R #” where # is the replicate sample number.

Sum peptide intensities of proteins observed in the UV-crosslinked SILAC channel (SILAC) or UVcrosslinked sample (non-SILAC) were represented by “SPICL” values, while the sum of all SPICL values was represented by “total SPICL”. Sum peptide intensities of proteins observed in the non-crosslinked SILAC channel (SILAC) or non-crosslinked sample (non-SILAC) were represented by “SPInCL” values, while the sum of all SPInCL values was represented by “total SPInCL”. Log 2(CL/nCL) and log 2(S/N) ratios were generated with SPICL values and average SPInCL values according to equations (2) and (3). Proteins only detected in UVcrosslinked samples were given the following pseudo-values: log 2(S/N)=10, log 2(CL/nCL)=10. Proteins displaying negative average log 2(CL/nCL) ratios were given pseudo-log 2(S/N) ratios of −10. Average SPI values and S/N ratios were used to estimate RNA-bound and free protein quantities which were represented by “SPIS” and “SPIN” values respectively.

Unless indicated otherwise, SPI=SPInCL+SPICL=SPIO=SPIS+SPIN for both SILAC and non-SILAC LC-MS/MS experiments. Additional information, examples, and equations for Excel were included in the provided Source Data for FIG. 5-d. The standard deviation of log 2(SPI) was used to calculate the detectable fold-change in observed quantities or detectable Δ log 2(O) representing the 95% confidence interval of log 2(SPI) as demonstrated in provided Source Data for FIG. 5e. The average log 2(S/N) ratio was used to calculate the detectable fold-change in RNA-bound quantity or detectable Δ log 2(S) representing the estimated change in log₂(SPIS) necessary to elicit a detectable change in log 2(SPI); log 2(2\hat{ }(SD/sqrt(n)*tcrit)−1/(S+1))˜log 2(S/(S+1)) where SD=SD of log 2(SPI), n=sample size, S=2\hat{ }log 2(S/N), and tcrit=the corresponding t-critical value for two-tailed 95% confidence interval. Detectable Δ log 2(S) was estimated by assuming N is constant. This assumption was proven false but was considered less impactful for proteins representative of RNA-bound quantities (Supplementary Note 6c, d). Additional information, examples, proofs, and full equations for Excel were included in the provided Source Data for FIG. 5e.

The total RNA-bound protein in the sample was estimated as the sum of all SPIS values and represented by “total SPIS”. The total free protein in the sample was estimated as the sum of all SPIN values and represented by “total SPIN”. The absolute quantity of total RNA-bound protein in the sample was represented by total lSI and was considered dependent on UV-crosslinking conditions (total lSI in starting samples) and signal recovery. Total SPIS of RNP fractions containing total protein-bound RNA was considered representative of total RNA-bound protein. Protein abundances were estimated using the TPA or ‘Total Protein Approach’ by dividing average SPI values with the average total SPI and were represented as a percentage of total protein in the sample (i.e., “% TP” values). % TP values and average S/N ratios were used to estimate the abundance of RNA-bound (“% TP_{S}”) and free protein (“% TPN”) quantities as a percentage of total SPI according to equations (9-11).

Cumulatively, % TP_{S} and % TPN represented the estimated abundance of total SPIS and total SPIN in the sample. Protein abundances estimated as a percentage of other total populations in the sample (e.g., total SPICL) were represented by “% TP(CL)” values, where the parenthetical text indicates the identity of the total protein population. S/N ratios generated by only considering the estimated noise contributions of UV-crosslinked samples were represented by S/N(CL) ratios (Supplementary Note 6a).

“Relative abundance” represents the ratio of protein abundances (% TP/% TP) or Δ log 10(% TP) and was considered equivalent to their relative quantities (SPI/SPI) or Δ log 10(SPI) (Source Data FIG. 7h, Supplementary Note 4f). A decrease in free protein recovery without a decrease in total |S| was expected to increase % TPS of RBPs and non-RBPs without altering their relative RNA-bound abundances (% TPS/% TPS). This was evidenced by comparable % TP(S) contributions from RBPS and non-RBPs in both INP and LEAPRBP fractions despite a large difference in % TPS (Supplementary Note 8a).

“Non-specific % TP(S) contributions” referred to the % TP(S) contributions of non-RBPs (Supplementary Note 7e). A “favorable increase in % TPS” was considered an increase in % TPS which did not appreciably increase non-specific % TP(S) contributions. “Non-specific UV-enrichment” referred to UV-enrichment of free protein and was expected to increase non-specific % TP(S) contributions. Similar non-specific % TP(S) contributions were observed for other RNA-centric methods utilizing SILAC LC-MS/MS to accurately quantify free protein recovery: 2.6 for XRNAX and ˜5.0 for TRAPP. Non-SILAC comparison resulted in high non-specific % TPS contributions: 24.4 for OOPs and 28.4 for Ptex fractions. Notably, non-specific % TP(S) contributions for the referenced RIC study were only 1.5%. This was attributed to high % TPS of the RIC method and the observation that current GO-annotations of RBPs are largely based on their UV-enrichment* status in prior RIC-like (non-SILAC) experiments (Supplementary Note 8b). For these reasons, % TPS was considered a key metric when evaluating method specificity for RNA-bound RBPs because it provided key information about S/N, free protein contributions (% TPN), and non-specific UV-enrichment (Supplementary Note 7, 8).

Observed abundances (% TP) for proteins displaying S/N ratios>3 were considered representative of their RNA-bound abundances (% TPS) (i.e., % TP≈% TPS), Because the total protein in LEAP-RBP fractions was considered representative of total RNA-bound protein (total SPI≈ total SPIS), % TP values of proteins displaying S/N ratios>3 were considered representative of their total RNA-bound abundances (% TP≈% TPS≈% TP(S)) (Supplementary Note 4f). % TP values were log 10 normalized and adjusted by subtracting the minimum log 10(% TP) value of the MS dataset. This adjustment of log 10(% TP) values was done for graphical and RCS ranking purposes.

Method specificity for RNA-bound RBPs was evaluated graphically by comparing the abundances (log 10(% TP)) of RBPs and non-RBPs as a function of their average log 2(S/N) ratios. A larger range of protein abundances (highest % TP-lowest % TP or “% TP range”) resulted in a larger log 10(% TP) range and was considered indicative of an improved (i.e., lower) limit of detection (“LOD”). Cumulative frequency curves for comparison of adj. log 10(% TP) values only included proteins detected in all UVcrosslinked samples; adjusted log 10(% TP) range (0-6) was divided into 50 bins and the median values for each bin were plotted as a function of their cumulative frequencies with increasing % TP and represented as a percentage of total protein IDs. Cumulative frequency curves for average log 2(S/N) ratios were generated in the same way but only included proteins displaying positive S/N ratios; S can't be negative, 0/N=B.

### 8. RCS Rank Analysis.

“RBP confidence scores” (denoted by RCS) were generated for proteins detected in all UV-crosslinked samples and represent the product of adj. log 10(% TP) values and average log 2(S/N) ratios. Because protein abundance was a substantial contributor, a lower RCS ranking may result from MS-based quantitation biases. For example, XRN1 was found to be enriched in clRNP fractions by SRA and immunoblot while LRRC59 was de-enriched. However, XRN1 ranked lower than LRRC59 by protein abundance (% TP rank) by SILAC LC-MS/MS; 866 vs 290 respectively (FIG. 10). Low-abundance proteins only detected in UVcrosslinked samples (red box; FIG. 6b) were not included because their S/N ratios were considered less meaningful and more indicative of their lower abundance and quantitative accuracy (Supplementary Note 8a). RCS rank analysis was performed by binning proteins according to their ordinal RCS rank (each bin=100 proteins) and calculating the number of GO-annotated RBPs, average RCS, cumulative % TP contributions, log 2(average S/N ratios), average number of unique peptides, and average detectable Δ log 2(S) for each bin. The primary purpose of RCS or % TP ranking was to identify UV-enriched proteins more likely to be orthogonally validated by SRA and immunoblot. Accuracy of RCS ranking was considered dependent on method specificity for RNA-bound RBPs or % TP_{S} (Supplementary Note 8a). Notably, RCS ranking assumed higher UV-crosslinking efficiency, indicated by higher RNA-bound protein abundance, was indicative of physiological relevance. A full list of RBPs and non-RBPs identified by SILAC LC-MS/MS of LEAPRBP fractions and ranked by RCS is included in the provided Source Data for FIG. 6g.

### 9. Additional Protocol Information.

9a. Repeated Guanidinium Thiocyanate-Phenol-Chloroform Extraction (AGPC) Protocol.

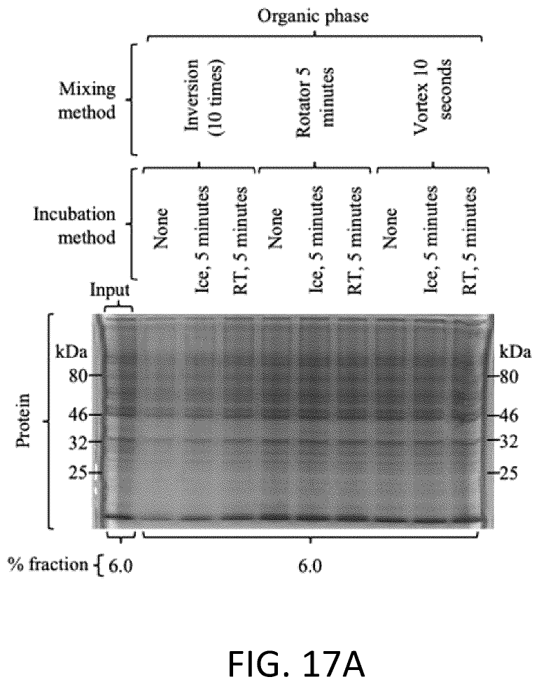

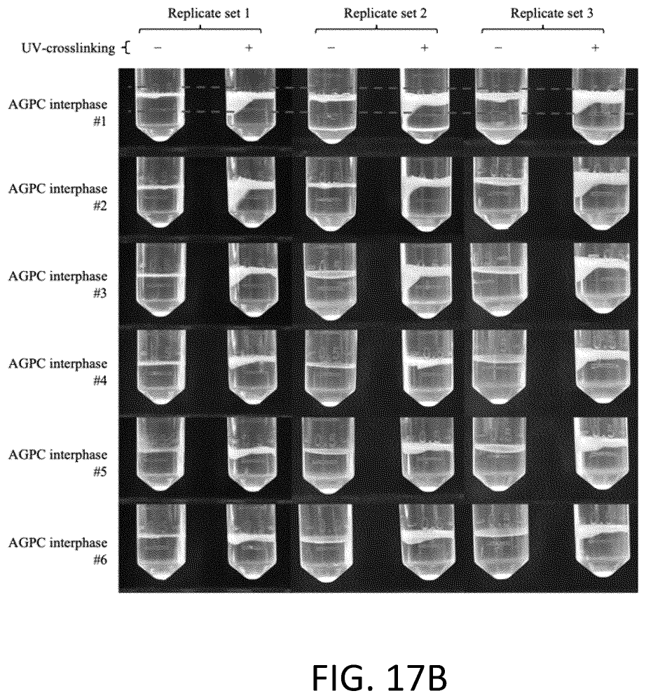

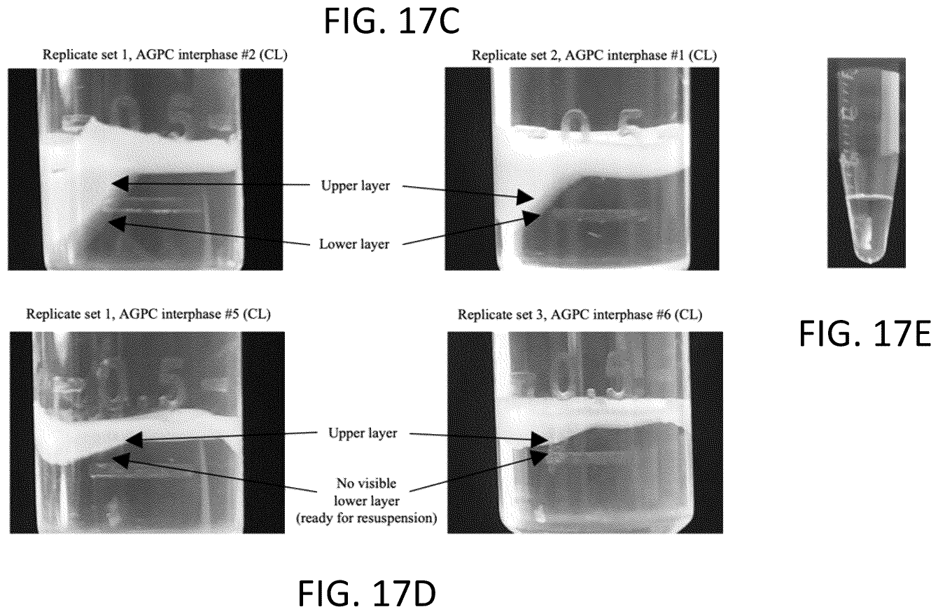

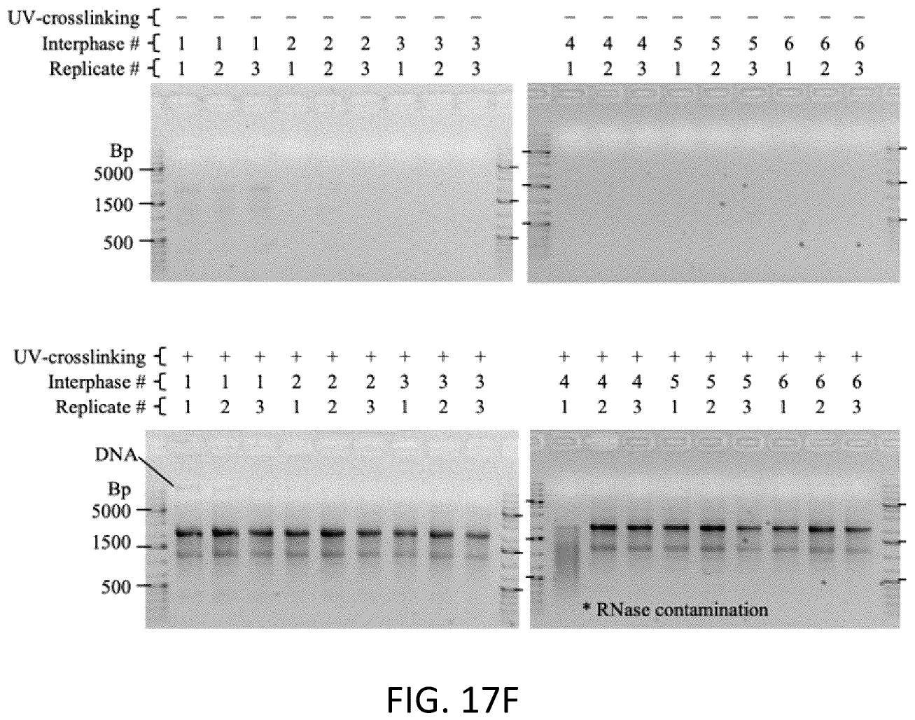

A gel loading pipette tip attached to a P1000 pipette tip was used to remove the aqueous and organic phases while leaving the interphase undisturbed. Resolubilizing the AGPC interphase in AGP by pipetting prior to adding chloroform and mixing was found to decrease the maximum % TPS of the final AGPC interphase (Supplementary Note 4e). Therefore, both were added sequentially, and samples were vigorously vortexed for 10 sec without pipetting. Residual organic or aqueous phase during repeated AGPC extraction did not impact results. However, most of the organic phase was removed prior to final suspension. To that point, the ability to remove most of the organic phase without disturbing the interphase served as a qualitative indicator that maximum % TPS has been reached (FIG. 17c-e, Supplementary Note 1a). Samples were not recentrifuged if the interphase was disturbed while removing the organic phase. In this scenario, a conservative removal of the organic phase was performed, fresh AGPC was added, and the samples were re-extracted.

9b. Isolation of RNP Fractions by LEAP-RBP.

LEAP-RBP was performed on 200 μL aliquots of AGP input suspensions (>6 parts AGP) or final AGPC interphase suspensions containing up to 55 μg RNA&DNA. Typically, most of the organic phase following repeated AGPC extraction was removed to avoid having to determine the optimal amount of chloroform to add. If necessary, one 200 μL aliquot of the final AGPC interphase suspension per sample was used to determine the appropriate volume of chloroform for precipitation of the remaining aliquots: 12 μL of chloroform were added and the sample was mixed by pulse vortexing several times; AGPC mixtures were kept off lid. If the AGPC mixture assumed a cloudy white appearance and retracted to the bottom of the tube (after step A; FIG. 2c), this indicated the optimal concentration of chloroform has been reached (14-16 μL). If necessary, an additional 2 μL of chloroform were added. Alternatively, most of the organic phase during repeated AGPC extractions and/or prior to the final resuspension of the AGPC interphase was found to speed up free protein depletion and eliminated the need to determine the optimal volume of chloroform to be added (14 μL). In these scenarios, signal loss which occurred during the repeated AGPC extraction step was corrected for using the strategy outlined in the provided Source Data for FIG. 15d-g.

Once AGPC mixtures assumed a cloudy appearance they were mixed by continuous vortexing for another 10 sec; AGPC mixtures were kept off lid. Aliquots of the AGP input suspension were processed using 14 μl chloroform. Unlike final AGPC interphase suspensions, a cloudy appearance before adding chloroform was not problematic. Using an appropriate volume of chloroform and keeping RNA concentrations above 10 ng/μL of AGP were necessary to ensure optimal recovery (Supplementary Note 1). Four parts of a precipitation solution containing 3.75 M LiCl (10515, VWR) and 50% isopropanol were gently added/layered onto the AGPC mixtures, and the tubes were closed. Using a sample rack, samples were slowly inverted to 90 degrees and/or until the AGPC mixture was displaced from the bottom of the tube, and then the rack was returned to an upright position followed by incubation on bench for 1 minute. This process was repeated at least four more times, switching the direction of inversion, increasing the angle, and increasing the speed during reversion.

Additional inversions were used if residual AGPC mixture remained at the bottom of the tube and the final reversion was performed forcefully. The protein and RNA composition of AGP suspensions were found to alter the optimal mixing speed and/or number of inversions. In all cases, performing the initial three inversions slowly before increasing speed resulted in optimal recovery while additional inversions did not diminish % TP_{S} (Supplementary Note 1). Samples were then homogenized by vigorous vortexing (5 sec), centrifuged at 14,000×g for 5 min at 20° C., and supernatants were removed. RNP pellets were rinsed twice with 1 mL RT 95% methanol by inverting the tube 2-3 times and removing the supernatant; a syringe equipped with a 19 ga 1½″ needle facilitates easy removal of the supernatant from multiple samples. Leaving the 1st, 2nd, or 3rd 95% methanol wash on the RNP pellets overnight at room temperature did not result in more free protein recovery; however, failing to remove the methanol washes rapidly after inverting sample tubes does.

Before removing the final methanol wash, RNP pellets which remained adhered to the bottom of the tube need to be dislodged. This was done by sliding a P1000 pipette tip down the side of the tube and against the top of the pellets until they started to move. Then, a small volume of the final methanol was pipetted to fully displace the RNP pellet off the bottom of the tube. Following removal of the final methanol wash, pellets were transferred to a new tube before resuspension. This was done by pouring ˜1 mL of 95% methanol from the new tube into the tube containing the RNP pellet and then immediately pouring it back into the new tube. The methanol was removed and RNP pellets were air dried by leaving the tube open and incubating for 10 min at RT. RNP pellets were resuspended at the desired concentration with 1% LiDS TE by incubating for 30 min at room temperature with occasional pipetting (90% sample volume, 8 times) at the 2-, 16-, and 30-min mark. If bubbles formed during resuspension, samples were incubated at 55° C. for 20 sec, mixed by vigorous vortexing for 5 see, and centrifuged at 3,000×g for 10 sec at 20° C. To mitigate the formation of bubbles, pipette tips were centered above the bottom of the tube while aspirating 90% of the sample volume and gently swirled against the bottom of the tube while ejecting. RNP suspension were then used immediately or stored at −80° C. for up to a year. Working concentrations of LEAP-RBP isolated RNPs ranged from 0.1-4.0 μg of protein bound RNA/μL.

9c. LEAP-RBP DNA Depletion Step. Turbo

DNase is strongly inhibited by LiDS and so for the DNA depletion step, RNP pellets were resuspended in TE buffer. Samples were gently resuspended while keeping the samples at the bottom of the tube; not doing so diminished recovery (Supplementary Note 1c). Samples were not quantified during this step. Using a new pipette tip for each sample suspension, 5 μL of a master mix containing TE buffer, 10× Turbo DNase buffer, and Turbo DNase were added and mixed by swirling the pipette tip for 3 sec. Samples were incubated at 37° C. for 15 min without agitation and nine parts (180 μL) fresh acid guanidinium thiocyanate-phenol (2:1) buffer were added. Samples were precipitated according to the LEAP-RBP protocol using 14 μL of chloroform and resuspended in 1% LiDS TE at the desired concentration; samples were vortexed for 10 sec before and after adding chloroform while keeping AGPC mixtures off lid.

9d. SDS-PAGE, SYBR Safe, Coomassie Blue, Silver Stain Staining.

LB WS was added to samples for a final detergent concentration of 2% (3.6 μL per 12.0 μL reaction containing 4 μL 1% LiDS TE). Samples were heated for 15 min at 65° C. in a thermocycler with heated lid (98° C.) and chilled on ice for at least 2 min prior to loading. Samples were not kept on ice for extended periods of time to avoid precipitation of SDS. Empty wells were loaded with LB WS to match the amount of detergent in samples (4.2 μL for prior example). Samples were separated on a 0.75 mm, 15-well, 4-12% gradient polyacrylamide gel (6, 8, 10, 12% (1:1:1:1) resolver, 4% stacker) at constant voltage (80 V) for 1.5 hours at RT (Supplementary Note 2c). For dual SYBR Safe (DNA&RNA) and Coomassie Blue (protein) staining, each polyacrylamide gel was incubated in 30 mL of 1× TBE containing 1.2 μL SYBR Safe (S33102, Invitrogen) on an orbital shaker (65 rpm) for 20 min at RT and rinsed three times with 50 mL DI water prior to imaging. Then, each gel was incubated in 30 mL Coomassie Blue stain (25% isopropanol (v/v), 10% glacial acetic acid, and 0.05% (m/v) Coomassie Brilliant Blue G-250 (1610406, Biorad)) on an orbital shaker (65 rpm) for 20 min at RT, rinsed three times with 50 mL DI water, and de-stained by incubating in 30 mL pre-warmed 10% acetic acid (v/v) on an orbital shaker (65 rpm) for 10 min at RT. The destaining step was repeated followed by overnight standing incubation in 30 mL fresh destain solution at RT. Then, each gel was rinsed three times with 50 mL DI water, incubated on bench in 50 mL DI water for 20 min twice, and rinsed an additional three times with 50 mL DI water prior to imaging. Silver Stain staining was performed on Coomassie Blue stained gels using a ProteoSilver Stain Plus Silver Stain Kit (PROTSIL2, Sigma). Imaging of SYBR Safe, Coomassie Blue, and Silver Stain-stained gels was performed using an Amersham Imager 600 (see corresponding Source Data).

9e. Immunoblot.

Following separation by SDS-PAGE (detailed above), samples were transferred to nitrocellulose membranes using Bjerrum and Schafer-Nielsen transfer buffer (48 mM Tris and 39 mM glycine) supplemented with 10% methanol (v/v) and 0.03% SDS. For each transfer, the gel was equilibrated in 100 mL of 1× transfer buffer on an orbital shaker (65 rpm) for 15 min at RT. Then, one 7×9 cm nitrocellulose membrane and six 9×11 cm thin filter paper sections were added individually to the gel container. Transfers were done at constant voltage (20 V) for 30 min at RT using a Trans-Blot SD semi-dry electrophoretic transfer cell (170-3940, Bio-Rad). Alternatively, samples were wet transferred to nitrocellulose membranes using 25 mM Tris, 96 mM glycine, 0.05% SDS, and 20% methanol (v/v). The gel, membrane, and filter papers were equilibrated in 100 mL of 1× transfer buffer as described previously. Transfers were done at constant voltage (24 V) overnight at 4° C. using a Bio-Rad Mini-Protean II system. Blocking and immunoblotting was performed for each protein target. Signal detection was performed using WesternBright ECL HRP substrate (K-12045, Advansta) and an Amersham Imager 600 (see corresponding Source Data).

9f. Sample Preparation for MS Proteomic Analysis.

For Turbo DNase digestion of each input sample containing 20 μg of total protein, 42.5 μL TE buffer were added and the sample was incubated on bench for 5 min at RT without pipetting. Then, 5 μL of 10× Turbo DNase buffer were added and the sample was incubated on bench for 2 min at RT without pipetting. Using a new pipette tip for each sample, 90% of the sample volume was pipetted 8 times while swirling the pipette tip against the bottom of the tube. Samples were incubated for 2 min at RT and the pipetting step was repeated using the same pipette tip following by incubation for an additional 2 min at RT. Using a new pipette tip for each sample, 2.5 μL of Turbo DNase were added and the sample was mixed by swirling the pipette tip for 5 sec. After RNase and Turbo DNase digestion steps and during methanol precipitation/washing steps, precipitates were less adherent to the side of microcentrifuge tubes. Therefore, supernatants were removed using a P200 pipette tip attached to a P1000 pipette tip or by using a syringe equipped with a 19 ga 1½″ needle while leaving 50-100 μL residual supernatant. Samples were centrifuged a second time after removing most of the final 95% methanol wash and before removing the residual 95% methanol (˜100-200 μL) using a P10 pipette tip attached to a P1000 pipette tip. Protein concentration of samples was kept above 25 ng/μL as estimated by BCA quantitation to ensure efficient protein recovery by 95% methanol v/v.

### Supplementary Notes

Supplementary Notes provide additional observations and rationale for successful applications of the methods herein, characterization of S/N, and S/N-based analyses.

### Supplementary Note 1

This note provides supporting information and technical considerations for LEAP-RBP and DNA depletion step. 1a. Repeated AGPC extraction concentrates clRNPs and makes them amenable to LEAP-RBP. The combination of AGPC extraction and LEAP-RBP is a powerful tool for rapid and efficient purification of RNA-bound protein from biological samples. AGP, more commonly known by its commercial name “Trizol”, is a universal method for simultaneous purification of RNA, protein, and DNA from biological samples. UV-crosslinked RNA-protein adducts efficiently partition to the AGPC interphase.

In practice, AGPC extraction allows concentration of covalently bound RNA-protein adducts from very dilute samples for subsequent LEAP-RBP; add 4 parts AGP, 1 parts chloroform, and perform AGPC extraction. When in doubt, purified clRNPs isolated by LEAP-RBP under optimal conditions were diluted with the untested sample buffer and processed accordingly. Recovery efficiency was tested by comparing the isolated clRNPs to an equivalent % of the purified clRNPs used as input. Shearing lysates with a syringe needle was necessary to allow removal of the aqueous phase without disturbing the interphase. It was empirically determined that syringe needle shearing of lysates or harsh vortexing of AGPC mixtures does not diminish UV-crosslinked RNP integrity. For example, clRNPs isolated from AGPC interphase suspensions following repeated AGPC extraction were comparable to RNP fractions isolated from AGP input suspensions without repeated AGPC extraction by SRA analysis (FIG. 3a, b); RNA samples isolated from clRNP fractions with repeated AGPC extractions were found to be of high quality by bioanalyzer (RIN>9). These data demonstrate that shearing and vortexing does not impact RNP integrity or results. However, AGPC mixing conditions were found to impact the rate at which free protein is depleted from the AGPC interphase (FIG. 17a).

Another factor found to impact the rate of free protein depletion is the ratio of phenol to chloroform. To ensure the proper ratio is maintained during repeat extractions, pipette tips were pre-wetted. This was done by pipetting to and from the stock solution a few times before adding solvents to samples in an identical fashion. During repeated AGPC extraction, residual aqueous and organic phase did not impact the % TP_{S} of final AGPC interphase samples evaluated by SRA with Coomassie Blue (protein) staining nor does letting the samples equilibrate to room temperature (RT) following centrifugation. If the interphase was disturbed, the samples were not re-centrifuged. Instead, fresh AGPC were added, and the samples were re-extracted. The organic phase contains phenol and chloroform, and the aqueous phase contains GT and aqueous buffers (PBS, etc.). Therefore, the ratio of phenol and chloroform was maintained even when different percentages of the aqueous and organic phases were removed if AGP and chloroform were added at the proper ratio.

For the repeated AGPC extraction experiment presented in this study (FIG. 1b, c), AGPC extractions were performed in 2 mL microcentrifuge tubes. Because the interphase of non-crosslinked cells was more dispersed, the volume of aqueous and organic phase removed was limited to prevent introduction of technical artifacts (red dashed line; FIG. 17c). The interphase of UV-crosslinked cells appeared to have two layers; a lower layer which appears more dispersed, and an inner or upper layer with higher apparent density and integrity (FIG. 17d). When removing the organic phase, the lower layer was found to break apart and fall into the organic phase. It is suspected that the upper layer contains clRNPs but removing was avoided either by limiting the volume of organic phase removed during the first few extractions. Repeated AGPC extractions appears to deplete the lower layer until only the upper layer remains (FIG. 17e). Removing most of the organic phase was performed for resuspension of the interphase in fresh AGP and LEAP-RBP (FIG. 17f). The lower layer impeded organic phase removal. Therefore, performing repeated AGPC extractions until the lower layer was depleted facilitated organic phase removal (FIG. 17e).

Repeated AGPC extractions is not necessary to isolate most RNA-bound protein with sufficient S/N by LEAP-RBP; RPN1 was the only exception observed (FIG. 3a, b). Therefore, the main purpose of repeated AGPC extraction is to remove free RNA and enhance sensitivity of downstream applications e.g., SRA (FIG. 1b, c, FIG. 17f) and concentrate clRNPs while making them amenable to LEAP-RBP (>6 parts AGP; Methods).

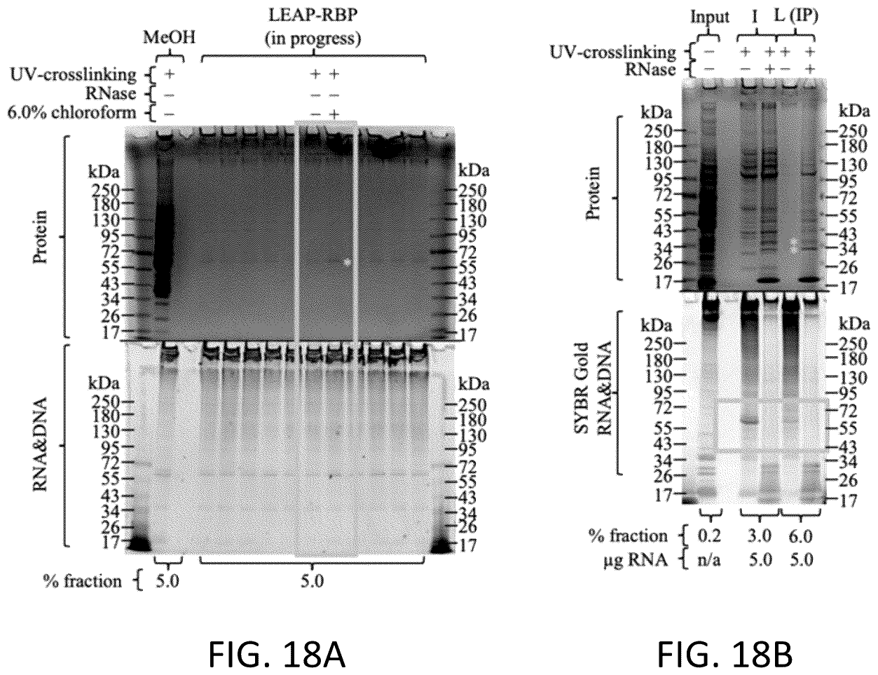

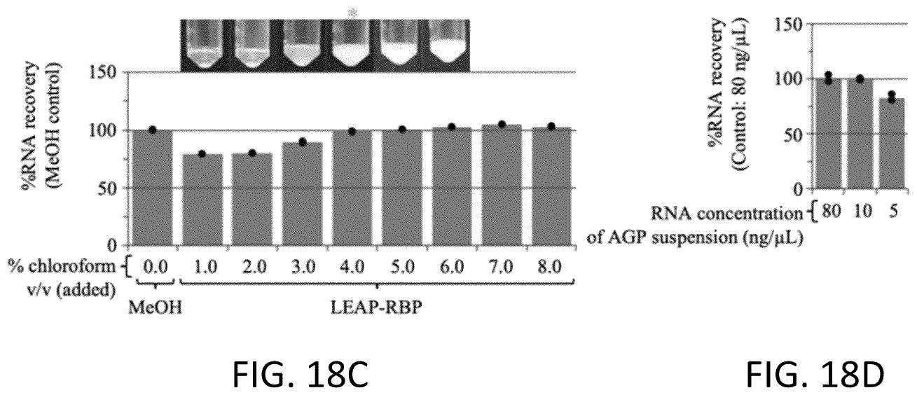

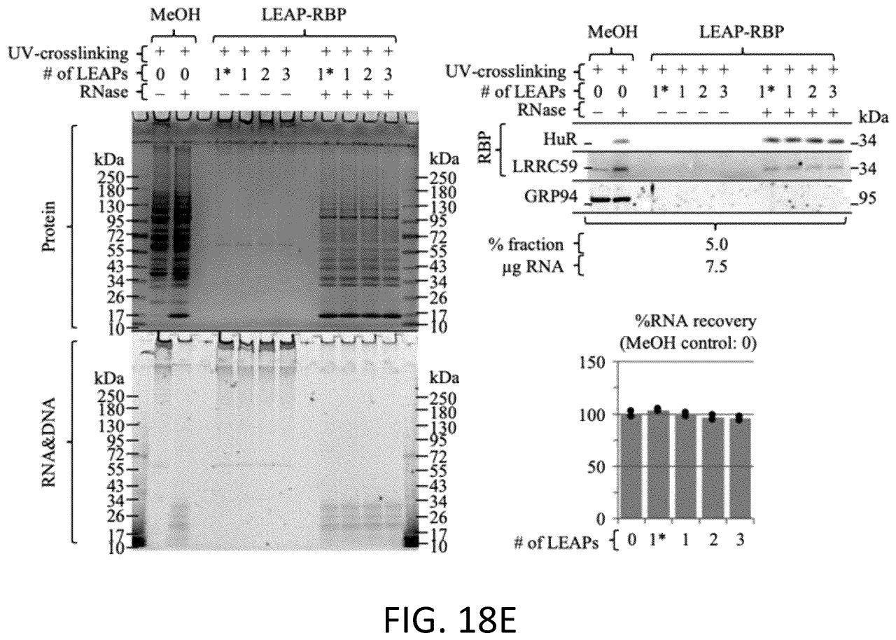

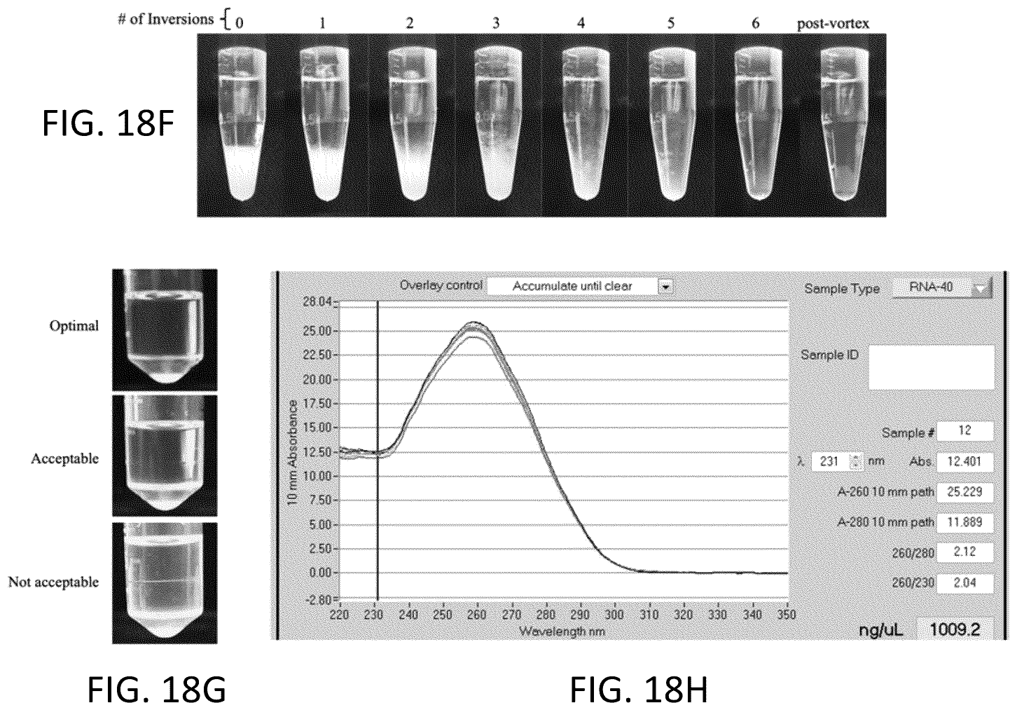

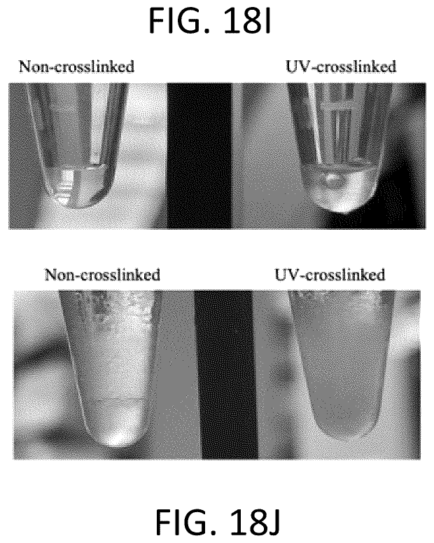

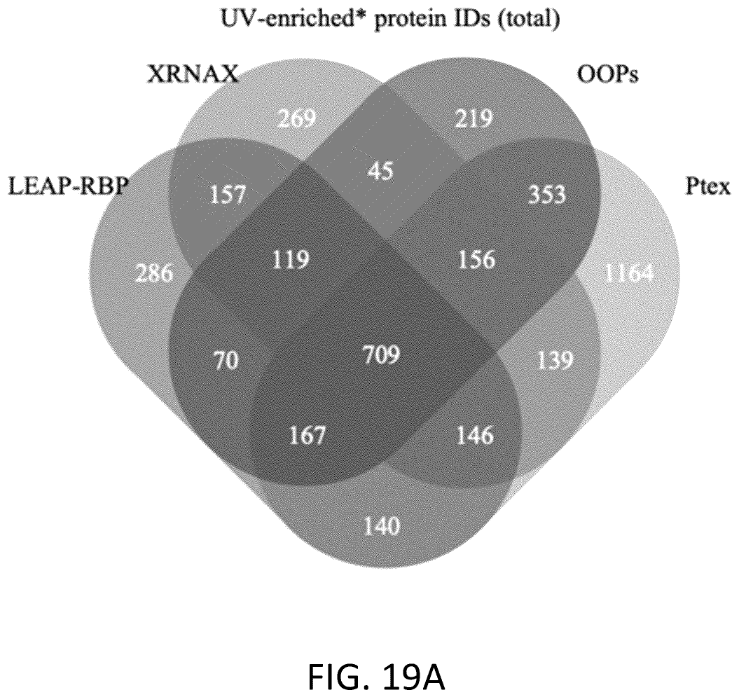

1b. LEAP-RBP provides rapid and efficient isolation of clRNPs from AGP suspensions. The behavior of samples during the LEAP-RBP step varies depending on the composition of the AGP suspension. Initially, LEAP-RBP was developed to work on final AGPC interphase suspensions. It was assumed that removal of chloroform and solubilization of the interphase in AGP was necessary for high % TP_{S}. However, when chloroform was intentionally added to test this assumption, it was found to increase yield without diminishing % TP_{S} (FIG. 18a). Under suboptimal conditions, proteins bound to small RNAs displayed decreased recovery when compared to an equivalent percentage of methanol (95% v/v) precipitated AGPC interphase samples. The dual-stained 65 kD clRNP (gold asterisk; FIG. 18a) was determined to be a reliable indicator of small RNA-bound protein recovery as quantified by RTqPCR. Earlier versions of LEAP-RBP without near 100% recovery showed diminished recovery of the 65 kD clRNP when compared INP fractions containing an equivalent amount of protein-bound RNA (gold box; FIG. 18b); differences in the relative intensities of RNase-sensitive bands illustrates RBPspecific signal loss (gold asterisks; FIG. 18b). The optimized LEAP-RBP method shows comparable recovery of the 65 kD clRNP from final AGPC interphase suspensions of UV-crosslinked cells as methanol (95% v/v) or INP precipitation methods (FIG. 2a).

Comparison of LEAP-RBP fractions isolated from AGP input suspensions containing UV-crosslinked or non-crosslinked cells by SRA and SYBR Safe (RNA&DNA) staining demonstrates that the 65 kD clRNP is formed by UV-crosslinking (SYBR Safe stained gel; FIG. 3a, FIG. 13b). Furthermore, separating proteinase K treated clRNP fractions by SDSPAGE results in a downward shift of the SYBR Safe (RNA&DNA) stained RNA interactor to ˜28 kD and is no longer visible with Coomassie Blue (protein) staining (FIG. 11D). Based on these data, the protein and RNA interactors were estimated to be roughly 36 kD and 100 bp (33 kD), respectively. The optimal volume of chloroform results in a noticeable qualitative change (FIG. 18c); the optimal final concentration of chloroform was determined to be between 6-8% (Supplementary Methods). Solubilization of the AGPC interphase and re-introduction of chloroform ensures reproducible results.

All LEAP-RBP steps for this study were performed in 1.5 mL microcentrifuge tubes with a rounded bottom (490003, VWR); microcentrifuge tubes with pointed ends were found to impede mixing during inversions. AGPC interphases were routinely resuspended in ˜1.3 mL fresh AGP and split across six 1.5 mL microcentrifuge tubes (200 μL each). Samples were centrifuged briefly to concentrate samples at the bottom of the tube (FIG. 17f). The minimum amount of RNA for efficient recovery was found to be 1 μg but routinely kept above 2 μg (FIG. 18d). It was found to be useful to estimate the expected yield under optimal conditions by diluting 20-50 μL of the original AGP suspension to 200 μL with fresh AGP and performing LEAP-RBP with the optimal volume of chloroform (14 μL). If the yield was above 2 μg, it was used to estimate the yield of the undiluted aliquots. For the undiluted aliquots, 12 μL of chloroform were added initially and samples were pulse vortexed several times; AGPC mixtures were kept off lid. If sample appearance did not change (FIG. 18c), an additional 2 μL were added and the samples were pulse vortexed again. In addition to the cloudy-white appearance, AGPC mixtures were found to retract to the bottom of the tube after brief vortexing if the optimal amount of chloroform has been added (FIG. 2c). Turbo DNase treated samples mixed with AGP were an exception and did not stick to the side of the tubes.

When performing LEAP-RBP on AGP suspensions with low protein-content, the emulsion of AGPC mixtures may separate prior to adding the precipitation solution. Extending the duration of most steps to 3 min did not impact the results (asterisk; FIG. 18e). During the LEAP step, samples are inverted to increase the surface area between the two solvents. Various mixing methods were tested, and the inversion method was found to be the most consistent. Gently mixing the two solvents during the first few inversions was critical to optimal yields (FIG. 18f). The appearance and behavior of samples were found to be dependent on sample composition; samples with lower protein levels mixed more readily (e.g., Turbo DNase treated RNP samples isolated by LEAP-RBP); samples with more protein required more forceful inversions after the first 3 inversions to fully mix (e.g., input AGP-suspensions containing 400 μg protein and upwards of 55 μg nucleic acids). Excess chloroform results in a visible phase-separation (FIG. 18g); samples with a sharp phase-boundary containing visible precipitates were not used. 1c. LEAP-RBP allows rapid and efficient isolation of total RNA from AGP input suspensions or following DNA-digestion. As noted, AGP input suspensions appear cloudy before chloroform addition; the optimal volume of chloroform (14 μL) was added for the LEAP-RBP step regardless of sample appearance. Samples appeared clarified following inversion(S) and mixing by vortex. The use of methanol vs other alcohols (e.g., EtOH) or aqueous-based washes (LiCl) was chosen because methanol is more efficient at removing lipids, saccharides, guanidinium thiocyanate, and phenol than ethanol or aqueous based solvent. After a single methanol rinse, samples were found free of contaminants such as GT and phenol, as determined by UV-spectrophotometry (FIG. 18h).

The integrity of microcentrifuge tubes was apparently diminished with each LEAP step. Therefore, pellets were transferred to a new tube before resuspension. This was done by pouring ˜1 mL of 95% methanol from the new tube into the tube containing the RNP pellet and then immediately pouring it back into the new tube. For experiments where precision was key, multiple aliquots were processed in parallel and pooled prior to resuspension to reduce technical variability. For example, samples for SILAC LC-MS/MS contained 150 μg of protein-bound RNA spread across six 1.5 mL microcentrifuge tubes. Following the initial LEAP-RBP step, pellets were combined into three tubes for DNA depletion. Following DNA depletion and the second LEAP, pellets were again pooled for resuspension and LC-MS/MS sample prep. During the DNA depletion step, samples were maintained at the bottom of the tube. TE suspended clRNPs adhere to the sides of microcentrifuge tubes; not keeping samples at the bottom of the tubes led to sample loss. Samples were incubated in 15 μL TE buffer for 2 min and then pipetted gently 8 times (5 μL). Samples were incubated at RT for 2 min and the process was repeated if necessary. Depending on RNA concentration and UV-crosslinking conditions (i.e., μg RNA-bound protein/μg RNA), samples may not resolubilize completely even after extended incubation and pipetting. This does not affect DNA digestion efficiency or recovery if samples are incubated in TE for a minimum of 5 min and triturated as noted previously.

Samples containing clRNPs take on a cloudy appearance upon addition of Turbo DNase buffer and adhere to pipette tips (FIG. 18i). Therefore, the master mix containing Turbo DNase buffer and Turbo DNase were added directly to the bottom of the sample suspension and instead of pipetting, samples were mixed by swirling the pipette tip. The DNA depletion step was designed to overcome UV-dependent alterations in sample physical properties and allow efficient DNA digestion and subsequent recovery by LEAP-RBP. Following Turbo DNase digestion and re-introduction of AGP, samples were vortexed for 10 sec at medium setting before and after adding chloroform for efficient recovery during the second LEAP. During the second LEAP, UV-crosslinked samples were found to behave differently than non-crosslinked samples containing equivalent amounts of RNA (FIG. 18j). A ˜10% decrease in quantifiable RNA by UV-spectrophotometry after performing the DNA depletion step on clRNP fractions was expected; no apparent differences in signal recovery or RNase-sensitive protein profiles were observed (FIG. 3a, b, FIG. 12b). Samples and buffers were kept at RT during all steps unless indicated otherwise.

### Supplementary Note 2

This note provides supporting information and technical considerations for the SDS-PAGE RNase-sensitivity Assay.

2a. Validation and Technical Considerations for RNase-Digestion of clRNPs.

Comparison of RNase-treated and untreated clRNPs by SDS-PAGE is a simple and cost-effective method for identification of RBPs based on their RNase-sensitive mobility in SDS-PAGE. However, the sensitivity, accuracy, and reproducibility of SRA depends both on the quality of the clRNP isolation method and the SRA conditions themselves. See Supplementary Note 1a-c and Supplementary Methods for suggestions and technical information regarding isolation of clRNPs by LEAP-RBP. RNase-digestion reactions were performed in thermocycler tubes as 10 or 12 μL reactions with 4 μL of 1% LiDS TE suspended samples. RNA integrity is maintained in untreated samples at 37° C. and 1% LiDS TE does not inhibit RNase when using the recommended digest conditions (Methods). LEAP-RBP fractions isolated from AGP input (RNPs), or final AGPC interphase (clRNPs) suspensions were resuspended in 1% LiDS TE, quantified, diluted, and clarified for SRA analysis as described in Supplementary Methods. Sample tubes were centrifuged briefly with a mini centrifuge prior to adding RNase-digestion components. Master mixes containing either untreated or RNase-digestion components were added to samples (FIG. 17b). Samples were briefly vortexed at medium setting and centrifuged again after adding master mixes or 5×LB WS for SDS-PAGE. Samples were stored at −80° C. or used immediately for SDS-PAGE. Frozen samples were thawed and incubated at RT for 30 min prior to denaturation.

2b. Technical Considerations for Identification of RNase-Sensitive RBPs by SRA and Immunoblot.

Protein bands in RNase-treated and untreated samples should have comparable dimensions fir confident detection of RNase-sensitive RBPs. Therefore, efforts were made to avoid artifacts that lead to lane narrowing or widening during SDS-PAGE of RNPs; this is mainly an issue when analyzing untreated samples. Following sample denaturation (Methods), samples were quickly moved and centrifuged briefly with a mini centrifuge (3 sec), vortexed for 2 sec at medium setting and centrifuged again before being placed on ice. When adding RNase treated and untreated samples in neighboring lanes, samples were added in one direction across the gel (FIG. 1b); alternating wells allowed samples from one well to diffuse towards neighboring wells before they were filled leading to lane narrowing artifacts. Loading RNase treated or untreated samples containing>5 μg of RNA in adjacent wells results in separation artifacts; this can lead to false apparent RNase-sensitivity. Therefore, RNase-treated and untreated samples were loaded in one of two configurations: alternating with gaps (Source Data FIG. 2a, Source Data FIG. 3a), or segregated clustering (FIG. 13b, c, 18e). When using the latter configuration, loading RNP fractions with large differences in RNA and/or protein composition adjacent to each other led to lane-narrowing artifacts.

2c. SDS-PAGE and Transfer Conditions for SRA and Immunoblot.

The composition of the polyacrylamide gel or SDS-PAGE and transfer conditions can affect SRA results. The transfer conditions used in this study were optimized to work for proteins ranging from ˜28-180 kDa. After transferring proteins to membranes, polyacrylamide gels were Coomassie Blue stained to assess transfer efficiency. Protein enrichment in RNP fractions was assessed by including input samples containing an equivalent amount of protein (μg) as RNP samples. If the protein of interest was detected in input samples, then it was assumed SDS-PAGE and transfer conditions would allow detection of the same protein in RNase-treated RNP fractions. Gradient polyacrylamide gels were necessary to ensure efficient transfer and simultaneous assessment of RBPs with different molecular weights.

### Supplementary Note 3

This note provides supporting information for estimating RNA, protein, and RBP-specific UV-crosslinking efficiencies.

3a. LEAP-RBP Allows Direct Quantitative Measurement of Total RNA and Protein UV-Crosslinking Efficiency.

UV-crosslinking conditions which maximized the amount of material (μg protein-bound RNA/total RNA) were selected for development of LEAP-RBP. 0.4 J/cm² (254 nm) maximized free RNA depletion from the aqueous phase during AGPC extraction (˜75-80%) (FIG. 1b). While indirect, the data suggests UV-irradiating cells with 0.4 J/cm² (254 nm) crosslinks roughly 75-80% of total RNA to protein. The fraction of RNA that is UV-crosslinked to protein can be used to measure UV-crosslinking efficiency of RNA. LEAP-RBP and repeated AGPC extraction may directly measure RNA UV-crosslinking efficiency by allowing simultaneous isolation of total protein-bound RNA and total RNA from UV-irradiated samples (RNA UV-crosslinking efficiency=total protein-bound RNA/total RNA*100). However, for this to be an accurate assessment, three prerequisites should be met: 1) repeated AGPC extractions should deplete free RNA and maintain protein-bound RNA with near 100% efficiency; 2) LEAP-RBP should recover near 100% of protein-bound and unbound RNA species; 3) DNA should be efficiently depleted without compromising the near 100% recovery of protein-bound and unbound RNA species. For these purposes, near 100% RNA recovery refers to a non-significant difference in RNA yield (μg RNA/% fraction) as quantified by UV-spectrophotometry when compared to a suitable control. Multiple lines of evidence (below) support that these prerequisites have been met.

### Free RNA is Efficiently Depleted During Repeated AGPC Extractions.

Two repeated AGPC extractions were sufficient to fully deplete RNA at the AGPC interphase of non-crosslinked cells as quantified by UV-spectrophotometry or analyzed by SYBR Safe (RNA&DNA) staining of AGPC interphase samples separated by SDS-PAGE and TBE (FIG. 1b, FIG. 10f). These data demonstrate that free RNA is rapidly depleted during repeated AGPC extraction.

LEAP-RBP Recovers Near 100% of Protein-Bound RNA from Final AGPC Interphase Suspensions.

No significant differences in RNA recovery were detected between LEAP-RBP and INP fractions isolated from final AGPC interphase suspensions and methanol (95% v/v) precipitated AGPC interphase samples by UV-spectrophotometry (FIG. 2a). INP is a more traditional RNA-precipitation method (isopropanol and NaCl) utilizing lengthier incubation times and co-precipitants (GlycoBlue) for efficient RNA-centric isolation of protein-bound RNA. Methanol is a highly efficient protein-centric method for orthogonal assessment of RNA-bound protein recovery by INP and LEAP-RBP (Methods). Methanol precipitated LEAP-RBP supernatants do not contain quantifiable RNA by UV-spectrophotometry and are devoid of RNase-sensitive proteins by SRA and Coomassie Blue (protein) staining (FIG. 11A); this includes the ˜65 kD clRNP indicative of small RNA-bound protein recovery SYBR Safe (RNA&DNA) stained gel (FIG. 3a, FIG. 13b, FIG. 18a, b). Lastly, LEAP-RBP fractions isolated from final AGPC interphase suspensions of UV-crosslinked cells can be subjected to 2 additional LEAP steps without a statistically significant decrease in RNA yield as quantified by UV-spectrophotometry, one-way ANOVA, F(3, 4)=0.73, p=0.584 (FIG. 11B), or apparent change in RNase-sensitive protein profiles evaluated by SRA with Coomassie Blue (protein) staining (FIG. 18e).

LEAP-RBP Recovers Near 100% of Protein-Bound and Unbound RNA Species from AGP Input Suspensions.

LEAP-RBP was performed on AGP input suspensions (without repeated AGPC extraction) containing equivalent amounts of UV-crosslinked or non-crosslinked cells and RNA yield was quantified by UV-spectrophotometry (FIG. 3a). No significant effect of UV-crosslinking on RNA yield (with or without DNA depletion step) was detected demonstrating unbiased recovery of protein-bound and unbound RNA species by LEAP-RBP; two-way ANOVA: non-significant main effect of UV-crosslinking on RNA yield, (F(1, 8)=0.59, p=0.463. This finding was further validated in a UV-dose response experiment and one-way ANOVA to analyze the effect of UV-dose (independent variable) on RNA yield (dependent variable) of LEAP-RBP w/DNA depletion step: non-significant main effect of UV-dose (0.0, 0.1, 0.2, 0.4, and 0.8 J/cm²; 254 nm) on RNA yield (FIG. 13a), F(4, 5)=0.89, p=0.534.

RNA-bound proteins exhibit efficient partitioning to the AGPC interphase.

AGPC interphase samples isolated by methanol (95% v/v) precipitation following up to 6 AGPC extractions display comparable RNase-sensitive protein profiles by SRA and immunoblot (FIG. 1c). Furthermore, LEAP-RBP fractions isolated from AGP input and final AGPC interphase suspensions from the same UV-crosslinked sample display comparable RNase-sensitive protein profiles and signal recovery by SRA with Coomassie Blue (protein) staining or immunoblot (FIG. 3a, b). These data indicate that RNA-bound proteins exhibit unbiased partitioning to the AGPC interphase and are efficiently maintained during repeated AGPC extractions.

### The LEAP-RBP DNA Depletion Step Efficiently Depletes DNA Contaminants.

Comparisons of DNase-treated and untreated samples by SRA shows near-complete depletion of RNase-insensitive SYBR Safe (RNA&DNA) stained species in the stacker of polyacrylamide gels during SDS-PAGE (FIG. 12b). The DNA depletion step removed>99% of DNA present in LEAP-RBP fractions isolated from final AGPC interphase suspensions of UV-crosslinked cells as quantified by qPCR (FIG. 12b). These data indicate that the SYBR Safe stained RNase-insensitive species in the stacker of polyacrylamide gels during SDS-PAGE is DNA; their higher intensity in LEAP-RBP fractions isolated from AGP input suspensions compared to final AGPC interphase suspensions supports this assessment (FIG. 3a) as repeated AGPC extraction depletes DNA relative to RNA (FIG. 17f).

### The Second LEAP Step Following the DNA Depletion Step Recovers Near 100% of Protein-Bound and Unbound RNA Species.

AGP input suspensions containing UV-crosslinked or non-crosslinked cells were subjected to LEAP-RBP with or without the DNA depletion step which includes a second LEAP step; RNA yield (dependent variable) was quantified by UV-spectrophotometry and analyzed by two-way ANOVA with DNA depletion and UV-crosslinking status as the independent variables (FIG. 3a); non-significant interaction between UV-crosslinking status and DNA depletion step on RNA yield, F(1, 8)=0.01, p=0.915; non-significant main effect of UV-crosslinking status on RNA yield, F(1, 8)=0.59, p=0.463. However, there was a statistically significant main effect of DNA depletion step on RNA yield; F(1, 8)=294.98, p<0.001. DNA contamination contributes to overall absorbance values during quantitation of RNA yield (UV-spectrophotometry). Therefore, the effect of DNA depletion step on RNA yield should not be taken as a decrease in RNA recovery. As evidence of this, comparison of these fractions by SRA with Coomassie Blue (protein) staining or immunoblot shows comparable RNase-sensitive protein profiles despite differences in RNA yield as quantified by UV-spectrophotometry (nanodrop) (FIG. 3a, c). The non-significant main effect of UV-crosslinking status on RNA yield indicates LEAP-RBP recovers both protein-bound and unbound RNA species in an unbiased manner, F(1, 8)=0.59, p=0.463.

Cumulatively, these data demonstrate that comparing the RNA yield of LEAP-RBP from AGP input suspensions with DNA depletion step (total RNA) and final AGPC interphase suspension with or without DNA depletion step (total protein-bound RNA) allows accurate and direct assessment of RNA UV-crosslinking efficiency. Using this approach, UV-irradiating HeLa cells with 0.4 J/cm² (254 nm) crosslinks ˜70% of RNA species (FIG. 3a, b). These results are close to indirect estimates obtained by the OOPs method (75%-80%). While total RNA yield was not quantitated for other experiments in this study, the ratio of total RNA/total protein (˜0.11 mg RNA/1.0 mg total protein) from the experiment above (FIG. 3a, b) can be used to make a rough estimate for other experiments: FIG. 2a: 77.32% (HeLa); FIG. 15c: 79.7% (HeLa), 73.6% (293T), 78.4% (Huh7), and 70.3% (832/13). Alternatively, RNA UV-crosslinking efficiency were estimated by dividing the RNA/protein ratio of RNP fractions (i.e., RNP composition) by the RNA/protein ratio of corresponding clRNP fractions. This was demonstrated to be a valid approach for correction of signal loss which may occur during repeated AGPC extractions if both fractions display comparable RNA-bound protein profiles by SRA with Coomassie Blue (protein) staining when normalized to μg of protein (strategy is detailed in the provided Source Date for FIG. 15b, d-g). Protein UV-crosslinking efficiency refers to the % of total protein that is recovered as RNA-bound (protein UV-crosslinking efficiency=total RNA-bound protein/total protein*100). Roughly 91% of the protein in LEAP-RBP fractions isolated from final AGPC interphase suspensions is estimated to be RNA-bound by SILAC LC-MS/MS analysis (FIG. 5g, h). No significant difference in protein yield was detected between LEAP-RBP fractions isolated from AGP input (with DNA depletion step) and final AGPC interphase suspensions (FIG. 3a), Fisher's PLSD (two-tailed, unpaired, homoscedastic t test), p=0.998. Cumulatively, these data suggest that comparing the protein yield of LEAP-RBP from AGP input suspensions with DNA depletion step or final AGPC interphase with or without DNA depletion (total RNA-bound protein), and methanol (95% v/v) precipitated input samples (total protein), provides a good approximation of protein UV-crosslinking efficiency. However, comparing protein yields of LEAP-RBP from AGP input suspensions with DNA depletion step and methanol (95% v/v) precipitated input samples (total protein) is the preferred approach for reasons noted above. Compared to estimated RNA UV-crosslinking efficiencies, estimated protein UV-crosslinking efficiencies are considerably lower. However, this is somewhat expected: not all proteins interact with RNA, but all RNA species are expected to contain one or more protein interactors. From this perspective, the difference between RNA and protein UV-crosslinking efficiencies is caused by the inclusion of proteins that don't interact with RNA.

3b. LEAP-RBP Allows Rapid and Comprehensive Assessment of UV-Crosslinking Conditions.

Together, RNA and protein UV-crosslinking efficiencies provide a way to evaluate UV-crosslinking conditions. Because these metrics are normalized to total RNA and protein yields, a relative assessment can be made by comparing LEAP-RBP fractions containing total protein-bound RNA and/or total clRNPs isolated from replicate samples subjected to different UV-crosslinking conditions. As an example, 10 cm plates containing ˜10 million HeLa cells were UV-crosslinked with 0.1, 0.2, 0.4, and 0.8 J/cm² (254 nm) and total clRNPs were isolated from final AGPC interphase suspensions. The effect of UV-dose on RNA UV-crosslinking efficiency was evaluated by comparing RNA yields. Maximum RNA UV-crosslinking efficiency was obtained by UV-irradiating cells with at least 0.4 J/cm² (254 nm) (gold box). As another example, LEAP-RPP fractions (with DNA depletion step) were isolated from AGP input suspensions containing equal amounts of HeLa cells UV-irradiated with 0.0, 0.1, 0.2, 0.4, and 0.8 J/cm² (254 nm). The effect of UV-dose on protein UV-crosslinking efficiency was evaluated by comparing protein yields (Extended Data FIG. 6a). Unlike RNA UV-crosslinking efficiency, maximum protein UV-crosslinking efficiency was obtained by UV-irradiating cells with 0.8 J/cm² (254 nm); protein UV-crosslinking efficiency was estimated as a percentage of total protein for comparison.

For the experiments in this study, cells were washed twice with ice-cold PBS and UV-crosslinked on ice to remove media components which might interfere with UV-crosslinking and to prevent excessive heating of samples, respectively. However, the effect of these sample preparation measures on UV-crosslinking efficiency has not been evaluated in the way that is afforded by LEAP-RBP. Therefore, 10 cm plates containing ˜10 million HeLa cells were UV-irradiated with 0.4 J/cm² (254 nm) with or without media removal and/or on or off ice and/or with or without ice-cold PBS washes and/or with or without extended incubation on ice (15 minutes). Maximum RNA UV-crosslinking efficiencies were obtained using the prescribed sample preparation method (gold box). Interestingly, placing cells on ice during UV-irradiation increases RNA UV-crosslinking efficiency with or without media-removal. RNase-sensitive protein profiles appear similar by SRA and Coomassie Blue (protein) staining but differ in total intensity. These results suggest consistent sample processing and UV-crosslinking conditions are necessary for reproducible results. Inconsistent sample processing and UV-crosslinking (i.e., batch effects) impact RBP-specific UV-crosslinking efficiency and change the amount of total RNA-bound protein in starting samples (total lSI). The effects of UV-crosslinking conditions and/or efficiencies on the ability to detect Δ log₂(S) should be considered (i.e., those that potentially effect dynamic range).

3c. RBP-Specific UV-Crosslinking Efficiencies as a Reproducible Metric for RBP Studies.

The UV-crosslinking efficiencies of individual RBPs range from less than 0.3% for non-canonical RBPs such as RPN1, and upwards of ˜20% for canonical RBPs such as nucleolin (NCL) or HuR (FIG. 10). The estimated protein UV-crosslinking efficiency for HeLa cells UV-irradiated with 0.4 J/cm² (254 nm) is estimated between 1.5-2.0% (FIG. 13a, 13c). For SRA analysis, input samples containing total protein and LEAP-RBP fractions representative of total clRNPs are normalized to μg of protein (˜50× % of LEAP-RBP fraction vs % of input samples) for evaluation of protein enrichment. Because the UV-crosslinking efficiency of XRN1 is comparable to protein UV-crosslinking efficiency under these conditions (FIG. 10), it displays comparable intensity in both fractions normalized in this way. Curiously, RBP-specific UV-crosslinking efficiencies were found to vary in their dose-responsiveness (FIG. 13c). This may be caused by differences in their RNA-binding mechanisms and/or kinetics in vivo; however, the mechanism of UV-crosslinking in macromolecular systems and how to interpret changes in RNA-bound protein abundance are still poorly understood. These findings illustrate the significance of consistent UV-crosslinking practices for generation of reproducible data and demonstrate the ability of LEAP-RBP to provide comprehensive assessment of UV-crosslinking conditions.

### Supplementary Note 4

This note provides additional observations and rationale for distinguishing UV-dependent enrichment of free protein and signal-dependent recovery of noise. Supporting documentation illustrating the role of method robustness and specificity for comparative studies is also included.

4a. UV-Dependent Enrichment of Free Protein Vs Signal-Dependent Recovery of Noise.

UV-dependent enrichment of free protein refers to increased recovery of free RBPs and non-RBPs from UV-crosslinked samples as compared to non-crosslinked controls. Signal-dependent recovery of noise refers to the recovery of unbound proteins during RNA-centric enrichment of their RNA-bound counterparts. Both are observable by SDS-PAGE when comparing equivalent amounts (% fraction) of RNase-treated and untreated RNP fractions isolated from UV-crosslinked and non-crosslinked samples. Increased recovery of RNase-insensitive protein is considered UV-dependent enrichment of free protein. UV-dependent recovery of unbound RBPs (noise) which is dependent on the presence of RNA-bound protein (signal) is considered signal-dependent recovery of noise.

4b. UV-Dependent Enrichment of Free Protein is a Widely Observed Phenomenon.

UV-dependent enrichment of free protein has been noted by others but the explanation for its occurrence has varied. In certain situations, UV-dependent enrichment of free protein appears to be a technical artifact. For example, when performing AGPC extraction on UV-crosslinked and non-crosslinked cells, the AGPC interphase of UV-crosslinked samples is larger than that of non-crosslinked cells (FIG. 17b). The authors of the AGPC-based method, XRNAX, reported the same observation. These authors also reported that the AGPC interphase of non-crosslinked samples is “fluid-like” whereas the AGPC interphase of UV-crosslinked samples is “one sticky blob in addition to the fluid-like regular interphase contents”. Continuing, “this blob and its fluid-like surrounding are not clearly distinct but seem more like a sponge, which soaks up the fluid it sits in” (https://www.xrnax.com/faq/2018/12/30/interphase-1-why-have-a-background-control-why-silica-enrichment-after-xrnax). Noting the significance of this observation, these authors emphasized the need for SILAC-based approach to avoid underestimating free protein recovered from UV-crosslinked samples. In this instance, UV-dependent enrichment of free protein=is thought to be an artifact caused by UV-dependent sample physical properties. However, while this likely contributes to the UV-dependent enrichment of free protein at the first AGPC interphase, repeated AGPC extraction would be expected to reduce or eliminate this protein fraction. For some RNase-insensitive proteins (e.g., β-tubulin and GAPDH), this appears to be true (FIG. 1c). For others (e.g., GRP94 or RPN1), the amount of free protein at the AGPC interphase reaches an apparent UV-dependent enrichment limit, resulting in the apparent UV-enrichment of “RNase-insensitive” or free proteins at the final AGPC interphase (FIG. 1c).

Similar observations were also reported by the authors of the OOPs method which utilizes 3 AGPC extractions as part of their methodological approach. In this method, interphase samples are precipitated with methanol following 3 AGPC extractions, RNase-treated, and subjected to a fourth AGPC extraction; untreated samples are processed in parallel as a control. Proteins are methanol precipitated from the organic phases and RBPs are then identified by their RNase-dependent enrichment in the organic phase. Roughly 96% of proteins were found to exhibit RNase-dependent enrichment in the 4^{th} organic phase (FIG. 2i). These authors also noted that “Proteins that migrated to the organic phase included those that were CL independent, suggesting their presence in the interface is RNA dependent, but their interaction with RNA was stable even in the absence of CL” (FIG. 2f, FIG. 10i). Here, UV-dependent enrichment of crosslink-independent (free protein) at the AGPC interphase is thought to be a result of stable interactions with the RNA components of protein-bound RNA at the AGPC interphase. Alternatively, it was postulated that insoluble clRNPs at the AGPC interphase might provide additional interaction surfaces for association of free protein. A prediction from this hypothesis is that the amount of protein recovered from the AGPC interphase suspensions is dependent on the amount of RNA-bound protein (total lSI), rather then the starting amount of protein. To test this prediction, samples containing 10 million UV-crosslinked (0.4 J/cm², 254 nm) or 10 million UV-crosslinked and 10 million non-crosslinked HeLa cells were subjected to 6 AGPC extractions. While the AGPC interphase of the pooled samples were larger than the non-pooled samples during the initial few AGPC extractions, they were of comparable size by the final AGPC extraction. Following precipitation of protein-bound RNA by INP, RNA and protein yields were quantified by UV-spectrophotometry and BCA respectively. No discernible differences in RNA or protein recovery were observed and both fractions displayed comparable signal recovery (e.g., XRN1, HuR, and RPN1) and free protein recovery (e.g., GRP94) by SRA and immunoblot. These results suggest UV-dependent enrichment of free protein at the AGPC interphase is dependent on the amount of RNA-bound protein in the sample (total ISI), not the amount of total protein in starting samples.

4c. High Method Specificity for RNA-Bound RBPs (High % TP_{S}) Reveals Signal-Dependent Recovery of Noise.

After development of LEAP-RBP, it was realized that UV-dependent enrichment of free protein at the AGPC interphase was not an isolated phenomenon. As shown in FIG. 3a, b and FIG. 13a-c, performing LEAP-RBP (with DNA depletion step) on AGP input suspensions containing UV-crosslinked or non-crosslinked cells resulted in UV-dependent recovery of unbound RBPs despite comparable (UV-independent) recovery of RNA. Therefore, UV-dependent enrichment of unbound RBPs was dependent on RNA-bound protein, not RNA itself. These results thus appeared to invalidate both prior explanations for UV-dependent enrichment of free protein at the AGPC interphase. For example, it was proposed that UV-enrichment of non-crosslinked protein at the AGPC interphase occurs through stable interactions with RNA which exhibits high UV-dependent enrichment at the AGPC interphase. A direct prediction of this hypothesis is that if the presence of RNA at the AGPC interphase is UV-independent, then free protein would be recovered in equal quantities under the same conditions. The harsh chaotropic conditions used during the LEAP-RBP method are comparable to AGPC extraction. Furthermore, AGP input suspensions containing equal amounts of UV-crosslinked or non-crosslinked cells have comparable amounts of RNA. Yet, recovery of unbound RBPs is clearly UV-dependent despite comparable (UV-independent) recovery of RNA (FIG. 3a, b, FIG. 13a, c). Alternatively, and as noted above, it was hypothesized that insoluble clRNPs at the AGPC-interphase of UV-crosslinked cells provide additional interaction surfaces for co-precipitation of free protein resulting in their apparent UV-dependent recovery. However, the UV-dependent selective recovery of unbound RBPs (noise) over non-RBPs (background) from AGP input suspensions as shown in FIG. 3b alludes to a different phenomenon at work: RNA-bound protein (signal)-dependent recovery of noise. This phenomenon likely evaded prior recognition because repeated AGPC extractions and the INP method have insufficient specificity to distinguish RNase-insensitive RBPs with low S/N from non-RBPs which partition to the AGPC interphase (FIG. 1c, 2b).

The physicochemical basis for signal-dependent recovery of noise is enigmatic. The harsh chaotropic conditions of AGPC mixtures denatures RBPs and disrupts RNA-protein interactions preventing their effective RNA-dependent recovery from non-crosslinked samples. Yet, they are recovered in appreciable quantities from UV-crosslinked cells. Additionally, the recovery of free proteins via non-covalent interactions with RNA-bound proteins under the harsh conditions would, at most, be expected to result in non-selective recovery of free RBPs and non-RBPs. Yet, unbound RBPs are selectively recovered over non-RBPs.

4d. UV-Dependent Enrichment of Free Protein and Signal-Dependent Recovery of Noise operate under different rules.

Despite their apparent similarities, signal-dependent recovery of noise is more appropriate for describing UV- and signal-dependent phenomena than UV-dependent enrichment of free protein. These differences are illustrated in the following hypothetical example: Consider a starting population of various RBPs and non-RBPs in a cell. Upon UV-irradiation, some RBPs will be UV-crosslinked to RNA interactors but non-RBPs will not. Because UV-crosslinking is not 100% efficient, RBPs will comprise two populations: RNA-bound (signal) and unbound (noise) counterparts. Theoretically, RNA-centric enrichment methods will enrich RNA-bound proteins over their unbound counterparts (enhance S/N). However, RNA-centric enrichment methods, by definition, do not enrich unbound RBPs over unbound non-RBPs. Thus, UV-dependent enrichment of non-RBPs likely results from low method specificity, UV-dependent changes in sample physical properties, or non-specific UV-crosslinking. For example, UV-independent partitioning of the unbound RBP nucleolin (NCL) to the AGPC interphase and the inability of INP to discernibly enhance the S/N of NCL are examples of low method specificity (gold boxes; FIG. 1c, 2b). The apparent UV-dependent enrichment of RNase-insensitive protein (eg., GAPDH, GRP78, and β-tubulin) at the fourth AGPC interphase despite their eventual depletion is likely a result of UV-dependent changes in sample physical properties (FIG. 1c). Lastly, non-RBPs which appear UV-enriched* by SILAC LC-MS/MS but remain undetectable or RNase-insensitive by SDS-PAGE and immunoblot are likely examples of low abundance non-specific UV-crosslinking. The experimental ideal would be a method capable of absolute specificity for RNA-bound proteins (N=0), but this is not evident in current methodologies. Such a method would yield no detectable peptides from non-crosslinked samples during a SILAC-based LC-MS/MS experiment (FIG. 4a). Based on these principles, recovery of noise will always be observed during RNA-centric isolation of their RNA-bound counterparts while enrichment of background protein is not guaranteed. Consequently, when evaluating methods with varying % TP_{S} but comparable recovery of RNA-bound protein, background proteins will often go from observable to undetectable while noise simply varies in abundance.

Comparison of proteins identified by SILAC LC-MS/MS analysis of INP and LEAP-RBP demonstrates the difference between background and noise. For example, the INP method recovers many background proteins displaying log₂(CL/nCL) ratios around 0 (FIG. 4f); comparatively, the LEAP-RBP method identified little to no background proteins, as these would display a similar distribution with mean log₂(CL/nCL) ratio of 0. As mentioned previously, background proteins are expected to go from observable to undetectable with increasing % TP_{S}. Indeed, 392 proteins displaying log₂(CL/nCL) ratios with a mean distribution centered around 0 in INP fractions are not re-identified in LEAP-RBP fractions, supporting their designation as background. Unlike proteins identified exclusively in LEAP-RBP fractions which are found almost entirely within the lower range of detection, many of the background proteins identified exclusively in INP fractions are found within the middle range of detection. This is consistent with observed differences in their abundances. More than half of the proteins identified exclusively in INP fractions are GO-annotated glycoproteins (200/391) which are poorly selected against by repeated AGPC extraction. These background proteins are undetectable in LEAP-RBP fractions by SILAC LC-MS/MS despite acquiring a limit of detection (% TP range) nearly one order of magnitude lower than the INP method (FIG. 6a, b). While the SILAC LC-MS/MS approach accurately identifies background proteins by their log₂(CL/nCL) ratios (˜0), non-SILAC comparisons between INP fractions isolated from independent UV-crosslinked and non-crosslinked cellular samples would result in their ubiquitous UV-enrichment. This would be an example of low method specificity resulting in UV-dependent enrichment of free proteins. Background proteins UV-enriched in this way can be highly variable and sensitive to slight differences in sample processing and method specificity. The recovery of different background protein profiles may be attributed to interactions between UV-crosslinking, method-specific RNA-centric enrichment conditions, and protein-specific physicochemical properties. These discrepancies during non-SILAC LC-MS/MS experiments may appear as method-specific RNA-binding proteins and hinder meaningful meta-analyses of UV-enriched* proteins identified by different RNA-centric methods.

4e. Signal-Dependent Recovery of Noise is the Primary Source of Free Protein for RNA-centric enrichment methods with high specificity for RNA-bound RBPs.

Compared to background proteins (S=0) which can change from observable to undetectable with increasing (% TP_{S}), unbound RBPs (noise) are only expected to vary in their abundance when recovery of signal is comparable (FIG. 2a, b). Indeed, 93% of RBPs identified in INP fractions are re-identified in LEAP-RBP fractions (813/875) but display higher S/N ratios resulting in higher % TP_{S} contributions (89.2 vs 46.1) and lower % TP_{N} contributions (7.7 vs 42.8). Contrarily, only 39% of non-RBPs identified in INP fractions are re-identified in LEAP-RBP fractions (209/538). While their % TP_{S} contributions are roughly 2-fold higher in LEAP-RBP fractions compared to INP fractions (1.6 vs 0.7), a 6-fold decrease in their % TP_{N} contributions (1.5 vs 10.5) results in a roughly 4-fold net decrease in their abundance in LEAP-RBP fractions. SILAC LC-MS/MS analysis of LEAP-RBP fractions identified two different populations of free proteins (RBPs and non-RBPs) exhibiting signal-dependent recovery as evidenced by their positive log₂(CL/nCL) ratios (FIG. 4f). Compared to RBPs, non-RBPs displayed lower enrichment efficiencies (S/N ratios) and were less abundant (% TP) (FIG. 6e). This might be due to differences in their physicochemical properties and/or UV-crosslinking efficiencies. For example, many RBPs displaying lower S/N ratios were also found to have lower UV-crosslinking efficiencies (e.g., RPN1; FIG. 10). Nonetheless, the average S/N ratios of non-RBPs (˜1) in LEAP-RBP fractions suggests an equivalent amount of free protein (N) is present and detectable by immunoblot. Indeed, when large quantities of clRNPs were evaluated by SRA, a small amount of RNase-insensitive protein was observed for the cytoskeletal protein β-tubulin identified as UV-enriched* in LEAP-RBP fractions by SILAC LC-MS/MS (FIG. 12a). This supports the idea that formation of such complexes is exceedingly rare while demonstrating the significance of this orthogonal validation method and SILAC LC-MS/MS to accurately quantify free protein recovery.

The percentage of total protein in the sample that is RNA-bound (% TP_{S}) can define enrichment limits when repeated utilization of a given enrichment method fails to further increase % TP_{S} (maximum % TP_{S}). The ability of a method to achieve maximum % TP_{S} despite differences in protein UV-crosslinking efficiency (starting % TP_{S}) and total |S| demonstrates method robustness. For individual RNA-binding proteins, enrichment limits are more appropriately described by the protein-specific metric S/N. In either case, enrichment limits are more readily evaluated when a method is capable of depleting free protein (N) without or signal loss. Repeated AGPC extraction, INP, LEAP-RBP, and methanol precipitation were demonstrated to recover near 100% of protein-bound RNA (Supplementary Note 3a). Methanol precipitation is a protein-centric method that is unbiased towards RNA-bound (S) and free protein (N) and so it doesn't contribute to % TP_{S}. INP and LEAP-RBP are RNA-centric enrichment methods which increase % TP_{S} compared to methanol, but LEAP-RBP achieves higher maximum % TP_{S} (FIG. 2a, b). Enrichment of clRNPs at the AGPC interphase during repeated AGPC extractions is RNA-centric or protein-centric depending on which interactor is considered responsible for their partitioning at the AGPC interphase. Maximum % TP_{S} for AGPC extraction typically requires multiple AGPC extractions depending on the % TP_{S} and total |S| of starting samples. Curiously, the AGPC mixing method affects both the rate of free protein partitioning into the organic phase (FIG. 17a), and the maximum % TP_{S} of the final AGPC interphase. Illustrating these points, the following results were obtained by UV-irradiating cellular samples with 0.4 J/cm² (254 nm); RNA and protein UV-crosslinking efficiency is expected to be between 70.0-80.0% and 1.5-2.0% respectively (Supplementary Note 3). Under these UV-crosslinking conditions, maximum % TP_{S} for INP fractions requires starting from final AGPC interphase suspensions (i.e., maximum % TP_{S} of repeated AGPC extraction); additional INP steps have minimal impact on % TP_{S}. Notably, AGP input suspensions under these UV-crosslinking conditions contain up to ˜70× the amount of free protein than the final AGPC interphase suspension. Yet, LEAP-RBP fractions isolated from either suspension display comparable RNase-sensitivity by SRA and Coomassie Blue (protein) staining (FIG. 3a, b); the RNase-sensitivity of total protein in the sample by SRA and Coomassie Blue (protein) staining is considered directly related to the % TP_{S} of the fraction. This represents a roughly 580-fold enrichment of RNA-bound (total SPI_{S}) over free (total SPI_{N}) protein in the starting sample: (% TP_{S}/% TP_{N})RNP/(% TP_{S}/% TP_{N})ᵢₙₚᵤₜ.

Most RBPs display comparable RNase-sensitivity (S/N) by SRA and immunoblot in both RNP fractions (FIG. 3b). This indicates that maximum % TP_{S} for LEAP-RBP fractions does not require starting from final AGPC interphase suspensions. Relating to points noted above, this suggests that recovery of noise for most RBPs is dependent on the amount of RNA-bound protein, not the amount of free protein. This finding was validated by performing a UV-dose response experiment and analyzing LEAP-RBP fractions (with DNA depletion step) by SRA and immunoblot (FIG. 13a-c). Assuming 91% of the total protein in LEAP-RBP fractions is RNA-bound in all UV-dose conditions, the fraction isolated from HeLa cells irradiated with 0.1 J/cm² represents a roughly 2,500-fold enrichment of RNA-bound protein over free protein in the starting sample. Interestingly, RPN1 displays low S/N in LEAP-RBP fractions isolated from cells irradiated with all UV-doses by SRA and immunoblot unless starting from the final AGPC interphase (FIG. 3b, FIG. 13c). Therefore, starting from AGP input suspensions is suitable for most RBPs, but not all. Starting from the final AGPC interphase ensures the maximum % TP_{S} and sensitivity for LC-MS/MS and SRA analysis regardless of % TP_{S} and total |S| of input samples. Additional LEAP-RBP steps don't discernibly increase % TP_{S} when starting from final AGPC interphase suspensions (FIG. 18e).

4f. Method Robustness and High % TP_{S} Facilitates Rigorous Assessment of RNA-Bound Protein Abundance.

Like S/N ratios, method robustness and high % TP_{S} are important for comparative LC-MS/MS studies aimed at identifying differences in RNA-bound protein abundance by limiting the contribution of free protein towards total MS signal (% TP_{N}) regardless of % TP_{S} and total |S| of input samples (robustness). This is particularly significant for non-SILAC LC-MS/MS experiments where samples are normalized to total SPI. For example, RNA-bound RBPs in INP and LEAP-RBP fractions display comparable RNA-bound abundance by SRA and immunoblot when they are normalized to μg of protein-bound RNA (FIG. 2b). This approach works because the quantity of RNA-bound protein is considered directly proportional to the μg of protein-bound RNA in the sample regardless of % TP_{S} when UV-crosslinking conditions (i.e., total |S| of input samples) and signal recovery are comparable (Supplementary Note 3). However, if INP and LEAP-RBP fractions were normalized to μg of protein (total SPI) for SRA and immunoblot, there would be more visible RNA-bound protein in LEAP-RBP fractions because INP recovers nearly twice as much quantifiable protein (FIG. 2a).

To evaluate how differences in free protein recovery affects accurate assessment of RNA-bound protein abundance (% TP_{S}), a label-free LC-MS/MS experiment was conducted comparing INP and LEAP-RBP fractions. Because both methods recover near 100% of RNA-bound protein and only differ in the amount of free protein recovered, the absolute quantity of RNA-bound protein in the sample (total ISI) is expected to be the same (I vs L; FIG. 2a). For this example, the observed quantities for 357 proteins displaying S/N ratios>3 in both INP and LEAP-RBP fractions by SILAC LC-MS/MS were considered representative of their RNA-bound quantities (>75% RNA-bound). Many of the RBPs which display comparable RNA-bound abundance in INP and LEAP-RBP fractions normalized to signal quantity by SRA and immunoblot fall into this category (e.g., HuR, RPL8, RPL4, TIA1, pAbPC1, PABPC4, XRN1, and LRRC59). The observed quantities (SPI values) for proteins displaying high S/N ratios are predicted to be more comparable between INP and LEAP-RBP fractions when normalizing samples to total RNA-bound protein quantity (total SPIs) vs total protein quantity (total SPI). To test this prediction, the log₂(S/N) ratios of proteins identified in LEAP-RBP fractions by SILAC LC-MS/MS were plotted as a function of their relative quantities (Δ log₁₀(SPI)) as compared to INP fractions. The relative quantities of proteins displaying S/N ratios>3 in both fractions appear increased when samples are normalized to total protein in the sample (total SPI) but comparable when samples are normalized to total RNA-bound protein in the sample (total SPI_{S}) (Supplementary Methods). The relative quantities of proteins when samples are normalized to total SPI is equivalent to their relative abundances (Δ log₁₀(SPI)=Δ log₁₀(% TP)). As such, the observed increase in relative quantities of proteins displaying S/N ratios>3 may be interpreted as an increase in RNA-bound abundance (% TP≈% TP_{S} and Δ log₁₀(% TP)≈Δ log₁₀(% TP_{S})). These data demonstrate how differences in free protein recovery, emulated here by comparing methods with varying % TP_{S} and comparable signal recovery, can appear as proteome-wide changes in RNA-bound protein abundances (% TP_{S}).

By SILAC LC-MS/MS, an estimated 91% of the total protein in LEAP-RBP fractions is RNA-bound (total SPI≈total SPI_{S}). Therefore, observed abundances for proteins displaying S/N ratios>3 (% TP≈% TP_{S}), can be considered representative of their RNA-bound abundances estimated as a percentage of total RNA-bound protein in the sample (% TP≈% TP_{S}≈% TP(S)). An increase in the observed abundance (% TP) of RBPs relative to non-RBPs with increasing % TP_{S} reflects decreased contributions from their unbound counterparts (i.e., % TP_{N}). Because LEAP-RBP recovers near 100% of total RNA-bound protein—or herein total cellular RNA-bound protein—observed % TP for proteins displaying S/N ratios>3 is considered representative of their total cellular RNA-bound abundances; notably, total RNA-bound protein abundance=% TP(S) only when total RNA-bound protein “in the sample” is considered representative of total RNA-bound protein (Supplementary Methods).

The high % TP_{S} (>90%) of LEAP-RBP fractions makes it possible to perform label-free comparative LC-MS/MS experiments aimed at identifying differences in RNA-bound protein abundance (FIG. 7b-h). For this, samples can be normalized to total SPI as in the previous example. Most label-free quantification (LFQ) algorithms, such as the MaxQuant LFQ algorithm MaxLFQ, assumes samples are relatively similar and do not include weighting factors that prioritize protein abundance. This is problematic, as LEAP-RBP fractions contain many low-abundance proteins with significant free protein contributions (i.e., low S/N). Because they only contribute 3% of total SPI, weighting them equally contributes additional sources variance that is unlikely to reflect the biological activity of interest (i.e., direct RNA-binding). Conversely, the most abundant proteins in LEAP-RBP fractions display high S/N and appear well-conserved (RNase, clRNP fractions; FIG. 15b). Therefore, they should be given more weight when normalizing samples for comparative LC-MS/MS studies. While it has been reported that LFQ algorithms can be successfully applied to samples following protein enrichment steps (e.g., co-immunoprecipitation), it was also noted that these studies benefit from a dominant and reproducible population of background proteins exhibiting minimal changes between experimental conditions. In contrast, RNA-centric enrichment of RNA-bound protein is arguably more of an indirect form of protein enrichment. As noted previously, recovery of free protein during RNA-centric enrichment of RNA-bound protein is enigmatic. The potential interactions between the UV-crosslinking, RNA-enrichment conditions, and protein-specific physicochemical properties currently cannot be predicted with certainty (Supplementary Note 4c-e). Recognizing this limitation, normalizing to total SPI makes the least assumptions about sample identities and leverages the high % TP_{S} and robustness of the LEAP-RBP method.

### Supplementary Note 5

This note provides supporting information and rationale for protein-specific S/N ratios, SILAC LC-MS/MS, and MS data handling.

5a. Rationale Behind Protein S/N Ratios.

In this study, S/N of proteins represents the ratio of RNA-bound to unbound counterparts, while proteins without RNA-bound counterparts represent background proteins (S=0). These designations were chosen because LC-MS/MS analysis will not differentiate whether a tryptic peptide originated from RNA-bound or unbound counterparts (red box. Notably, peptide UV-crosslinked to RNA moieties can be distinguished, but most tryptic peptides are not expected to be directly crosslinked to RNA. Conceptually, S/N of proteins is similar to the S/N of MS-peak intensities. Therefore, comparisons between the two helps illustrate the scientific rationale behind S/N ratios as a protein-specific metric herein.

In a typical LC-MS/MS experiment, peptides derived from proteolytic digestion generate peak ion intensities over their expected time window. All mass spectrometers detect a background signal in the absence of peptides which fluctuates over time. The background signal is indistinguishable from the signal generated by ionized peptides during their expected time window. Therefore, accurate quantification of peptide intensities (S) requires estimating the contributions of background noise (N) to the total peak ion intensities (S+N). To do this, background noise (N) is estimated “off-peak” as the distance between peak background signal and the average background noise (X-bar)_{B}. Integrated peptide intensities (S) are generated by subtracting the integrated noise intensities (N) from the integrated peak ion intensity (S+N) over the same time window. The S/N ratio can be estimated by dividing the integrated peptide intensities by the integrated noise intensities. When S=0, N−N=0 (background) and S/N is undefined.

The same basic S/N principles were used herein to represent the ratio of RNA-bound to unbound counterparts. Necessarily, this represents an additional noise contribution above the background noise of the mass spectrometer. Quantified peptide intensities of protein (O) represent the sum of peptide intensities from RNA-bound (S) and unbound (N) counterparts. However, the contributions from unbound protein cannot be estimated “off-peak” because the peptides from RNA-bound counterparts have the same retention time and will map to the same protein. Estimating noise contributions in UV-crosslinked samples by performing LC-MS/MS on independent non-crosslinked samples will underestimate the amount of free protein (Supplementary Note 4d). Therefore, noise is more accurately estimated using a SILAC-based approach, where SILAC-labeled UV-crosslinked and non-crosslinked (nCL, samples are pooled prior to RNA-centric enrichment. Because peptides from UV-crosslinked cells will have longer retention times than peptides from non-crosslinked cells, they can now be independently quantified. The peptide intensities observed in the non-crosslinked SILAC channel provide an “off-peak” equivalent to background noise from the previous example by assuming equal noise-partitioning between SILAC channels (Supplementary Note 6a).

5b. Strategies for Estimating Noise Contributions.

Estimating background noise contributions of the mass spectrometer benefits from a large sampling size. However, estimating noise contributions for individual peptides in the UV-crosslinked SILAC channels based on a single peak from the non-crosslinked SILAC channel would introduce additional variance or fail when peptides in the non-crosslinked SILAC channel are undetected above the background instrument noise. The concerning issue of missing peptide in the non-crosslinked SILAC channel has been previously noted. For example, the authors of the TRAPP (purple arrows & boxes) and XRNAX (blue arrows & boxes) methods used a comparable SILAC-based approach for identification of UV-enriched* proteins and provide solutions for absent peptides in non-crosslinked samples. In one approach, missing peptide data is imputed computationally, via random selection of a peptide intensity value from the bottom percentile of all peptide intensities. This approach rarely introduces additional variance if sum peptide intensities (SPI) are used for calculating log(CL/nCL) ratios because the SPI values mainly reflect the more abundant peptides identified across all samples and in both SILAC channels (purple vs gold boxed SPI bar charts). However, if UV-enriched* proteins are identified by calculating the log(CL/nCL) ratio of each peptide, as done for XRNAX, imputing values can introduce unmeaningful variance. This strategy is used for experiments where replicates are limited (n=1-2) and treating peptides as independent observations enables hypothesis testing. While peptide ratios are generally more variable, the larger sampling size (# of peptides) compensates by increasing statistical power (SEM). Nonetheless, the additional variance from log(CL/nCL) ratios calculated using imputed peptide values is unmeaningful. Noting this, the authors of XRNAX filter for peptides only detected in the UV-crosslinked SILAC channel and use the same pseudo-count as the denominator for all “super-enriched” peptides (FIG. S1G). This approach is equivalent to subtracting the identical background noise from all peak intensities so that only peptide intensities in the UV-crosslinked SILAC channel contributes variance.

Both strategies have merit and for different reasons. The strategy used by the authors of the TRAPP method avoids underestimating the amount of free protein in the sample (% TP_{N}), and the strategy used by the authors of the XRNAX method avoids unmeaningful variance introduced by free proteins observed in the non-crosslinked SILAC channel. In the current study, protein quantities in each SILAC channel were estimated as the sum of their identified peptide intensities without imputing missing peptide intensities. Estimating relative protein quantities using sum peptide intensities does not require that the same number of peptides are quantified in each sample; SPI values mostly reflect the most abundant peptides identified across all samples and in both SILAC channels. After normalizing samples to total SPI, SPI_{nCL} values equal to 0 were replaced with the average non-zero SPI_{nCL} value (Supplementary Methods). Log₂(CL/nCL) and log₂(S/N) ratios were generated using SPI_{CL} values and average SPI_{nCL} values according to equations (2) and (3) respectively. This analytical approach has the benefit of avoiding unmeaningful variance introduced by free proteins observed in the non-crosslinked SILAC channel (Supplementary Note 6b).

### Supplementary Note 6

This note provides supporting information for S/N-based analyses and additional considerations for comparative LEAP-RBP experiments and other downstream applications.

6a. Accurate Evaluation of S/N by LC-MS/MS Analysis Requires a SILAC-Based Approach.

If only RNA-bound proteins exhibit UV-dependent enrichment, then S/N can be readily quantified by label-free LC-MS/MS analysis of independent UV-crosslinked and non-crosslinked samples. However, UV-dependent enrichment of free protein is a widely observed phenomenon (Supplementary Note 4b, c). As an example, performing repeated AGPC extraction and LEAP-RBP on independent UV-crosslinked and non-crosslinked samples only yields detectable protein from UV-crosslinked samples (FIG. 12a). If the assumption that only RNA-bound proteins exhibit UV-dependent enrichment is true, then all the isolated protein is RNA-bound. However, when differentially SILAC-labeled non-crosslinked and UV-crosslinked cells are pooled prior to isolation of the clRNP fraction and analyzed by LC-MS/MS, many peptides originating from non-crosslinked cells were observed (FIG. 4a). As these peptides do not originate from UV-crosslinked RNA-bound protein, the assumption that only RNA-bound proteins exhibit UV-dependent enrichment is false. Because the recovery of free protein in LEAP-RBP fractions is dependent on signal quantity and is independent of total protein and RNA (FIG. 3a, b, FIG. 13a-c), S/N ratios were calculated assuming equal noise-partitioning between SILAC channels.

The analyses presented in this study considered protein quantities observed in both SILAC channels. However, if SPL_{CL} values were used to estimate free protein contributions in the UV-crosslinked SILAC channel, but ignored during comparative analyses, this would effectively increase the log₂(S/N) ratio of all proteins by 1 (log₂(S/N_{(CL)}=log 2(S/N)+1). Additionally, if protein abundances were estimated as a percentage of total protein in the UV-crosslinked SILAC channel (total SPI_{CL}), this would effectively increase the observed abundance of RNA-bound proteins by halving free protein contributions (% TP(CL), S=% TP_{S}+% TP_{N}/2). Because the % TP_{(S)} contributions of RBPs (98.3) are higher than their % TP_{(N)} contributions (83.4), and % TP_{(S)} contributions of non-RBPs (1.7) are lower than their % TP_{(N)} contributions (16.6), the observed abundance of RBPs relative to non-RBPs will increase (Source Data FIG. 5h).

$$\% {TP}_{( S ) , RBPs} / \% {TP}_{( S ) , non - RBPs} > \% {TP}_{( CL ) , RBPs} / \% {TP}_{( CL ) , non - RBPs} > \% {TP}_{RBPs} / \% {TP}_{non - RBPs}$$

Indeed, performing this type of data handling results in the expected transformations. This may be employed as an analytical strategy for comparative LEAP-RBP experiments utilizing a SILAC-labeling approach to further enhance S/N. However, normalizing samples to total SPI_{CL} as compared to total SPI is not expected to provide significant benefits (Supplementary Note 6b).

6b. S/N Ratios Serve as a Key Metric for Identifying Δ Log₂(S) and Avoiding Δ Log₂(N).

From an S/N perspective, UV-enrichment* indicates there is more protein recovered from UV-crosslinked (S+N) than non-crosslinked samples (N). This is comparable to testing whether a given MS peak intensity (S+N) can be distinguished from background noise (N) of the mass spectrometer (Supplementary Note 5a); and is often referred to as the “limit of detection” or LOD However, the point at which a change in peptide intensity (S) can be reliably detected is much higher and often called the “limit of quantification” or LOQ; here, S/N describes the relative contributions of peptide intensity (S) and background noise (N) towards the observed MS peak intensity (O). Because they have different sources of variance, the S/N ratio also describes their relative contributions towards the observed variance. Similarly, the S/N ratio of proteins describes the relative contributions of their RNA-bound (SPI_{S} or S) and unbound counterparts (SPI_{N} or N) towards their observed quantities (SPI_{O}, SPI, or S+N). Log₂(S/N) ratios therefore provide a means to evaluate their contributions in a way that log₂(CL/nCL) ratios can't by providing a total function. Theoretically, proteins with log₂(CL/nCL) ratios less than 0 cannot contain signal, just as MS peak intensities (S+N) below background noise (N) cannot contain peptide intensities (S). At a log₂(S/N) ratio of 0, RNA-bound and unbound counterparts contribute equally to the observed quantity and variance of proteins. In this study, the importance of having sufficient S/N to detect a change in log₂(S+N) in response to Δ log₂(S) is emphasized (FIG. 5d, e). Conversely, having sufficient S/N is important for avoiding a change in log₂(S+N) in response to Δ log₂(N). Therefore, S/N is a key metric for statistical analysis during comparative experiments because it helps identify differences in protein recovery more likely to reflect Δ log₂(S).

To test whether RNA-bound and unbound counterparts have similar variability, log₂(SPI_{nCL}) and log₂(SPI_{CL}) values generated during SILAC LC-MS/MS analysis of LEAP-RBP fractions (n=3) were used to assess the variability of log₂(N) and log₂(S) values respectively. For LEAP-RBP fractions, the variability of log₂(SPI_{CL}) values provide a good approximation for the variability of log₂(S) values because an estimated 95% of the total protein observed in the UV-crosslinked SILAC-channel is RNA-bound (% TP_{(CL), S}=95; Supplementary Note 6a). Only proteins detected in all three LEAP-RBP fractions and across both SILAC-channels were included (n=1743, ˜90% of protein IDs). SPI values were log₂ normalized and adjusted by subtracting the mean log₂ normalized value of all three replicates for each protein ID. Values for each replicate were treated as independent observations (n=5229). The probability density distribution of log₂(SPI_{nCL}) values is wider (SD=˜0.5) than the probability density distribution of log₂(SPI_{CL}) values (SD=˜0.3) regardless of the normalization method used. Furthermore, the density distribution of log₂(SPI_{nCL}+SPI_{CL}) values is more comparable to the density distribution of log₂(SPI_{CL}) values. This reflects the larger contribution of UV-crosslinked samples towards total SPI (SPI_{nCL}+SPI_{CL}), and the larger contribution of RNA-bound protein towards the observed variance of log₂(SPI_{CL}) values. Indeed, Levene's test for equality of variances did not detect a significant difference in variance between log₂(SPI_{nCL}+SPI_{CL}) and log₂(SPI_{CL}) values: F(1, 10456)=0.54, p=0.464, but there was a significant difference in variance between log₂(SPI_{CL}) and log₂(SPI_{nCL}) values F(1, 10456)=748.96, p<0.001. Based on these data, normalizing samples to total SPI_{CL} as compared to total SPI during SILAC LC-MS/MS experiments is not expected to provide significant benefits. Additionally, the variability of observed quantities log₂(S+N) is expected to increase with decreasing S/N.

6c. Setting S/N Limits for Comparative LEAP-RBP Experiments.

RNA-bound proteins and their unbound counterparts have different physicochemical properties and sources of variance. While UV-crosslinking conditions are the main source of variance for RNA-bound proteins (Supplementary Note 3), the sources of variance for their unbound counterparts are enigmatic (Supplementary Note 4). For example, more unbound RPL4 is recovered by LEAP-RBP from HeLa cells than the other three cell lines examined without a discernible difference in RNA-bound abundance (untreated (N) vs RNase (S+N), clRNP fraction; FIG. 7j). Furthermore, less unbound TIA-1a (upper band) is recovered from 832/13 cells than the other three cell lines without a discernible difference in RNA-bound abundance (FIG. 7j). Because both RPL4 and TIA-1 display high S/N in LEAP-RBP fractions, however, differences in noise recovery is less likely to obscure changes in RNA-bound quantity. By setting rigorous criteria for comparative experiments, signal-dependent recovery of noise can be reconciled. In this study, S/N ratios of proteins were estimated by SILAC LC-MS/MS analysis of LEAP-RBP fractions; however, S/N ratios can also be estimated by SRA and immunoblot. The limit of quantification (LOQ) for MS-peak intensities represents the acceptable S/N for detecting a change in peptide intensity (S). For reliable measurement of analyte concentration, a S/N ratio of 10 or higher is recommended as the LOQ. However, a S/N between 3-10 has been proposed as a more practical quantification limit for simply detecting a significant difference in analyte concentration when comparing experimental and control samples. For RBPs, an S/N ratio of 3-10 indicates 75-91% of observed protein quantities are RNA-bound (Source Data FIG. 5c, Supplementary Note 6b). Based on SILAC LC-MS/MS analysis of LEAP-RBP fractions, 911 proteins display S/N ratios>3; 770 of these were re-identified during the comparative LEAP-RBP experiment utilizing a label-free LC-MS/MS approach (FIG. 7b-h).

6d. Application and Utility of LEAP-RBP.

Because of its high selectivity for RNA-bound species, LEAP-RBP is a valuable tool for RBP studies. Principally, LEAP-RBP and the methods herein allow quantitative recovery of total protein, RNA, and clRNPs and estimation of protein, RNA, and RBP-specific UV-crosslinking efficiencies (Supplementary Note 3a-c, Supplementary Methods), which provides a sound basis for optimization of UV-crosslinking conditions and provides useful metrics to verify reproducibility. The robustness and high specificity of LEAP-RBP supports confident validation of RNA-binding and identification of conditions that alter RNA-bound protein abundance (Supplementary Note 4e, f). While LEAP-RBP can be paired with MS-based proteomic approaches to analyze global RNA-bound protein dynamics, SRA and immunoblot provides a cost-effective way to analyze regulation of in vivo RNA-binding for individual RBPs of interest. For example, it was observed that the TIA-1a isoform (top band) exhibits higher UV-crosslinking efficiency (i.e., total RNA-bound abundance) than the TIA-1b isoform (bottom band) in vivo despite comparable total abundance (gold boxes; FIG. 10, Supplementary Note 3c). These data contrast with in vitro studies where it was previously reported that that the RNA-binding properties of TIA-1a and TIA-1b do not differ significantly. These prior studies were performed in vitro using established electrophoretic mobility shift assays (EMSA) to analyze interactions between recombinant GST-tagged proteins and two RNA substrates and thus highlight the value in orthogonal experimental approaches to evaluating RNA binding function. Curiously, TIA-1a displayed comparable RNA-bound abundance in rat pancreatic insulinoma cell line (832/13) and three human cell lines (i.e., HeLa, 293T, and Huh7) despite it being nearly undetectable in 832/13 input samples (untreated (N) vs RNase (S+N) clRNP fraction, or RNase input; FIG. 7j). These findings suggests that TIA-1a performs comparable RNA-binding functions in 832/13 cells despite its relatively lower total abundance. Like TIA-1, nucleolin (NCL) displays isoform-specific differences in UV-crosslinking efficiency (Source Data FIG. 10). These results illustrate how LEAP-RBP and SRA provide a rapid and cost-effective way to assess the impact of protein modifications and/or alternative splicing isoforms on in vivo RNA-binding.

LEAP-RBP provides a new methodological approach to the orthogonal validation of observed differences in RNA-bound abundance. As an example, previous studies have reported global changes in RNA-binding activity during cellular stress-responses based on differences in protein recovery (performed by TRAPP, OOPs, XRNAX, RIC/eRIC approaches). However, the inclusion of proteins with significant free protein contributions and the lack of orthogonal validation showing that observed differences in protein recovery are due to a change in their RNA-bound abundance hampers meaningful interpretation. LEAP-RBP enables the use of SRA as a cost-effective and robust orthogonal validation approach to traditional validation methods such as CLIP-seq or radioisotopic T4 PNK assays. Indeed, neither CLIP-seq or radioisotopic T4 PNK assays have been demonstrated to accurately assess changes in RNA-bound protein abundance, thereby compromising their utility for validating observed RBP dynamics. The utility of the LEAP-RBP method and SRA extends to RIP- or CLIP-seq experiments which are typically performed without existing validation of RNA-binding. By providing a cost-effective means to validate in vivo RNA-binding activity of putative RBPs, LEAP-RBP is a valuable tool for focusing investigations on high confidence candidates. As a useful example for this suggestion, β-tubulin was identified as UV-enriched by LEAP-RBP, INP, XRNAX, OOPs, Ptex, TRAPP, RIC, and eRIC methods. Traditionally, this high degree of overlap suggests it's a good candidate for CLIP studies. However, SRA and immunoblot analysis of LEAP-RBP fractions suggests this is a widely observed false positive, perhaps reflecting RNP cargo/motor protein complex association with the microtubule cytoskeleton (FIG. 12a).

Beyond these applications, LEAP-RBP fractions could serve as a useful starting point for downstream interrogation of RNA-protein interactions. In these approaches, free RNA and protein components of lysates contribute substantial background contamination. LEAP-RBP overcomes these difficulties by removing free RNA, protein, and DNA while allowing scaling of clRNPs in an optimized buffer of choice (Supplementary Note 1).

### Supplementary Note 7

This note provides comparative analysis of proteins identified as UV-enriched* by LEAP-RBP and reference RNA-centric methods.

7a. LEAP-RBP Fractions Contain Many Previously Identified UV-Enriched* Proteins.

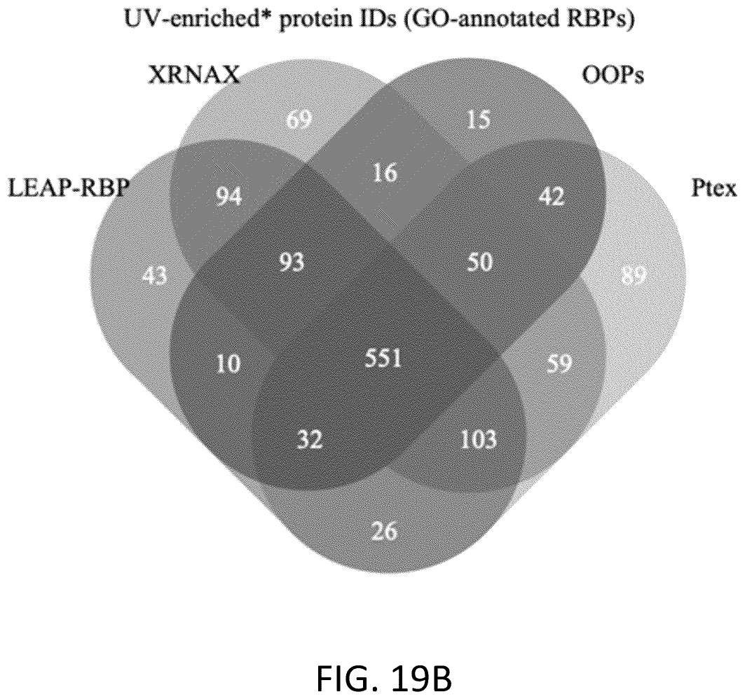

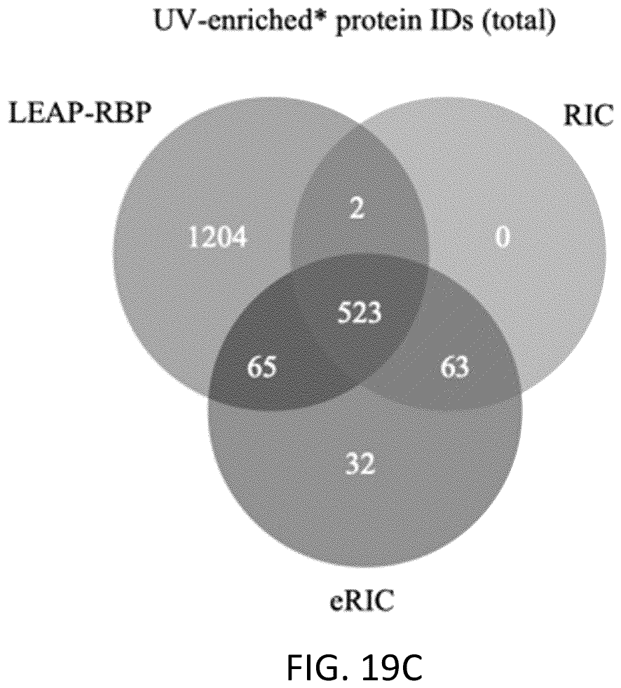

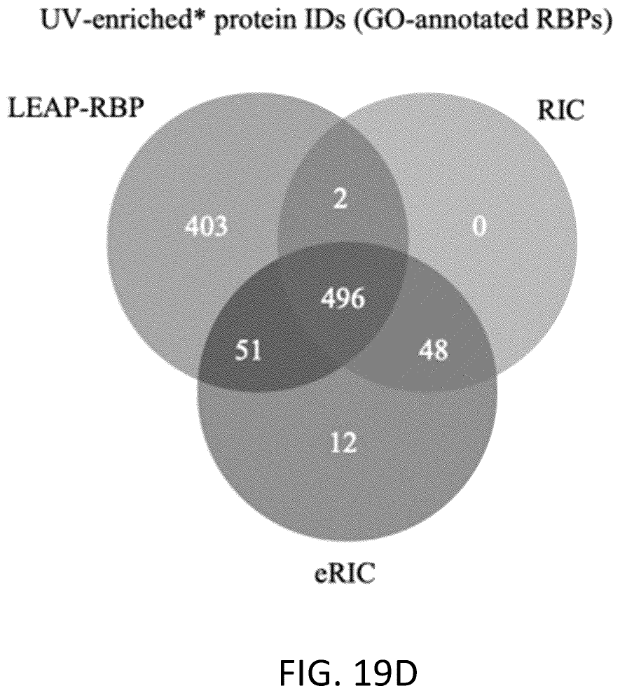

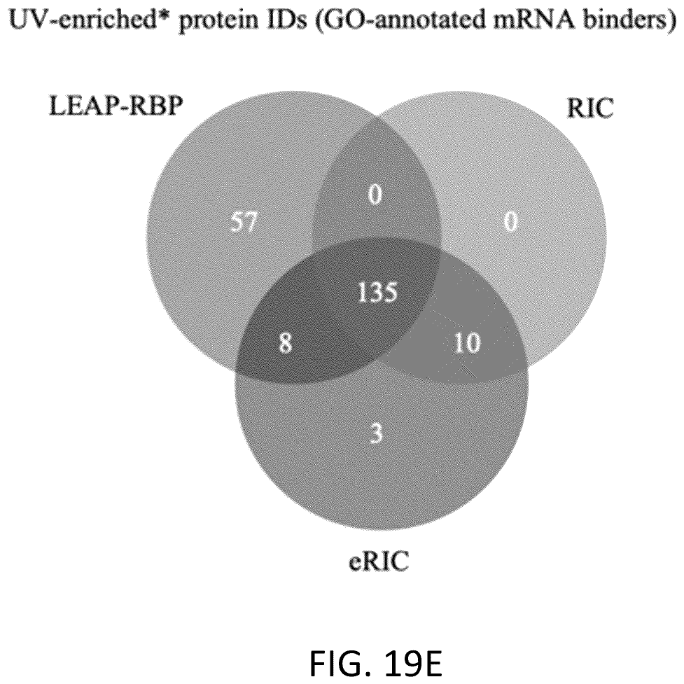

Many of the proteins identified as UV-enriched* in LEAP-RBP fractions by SILAC LC-MS/MS analysis were identified previously by XRNAX, OOPs, and pTEX methods as being UV-enriched* (FIG. 19A). Most of the proteins with shared UV-enrichment* status (˜78%) are GO-annotated as RBPs (FIG. 19B). Over 86% of proteins identified as UV-enriched* by RIC and/or eRIC methods were re-identified in LEAP-RBP fractions as UV-enriched* (FIG. 19c). Because GO-annotations of RBPs are largely based on UV-enrichment* status in prior RIC-like experiments, the overlap of GO-annotated RBPs largely mirrors the overlap of total UV-enriched* proteins (FIG. 19d, Supplementary Note 8b). Remarkably, 94% of GO-annotated mRNA binders (n=157) identified by RIC and/or eRIC as UV-enriched* were identified in LEAP-RBP fractions as UV-enriched* (FIG. 19e). An additional 57 GO-annotated mRNA-binders not identified in the referenced RIC and/or eRIC study were identified as UV-enriched* in LEAP-RBP fractions. These data demonstrate LEAP-RBP can achieve broad UV-enrichment*.

7b. Enhanced S/N Decreases UV-Enrichment* Specificity for RBPs.

SILAC LC-MS/MS analysis of INP and LEAP-RBP fractions demonstrated that enhanced enrichment of RNA-bound protein (S/N) increases the percentage of total protein in the sample that is RNA-bound (% TP_{S}) but decreases UV-enrichment* specificity for GO-annotated RBPs (FIG. 5h). This was attributed to a decrease in free protein recovery (% TP_{N}), lower limit of detection, and UV-enrichment* of low-abundance non-RBPs (FIG. 6c, Supplementary Note 4d, e).

LEAP-RBP displays increased % TP_{S} compared to other RNA-centric methods (FIG. 5h, 8e, 9c). Therefore, proteins with exclusive LEAP-RBP UV-enrichment* status are predicted to be less abundant (% TP), display lower S/N ratios, and be underrepresented by GO-annotated RBPs. Conversely, proteins with shared UV-enrichment* status are expected to be more abundant (% TP), display higher S/N ratios, and be overrepresented by GO-annotated RBPs. To test these predictions, proteins identified as UV-enriched* in LEAP-RBP fractions were binned based on the number of additional (0-5) RNA-centric methods that identified them as UV-enriched*; these include the INP method and referenced XRNAX, OOPs, Ptex, and RIC studies. The abundance (log₁₀(% TP)) of protein IDs from each bin were analyzed as a function of their log₂(S/N) ratios. As expected, proteins with shared UV-enrichment* status were of higher abundance, displayed higher S/N ratios, and were overrepresented by RBPs (+5 methods). Similar trends were observed when evaluating the abundances and enrichment efficiencies (S/N) of proteins with shared UV-enriched* status in any of the referenced MS datasets. Conversely, those with exclusive LEAP-RBP UV-enrichment* status were less abundant, displayed lower S/N ratios, and were underrepresented by RBPs. While proteins identified exclusively by other methods as being UV-enriched* were underrepresented by RBPs, they were not appreciably less abundant. These data support the view that increased method specificity results in significant UV-enrichment* of low-abundance RBPs, and reillustrates the main points of Supplementary Note 4e discussed below (Supplementary Note 7c). Additional information on the analyses were provided in the Supplementary Methods. Summary statistics (e.g., average log₂(S/N) ratios, # of protein IDs, # of UV-enriched* IDs, % TP contributions, etc.) for each of the categories (e.g., unique UV-enriched*, shared UV-enriched*) were made available in the provided MS datasets. For additional analyses of MS datasets, see Supplementary Note 7a, b.

7c. GO-Analysis of Protein IDs with Exclusive LEAP-RBP UV-Enrichment* Status Identifies Many Metabolic Enzymes.

GO-analysis was performed on proteins with exclusive LEAP-RBP UV-enrichment* status (n=293) or those with shared UV-enrichment* status (n=257). As expected, proteins with shared UV-enrichment* status were highly enriched for RNA-related functions and processes. Conversly, proteins with exclusive LEAP-RBP UV-enrichment* status were enriched for catalytic activities and metabolic processes. Indeed, many proteins with exclusive LEAP-RBP UV-enrichment* status are metabolic enzymes. Although their observed UV-enrichment* merits consideration as bona fide RNA-binding proteins, their low enrichment (S/N) and abundance (% TP) should also be considered. Indeed, many non-RBPs identified as UV-enriched* in LEAP-RBP fractions by SILAC LC-MS/MS were undetected by SRA and immunoblot (FIG. 12a). To this point, there has been a recent expansion in the number of UV-enriched* putative RBPs including metabolic enzymes, regulatory kinases, and others without previously known RNA-binding functions and/or which lack canonical RNA binding domains. Intriguing as these findings may be, without rigorous assessment of their enrichment efficiencies (S/N) and abundances (% TP), it remains unclear if these findings are biologically relevant. Low-abundance, non-specific UV-crosslinking of non-RBPs to non-RNA substrates may result in their significant UV-enrichment* in LEAP-RBP fractions. In principle, RNA-centric enrichment of RNA-bound protein can be viewed as a purification process aimed at the selective enrichment of proteins bound to sizable polyanions. However, UV-crosslinking of non-RBPs to other negatively charged small molecules (e.g., nicotinamide- and flavin-bearing dinucleotides) which are common co-factors of dehydrogenases could endow similar physicochemical properties, given the high enrichment efficiency and sensitivity of the LEAP-RBP method, this may result in their significant, albeit lowly efficient, UV-enrichment*.

Current high throughput methods for validation of UV-enriched* putative RBPs involve partial trypic-digestion of RNP fractions and TiO₂/SiO₂ or affinity-based enrichment of RNA-bound peptides. However, UV-enriched* proteins identified using these approaches include those which were undetectable in LEAP-RBP fractions by SRA and immunoblot (e.g., GRP78, GRP94, GAPDH; FIG. 12a). While considerably lower throughput, LEAP-RBP and SRA provide necessary orthogonal evidence of direct RNA-binding; RNase-dependent mobility shifts to the molecular weight of the unbound counterpart during SDS-PAGE is only expected for proteins bound to sizable RNA substrates through a single UV-crosslinking event (RNA-protein). Although the probability of multiple UV-crosslinked events is presumably lower than single UV-crosslinking events, rigorous testing of this assumption in vivo is lacking and likely depends on the RBP of interest.

7d. LEAP-RBP and SRA Reveal Discordance with In Vitro Validation Methods.

Despite several studies demonstrating its RNA-binding potential, RNA-binding activity for GAPDH was not validated herein. In prior studies, GAPDH was found to bind wild-type tRNA^{Met} in HeLa cells but not a mutant tRNA^{Met} version defective in nucleocytoplasmic transport. GAPDH was later found to exhibit increased binding to AU-rich elements on colony-stimulating factor-1 (CSF-1) mRNA in malignant (Hey) ovarian epithelial cells compared to normal (NOSE.1) ovarian epithelial cells. However, the interactions between GAPDH and RNA species were all observed post-lysis, in non-cellular contexts. Given the significance of buffer conditions for maintaining RNA-protein complexes during in vitro mobility-shift assays, RNA-protein interactions observed in vitro provide supportive but not conclusive evidence of an in situ RNA-binding function. In contrast, UV-crosslinking provides a way to stabilize physiologically relevant RNA-protein interactions occurring in vivo at zero-order distances. As demonstrated in this study, direct UV-crosslinking of RNA to protein via a single UV-crosslinking events is highly specific for RBPs. Because LEAP-RBP recovers near 100% of RNA-bound protein (Supplementary Note 3), the inability to detect GAPDH-RNA complexes is unlikely to reflect a unique bias in the isolation of RNA-bound proteins. Consistent with this view, it was observed that GAPDH behaved similarly to other non-RBPs during repeated AGPC extraction (FIG. 1c). While some amino acids are known to be less reactive towards RNA during UV-crosslinking, all should be considered susceptible to UV-crosslinking. RBPs in LEAP-RBP fractions displaying UV-crosslinking efficiencies<0.3% by SRA analysis were identified (e.g., RPN1; FIG. 10). Because many well-established RBPs were found to have UV-crosslinking efficiencies between 1-20% (0.4 J/cm², 254 nm), the physiological relevance of proteins in LEAP-RBP fractions undetected by SRA and immunoblot is questionable. Lastly, LEAP-RBP fractions isolated from HeLa cells that were UV-crosslinked under different conditions (e.g., without media removal and/or incubation on ice) display comparable RNase-sensitive profiles by SRA and Coomassie Blue (Protein) staining (Supplementary Note 3b). Therefore, the inability to detect GAPDH as RNase-sensitive by SRA and immunoblot is unlikely to be caused by UV-crosslinking or methodological artifacts. Indeed, if GAPDH were detected in LEAP-RBP fractions, it's not expected to appear RNase-sensitive (S/N=0.1). While GAPDH is routinely scored as UV-enriched* during RNA-centric RBP-capture experiments, it typically displays lower S/N. For example, while GAPDH was identified as UV-enriched* in the original RIC publication, it was not identified as UV-enriched* during the RIC experiment referenced in this study (non-SILAC, S/N=0.5). These data again demonstrate the utility of S/N-based analysis to assess RBP confidence and the ability of LEAP-RBP and SRA to rigorously validate RNA-binding occurring in vivo.

7e. Non-SILAC Comparison of RNP Fractions Isolated from UV-Crosslinked and Non-Crosslinked Cells Results in UV-Enrichment of Free Proteins Evidenced by Non-Specific % TP_{(S)} Contributions.

Overlap analysis of proteins identified in INP and LEAP-RBP by SILAC LC-MS/MS analysis showed many background proteins exclusively identified in INP fractions and displaying a log₂(CL/nCL) ratios with a mean distribution of 0 (Supplementary Note 4d). Comparison of INP fractions isolated from UV-crosslinked or non-crosslinked cells showed high UV-dependent enrichment of these background proteins which appear as “RNase-insensitive” bands by SRA and Coomassie Blue (protein) staining. Therefore, non-SILAC comparison of INP fractions isolated from independently processed UV-crosslinked and non-crosslinked cells would likely result in their apparent UV-enrichment*. Because most of these background proteins are non-RBPs (329/391), this is expected to decrease UV-enrichment* specificity. However, unlike the decrease in UV-enrichment* specificity caused by enhanced S/N and high % TP_{S}, a decrease in UV-enrichment* specificity caused by UV-dependent enrichment of free protein is evidenced by non-specific (non-RBP) % TP_{(S)} contributions. This can be explained by the following:
1) UV-crosslinking is inefficient, with an estimated 0.5-2.5% of the total protein being crosslinked to RNA depending on the UV-dose (0.1-0.8 J/cm², 254 nm; Supplementary Note 3a-c); even for the most abundant RBPs identified in LEAP-RBP fractions by SILAC LC-MS/MS (% TP rank), UV-crosslinking (0.4 J/cm², 254 nm) efficiency was estimated between 10-20% (gold boxes; FIG. 10). Therefore, relative % TP contributions of RBPs vs non-RBPs in input samples (i.e., total protein) is more reflective of their relative free protein abundances than relative RNA-bound abundances.

### Input (Total Protein):

$$\% {TP}_{( N ) , RBPs} / \% {TP}_{( N ) , non - RBPs} \approx \% {TP}_{RBPs} / \% {TP}_{non - RBPs} < \% {TP}_{( S ) , RBPs} / \% {TP}_{( S ) , non - RBPs}$$

2) Based on SILAC LC-MS/MS analysis of INP (% TP_{S}=47) and LEAP-RBP (% TP_{S}=91) fractions, an estimated 98.3-98.6% of the total RNA-bound protein (% TP_{(S)}) in either fraction is contributed by GO-annotated RBPs (% TP_{(S), non-RBPs}<2; Source Data FIG. 5h). Similar non-specific % TP_{(S)} contributions were observed for other RNA-centric methods utilizing SILAC LC-MS/MS approaches: 2.6 for XRNAX and ˜5.0 for TRAPP (Source Data FIG. 8e, 9c, Supplementary Note 8b). Therefore, differences in relative % TP_{(S)} contributions of RBPs and non-RBPs is likely indicative of high UV-crosslinking specificity.

### Input (Total Protein):

$$\% {TP}_{( N ) , RBPs} / \% {TP}_{( N ) , non - RBPs} < \% {TP}_{( S ) , RBPs} / \% {TP}_{( S ) , non - RBPs}$$

3) UV-dependent enrichment of free protein and signal-dependent recovery of noise are widely observed phenomenon; both can be observed by SDS-PAGE when comparing equivalent amounts (% fraction) of RNase-treated and untreated RNP fractions isolated from UV-crosslinked and non-crosslinked samples (Supplementary Note 4); repeated AGPC extraction: FIG. 1b; INP: FIG. 16; LEAP-RBP: FIG. 13c; XRNAX, OOPs, and Ptex: FIG. 8b, c; RIC and TRAPP: gold boxes, Source Data FIG. 8c. Free protein exhibiting UV-dependent enrichment during non-SILAC comparison will be perceived as false signal. This is expected to overestimate S/N ratios and % TP_{S} contributions of both RBPs and non-RBPs:

$$False \% {TP}_{( S )} = false signal ; ( false \% {TP}_{( S ) , RBPs} + \% {TP}_{( S ) , RBPs} ) / ( false \% {TP}_{( S ) , non - RBPs} + \% {TP}_{( S ) , non - RBPs} )$$

Depending on method specificity (% TP_{S}) and interactions between UV-crosslinking, method-specific RNA-enrichment conditions, and protein-specific physicochemical properties (Supplementary Note 4c), the relative false % TP_{(S)} contributions of RBPs vs non-RBPs is expected to vary. However, given the large difference between their relative % TP_{(S)} and % TP_{(N)} contributions in input samples, the following is likely:

$$\% {TP}_{( N ) , RBPs} / \% {TP}_{( N ) , non - RBPs} < false \% {TP}_{( S ) , RBPs} / false \% {TP}_{non - RBPs} < \% {TP}_{( S ) , RBPs} / \% {TP}_{( S ) , non - RBPs}$$

Affirmatively, non-specific % TP_{(S)} contributions for non-SILAC experiments were discernibly higher: 24.4 for OOPs and 28.4 for Ptex fractions. However, non-specific % TP_{(S)} contributions for the referenced RIC study (non-SILAC) were only 1.5%. This was attributed to high % TP_{S} of the RIC method and the observation that current GO-annotations of RBPs are largely based on their UV-enrichment* status in prior RIC-like (non-SILAC) experiments (Supplementary Note 8b).

### Supplementary Note 8

This note provides extended S/N-based analysis of LEAP-RBP, INP, and referenced RNA-centric methods.

8a. SRA and SILAC LC-MS/MS Serve as Complementary “S/N-Based” Analytical Approaches when Evaluating Method Specificity for RNA-Bound RBPs.

In this study, S/N and % TP_{S} serve as key metrics for evaluating RNA-bound protein enrichment and method specificity for RNA-bound RBPs. Both SRA and SILAC LC-MS/MS are considered “S/N-based” analytical approaches because they distinguish RNA-bound proteins from their unbound counterparts and evaluate S/N. Comparison of INP (% TP_{S}=47) and LEAP-RBP (% TP_{S}=91) fractions by SRA and SILAC LC-MS/MS analysis identified distinguishing features for methods with high specificity for RNA-bound RBPs. This primarily includes a lack of background proteins (S=0) appearing as RNase-insensitive bands by SRA and Coomassie Blue (protein) staining or as a distribution with mean log₂(CL/nCL) ratios of 0 by SILAC LC-MS/MS analysis (FIG. 2a, b, 4f, Supplementary Note 4d). When plotting the abundance (% TP) of proteins identified by SILAC LC-MS/MS analysis as a function of their log₂(S/N) ratios, methods with high % TP_{S} (e.g., LEAP-RBP) are distinguishable from methods with low % TP_{S} (e.g., INP) by the lack of high abundance proteins displaying negatives log₂(S/N). The observed quantities for proteins displaying positive or negative log₂(S/N) ratios are thus more representative of their RNA-bound or free protein quantities respectively (Supplementary Note 6b). GO-annotated RBPs contribute>98% of the total RNA-bound protein in INP and LEAP-RBP fractions (% TP_{(S)}; Source Data FIG. 5h), likely reflecting high UV-crosslinking specificity for RNA-binding proteins. When % TP_{S}=100%, % TP_{S}=% TP_{(S)} and observed protein abundances will reflect their true RNA-bound abundance (Supplementary Note 4e, f, 6a). The relative % TP_{(S)} contributions of RBPs vs non-RBPs is higher than their relative % TP_{(N)} contributions in input or RNP fractions (Supplementary Note 7e). Therefore, methods with high % TP_{S} (e.g., LEAP-RBP) are distinguished from methods with low % TP_{S} (e.g., INP) by an increase in the abundance (% TP) of RBPs as compared to non-RBPs. Contrarily, enhanced S/N was found to result in higher enrichment efficiencies of both RBPs and non-RBPs despite continued differences in their enrichment efficiencies. Therefore, methods with high % TP_{S} (e.g., LEAP-RBP) are distinguishable from methods with low % TP_{S} (e.g., INP) by the enhanced enrichment of both RBPs and non-RBPs.

RBP confidence score ranking exploits the observed differences in UV-enrichment efficiency (S/N) and abundance (% TP) of GO-annotated RBPs to distinguish them from non-RBP (FIG. 6e-g). As noted in the previous section, the relative abundance of RBPs as compared to non-RBPs is dependent on % TP_{S}. Therefore, RCS ranking is more accurate for methods with high specificity for RNA-bound proteins (% TP_{S}). For example, many of the RNA-binding proteins (red text) identified as RNase-sensitive in LEAP-RBP fractions (high % TP_{S}) by SRA and immunoblot (e.g., NCL, TRAPα, and RPN1; FIG. 2b), displayed log₂(S/N) ratios less than 0 in INP fractions (low % TP_{S}) by SILAC LC-MS/MS. Because RCS ranking automatically places proteins with negative log₂(S/N) ratios at lower ordinal rank (FIG. 6f, g, Supplementary Methods), these would be considered low confidence RBPs (low RCS). The placement of proteins displaying negative log₂(S/N) ratios at lower ordinal rank is significant for comparative studies because their observed abundance is more representative of their free protein abundance (Supplementary Note 6b, c). However, the placement of GO-annotated RBPs at lower RCS rank is indicative of low specificity, as most RBPs display positive log₂(S/N) in LEAP-RBP fractions (high % TP_{S}). Therefore, the ability of both RCS and % TP ranking to accurately place GO-annotated RBPs at higher ordinal rank than non-RBPs is indicative of high % TP_{S}.

Comparison of observed protein quantities (SPI) between biological replicates after mean-normalized to total SPI illustrates the expected correlation for proteomic samples with similar protein profiles (i.e., relative protein quantities or Δ log₁₀(SPI); Supplementary Methods). Conversely, observed protein profiles in the UV-crosslinked (SPI_{CL}) and non-crosslinked (SPI_{nCL}) SILAC channels are dissimilar. This is expected; the observed protein quantities in the UV-crosslinked SILAC channel represent RNA-bound and free protein quantities (S+N) while observed protein quantities in the non-crosslinked SILAC channel represent free protein quantities (N; Supplementary Note 5). Therefore, the profile of proteins displaying high RCS (high S/N) are expected to be more dissimilar: (S+N)_{CL}>N_{nCL}. Conversely, the profile of proteins displaying low RCS (low S/N) are expected to be more similar: (S+N)_{CL}≈N_{CL}≈N_{nCL}. Indeed, proteins displaying lower RCS are identifiable in methods with low % TP_{S} by their similar profiles in both SILAC channels. For methods with high % TP_{S}, the most abundant proteins in both SILAC channels display high RCS (log₂(S/N)>0). During non-SILAC comparisons, free proteins are expected to appear UV-enriched (Supplementary Note 7e), but they can be identified by the similarity of their (free) protein profiles in both UV-crosslinked and non-crosslinked samples (Supplementary Note 8b). Proteins identified exclusively in the UV-crosslinked SILAC channel during SILAC LC-MS/MS analysis of LEAP-RBP and INP fractions were also the least abundant. While these proteins were given pseudo-log₂(S/N) ratios of 10 and considered high-confidence RBPs in the traditional sense (Supplementary Methods), their enrichment is not considered meaningful. For example, XRN1 displays enhanced RNase-sensitivity (S/N) in LEAP-RBP (L) fractions compared to INP (I) fractions by SRA and immunoblot (FIG. 2b). This should equate to a higher S/N ratio for XRN1 in LEAP-RBP fractions as compared to INP fractions by SILAC LC-MS/MS analysis. However, XRN1 was identified in both SILAC channels for LEAP-RBP (high % TP_{S}) and given a log₂(S/N) ratio of 3.5, but only detected in the UV-crosslinked SILAC channel for INP (low % TP_{S}) and given a pseudo-log₂(S/N) ratio of 10 (XRN1). This is attributed to improved sensitivity by the LEAP-RBP method and lower limit of detection for noise in the non-crosslinked SILAC channel (Supplementary Note 5). Indeed, proteins only identified in the UV-crosslinked SILAC channel of LEAP-RBP (high % TP_{S}) fractions were overrepresented by non-RBPs as compared to those identified in INP (low % TP_{S}) fractions. Therefore, the inability to detect noise for GO-annotated RBPs is indicative of low sensitivity and % TP_{S}. Because of this, proteins undetected in the non-crosslinked SILAC channel or non-crosslinked sample were not included in RCS rank analysis because of their less meaningful S/N ratios and lower quantitative accuracy (i.e., # of peptides; Supplementary Methods).

8b. Additional Analyses of Referenced MS Datasets.

Additional analyses of the referenced MS data (bold) are provided below. Additional information on MS data processing and analysis of referenced datasets are included in the Supplementary Methods.

### XRNAX

Analyses were performed with available MS data generated by SILAC LC-MS/MS analysis of 12 XRNAX fractions isolated from pooled UV-crosslinked (0.2 J/cm², 254 nm) and non-crosslinked cells (MCF7, HeLa, and HEK293) grown to either half-confluence or confluence and digested for 15 minutes or 30 minutes prior to silica enrichment. In this study, XRNAX fractions were isolated from 30 million UV-crosslinked (0.2 J/cm², 254 nm) or non-crosslinked HeLa cells (Supplementary Note 9a). Evaluation of XRNAX fractions by SRA and SYBR Safe (RNA&DNA) staining demonstrated high UV-enrichment of RNA and efficient digestion of DNA (TBE gel analysis or polyacrylamide gel; FIG. 8a, b), consistent with published data (FIG. 1b). Comparison of XRNAX fractions isolated from UV-crosslinked and non-crosslinked cells showed moderate UV-enrichment of RNase-insensitive (free) protein by SRA and Coomassie Blue (protein) staining (FIG. 8 b). This is consistent with reported observations and rationale for using a SILAC LC-MS/MS approach (Supplementary Note 4b). XRNAX and LEAP-RBP display comparable signal recovery by SRA and immunoblot (e.g., HuR, X vs L; FIG. 8c) but some RBPs display discernibly lower RNase-sensitivity (S/N) in XRNAX fractions (e.g., RPL4). This is consistent with reported observations and rationale for using partial tryptic digestion and repeated TiO₂/SiO₂ enrichment to further enrich RNA-bound peptides prior to LC-MS/MS analysis. Available MS data indicates ˜71% of the total protein in XRNAX fractions is RNA-bound (% TP_{S}) (Source Data FIG. 8e). Comparing the observed RNase-sensitivity of total protein in XRNAX (pre-cleanup) and INP (% TP_{S}=47; Source Data FIG. 5h) fractions by SRA and Coomassie Blue (protein) staining (FIG. 2a, 8b) suggests this is highly effective at increasing % TP_{S} of XRNAX fractions. This can be considered a favorable increase in % TP_{S} (Supplementary Methods); i.e., it doesn't discernibly increase non-specific % TP_{(S)} contributions (2.6; Source Data FIG. 8e). The ability of both RCS and % TP ranking to accurately place GO-annotated RBPs at higher ordinal rank than non-RBPs is considered indicative of methods with high % TP_{S} (Supplementary Note 8a). Indeed, compared to non-RBPs, RNase-sensitive RBPs were of mostly higher abundance in XRNAX fractions and scored as higher confidence. A similar trend was observed for all GO-annotated RBPs and non-RBPs. In this study, protein quantities were estimated using sum peptide intensities (SPI) rather than iBAQ intensities as done in the original XRNAX study. The 30 most abundant proteins in XRNAX fractions estimated as a percentage of total iBAQ intensity were mostly comparable to the 30 most abundant proteins in XRNAX fractions estimated as a percentage of total SPI (% TP). This suggests either label-free quantification method is suitable for MS data analyses. Comparison of observed protein quantities in the UV-crosslinked and non-crosslinked SILAC channels shows similar free (low RCS) and dissimilar RNA-bound (high RCS) protein profiles. These data and observations are reasonably consistent with those reported by the original XRNAX study.

### OOPs

Analyses were performed with available MS data (3 out of 4 replicates) generated by LC-MS/MS analysis of OOPs fractions isolated from the organic phase following AGPC extraction of RNase-treated 5^{th} AGPC interphase samples isolated from UV-crosslinked (0.8 J/cm², 254 nm) or non-crosslinked human CD4⁺ T cells. In this study, OOPs fractions were isolated from the organic phase following AGPC extraction of RNase-treated and untreated 3^{rd} AGPC interphase samples isolated from 30 million UV-crosslinked (0.2 J/cm², 254 nm) or non-crosslinked HeLa cells (Supplementary Note 9b). Evaluation of OOPs fractions by SRA and Coomassie Blue (protein) staining demonstrated moderate UV-enrichment of proteins exhibiting ubiquitous RNase-dependent enrichment (OOPs; FIG. 8b). This is consistent with observations of the original OOPs study that 96% of proteins UV-enriched* at the 3^{rd} AGPC interphase (CL), including CL-independent proteins (RNA-free), exhibit RNase-dependent migration into the organic phase (FIG. 2i, FIG. 10h). Therefore, observed RNase-dependent migration of free protein into the organic phase is expected to overestimate S/N and % TP_{S} of OOPs fractions by SRA analysis (FIG. 8b, c, Supplementary Note 7e). This is less likely to impact the observed S/N of RBPs with higher UV-crosslinking efficiencies and/or those more readily enriched by repeated AGPC extraction (Supplementary Note 4e). As noted by the authors of the original OOPs study (FIG. 10B), additional repeated AGPC extraction would be expected to improve UV-dependent enrichment of RNA-bound and free proteins at the AGPC interphase (FIG. 1b, c). Analyses were performed with MS data generated by a non-SILAC LC-MS/MS experiment. Therefore, the observed enrichment efficiencies (S/N) of RBPs and non-RBPs are expected to reflect their observed UV-enrichment in RNase-treated OOPs fractions by SRA and immunoblot (O, CL vs nCL; FIG. 8c). Additionally, the observed abundances (% TP) of RBPs and non-RBPs are expected to mostly reflect their observed abundance in RNase-treated OOPs fractions isolated from UV-crosslinked cells by SRA and immunoblot (O, CL and RNase; FIG. 8c). Available MS data (non-SILAC) showing higher enrichment efficiencies and abundances for RBPs as compared to non-RBPs (O; FIG. 8d), and high % TP_{S} (73.4) with high non-specific % TP_{(S)} contributions (24.4) match these predictions (Source Data FIG. 8e). In this study, protein quantities were estimated using sum peptide intensities (SPI) rather than LFQ intensities generated by MaxLFQ as reported in the referenced OOPs study. As discussed in Supplementary Note 4f, LFQ algorithms assumes protein profiles are mostly comparable between experimental samples (i.e., CL and nCL). However, protein recovered from UV-crosslinked cells contains both RNA-bound and free protein while protein recovered from non-crosslinked cells contains only free protein; therefore, this LFQ algorithms are expected to underestimate UV-enrichment efficiencies (S/N; Supplementary Note 8a). Indeed, many proteins showing clear UV-enrichment by SRA and immunoblot appear non-enriched using LFQ intensities (e.g., NCL) and % TP_{S} is discernibly lower (22.1). In both datasets, similar protein profiles are observed in both UV-crosslinked and non-crosslinked samples. This finding is consistent with reported RNase-dependent enrichment of CL independent (RNA free) protein in OOPs fractions and the expectation that free protein quantities display more similar profiles in UV-crosslinked vs non-crosslinked samples (Supplementary Note 8a). Overall, the data and observations presented in this study are consistent with those reported by the original OOPs study and the referenced MS data.

### Ptex.

Analyses were performed with available MS data generated by LC-MS/MS analysis of Ptex fractions isolated from UV-crosslinked (1.5 J/cm², 254 nm) or non-crosslinked HEK293 cells. In this study, Ptex fractions were isolated from 30 million UV-crosslinked (0.2 J/cm², 254 nm) or non-crosslinked HeLa cells (Supplementary Note 9c). Evaluation of Ptex fractions by SRA and Coomassie Blue (protein) staining demonstrating moderate UV-enrichment of RNase-insensitive protein displaying comparable protein profiles in both UV-crosslinked and non-crosslinked samples (blue boxes, Ptex; FIG. 8b). Indeed, comparing the observed protein quantities in UV-crosslinked and non-crosslinked samples shows highly similar profiles. Additionally, the observed profile of total protein in Ptex fractions by SDS-PAGE and Silver Stain (RNA, DNA, and protein) staining (Silver Stain, Ptex; FIG. 8b) is consistent with reported polyacrylamide gels showing SDS-PAGE separated Ptex fractions stained with Silver Stain. Evaluation of Ptex fractions by SRA and immunoblot showed UV-enrichment of RNase-insensitive RBPs (e.g., NCL and TRAPα) and GRP94 (P; FIG. 8c). This is consistent with published immunoblots showing similar non-specific UV-enrichment of RNase-insensitive proteins in Ptex fractions (FIG. 1f). Analyses were performed with MS data generated by a non-SILAC LC-MS/MS experiment. Therefore, the observed enrichment efficiencies (S/N) of RBPs and non-RBPs are expected to reflect their observed UV-enrichment in RNase-treated Ptex fractions by SRA and immunoblot (P, CL vs nCL; FIG. 8c). Additionally, the observed abundances (% TP) of RBPs and non-RBPs are expected to mostly reflect their observed abundances in RNase-treated Ptex fractions isolated from UV-crosslinked cells by SRA and immunoblot (P, CL and RNase; FIG. 8c). Available MS data (non-SILAC) showing non-specific S/N, and non-specific % TP_{(S)} contributions (28.4) match these predictions (Source Data FIG. 8e). The observed abundance of proteins (e.g., NCL and GRP94) in Ptex fractions by SRA and immunoblot are consistent with their observed abundance in available MS data. Comparable analyses of MS data generated by LC-MS/MS analysis of Ptex fractions isolated from HEK293 cells UV-irradiated with 0.015 or 0.15 J/cm² (254 nm) conducted. In this study, protein quantities were estimated using sum peptide intensities (SPI) rather than iBAQ intensities as done in the original Ptex study. Nonetheless, published scatterplots showing protein abundances estimated using iBAQ intensities are comparable to protein abundances estimated using SPI values (FIG. 5f). These data and observations are reasonably consistent with those reported by the original Ptex study.

### TRAPP.

Analyses were performed with available MS data generated by SILAC LC-MS/MS analysis of TRAPP fractions isolated from pooled samples containing non-crosslinked and UV-crosslinked (400, 800, or 1360 mJ/cm²; 254 nm) yeast cells. In this study, TRAPP fractions were isolated from 30 million UV-crosslinked (0.2 J/cm², 254 nm) or non-crosslinked HeLa cells (Supplementary Note 9d). Evaluation of TRAPP fractions (CL vs nCL) by SRA and Coomassie Blue (protein) staining demonstrated high UV-dependent enrichment of RNase-sensitive proteins (blue boxes, TRAPP; FIG. 8b). Further evaluation by SRA and immunoblot showed high specificity for RNase-sensitive RBPs and signal-dependent recovery of noise (TRAPP, gold box and red (signal) or blue (noise) arrows; Source Data FIG. 8c). The observed RNase-sensitivity of total protein in TRAPP fractions (CL) by SRA and Coomassie Blue (protein) staining is suggestive of high % TP_{S} (FIG. 9a, Supplementary Note 8a). Indeed, analysis of available SILAC LC-MS/MS data (1360 mJ/cm², 254 nm) indicates 86% of the total protein in the sample is RNA-bound (% TP_{(S)}; Source Data FIG. 9c). The observed % TP_{S} of TRAPP fractions was found to depend on the UV-dose, with only 60% or 67% of total protein originating from RNA-bound protein when yeast cells were UV-irradiated with 400 and 800 mJ/cm² (254 nm) respectively (Source Data FIG. 9j). The apparent non-specific % TP_{(S)} of TRAPP fractions (400, 800, and 1360) is due to incomplete GO-annotation (GO:RBP) of ribosomal proteins (Source Data FIG. 9j). When accounting for ribosomal proteins lacking RBP-annotations (i.e., proteins whose primary gene name starts with “RPS” or “RPL”), non-specific % TP_{(S)} contributions are greatly reduced. Curiously, there is a larger improvement for TRAPP 400 (95) and TRAPP 800 (95) fractions as compared to TRAPP 1360 (79) fractions despite their lower % TP_{S} (60, 67, and 86 respectively). Because RBPs contributed most of the total RNA-bound protein in XRNAX (% TP_{(S), RBPs}=97), LEAP-RBP (% TP_{(S), RBPs}=98), and INP (% TP_{(S), RBPs}=99) fractions by SILAC LC-MS/MS (Source Data FIG. 5h, 8e), this likely reflect a UV-dependent decrease in % TP_{(S)} specificity. To test this, 30 most abundant RNA-bound non-RBPs (% TP_{S} rank) identified in TRAPP 1360 fractions were binned their estimated % TP_{S} and % TP_{N} contributions were compared in TRAPP 400, 800, and 1360 fractions. Indeed, there was a clear UV-dose dependent decrease in % TP_{S} contributions of most ribosomal protein (red text) and an increase in % TP_{S} contributions for all non-ribosomal proteins. High levels of UV-crosslinking may result in more non-specific UV-crosslinking and/or RBP-specific signal loss during the stringent purification procedures. In support of this, a decreased in RNA yield was observed in TRAPP fractions isolated from UV-crosslinked vs non-crosslinked cells (μg of RNA, CL vs nCL; FIG. 8b). The effect of UV-dose on RNA yield was not evaluated in the original TRAPP study. Comparison of TRAPP and LEAP-RBP fractions isolated from UV-crosslinked cells by SRA and immunoblot showed comparable recovery of ribosomal proteins (e.g., RPL4 and RPL8) displaying high RNase-sensitivity (S/N), but a large difference in recovery of non-ribosomal proteins (FIG. 9b). Indeed, available MS data shows RPL4 and RPL8, among other ribosomal proteins, as the most abundant RBPs in TRAPP (Source Data FIG. 9h). These observations illustrate how variations in signal recovery can introduce potential biases (Supplementary Note 1). These data and observations are reasonably consistent with those reported by the original TRAPP study.

RIC and eRIC.

Analyses were performed with available MS data by LC-MS/MS analysis of RIC and eRIC fractions isolated from UV-crosslinked (0.15 J/cm², 254 nm) or non-crosslinked Jurkat cells. In this study, RIC fractions were isolated from 30 million UV-crosslinked (0.2 J/cm², 254 nm) or non-crosslinked HeLa cells (Supplementary Note 9e). Evaluation of RIC fractions (CL vs nCL) by SRA and Coomassie Blue (protein) staining demonstrated high UV-enrichment of RNase-sensitive proteins (blue boxes, RIC; FIG. 8b). Further evaluation by SRA and immunoblot showed high specificity for RNase-sensitive RBPs and signal-dependent recovery of noise (RIC, gold box and red (signal) or blue (noise) arrows; Source Data FIG. 8c). S/N-based analyses of RIC fractions by SRA and SILAC LC-MS/MS analysis have not yet been reported. However, the RNase-sensitivity of total protein in RIC fractions (CL) by SRA and Coomassie Blue (protein) staining is indicative of high % TP_{S} (FIG. 9a, b, Supplementary Note 8a). This is expected to limit UV-enriched free protein contributions towards observed enrichment efficiencies (S/N) and RNA-bound abundances (% TP_{S}) of RBPs and non-RBPs (Supplementary Note 7e). Nonetheless, the observed enrichment efficiencies (S/N) of RBPs and non-RBPs in available MS data are expected to reflect their observed UV-enrichment in RNase-treated RIC fractions by SRA and immunoblot (R, +CL vs nCL; FIG. 8c). Additionally, the abundances (% TP) of RBPs and non-RBPs are expected to mostly reflect their observed abundances in RNase-treated RIC fractions isolated from UV-crosslinked cells by SRA and immunoblot (R, CL and RNase; FIG. 5c). Available MS data (non-SILAC) showing high enrichment efficiencies and abundances for most RBPs over non-RBPs match these predictions (R; FIG. 8d). Most of the proteins identified in RIC fractions by LC-MS/MS have prior RBP-annotations (GO:RBP) regardless of their individual enrichment efficiencies (S/N). It was postulated that RNA-binding activity for many of the GO-annotated RBPs displaying low S/N ratios was inferred because of their UV-enrichment* status in prior RIC studies (non-SILAC). Indeed, the RNA-binding activity for 87 of the bottom 100 RBPs ranked by RCS and identified in the referenced RIC study was inferred by their UV-enrichment* status in prior RIC studies. As demonstrated in this study, UV-enrichment* alone is insufficient evidence for assigning RNA-binding activity. Therefore, the accuracy of RBP-annotations (GO:RBP) would likely benefit from additional criteria such as demonstrating their RNase-sensitivity by SRA and immunoblot. These observations suggest the number of RNA-binding proteins has been significantly overestimated (Supplementary Note 7c, d). The 300 most abundant proteins identified in LEAP-RBP fractions by SILAC LC-MS/MS contribute 95% of total RNA-bound protein in the sample (% TP_{(S)}). Therefore, it's unlikely that a decrease in RBP-annotations (GO:RBP) would diminish the ability of % TP_{S} to reflect method specificity.

Compared to the TRAPP method, RIC displayed more efficient recovery of non-ribosomal proteins (e.g., HuR, pAbPC1, and PABPC4) and less efficient recovery of ribosomal protein (e.g., RPL4 and RPL8) by SRA and immunoblot (RNase T vs R; FIG. 9b). However, when compared to LEAP-RBP, the selective recovery of non-ribosomal proteins by RIC is no longer apparent (RNase, R vs L; FIG. 9b). Indeed, the abundance of ribosomal (e.g., RPL4 and RPL8) relative to non-ribosomal proteins (e.g., HuR, pAbPC1, and PABPC4) in RIC fractions is comparable to LEAP-RBP fractions. The high abundance of NCL relative to other proteins (˜one order of magnitude) identified RIC fractions by LC-MS/MS analysis is consistent with its observed abundance (˜110 kD band) by SRA and Coomassie Blue (protein) staining (RNase, R; FIG. 9a). The abundance of ribosomal proteins identified in RIC fractions by LC-MS/MS is consistent with TBE gel analysis of RIC fractions (CL) showing appreciable recovery of rRNA (RNP fraction, RIC; FIG. 8a). Indeed, these observations were reported by others and served as the driving force behind development of more stringent protocols utilizing LNA probes (eRIC) ([FIG. 2a). Compared to RIC, the abundance of ribosomal proteins (e.g., RPL4 and RPL8) identified in eRIC fractions by LC-MS/MS are decreased relative to non-ribosomal proteins (e.g., HuR and TIA1). This includes the highly abundant nucleolar RBP, nucleolin (NCL), consistent with published polyacrylamide gels stained by Silver Stain showing a decrease in observable NCL migrating at its expected weight (˜110 kD) in eRIC fractions (RNase) when compared to RIC fractions (RNase) (FIG. 2c). These data and observations are reasonably consistent with those reported by the referenced RIC and eRIC stud.

### Supplementary Note 9

This note includes extended protocols for referenced RNA-centric methods.

9a. XRNAX

XRNAX was performed on samples containing either 30 million UV-crosslinked (0.2 J/cm², 254 nm) or 30 million non-crosslinked HeLa cells according to the published protocol. Cells were harvested with two 800 μL aliquots of GT buffer and transferred to a 15 mL conical tube. Then, 800 μL phenol (acidic) were added and samples were triturated until no visible clumps remained. The samples were split between three 2 mL microcentrifuge tubes and 160 μL chloroform were added to each. Samples were inverted four times, incubated standing for 5 min at RT, and centrifuged at 7,000×g for 10 min at 4° C. Aqueous phases were removed and the interphase fractions were transferred to a 2 mL microcentrifuge tube. Interphase samples were washed twice with 0.3 mL TE+0.1% SDS. The remaining interphase fractions were disintegrated using two 0.3 mL aliquots of TE+0.1% SDS and two 0.3 mL aliquots of TE+0.5% SDS by pipetting with each aliquot and transferring solubilized fractions to a 2 mL tube. Pooled solubilized interphase samples were mixed briefly and aliquoted between two 2 mL microcentrifuge tubes for isopropanol precipitation. To each aliquot, 36 μL 5.0 M NaCl, 0.6 μL Glycoblue, and 600 μL isopropanol were added. Samples were inverted several times and centrifuged at 18,000×g for 15 min at 4° C. Supernatants were removed and precipitates were washed with 0.3 mL RT 70% ethanol. Samples were spun down at 18,000×g for 1 min at RT. Precipitates were air dried, 270 μL of DEPC-treated water were added, and samples were incubated overnight at 4° C. Then, 30 μL 10× DNase I buffer, 0.3 μL RNaseOUT (10777019, Invitrogen), and 15 μL DNase I (M0303S, NEB) were added to each sample. Samples were incubated in a thermomixer for 90 min at 37° C. (700 rpm) and precipitated with 18 μL 5.0 M NaCl, 0.3 μL GlycoBlue, and 300 μL isopropanol. Samples were inverted several times and centrifuged at 18,000×g for 15 min at 4° C. Supernatants were removed and precipitates were washed with 0.15 mL room-temperature 70% ethanol by pipetting. Samples were centrifuged down at 18,000×g for 1 min at RT. An additional transfer/washing step was used to improve solubilization of precipitates: three 400 μL aliquots of RT 95% methanol were used to recover precipitates adhering to the sides of the tubes and combined in a 1.5 mL microcentrifuge tube. The tubes were then placed vertically at 4° C. to allow precipitate settling at the bottom of the tube. Samples were centrifuged at 20,000×g for 10 min at 20° C. and supernatants were removed. Pellets were air dried and resuspended at the desired concentration with 1% LiDS TE.

9b. OOPs

OOPs was performed on samples containing either 30 million UV-crosslinked (0.2 J/cm², 254 nm) or 30 million non-crosslinked HeLa cells according to the published protocol. Cells were harvested with two 1 mL aliquots of GT buffer and transferred to a 15 mL conical tube. Then, 1 mL phenol (acidic) was added and samples were triturated until no visible clumps remained. The samples were split between three 2 mL microcentrifuge tubes and 200 μL chloroform were added to each. Samples were vortexed (max) for 15 sec and centrifuged at 12,000×g for 15 min at 4° C. A gel loading pipette tip was used to remove the aqueous and organic phases while leaving the interphase undisturbed. 1 mL of fresh acidic guanidinium thiocyanate-phenol (2:1) buffer was added and interphase samples were resolubilized by pipetting. Then, 200 μL chloroform were added and samples were AGPC extracted as before. This process was repeated for a total of three AGPC extractions. Then, 9 volumes of RT 100% methanol (˜1.35 mL) were added to interphase samples and immediately centrifuged at 14,000×g for 10 min at 4° C. Precipitates were washed twice with 1 mL RT 95% methanol by pipetting and centrifuged at 14,000×g for 10 min at 4° C. Three 400 μL aliquots of RT 95% methanol were used to recover precipitates adhering to the sides of the tubes and combined (pool aliquots) in a 1.5 mL microcentrifuge tube. The tubes were then placed vertically and incubated for 30 min at RT to allow precipitate settling at the bottom of the tube. Samples were centrifuged at 14,000×g for 10 min, supernatants were removed, and precipitated were air dried. 70 μL TE buffer were added and samples were incubated overnight at 4° C. followed by pipetting until precipitates solubilized. RNase-digestion was performed in separate 1.5 mL microcentrifuge tubes using 30 μL of TE-suspended interphase samples. RNase Cocktail (AM2286, Invitrogen), 10× RNase digest buffer (100 mM Tris-HCl pH 7.5, 1 M NaCl, and 10 mM EDTA), and 25× protease inhibitors (11836153001, Roche) were added at the same time to a final concentration of 2 μL RNase Cocktail/15 μg protein-bound RNA, 1× RNase digestion buffer, and 1× protease inhibitors (100 μL total reaction volume). Untreated control samples were set up without RNase Cocktail, and both were incubated for 2 hours at 37° C. The recommended RNase-digest conditions do not include RNase digestion buffer and involve overnight incubation at 37° C. The additional (optional) MeOH washes were found to improve subsequent solubilization. This, along with the addition of RNase digestion buffer, was found to facilitate efficient digestion of RNA within a timeframe that avoided protein degradation. 1 mL of fresh acidic guanidinium thiocyanate-phenol (2:1) were added to each sample followed by brief vortex. 200 μL chloroform were added and samples were vortexed for 15 sec. Samples were centrifuged at 12,000×g for 15 min at 4° C. The upper aqueous phase and interphase fractions were removed, and three 150 μL aliquots of the organic phase were each transferred to 1.5 mL microcentrifuge tubes containing 1.35 mL RT 100% methanol. Samples were vortexed for 15 sec and centrifuged at 20,000×g for 10 min at 4° C. Precipitates were washed twice with 1 mL RT 95% methanol by pipetting and centrifuged at 14,000×g for 10 min at 4° C. Three 400 μL aliquots of RT 95% methanol were used to recover precipitates adhering to the sides of the tubes and combined in a 1.5 mL microcentrifuge tube. The tubes were then placed vertically and incubated for 30 min at RT to allow precipitate settling at the bottom of the tube. Samples were centrifuged at 14,000×g for 10 min and supernatants were removed. Pellets were air dried and resuspended at the desired concentration with 1% LiDS TE.

9c. Ptex

Ptex was performed on samples containing either 30 million UV-crosslinked (0.2 J/cm², 254 nm) or 30 million non-crosslinked HeLa cells according to the published protocol. Cells were harvested with two 1 mL aliquots of ice-cold 1×PBS and transferred to a 15 mL conical tube. Additional 1×PBS was added to each sample for a final volume of 2.25 mL. 750 μL neutral phenol, 750 μL toluol (244511, Sigma-Aldrich), and 750 μL 1,3-bromochloropropane (BCP) (B9673, Sigma-Aldrich) were added and samples were triturated until no visible clumps remained. Samples were aliquoted between three 2 mL microcentrifuge tubes and mixed at 2,000 rpm for 1 minute at RT. Samples were centrifuged at 20,000×g for 3 min at 4° C. The aqueous phases were each transferred to 2 mL microcentrifuge tubes containing 300 μL solution D. 600 μL neutral phenol and 200 μL BCP were added. Samples were mixed at 2,000 rpm for 1 min at RT and centrifuged at 20,000×g for 3 min at 4° C. ¾^{th} of the aqueous and organic phases were removed and 400 μL DEPC-treated water, 200 μL 100% ethanol, 400 μL neutral phenol, and 200 μL BCP were added to each sample. Samples were mixed at 2,000 rpm for 1 min at RT and centrifuged at 20,000×g for 3 min at 4° C. A gel loading pipette tip was used to remove the aqueous and organic phases while leaving the interphase undisturbed. 9 volumes of 100% ethanol were added to each sample and incubated overnight at −20° C. The next day, samples were spun down at 20,000×g for 30 min at 4° C. and supernatants were removed. To ensure removal of salts prior to resuspension and RNase-digestion, pellets were washed twice with 1.0 mL ice-cold 75% ethanol. For each wash, samples were incubated on ice for 5 min followed by centrifugation at 18,000×g for 5 min at 4° C. and removal of supernatant. Then, three 400 μL aliquots of ice-cold 75% ethanol were used to recover precipitates adhering to the sides of the tubes and combined (pool aliquots) in a 1.5 mL microcentrifuge tube. The tubes were then placed vertically and incubated for 30 min on ice to allow precipitate settling at the bottom of the tube. Samples were centrifuged at 18,000×g for 5 min at 4° C. and supernatants were removed. Pellets were air dried and resuspended at the desired concentration with 1% LiDS TE.

9d. TRAPP

TRAPP was performed on samples containing either 30 million UV-crosslinked (0.2 J/cm², 254 nm) or 30 million non-crosslinked HeLa cells according to the published protocol. Cells were harvested with two 600 μL aliquots of GT buffer and transferred to a 15 mL conical tube. Then, 1.2 mL phenol (acidic) were added and lysates were sheered by passaging through a 19 ga 1½″ needle fifteen times. Samples were centrifuged at 4,600×g for 5 min at 4° C. and supernatants were transferred to 15 mL conical tubes. Samples were centrifuged at 13,000×g for 10 min at 4° C. and supernatants were transferred to a 15 mL conical tube (˜2.65 mL per clarified sample with residual PBS). 270 μL 3 M sodium acetate-acetic acid pH 4.0 were added to each tube and samples were mixed briefly. 3 mL RT 100% ethanol were added slowly to samples and then mixed by vortex (5 sec). 1.5 mL of equilibrated 50% silica bead slurry (S5631, Sigma Aldrich) were added to each sample followed by 1.5 mL RT 100% ethanol; silica beads were equilibrated by incubating overnight in 1 M HCl and washed several times with DEPC-treated water. Samples were vortexed briefly to fully resuspend beads and incubated on a rotator for 60 min at RT. Samples were centrifuged at 2,500×g for 2 min at 4° C. and supernatants were removed. Silica beads were resuspended by vigorous vortexing in 4.5 mL wash buffer I (4 M guanidine thiocyanate, 1 M sodium acetate-acetic acid pH 4.0, and 30% ethanol); 30 sec. Samples were centrifuged at 2,500×g for 2 min at 4° C. and supernatants were removed. This wash step was repeated two more times (wash buffer I), followed by three washes using 4.5 mL wash buffer II (100 mM NaCl, 50 mM Tris-HCl pH 6.4, and 80% ethanol). After the 3^{rd} wash with wash buffer II, silica beads were transferred to 2.0 mL microcentrifuge tubes using three 500 μL aliquots of WB2 and centrifuged at 2,500×g for 2 min at 4° C. Supernatants were removed and silica beads were dried. RNPs were heat eluted using four 500 μL aliquots of 20 mM Tris-HCl pH 7.5+1 mM EDTA pH 8.0. Each time, samples were incubated at 55° C. for 2 min, vortexed for 10 sec and centrifuged at 4,000×g for 1 min at 20° C. Supernatants were transferred to a 2 ml microcentrifuge tube. After pooling all four aliquots, samples were incubated at 55° C. for 2 min, vortexed for 10 sec, and centrifuged at 14,000×g for 1 min at 20° C. This sequence was repeated. Samples were split between four 2 mL microcentrifuge tubes each containing 3.0 μL GlycoBlue and mixed by brief vortex. Then, 68.6 μL 5 M NaCl were added to each (0.6 M final) and mixed by brief vortex. 1.143 mL RT 100% isopropanol were added to each fraction, vortexed, and incubated on rotator overnight at 4° C. Samples were centrifuged at 18,000×g for 15 min at 4° C. and supernatants were removed. To ensure removal of salts prior to resuspension and RNase-digestion, pellets were washed twice with 1 mL ice-cold 75% ethanol. For each wash, samples were incubated on ice for 5 min followed by centrifugation at 18,000×g for 5 min at 4° C. and removal of supernatant. Then, three 400 μL aliquots of ice-cold 75% ethanol were used to recover precipitates adhering to the sides of the tubes and combined in a 1.5 mL microcentrifuge tube. The tubes were then placed vertically and incubated for 30 min on ice to allow precipitate settling at the bottom of the tube. Samples were centrifuged at 18,000×g for 5 min at 4° C. and supernatants were removed. Pellets were air dried and resuspended at the desired concentration with 1% LiDS TE. An RNase elution was also performed following heat elution as a control. Beads were resuspended in 430 μL DEPC-treated water, 50 μL 10× RNase buffer, and 20 μL RNase Cocktail (AM2286, Invitrogen). Samples were incubated on a rotator at 37° C. for 2 hr, incubated at 55° C. for 2 min, vortexed for 10 sec, and centrifuged at 4,000×g for 1 min at 20° C. Supernatants were transferred to a fresh 2 mL microcentrifuge tube and incubated at 55° C. for 2 min, vortexed for 10 sec, and centrifuged at 14,000×g for 1 min at 20° C. This was repeated and clarified supernatants were transferred to 15 mL conical tubes containing 10 mL RT 100% methanol. Samples were incubated on a rotator overnight at RT. Samples were transferred to a 2.0 mL microcentrifuge tube and centrifuged at 20,000×g for 10 min at 20° C.; supernatants were removed and discarded after each spin. Precipitates were washed twice with 1.0 mL RT 95% methanol. For each wash, samples were vortexed for at least 5 sec, incubated on a rotator for at least 10 min at RT, and centrifuged at 20,000×g for at least 10 min at 20° C. Then, three 400 μL aliquots of RT 95% methanol were used to recover precipitates adhering to the sides of the tubes and combined in a 1.5 mL microcentrifuge tube. The tubes were then placed vertically for 1 hour at RT allow precipitates to settle at the bottom of the tube. Samples were centrifuged at 20,000×g for at least 10 min at 20° C. and supernatants were removed. Pellets were air dried and resuspended at the desired concentration with 1% LiDS TE.

9e. RNA-Interactome Capture RIC was performed on samples containing either 30 million UV-crosslinked (0.2 J/cm², 254 nm) or 30 million non-crosslinked HeLa cells according to the published protocol using Oligo d(T)₂₅ magnetic beads (S1419S, NEB). Cells were harvested with two 15 mL aliquots of lysis/binding buffer (100 mM Tris-HCl, pH 7.5, 500 mM LiCl, 0.5% LiDS, 1 mM EDTA pH 8.0, and 5 mM DTT) and transferred to 50 mL conical tubes. Lysates were sheered by passaging through a 19 ga 1½″ needle fifteen times. 3 mL equilibrated oligo d(T)₂₅ magnetic bead slurry were added to each sample and incubated on agitator for 10 min at RT. A magnet was used to recover beads and supernatants were removed. Beads were washed twice with 15 mL wash buffer 1 (20 mM Tris-HCl pH 7.5, 500 mM LiCl, 0.1% LiDS, 1 mM EDTA pH 8.0, and 5 mM DTT), twice with 15 mL wash buffer 2 (20 mM Tris-HCl pH 7.5, 500 mM LiCl, and 1 mM EDTA pH 8.0), and once 15 mL low salt buffer (20 mM Tris-HCl pH 7.5, 200 mM LiCl, and 1 mM EDTA pH 8.0). For each wash, samples were mixed with agitation for 1 min, beads were recovered using a magnet, and supernatants were removed. Two 2 mL aliquots of wash buffer 2 were used to transfer beads to a fresh 2 mL microcentrifuge tube, a magnet was used to recover beads and remove supernatant each time. RNPs were heat eluted using four 500 μL aliquots of 20 mM Tris-HCl pH 7.5, 1 mM EDTA pH 8.0. Each time, samples were incubated at 55° C. for 2 min and vortexed for 10 sec (max). After pooling all four aliquots, samples were incubated at 55° C. for 2 min and vortexed for 10 sec (max). Beads were recovered and supernatants were transferred to a 2 mL microcentrifuge tube. Samples were split between four 2 mL microcentrifuge tubes each containing 3.0 μL GlycoBlue and mixed by brief vortex. Then, 68.6 μL 5 M NaCl were added to each (0.6 M final) and mixed by brief vortex. 1.143 mL RT 100% isopropanol were added to each fraction, vortexed, and incubated on rotator overnight at 4° C. Samples were centrifuged at 18,000×g for 15 min at 4° C. and supernatants were removed. To ensure removal of salts prior to resuspension and RNase-digestion, pellets were washed twice with 1 mL ice-cold 75% ethanol. For each wash, samples were incubated on ice for 5 min followed by centrifugation at 18,000×g for 5 min at 4° C. and removal of supernatant. Then, three 400 μL aliquots of ice-cold 75% ethanol were used to recover precipitates adhering to the sides of the tubes and combined in a 1.5 mL microcentrifuge tube. The tubes were then placed vertically and incubated for 30 min on ice to allow precipitate settling at the bottom of the tube. Samples were centrifuged at 18,000×g for 5 min at 4° C. (soft brake) and supernatants were removed. Pellets were air dried and resuspended at the desired concentration with 1% LiDS TE. An RNase elution was also performed following heat elution as a control. Beads were resuspended in 430 μL DEPC-treated water, 50 μL 10× RNase buffer, and 20 μL RNase Cocktail (AM2286, Invitrogen). Samples were incubated on a rotator at 37° C. for 2 hr, incubated at 55° C. for 2 min, and vortexed for 10 sec. Beads were recovered and supernatants were transferred to a 2 ml microcentrifuge tube. Samples were incubated again at 55° C. for 2 min and vortexed for 10 sec. Beads were recovered and supernatants were transferred to 15 mL conical tubes containing 10 mL RT 100% methanol. Samples were incubated on rotator overnight at RT. Samples were transferred to a 2 mL microcentrifuge tube and centrifuged at 20,000×g for 10 min at 20° C.; supernatants were removed and discarded after each spin. Precipitates were washed twice with 1 mL RT 95% methanol. For each wash, samples were vortexed for at least 5 sec, incubated on a rotator for at least 10 min at RT, and centrifuged at 20,000×g for at least 10 min at 20° C. Then, three 400 μL aliquots of RT 95% methanol were used to recover precipitates adhering to the sides of the tubes and combined in a 1.5 mL microcentrifuge tube. The tubes were then placed vertically for 1 hour at RT allow precipitates to settle at the bottom of the tube. Samples were centrifuged at 20,000×g for at least 10 min at 20° C. and supernatants were removed. Pellets were air dried and resuspended at the desired concentration with 1% LiDS TE.

What is claimed is:

1. A method for isolating nucleic acids from a sample comprising contacting the sample with a precipitation buffer comprising a monovalent salt and an alcohol, thereby isolating the nucleic acids.

2. The method of claim 1, wherein the monovalent salt is sodium chloride (NaCl) or lithium chloride (LiCl) and wherein the alcohol is isopropanol or ethanol.

3. The method of claim 2, wherein the precipitation buffer comprises from about 0.5M to about 7.5M LiCl or NaCl, and about 25% to about 75% isopropanol or ethanol.

4. The method of claim 2, wherein the precipitation buffer comprises from about 0.5M to about 2M NaCl and about 25% to about 75% isopropanol or ethanol.

5. The method of claim 2, wherein the precipitation buffer comprises from about 1M-5M LiCl and about 25%-75% isopropanol or ethanol.

6. The method of claim 2, wherein the precipitation buffer comprises from about 3M-4.5M LiCl and about 40%-60% isopropanol or ethanol.

7. The method of claim 1, wherein the nucleic acids comprise RNA or RNA-protein complexes (RNPs).

8. The method of claim 1, wherein the sample comprises acidic guanidium thiocyanate and phenol (AGP).

9. A method of isolating RNA-protein complexes (RNPs) from a sample, the method comprising:
a) performing at least one round of acidic guanidium thiocyanate-phenol-chloroform (AGPC) biphasic extraction on the sample and isolating an interphase portion after each round of extraction, thereby obtaining a sample fraction enriched in RNA-protein complexes (RNPs); and
b) contacting the sample fraction enriched in RNPs with a precipitation buffer comprising a monovalent salt and an alcohol, thereby isolating the RNPs.

10. The method of claim 9, wherein the monovalent salt is sodium chloride (NaCl) or lithium chloride (LiCl) and wherein the alcohol is isopropanol or ethanol.

11. The method of claim 10, wherein the precipitation buffer comprises from about 0.5M to about 7.5M LiCl or NaCl, and about 25% to about 75% isopropanol or ethanol.

12. The method of claim 10, wherein the precipitation buffer comprises from about 0.5M to about 2M NaCl and about 25% to about 75% isopropanol or ethanol.

13. The method of claim 10, wherein the precipitation buffer comprises from about 1M-5M LiCl and about 25%-75% isopropanol or ethanol.

14. The method of claim 10, wherein the precipitation buffer comprises from about 3M-4.5M LiCl and about 40%-60% isopropanol or ethanol.

15. A method of identifying RNA binding proteins in a sample, the method comprising:
a) obtaining a composition comprising RNA-protein complexes (RNPs) isolated from a sample;
b) depleting DNA from the composition, and
c) identifying RNA binding proteins in the composition by mass spectrometry,
wherein RNPs are isolated from the sample by:
i) performing at least one round of acidic guanidium thiocyanate-phenol-chloroform (AGPC) biphasic extraction on the sample and isolating an interphase portion after each round of extraction, thereby obtaining a sample fraction enriched in RNA-protein complexes (RNPs); and
ii) contacting the sample fraction enriched in RNPs with a precipitation buffer comprising a monovalent salt and an alcohol, thereby isolating the RNPs.

16. The method of claim 15, wherein the monovalent salt is sodium chloride (NaCl) or lithium chloride (LiCl) and wherein the alcohol is isopropanol or ethanol.

17. The method of claim 16, wherein the precipitation buffer comprises from about 0.5M to about 7.5M LiCl or NaCl, and about 25% to about 75% isopropanol or ethanol.

18. The method of claim 16, wherein the precipitation buffer comprises from about 0.5M to about 2M NaCl and about 25% to about 75% isopropanol or ethanol.

19. The method of claim 16, wherein the precipitation buffer comprises from about 1M-5M LiCl and about 25%-75% isopropanol or ethanol.

20. The method of claim 16, wherein the precipitation buffer comprises from about 3M-4.5M LiCl and about 40%-60% isopropanol or ethanol.
